SEQUENCE LISTING (756 sequences)

SEQ ID NO: 1 - protein, 183 aa, Homo sapiens
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 119 aa, synthetic construct
  REGION 1..119: note = antibody
EVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSS

SEQ ID NO: 3 - protein, 106 aa, synthetic construct
  REGION 1..106: note = antibody
DIQLTQSPSSLSASVGDRVTITCSASISVSYLYWYQQKPGKAPKLLIYDDSSLASGVPSRFSGSGSGTDFTLTISSLQPEDFATYYCQQWSGYPYTFGQGTKVEIK

SEQ ID NO: 4 - protein, 205 aa, synthetic construct
  REGION 1..205: note = trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLIHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 5 - protein, 205 aa, synthetic construct
  REGION 1..205: note = trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTIRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 6 - protein, 205 aa, synthetic construct
  REGION 1..205: note = trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLIIRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 7 - protein, 119 aa, synthetic construct
  REGION 1..119: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSS

SEQ ID NO: 8 - protein, 119 aa, synthetic construct
  REGION 1..119: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQSWGYYALDIWGQGTLVTVSS

SEQ ID NO: 9 - protein, 119 aa, synthetic construct
  REGION 1..119: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQGWGYYALDIWGQGTLVTVSS

SEQ ID NO: 10 - protein, 119 aa, synthetic construct
  REGION 1..119: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQTWGYYALDIWGQGTLVTVSS

SEQ ID NO: 11 - protein, 119 aa, synthetic construct
  REGION 1..119: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQVWGYYALDIWGQGTLVTVSS

SEQ ID NO: 12 - protein, 119 aa, synthetic construct
  REGION 1..119: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQQWGYYALDIWGQGTLVTVSS

SEQ ID NO: 13 - protein, 119 aa, synthetic construct
  REGION 1..119: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQKWGYYALDIWGQGTLVTVSS

SEQ ID NO: 14 - protein, 205 aa, synthetic construct
  REGION 1..205: note = trap
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 15 - protein, 205 aa, synthetic construct; identical to SEQ ID NO: 4
  REGION 1..205: note = trap

SEQ ID NO: 16 - protein, 205 aa, synthetic construct; identical to SEQ ID NO: 5
  REGION 1..205: note = trap

SEQ ID NO: 17 - protein, 205 aa, synthetic construct; identical to SEQ ID NO: 6
  REGION 1..205: note = trap

SEQ ID NO: 18 - protein, 10 aa, synthetic construct
  REGION 1..10: note = linker
GGGGSGGGGS

SEQ ID NO: 19 - protein, 449 aa, synthetic construct
  REGION 1..449: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 20 - protein, 449 aa, synthetic construct
  REGION 1..449: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQSWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMLSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEAIHNHYTQKSLSCSPGK

SEQ ID NO: 21 - protein, 449 aa, synthetic construct
  REGION 1..449: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQGWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPRSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 22 - protein, 449 aa, synthetic construct
  REGION 1..449: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQTWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDLAVEWESNGQPENNYKTTPPVLDSDGSFFLYSKLTVDKSRWQQGNVFSCSVMHEALHNHYTQKSLSCSPGK

SEQ ID NO: 23 - protein, 449 aa, synthetic construct
  REGION 1..449: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQVWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 24 - protein, 449 aa, synthetic construct
  REGION 1..449: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQQWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 25 - protein, 448 aa, synthetic construct
  REGION 1..448: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQKWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNYTQKSLSCSPGK

SEQ ID NO: 26 - protein, 449 aa, synthetic construct
  REGION 1..449: note = antibody
EVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 27 - protein, 447 aa, synthetic construct
  REGION 1..447: note = antibody
EVQLVESGGGLVQPGGSLRLSCAASGFTFSPFAWSVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 28 - protein, 213 aa, synthetic construct
  REGION 1..213: note = antibody
DIQLTQSPSSLSASVGDRVTITCSASISVSYLYWYQQKPGKAPKLLIYDDSSLASGVPSRFSGSGSGTDFTLTISSLQPEDFATYYCQQWSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 213 aa, synthetic construct
  REGION 1..213: note = antibody
DIQLTQSPSSLSASVGDRVTITCSASISVSYLYWYQQKPGKAPKLLIYDASSLASGVPSRFSGSGSGTDFTLTISSLQPEDFATYYCQQWSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 213 aa, synthetic construct
  REGION 1..213: note = antibody
DIQLTQSPSSLSASVGDRVTITCSASISVSYLYWYQQKPGKAPKLLIYDDSNLASGVPSRFSGSGSGTDFTLTISSLQPEDFATYYCQQWSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 223 aa, synthetic construct
  REGION 1..223: note = antibody
EVQLVESGGGLVQPGGSLRLSCAASGFTFSPFAMHWVRQAPGKGLEWVAKISPGGSWTYYSDTVIDRFTFSLDTSKSTAYLQMNSLRAEDTAVYYCARQAWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYTICNVNHKPSNTKVDKKVEPKSC

SEQ ID NO: 32 - protein, 222 aa, synthetic construct
  REGION 1..222: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQSWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 221 aa, synthetic construct
  REGION 1..221: note = antibody
EVQLVESGGLVQPGGSLRLSCAASGFTFSPFAMHWVRQAPGKGLEWVAKISPGGSWTYYSDTVTDRFTFSLDTSKSTAYLQMNSLRAEDTAVYYCARQGWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 34 - protein, 222 aa, synthetic construct
  REGION 1..222: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQTWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 35 - protein, 222 aa, synthetic construct
  REGION 1..222: note = antibody
EVQLVESGGGLVQPGGSLRLSCAASGFTFSPFAMHWVRQAPGKGLEWVAKISPGGSWTYYSDTVIDRFTFSLDTSKSTAYLQMNSLRAEDTAVYYCARQVWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - protein, 222 aa, synthetic construct
  REGION 1..222: note = antibody
EVQLVESGGGLVQPGGSLRLSCAASGFTFSPFAMHWVRQAPGKGLEWVAKISPGGSWTYYSDTVIDRFTFSLDTSKSTAYLQMNSLRAEDTAVYYCARQQWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 37 - protein, 222 aa, synthetic construct
  REGION 1..222: note = antibody
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQKWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 38 - protein, 222 aa, synthetic construct
  REGION 1..222: note = antibody
EVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 39 - protein, 222 aa, synthetic construct
  REGION 1..222: note = antibody
EVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGHTFPAVLQSSGLYSLSSVVTPVPSSSLGTQTYICNVNHKPSNTKVDKKVEPKSC

SEQ ID NO: 40 - protein, 226 aa, synthetic construct
  REGION 1..226: note = antibody
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNFSCSVMHEALHNHYTQKSLSCSPGK

SEQ ID NO: 41 - protein, 226 aa, synthetic construct
  REGION 1..226: note = antibody
DKTHTCPPCPAPEAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 42 - protein, 226 aa, synthetic construct
  REGION 1..226: note = antobyd
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 43 - protein, 227 aa, synthetic construct
  REGION 1..227: note = antibody
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 44 - protein, 227 aa, synthetic construct; identical to SEQ ID NO: 43
  REGION 1..227: note = antibody

SEQ ID NO: 45 - protein, 227 aa, synthetic construct; identical to SEQ ID NO: 43
  REGION 1..227: note = antibody

SEQ ID NO: 46 - protein, 227 aa, synthetic construct; identical to SEQ ID NO: 43
  REGION 1..227: note = antibody

SEQ ID NO: 47 - protein, 227 aa, synthetic construct; identical to SEQ ID NO: 43
  REGION 1..227: note = antibody

SEQ ID NO: 48 - protein, 225 aa, synthetic construct
  REGION 1..225: note = antibody
DKTHTCPPCPAPEAAGAPSVFLFPPKPKDTLMISRTPEVTCVVVDVSHEDPEVKFNWYVDVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNFSCSVMHEALHNHYTQKSLSCSPGK

SEQ ID NO: 49 - protein, 10 aa, synthetic construct
  REGION 1..10: note = antibody
GFTFSPFAMH

SEQ ID NO: 50 - protein, 19 aa, synthetic construct
  REGION 1..19: note = antibody
VAKISPGGSWTYYSDTVTD

SEQ ID NO: 51 - protein, 12 aa, synthetic construct
  REGION 1..12: note = antibody
ARQAWGYYALDI

SEQ ID NO: 52 - protein, 10 aa, synthetic construct
  REGION 1..10: note = antibody
GFTFSPFAMH

SEQ ID NO: 53 - protein, 19 aa, synthetic construct
  REGION 1..19: note = antibody
VAKISPGGSWTYYSDTVTD

SEQ ID NO: 54 - protein, 12 aa, synthetic construct
  REGION 1..12: note = antibody
ARQAWGYYALDI

SEQ ID NO: 55 - protein, 10 aa, synthetic construct
  REGION 1..10: note = antibody
GFTFSPFAMH

SEQ ID NO: 56 - protein, 19 aa, synthetic construct
  REGION 1..19: note = antibody
VAKISPGGSWTYYSDTVTD

SEQ ID NO: 57 - protein, 12 aa, synthetic construct
  REGION 1..12: note = antibody
ARQAWGYYALDI

SEQ ID NO: 58 - protein, 10 aa, synthetic construct
  REGION 1..10: note = antibody
GFTFSPFAMH

SEQ ID NO: 59 - protein, 19 aa, synthetic construct
  REGION 1..19: note = antibody
VAKISPGGSWTYYSDTVTD

SEQ ID NO: 60 - protein, 12 aa, synthetic construct
  REGION 1..12: note = antibody
ARQAWGYYALDI

SEQ ID NO: 61 - protein, 10 aa, synthetic construct
  REGION 1..10: note = antibody
GFTFSPFAMH

SEQ ID NO: 62 - protein, 19 aa, synthetic construct
  REGION 1..19: note = antibody
VAKISPGGSWTYYSDTVTD

SEQ ID NO: 63 - protein, 12 aa, synthetic construct
  REGION 1..12: note = antibody
ARQAWGYYALDI

SEQ ID NO: 64 - protein, 10 aa, synthetic construct
  REGION 1..10: note = antibody
GFTFSPFAMH

SEQ ID NO: 65 - protein, 19 aa, synthetic construct
  REGION 1..19: note = antibody
VAKISPGGSWTYYSDTVTD

SEQ ID NO: 66 - protein, 12 aa, synthetic construct
  REGION 1..12: note = antibody
ARQAWGYYALDI

SEQ ID NO: 67 - protein, 10 aa, synthetic construct
  REGION 1..10: note = antibody
GFTFSPFAMH

SEQ ID NO: 68 - protein, 19 aa, synthetic construct
  REGION 1..19: note = antibody
VAKISPGGSWTYYSDTVTD

SEQ ID NO: 69 - protein, 12 aa, synthetic construct
  REGION 1..12: note = antibody
ARQAWGYYALDI

SEQ ID NO: 70 - protein, 10 aa, synthetic construct
  REGION 1..10: note = antibody
GFTFSPFAIS

SEQ ID NO: 71 - protein, 19 aa, synthetic construct
  REGION 1..19: note = antibody
VAKISPGGSWTYYSDTVTD

SEQ ID NO: 72 - protein, 12 aa, synthetic construct
  REGION 1..12: note = antibody
ARQAWGYYALDV

SEQ ID NO: 73 - protein, 10 aa, synthetic construct
  REGION 1..10: note = antibody
GFTFSPFAWS

SEQ ID NO: 74 - protein, 19 aa, synthetic construct
  REGION 1..19: note = antibody
VAKISPGGSWTYYSDTVTD

SEQ ID NO: 75 - protein, 12 aa, synthetic construct
  REGION 1..12: note = antibody
ARQAWGYYALDV

SEQ ID NO: 76 - protein, 10 aa, synthetic construct
  REGION 1..10: note = antibody
SASISVSYLY

SEQ ID NO: 77 - protein, 11 aa, synthetic construct
  REGION 1..11: note = antibody
LLIYDDSSLAS

SEQ ID NO: 78 - protein, 9 aa, synthetic construct
  REGION 1..9: note = antibody
QQWSGYPYT

SEQ ID NO: 79 - protein, 10 aa, synthetic construct
  REGION 1..10: note = antibody
SASISVSYLY

SEQ ID NO: 80 - protein, 11 aa, synthetic construct
  REGION 1..11: note = antibody
LLIYDDSSLAS

SEQ ID NO: 81 - protein, 9 aa, synthetic construct
  REGION 1..9: note = antibody
QQWSGYPYT

SEQ ID NO: 82 - protein, 10 aa, synthetic construct
  REGION 1..10: note = antibody
SASISVSYLY

SEQ ID NO: 83 - protein, 11 aa, synthetic construct
  REGION 1..11: note = antibody
LLIYDDSSLAS

SEQ ID NO: 84 - protein, 9 aa, synthetic construct
  REGION 1..9: note = antibody
QQWSGYPYT

SEQ ID NO: 85 - protein, 432 aa, synthetic construct
  REGION 1..432: note = antibody
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 86 - protein, 432 aa, synthetic construct
  REGION 1..432: note = antibody
SDTGRPFVEMYSEIPEIIHMTEGRELVIPCRVTSPNITVTLKKFPLDTLIPDGKRIIWDSRKGFIISNATYKEIGLLTCEATVNGHLYKTNYLIHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 87 - protein, 432 aa, synthetic construct
  REGION 1..432: note = antibody
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTIRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 88 - protein, 432 aa, synthetic construct
  REGION 1..432: note = antibody
SDTGRPFVEMYSEIPEIIHMTEGRELVIPCRVTSPNITVTLKKFPLDTLIPDGKRIIWDSRKGFIISNATYKEIGLLTCEATVNGHLYKTNYLIIRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 89 - protein, 119 aa, synthetic construct; identical to SEQ ID NO: 2
  REGION 1..119: note = antibody

SEQ ID NO: 90 - protein, 119 aa, synthetic construct
  REGION 1..119: note = antibody
EVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSS

SEQ ID NO: 91 - protein, 106 aa, synthetic construct; identical to SEQ ID NO: 3
  REGION 1..106: note = antibody

SEQ ID NO: 92 - protein, 106 aa, synthetic construct
  REGION 1..106: note = antibody
DIQLTQSPSSLSASVGDRVTITCSASISVSYLYWYQQKPGKAPKLLIYDASSLASGVPSRFSGSGSGTDFTLTISSLQPEDFATYYCQQWSGYPYTFGQGTKVEIK

SEQ ID NO: 93 - protein, 106 aa, synthetic construct
  REGION 1..106: note = antibody
DIQLTQSPSSLSASVGDRVTITCSASISVSYLYWYQQKPGKAPKLLIYDDSNLASGVPSRFSGSGSGTDFTLTISSLQPEDFATYYCQQWSGYPYTFGQGTKVEIK

SEQ ID NO: 94 - protein, 10 aa, synthetic construct
  REGION 1..10: note = antibody
GFTFSPFAMS

SEQ ID NO: 95 - protein, 17 aa, synthetic construct
  REGION 1..17: note = antibody
KISPGGSWTYYSDTVTG

SEQ ID NO: 96 - protein, 10 aa, synthetic construct
  REGION 1..10: note = antibody
SASISVSYMY

SEQ ID NO: 97 - protein, 7 aa, synthetic construct
  REGION 1..7: note = antibody
DMSNLAS

SEQ ID NO: 98 - protein, 9 aa, synthetic construct
  REGION 1..9: note = antibody
MQWSGYPYT

SEQ ID NO: 99 - protein, 10 aa, synthetic construct
  REGION 1..10: note = artifical
GFTFSPFAIS

SEQ ID NO: 100 - protein, 19 aa, synthetic construct
  REGION 1..19: note = antibody
VAKISPGGSWTYYSDTVTD

SEQ ID NO: 101 - protein, 12 aa, synthetic construct
  REGION 1..12: note = antibody
ARQLWGYYALDV

SEQ ID NO: 102 - protein, 10 aa, synthetic construct
  REGION 1..10: note = antibody
GFTFSPFAWS

SEQ ID NO: 103 - protein, 19 aa, synthetic construct
  REGION 1..19: note = antibody
VAKISPGGSWTYYSDTVTD

SEQ ID NO: 104 - protein, 12 aa, synthetic construct
  REGION 1..12: note = antibody
ARQLWGYYALDV

SEQ ID NO: 105 - protein, 10 aa, synthetic construct
  REGION 1..10: note = antibody
SASISVSYLY

SEQ ID NO: 106 - protein, 11 aa, synthetic construct
  REGION 1..11: note = antibody
LLIYDDSSLAS

SEQ ID NO: 107 - protein, 9 aa, synthetic construct
  REGION 1..9: note = antibody
QQWSGYPYT

SEQ ID NO: 108 - protein, 10 aa, synthetic construct
  REGION 1..10: note = antibody
SASISVSYLY

SEQ ID NO: 109 - protein, 11 aa, synthetic construct
  REGION 1..11: note = antibody
LLIYDASSLAS

SEQ ID NO: 110 - protein, 9 aa, synthetic construct
  REGION 1..9: note = antibody
QQWSGYPYT

SEQ ID NO: 111 - protein, 10 aa, synthetic construct
  REGION 1..10: note = antibody
SASISVSYLY

SEQ ID NO: 112 - protein, 11 aa, synthetic construct
  REGION 1..11: note = antibody
LLIYDDSNLAS

SEQ ID NO: 113 - protein, 9 aa, synthetic construct
  REGION 1..9: note = antibody
QQWSGYPYT

SEQ ID NO: 114 - protein, 205 aa, synthetic construct; identical to SEQ ID NO: 14
  REGION 1..205: note = fusion

SEQ ID NO: 115 - protein, 10 aa, synthetic construct
  REGION 1..10: note = antibody
GGGGSGGGGS

SEQ ID NO: 116 - protein, 666 aa, synthetic construct
  REGION 1..666: note = fuison
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGKGK

SEQ ID NO: 117 - protein, 213 aa, synthetic construct; identical to SEQ ID NO: 28
  REGION 1..213: note = light chain

SEQ ID NO: 118 - protein, 664 aa, synthetic construct
  REGION 1..664: note = fusion
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 119 - protein, 213 aa, synthetic construct; identical to SEQ ID NO: 29
  REGION 1..213: note = light chain

SEQ ID NO: 120 - protein, 664 aa, synthetic construct; identical to SEQ ID NO: 118
  REGION 1..664: note = fusion

SEQ ID NO: 121 - protein, 213 aa, synthetic construct; identical to SEQ ID NO: 30
  REGION 1..213: note = light chain

SEQ ID NO: 122 - protein, 664 aa, synthetic construct
  REGION 1..664: note = fusion
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 123 - protein, 213 aa, synthetic construct; identical to SEQ ID NO: 28
  REGION 1..213: note = light chain

SEQ ID NO: 124 - protein, 664 aa, synthetic construct; identical to SEQ ID NO: 122
  REGION 1..664: note = fusion

SEQ ID NO: 125 - protein, 213 aa, synthetic construct; identical to SEQ ID NO: 29
  REGION 1..213: note = light chain

SEQ ID NO: 126 - protein, 664 aa, synthetic construct; identical to SEQ ID NO: 122
  REGION 1..664: note = fusion

SEQ ID NO: 127 - protein, 213 aa, synthetic construct; identical to SEQ ID NO: 30
  REGION 1..213: note = light chain

SEQ ID NO: 128 - protein, 664 aa, synthetic construct
  REGION 1..664: note = fusion
EVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 129 - protein, 213 aa, synthetic construct; identical to SEQ ID NO: 28
  REGION 1..213: note = light chain

SEQ ID NO: 130 - protein, 664 aa, synthetic construct; identical to SEQ ID NO: 128
  REGION 1..664: note = fusion

SEQ ID NO: 131 - protein, 213 aa, synthetic construct; identical to SEQ ID NO: 29
  REGION 1..213: note = light chain

SEQ ID NO: 132 - protein, 664 aa, synthetic construct; identical to SEQ ID NO: 128
  REGION 1..664: note = fusion

SEQ ID NO: 133 - protein, 213 aa, synthetic construct; identical to SEQ ID NO: 30
  REGION 1..213: note = fusion

SEQ ID NO: 134 - protein, 664 aa, synthetic construct
  REGION 1..664: note = fusion
EVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 135 - protein, 213 aa, synthetic construct; identical to SEQ ID NO: 28
  REGION 1..213: note = light chain

SEQ ID NO: 136 - protein, 664 aa, synthetic construct; identical to SEQ ID NO: 134
  REGION 1..664: note = light chain

SEQ ID NO: 137 - protein, 213 aa, synthetic construct; identical to SEQ ID NO: 29
  REGION 1..213: note = light chain

SEQ ID NO: 138 - protein, 664 aa, synthetic construct; identical to SEQ ID NO: 134
  REGION 1..664: note = fusion

SEQ ID NO: 139 - protein, 213 aa, synthetic construct; identical to SEQ ID NO: 30
  REGION 1..213: note = light chain

SEQ ID NO: 140 - protein, 119 aa, synthetic construct; identical to SEQ ID NO: 2
  REGION 1..119: note = antibody

SEQ ID NO: 141 - protein, 119 aa, synthetic construct; identical to SEQ ID NO: 90
  REGION 1..119: note = antibody

SEQ ID NO: 142 - protein, 106 aa, synthetic construct; identical to SEQ ID NO: 3
  REGION 1..106: note = antibody

SEQ ID NO: 143 - protein, 106 aa, synthetic construct; identical to SEQ ID NO: 92
  REGION 1..106: note = antibody

SEQ ID NO: 144 - protein, 106 aa, synthetic construct; identical to SEQ ID NO: 93
  REGION 1..106: note = antibody

SEQ ID NO: 145 - protein, 205 aa, synthetic construct; identical to SEQ ID NO: 14
  REGION 1..205: note = trap

SEQ ID NO: 146 - protein, 213 aa, synthetic construct; identical to SEQ ID NO: 28
  REGION 1..213: note = light chain

SEQ ID NO: 147 - protein, 664 aa, synthetic construct; identical to SEQ ID NO: 118
  REGION 1..664: note = fusion

SEQ ID NO: 148 - protein, 213 aa, synthetic construct; identical to SEQ ID NO: 29
  REGION 1..213: note = light chain

SEQ ID NO: 149 - protein, 664 aa, synthetic construct; identical to SEQ ID NO: 122
  REGION 1..664: note = fusion

SEQ ID NO: 150 - protein, 213 aa, synthetic construct; identical to SEQ ID NO: 30
  REGION 1..213: note = light chain

SEQ ID NO: 151 - protein, 664 aa, synthetic construct; identical to SEQ ID NO: 122
  REGION 1..664: note = fusion

SEQ ID NO: 152 - protein, 213 aa, synthetic construct; identical to SEQ ID NO: 28
  REGION 1..213: note = light chain

SEQ ID NO: 153 - protein, 664 aa, synthetic construct; identical to SEQ ID NO: 122
  REGION 1..664: note = fusion

SEQ ID NO: 154 - protein, 213 aa, synthetic construct; identical to SEQ ID NO: 29
  REGION 1..213: note = light chain

SEQ ID NO: 155 - protein, 664 aa, synthetic construct; identical to SEQ ID NO: 122
  REGION 1..664: note = fusion

SEQ ID NO: 156 - protein, 213 aa, synthetic construct; identical to SEQ ID NO: 30
  REGION 1..213: note = light chain

SEQ ID NO: 157 - protein, 664 aa, synthetic construct; identical to SEQ ID NO: 128
  REGION 1..664: note = fusion

SEQ ID NO: 158 - protein, 213 aa, synthetic construct; identical to SEQ ID NO: 28
  REGION 1..213: note = light chain

SEQ ID NO: 159 - protein, 664 aa, synthetic construct; identical to SEQ ID NO: 128
  REGION 1..664: note = fusion

SEQ ID NO: 160 - protein, 213 aa, synthetic construct; identical to SEQ ID NO: 29
  REGION 1..213: note = light chain

SEQ ID NO: 161 - protein, 664 aa, synthetic construct; identical to SEQ ID NO: 128
  REGION 1..664: note = fusion

SEQ ID NO: 162 - protein, 213 aa, synthetic construct; identical to SEQ ID NO: 30
  REGION 1..213: note = light chain

SEQ ID NO: 163 - protein, 664 aa, synthetic construct; identical to SEQ ID NO: 134
  REGION 1..664: note = fusion

SEQ ID NO: 164 - protein, 213 aa, synthetic construct; identical to SEQ ID NO: 28
  REGION 1..213: note = light chain

SEQ ID NO: 165 - protein, 664 aa, synthetic construct; identical to SEQ ID NO: 134
  REGION 1..664: note = fusion

SEQ ID NO: 166 - protein, 213 aa, synthetic construct; identical to SEQ ID NO: 29
  REGION 1..213: note = light chain

SEQ ID NO: 167 - protein, 667 aa, synthetic construct
  REGION 1..667: note = fusion
EVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGKPGK

SEQ ID NO: 168 - protein, 213 aa, synthetic construct; identical to SEQ ID NO: 30
  REGION 1..213: note = light chain

SEQ ID NO: 169 - protein, 213 aa, synthetic construct; identical to SEQ ID NO: 28
  REGION 1..213: note = antibody

SEQ ID NO: 170 - protein, 664 aa, synthetic construct
  REGION 1..664: note = antibody
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 171 - protein, 10 aa, synthetic construct
  REGION 1..10: note = antibody
QLWGYYALDI

SEQ ID NO: 172 - protein, 5 aa, synthetic construct
  REGION 1..5: note = antibody
PFAMH

SEQ ID NO: 173 - protein, 17 aa, synthetic construct
  REGION 1..17: note = antibody
KISPGGSWTYYSDTVTD

SEQ ID NO: 174 - protein, 10 aa, synthetic construct
  REGION 1..10: note = antibody
QAWGYYALDI

SEQ ID NO: 175 - protein, 5 aa, synthetic construct
  REGION 1..5: note = antibody
PFAMH

SEQ ID NO: 176 - protein, 17 aa, synthetic construct
  REGION 1..17: note = antibody
KISPGGSWTYYSDTVTD

SEQ ID NO: 177 - protein, 10 aa, synthetic construct
  REGION 1..10: note = antibody
QSWGYYALDI

SEQ ID NO: 178 - protein, 5 aa, synthetic construct
  REGION 1..5: note = antibody
PFAMH

SEQ ID NO: 179 - protein, 17 aa, synthetic construct
  REGION 1..17: note = antibody
KISPGGSWTYYSDTVTD

SEQ ID NO: 180 - protein, 10 aa, synthetic construct
  REGION 1..10: note = antibody
QGWGYYALDI

SEQ ID NO: 181 - protein, 5 aa, synthetic construct
  REGION 1..5: note = antibody
PFAMH

SEQ ID NO: 182 - protein, 17 aa, synthetic construct
  REGION 1..17: note = antibody
KISPGGSWTYYSDTVTD

SEQ ID NO: 183 - protein, 10 aa, synthetic construct
  REGION 1..10: note = antibody
QTWGYYALDI

SEQ ID NO: 184 - protein, 5 aa, synthetic construct
  REGION 1..5: note = antibody
PFAMH

SEQ ID NO: 185 - protein, 17 aa, synthetic construct
  REGION 1..17: note = antibody
KISPGGSWTYYSDTVTD

SEQ ID NO: 186 - protein, 10 aa, synthetic construct
  REGION 1..10: note = antibody
QVWGYYALDI

SEQ ID NO: 187 - protein, 5 aa, synthetic construct
  REGION 1..5: note = antibody
PFAMH

SEQ ID NO: 188 - protein, 17 aa, synthetic construct
  REGION 1..17: note = antibody
KISPGGSWTYYSDTVTD

SEQ ID NO: 189 - protein, 10 aa, synthetic construct
  REGION 1..10: note = antibody
QQWGYYALDI

SEQ ID NO: 190 - protein, 5 aa, synthetic construct
  REGION 1..5: note = antibody
PFAMH

SEQ ID NO: 191 - protein, 17 aa, synthetic construct
  REGION 1..17: note = antibody
KISPGGSWTYYSDTVTD

SEQ ID NO: 192 - protein, 10 aa, synthetic construct
  REGION 1..10: note = antibody
QKWGYYALDI

SEQ ID NO: 193 - protein, 5 aa, synthetic construct
  REGION 1..5: note = antibody
PFAIS

SEQ ID NO: 194 - protein, 17 aa, synthetic construct
  REGION 1..17: note = antibody
KISPGGSWTYYSDTVTD

SEQ ID NO: 195 - protein, 10 aa, synthetic construct
  REGION 1..10: note = antibody
QLWGYYALDV

SEQ ID NO: 196 - protein, 5 aa, synthetic construct
  REGION 1..5: note = antibody
PFAWS

SEQ ID NO: 197 - protein, 17 aa, synthetic construct
  REGION 1..17: note = antibody
KISPGGSWTYYSDTVTD

SEQ ID NO: 198 - protein, 10 aa, synthetic construct
  REGION 1..10: note = antibody
QLWGYYALDV

SEQ ID NO: 199 - protein, 10 aa, synthetic construct
  REGION 1..10: note = antibody
SASISVSYLY

SEQ ID NO: 200 - protein, 7 aa, synthetic construct
  REGION 1..7: note = antibody
DDSSLAS

SEQ ID NO: 201 - protein, 9 aa, synthetic construct
  REGION 1..9: note = antibody
QQWSGYPYT

SEQ ID NO: 202 - protein, 10 aa, synthetic construct
  REGION 1..10: note = antibody
SASISVSYLY

SEQ ID NO: 203 - protein, 7 aa, synthetic construct
  REGION 1..7: note = antibody
DASSLAS

SEQ ID NO: 204 - protein, 9 aa, synthetic construct
  REGION 1..9: note = antibody
QQWSGYPYT

SEQ ID NO: 205 - protein, 10 aa, synthetic construct
  REGION 1..10: note = antibody
SASISVSYLY

SEQ ID NO: 206 - protein, 7 aa, synthetic construct
  REGION 1..7: note = antibody
DDSNLAS

SEQ ID NO: 207 - protein, 9 aa, synthetic construct
  REGION 1..9: note = antibody
QQWSGYPYT

SEQ ID NO: 208 - protein, 10 aa, synthetic construct
  REGION 1..10: note = antibody
GFTFSPFAMH

SEQ ID NO: 209 - protein, 19 aa, synthetic construct
  REGION 1..19: note = antibody
VAKISPGGSWTYYSDTVTD

SEQ ID NO: 210 - protein, 12 aa, synthetic construct
  REGION 1..12: note = antibody
ARQAWGYYALDI

SEQ ID NO: 211 - protein, 10 aa, synthetic construct
  REGION 1..10: note = antibody
GFTFSPFAMH

SEQ ID NO: 212 - protein, 19 aa, synthetic construct
  REGION 1..19: note = antibody
VAKISPGGSWTYYSDTVTD

SEQ ID NO: 213 - protein, 12 aa, synthetic construct
  REGION 1..12: note = antibody
ARQSWGYYALDI

SEQ ID NO: 214 - protein, 10 aa, synthetic construct
  REGION 1..10: note = antibody
GFTFSPFAMH

SEQ ID NO: 215 - protein, 19 aa, synthetic construct
  REGION 1..19: note = antibody
VAKISPGGSWTYYSDTVTD

SEQ ID NO: 216 - protein, 12 aa, synthetic construct
  REGION 1..12: note = antibody
ARQGWGYYALDI

SEQ ID NO: 217 - protein, 10 aa, synthetic construct
  REGION 1..10: note = antibody
GFTFSPFAMH

SEQ ID NO: 218 - protein, 19 aa, synthetic construct
  REGION 1..19: note = antibody
VAKISPGGSWTYYSDTVTD

SEQ ID NO: 219 - protein, 12 aa, synthetic construct
  REGION 1..12: note = antibody
ARQTWGYYALDI

SEQ ID NO: 220 - protein, 10 aa, synthetic construct
  REGION 1..10: note = antibody
GFTFSPFAMH

SEQ ID NO: 221 - protein, 19 aa, synthetic construct
  REGION 1..19: note = antibody
VAKISPGGSWTYYSDTVTD

SEQ ID NO: 222 - protein, 12 aa, synthetic construct
  REGION 1..12: note = antibody
ARQVWGYYALDI

SEQ ID NO: 223 - protein, 10 aa, synthetic construct
  REGION 1..10: note = antibody
GFTFSPFAMH

SEQ ID NO: 224 - protein, 19 aa, synthetic construct
  REGION 1..19: note = antibody
VAKISPGGSWTYYSDTVTD

SEQ ID NO: 225 - protein, 12 aa, synthetic construct
  REGION 1..12: note = antibody
ARQQWGYYALDI

SEQ ID NO: 226 - protein, 10 aa, synthetic construct
  REGION 1..10: note = antibody
GFTFSPFAMH

SEQ ID NO: 227 - protein, 19 aa, synthetic construct
  REGION 1..19: note = antibody
VAKISPGGSWTYYSDTVTD

SEQ ID NO: 228 - protein, 12 aa, synthetic construct
  REGION 1..12: note = antibody
ARQKWGYYALDI

SEQ ID NO: 229 - protein, 10 aa, synthetic construct
  REGION 1..10: note = antibody
GFTFSPFAIS

SEQ ID NO: 230 - protein, 19 aa, synthetic construct
  REGION 1..19: note = antibody
VAKISPGGSWTYYSDTVTD

SEQ ID NO: 231 - protein, 12 aa, synthetic construct
  REGION 1..12: note = antibody
ARQLWGYYALDV

SEQ ID NO: 232 - protein, 10 aa, synthetic construct
  REGION 1..10: note = antibody
GFTFSPFAWS

SEQ ID NO: 233 - protein, 20 aa, synthetic construct
  REGION 1..20: note = antibody
WVAKISPGGSWTYYSDTVTD

SEQ ID NO: 234 - protein, 12 aa, synthetic construct
  REGION 1..12: note = antibody
ARQLWGYYALDV

SEQ ID NO: 235 - protein, 10 aa, synthetic construct
  REGION 1..10: note = antibody
SASISVSYLY

SEQ ID NO: 236 - protein, 11 aa, synthetic construct
  REGION 1..11: note = antibody
LLIYDDSSLAS

SEQ ID NO: 237 - protein, 9 aa, synthetic construct
  REGION 1..9: note = antibody
QQWSGYPYT

SEQ ID NO: 238 - protein, 10 aa, synthetic construct
  REGION 1..10: note = antibody
SASISVSYLY

SEQ ID NO: 239 - protein, 11 aa, synthetic construct
  REGION 1..11: note = antibody
LLIYDASSLAS

SEQ ID NO: 240 - protein, 9 aa, synthetic construct
  REGION 1..9: note = antibody
QQWSGYPYT

SEQ ID NO: 241 - protein, 10 aa, synthetic construct
  REGION 1..10: note = antibody
SASISVSYLY

SEQ ID NO: 242 - protein, 11 aa, synthetic construct
  REGION 1..11: note = antibody
LLIYDDSNLAS

SEQ ID NO: 243 - protein, 9 aa, synthetic construct
  REGION 1..9: note = antibody
QQWSGYPYT

SEQ ID NO: 244 - protein, 5 aa, synthetic construct
  REGION 1..5: note = antibody
PFAMS

SEQ ID NO: 245 - protein, 17 aa, synthetic construct
  REGION 1..17: note = antibody
KISPGGSWTYYSDTVTG

SEQ ID NO: 246 - protein, 10 aa, synthetic construct
  REGION 1..10: note = antibody
QLWGYYALDI

SEQ ID NO: 247 - protein, 10 aa, synthetic construct
  REGION 1..10: note = antibody
SASISVSYMY

SEQ ID NO: 248 - protein, 7 aa, synthetic construct
  REGION 1..7: note = antibody
DMSNLAS

SEQ ID NO: 249 - protein, 9 aa, synthetic construct
  REGION 1..9: note = antibody
MQWSGYPYT

SEQ ID NO: 250 - protein, 10 aa, synthetic construct
  REGION 1..10: note = antibody
GFTFSPFAMS

SEQ ID NO: 251 - protein, 20 aa, synthetic construct
  REGION 1..20: note = antibody
WVAKISPGGSWTYYSDTVTG

SEQ ID NO: 252 - protein, 12 aa, synthetic construct
  REGION 1..12: note = antibody
ARQLWGYYALDI

SEQ ID NO: 253 - protein, 10 aa, synthetic construct
  REGION 1..10: note = antibody
SASISVSYMY

SEQ ID NO: 254 - protein, 11 aa, synthetic construct
  REGION 1..11: note = antibody
LLIYDMSNLAS

SEQ ID NO: 255 - protein, 9 aa, synthetic construct
  REGION 1..9: note = antibody
MQWSGYPYT

SEQ ID NO: 256 - protein, 119 aa, synthetic construct; identical to SEQ ID NO: 7
  REGION 1..119: note = antibody

SEQ ID NO: 257 - protein, 119 aa, synthetic construct; identical to SEQ ID NO: 8
  REGION 1..119: note = antibody

SEQ ID NO: 258 - protein, 119 aa, synthetic construct; identical to SEQ ID NO: 9
  REGION 1..119: note = antibody

SEQ ID NO: 259 - protein, 119 aa, synthetic construct; identical to SEQ ID NO: 10
  REGION 1..119: note = antibody

SEQ ID NO: 260 - protein, 119 aa, synthetic construct; identical to SEQ ID NO: 11
  REGION 1..119: note = antibody

SEQ ID NO: 261 - protein, 119 aa, synthetic construct; identical to SEQ ID NO: 12
  REGION 1..119: note = antibody

SEQ ID NO: 262 - protein, 119 aa, synthetic construct; identical to SEQ ID NO: 13
  REGION 1..119: note = antibody

SEQ ID NO: 263 - protein, 664 aa, synthetic construct; identical to SEQ ID NO: 170
  REGION 1..664: note = fusion

SEQ ID NO: 264 - protein, 664 aa, synthetic construct; identical to SEQ ID NO: 170
  REGION 1..664: note = fusion

SEQ ID NO: 265 - protein, 664 aa, synthetic construct; identical to SEQ ID NO: 170
  REGION 1..664: note = trap

SEQ ID NO: 266 - protein, 664 aa, synthetic construct
  REGION 1..664: note = fusion
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 267 - protein, 664 aa, synthetic construct; identical to SEQ ID NO: 266
  REGION 1..664: note = fusion

SEQ ID NO: 268 - protein, 664 aa, synthetic construct; identical to SEQ ID NO: 266
  REGION 1..664: note = fusion

SEQ ID NO: 269 - protein, 664 aa, synthetic construct; identical to SEQ ID NO: 118
  REGION 1..664: note = fusion

SEQ ID NO: 270 - protein, 453 aa, synthetic construct
  REGION 1..453: note = antibody
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 271 - protein, 452 aa, synthetic construct
  REGION 1..452: note = antibody
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPG

SEQ ID NO: 272 - protein, 451 aa, synthetic construct
  REGION 1..451: note = antibody
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSP

SEQ ID NO: 273 - protein, 450 aa, synthetic construct
  REGION 1..450: note = antibody
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCS

SEQ ID NO: 274 - protein, 449 aa, synthetic construct
  REGION 1..449: note = antibody
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSC

SEQ ID NO: 275 - protein, 214 aa, synthetic construct
  REGION 1..214: note = antibody
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 120 aa, synthetic construct
EVKLVESEGGLVQPGSSMKLSCTASGFTFSDYYMAWVRQVPEKGLEWVGNINYDGSSTYYLDSLKSRFIISRDSAKNILYLQMSSLKSEDTATYFCARGEDFYLYAMDYWGQGTSVTVSS

SEQ ID NO: 277 - protein, 116 aa, synthetic construct
EVQLQQSRPELVKPGASVKIFCKASGYTFTDYYMNWMRQRHGETLEWIGDINPNNGDPSYNQKFKDKATLTVDKSSSTASMELRSLTSDDSAVYYCAREGPSFAYWGQGTLVTVSA

SEQ ID NO: 278 - protein, 116 aa, synthetic construct
EVQLQQSGPELVKPGASVKISCKASGYTFTDHYMNWVKQSHGKSLEWIGDINPNNGGTSCNQKFKGKATLTVDKSSSTAYMELRSLTSGDSAVYYCTREGASFAFWGQGTLVTVSA

SEQ ID NO: 279 - protein, 116 aa, synthetic construct
QIQLVQSGPELKKPGETVKISCKASGYIFRNYGMNWVKQGPGKGLKWMGWINTYTGEPTYADDFKGRFAFSLETSASTAYLQISNLKNEDTATYFCVRDGPGFAYWGQGTLVTVSA

SEQ ID NO: 280 - protein, 114 aa, synthetic construct
EVLLQQSGPELVKPGASVKIPCKASGYTFTDYDMDWVKQSHGKSLEWIGHINPNNGGTIYNQKFKGKATLTVDKSSSTAYMELRSLTSEDTAVYYCGTGDFAYWGHGTLVTVSA

SEQ ID NO: 281 - protein, 114 aa, synthetic construct
EVLLQQSGPELVKPGASVKIPCKASGYTFTDYNIDWVKQSHGKSLEWIGDINPNNGGINYNQKFKGKATLTVDKSSSTAYMELRSLTSEDTAVYYCGTGDYAYWGQGTLVTVSA

SEQ ID NO: 282 - protein, 118 aa, synthetic construct
EVQLQQSGPELVKPGASMKISCKASGYSFTGYTMTWVKQSHGKNLEWIGLINPYNGGTNYNQKFKGKATFTVDKSSSTAYMELLSLTSEDSAVYYCARRHYGSSWDYWGQGTTLTVSS

SEQ ID NO: 283 - protein, 113 aa, synthetic construct
EVQLQQSGAELVKPGASVKLSCTASDFNIKDTYMHWVMQRPEQGLEWIGKIDPANGNTEFDPKFQGKATITADTSSNTAYLQLTSLTSEDTAVYYCTRAMDYWGQGTSVTVSS

SEQ ID NO: 284 - protein, 113 aa, synthetic construct
EVQLQQSGAELVKPGASVRLSCTASGFNIKHTYIHWVSQRPEQGLEWIGKIDPANGNTKYDPKFQGKATITADTSSNTAYLQLSSLTSEDTAVYYCVNAMEYWGQGTSVTVSS

SEQ ID NO: 285 - protein, 113 aa, synthetic construct
EVQLQQSGAELVKPGASVRLSCTASGFNIKHTYMHWVSQRPERGLEWIGKIDPANGNTKYDPKFQGKATITADTSSNTVYLQLSSLTSEDTAVYYCLNAMEYWGQGTSVTVSS

SEQ ID NO: 286 - protein, 113 aa, synthetic construct
EVQLQQSGAEFVKPGASVRLSCTASGFNIKDTYMHWVKQRPEQGLEWIGRIDPANGYTKDDPKFQGKATITADTSSNTAYLQLSSLTSEDTAVYYCASAMDYWGQGTSVTVSS

SEQ ID NO: 287 - protein, 113 aa, synthetic construct
EVQLQQSGADLVKPGASVKLSCTASGFNIKATYMHWVRQRPEKGLEWIGRIDPANGHTIYDPQFQGKATITSDTSSNTAYLQLNSLTSEDTAVYYCAEAMDYWGQGTSVTVSS

SEQ ID NO: 288 - protein, 118 aa, synthetic construct
EIQLQQTGPELVKPGASVKISCKASGYSFTDYIILWVKQSHGKSLEWIGNINPYYDYTSYNLKFKGKATLTVDKSSSTAYMQLNSLTSEDSAVYYCARSDGYYGGDYWGQGTSVTVSS

SEQ ID NO: 289 - protein, 118 aa, synthetic construct
QVQLQQPGAELVRPGTSVKLSCKASGYTFTSYWMHWVKQRPGQGLEWIGVIDPSDSYTNYNQKFKGKATLTVDTSSSTAYMQLSSLTSEDSAVYYCGRNGYDGSMDYWGQGTSVTVSS

SEQ ID NO: 290 - protein, 118 aa, synthetic construct
QVQLQQPGAELVRPGTSVKLSCKASGYTFTSYWMHWVKQRPGQGLEWIGVIDPSDSYTNYNQKFKGKATLTVDTSSSTAYMQLSSLTSEDSAVYYCARNGYDGSMDYWGQGTSVTVSS

SEQ ID NO: 291 - protein, 118 aa, synthetic construct
QVQLQQPGAELVRPGTSVKLSCKASGYTFTSYWMHWVKQRPGQGLEWIGVIDPSDSYTKYNQKFKDKATLTVETSSSTAYMQLSSLTSEDSAVYYCAGNGYDGSMDYWGQGTSVTVSS

SEQ ID NO: 292 - protein, 118 aa, synthetic construct
QVQLQQPGAELVRPGTSVKLSCKASGYTFTSYWMHWVKQRPGQGLEWIGVIDPSDSYTYYNQKFKGKATLTVDTSSSTAYMQLSSLTSEDSAVYYCARNGYDGAMDYWGQGTSVTVSS

SEQ ID NO: 293 - protein, 116 aa, synthetic construct
EVQLQQSGPELVKPGASVKISCKAFGYTFTDYYKNWMRQRHGESLEWIGDINPNSGDANYNQKFKGKATLTVDKSSSTAYMELRSLTSEDSAVYYCAREGPSFAYWGHGTLVTVSA

SEQ ID NO: 294 - protein, 116 aa, synthetic construct
EVQLQQSGPELVKPGASVKISCKASGYTFTDYYTNWMRQRHGESLEWIGDINPNTGDTSYNQKFRVKATLTVDKSSGTAYMGLRSLTSEDSAVYYCTREGPSFAYWGQGTLVTVSA

SEQ ID NO: 295 - protein, 116 aa, synthetic construct
EVQLQQSGPELVKPGASVKISCKASGYTFTDYYKNWMRQRHGESLEWIGDINPNNGDTSYNQKFRGKATLTVDKSSSTAFMELRSLTSEDSAVYYCAREGPSFAYWGQGTLVTVSA

SEQ ID NO: 296 - protein, 116 aa, synthetic construct
EVQLQQSGPELVKPGASVKISCKASGYTFTDYYKNWMRQRHGESLEWIGDINPNNGDANYNQKFKGKATLTVDKSSSTAYMELRSLTSEDSAVYFCAREGPSFAYWGHGTLVTVSA

SEQ ID NO: 297 - protein, 116 aa, synthetic construct
EVHLQQSGPELVKPGASVKISCKASGYTFTDFYKNWMRQRHGESLEWIGDINPNNGGTNYNQKFKGKATLTVDKSSSTAYMELRSLTSEDSAVYYCAREGPSFAYWGQGTLVTVSA

SEQ ID NO: 298 - protein, 116 aa, synthetic construct
EVQLQQSGPELVKPGASVKISCKASGYTFTDHYMNWVKQSHGKSLEWIGDINPNNGGTSYNQKFKGKATLTVDKSSSTAYMELRSLTSGDSAVYYCTREGASFAFWGQGTLVTVSA

SEQ ID NO: 299 - protein, 122 aa, synthetic construct
QVQLQQSGPELVKPGASVKISCKASAYTFTDYYINWVKQRPGQGPEWIGWIFPGSNSTYSNEKFEVKATLTVDESSSTAYMLLSSLTSEDSAVYFCARLGYFGSSYHALDYWGQGTSVTVSS

SEQ ID NO: 300 - protein, 113 aa, synthetic construct
EVQLQQSVAELVRPGASVKLSCSASGFNIKNTYMHWVNQRPEQGLEWIGRIDPANGITKYAPNFQGKATITADTSSNTAYLQLSNLTSEDTAIYYCTRAMDYWGQGTSVTVSS

SEQ ID NO: 301 - protein, 116 aa, synthetic construct
EVQLQQSGPELVKPGASVKISCKASGYTFTSYYKNWMRQRHGESLEWIGDINPNSGDTAYNQKFKGKATLTVDRSSSTAYMELRSLTSEDSAVYYCAREGPSFAYWGQGTLVTVSA

SEQ ID NO: 302 - protein, 115 aa, synthetic construct
EVQLQQSVAELVRPGASVKLSCTVSGFNIKNTYMHWVKQRPEQGLEWIGRIDPANGDTTYAPKFQGKATITADTSSNSAYLHLSRLTSEDTAIYYCSLYDYDGYWGQGTTLTVSS

SEQ ID NO: 303 - protein, 115 aa, synthetic construct
EVQLQQSVAEFVRPGASVKLSCTASGFNIKNTYMHWVKQRPEQGLEWIGRIDPANGNTEYAPKFQGKATITADTSSNTAYLQLSSLTSEDTAIYYCALYDYDGYWGQGTTLTVSS

SEQ ID NO: 304 - protein, 119 aa, synthetic construct
QVQLQQSGAELMEPGASVKLSCKATGYTFTGYWIEWVKQRPGHGLEWIGETLPGSDSNNYNEKFKGKATFTADTSSNTAYMQLSSLTTEDSAIYYCARDYSNYWYFDVWGTGTTVTVSS

SEQ ID NO: 305 - protein, 119 aa, synthetic construct
QVQLKESGPGLVAPSQSLSITCTVSGFSLTSYGVDWIRQSPGKGLEWLGVIWGVGSTNYNSALKSRLSISKDNSKSQVFLKMNSLQTDDTAMYYCARSYDGSYWYFDVWGTGTTVTVSS

SEQ ID NO: 306 - protein, 116 aa, synthetic construct
EVQLVESGGGLVQPGGSLRLSCAASGYTFTDYYMNWVRQAPGKGLEWIGDINPNTGDTSYNADFKRRFTFSLDTSKSTAYLQMNSLRAEDTAVYYCTREGPSFAYWGQGTLVTVSS

SEQ ID NO: 307 - protein, 117 aa, synthetic construct
EVQLVESGGGLVQPGGSLRLSCAASGYTFTDYYMSWVRQAPGKGLEWIGDINPNTGDTSYNADSVKGRFTISRDNSKNTLYLQMNSLRAEDTAVYYCTREGPSFAYWGQGTLVTVSS

SEQ ID NO: 308 - protein, 107 aa, synthetic construct
EVKKPGASVKVSCKASGYTFTDYYMHWVRQAPGQGLEWIGDINPNTGDTSYNQKFQGRVTSTRDTSISTAYMELSRLRSDDTVVYYCTREGPSFAYWGQGTLVTVSS

SEQ ID NO: 309 - protein, 107 aa, synthetic construct
DIQMTQSPASLSASVGETVTITCRASENIHSYLAWYQQKQGKSPQLIVYNTKTLAEGVPSRFSGSGSGTQFSLKINSLQPEDFGSYYCQHHYGIPPTFGGGTKLEIK

SEQ ID NO: 310 - protein, 112 aa, synthetic construct
DVLMTQTPLSLPVSLGEQASISCRSSQTIVHSNGDTYLEWYLQKPGQSPNLLIYKVSNRFSGVPDRFSGSGSGTDFTLKISRVEAEDLGVYYCFQGSHVPPTFGGGTKLEIK

SEQ ID NO: 311 - protein, 112 aa, synthetic construct
DVLMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPVTFGAGTKLELK

SEQ ID NO: 312 - protein, 112 aa, synthetic construct
DVLMTQTPLSLPVSLGDQASISCRSSLIIEHSDGNTYLEWYLQKPGQSPKLLIYKVSNRFSGVPDRFSGSGSGTDFTLKISRVEADDLGVYYCFQGSHVPVTFGAGTNLELK

SEQ ID NO: 313 - protein, 108 aa, synthetic construct
QIVLTQSPAIMSVSPGEKVTLTCSASSSVSSSYLYWYQKKPGSSPKLWIYSTSNLASGVPARFSGSGSGTSYSLTISSMEAEDAASYFCHQWSSYPPTFGAGTKLELK

SEQ ID NO: 314 - protein, 108 aa, synthetic construct
QIVLTQSPAFMSASPGEKVTLTCSASSSVSSSYLYWYQQKPGSSPKLWIYSTSNLASGVPGRFSGSGSGTSYSLTISSMEAEDAASYFCHQWTSYPPTFGAGTKLELK

SEQ ID NO: 315 - protein, 107 aa, synthetic construct
DIKLTQSPSSMYASLGERVTITCKASQDINTYLSWFQQKPGKSPKTLIYRANRLVDGVPSRFSGSGSGQDYSLTISSLEYEEMGIYYCLQYDEFPYTFGGGTKLEIK

SEQ ID NO: 316 - protein, 106 aa, synthetic construct
QIVLTQSPAIMSASPGEKVTMTCSAGSSVSYMYWYQQKPGSSPRVLIYDTSNLASGVPVRFSGSGSGTSYSLTISRMEAEDAATYYCQQWSNYPYTFGGGTKLEIK

SEQ ID NO: 317 - protein, 106 aa, synthetic construct
QSVLTQSPAIMSASPGEKVTMTCSANSSVSDMYWFQQRPGSSPRLLIYDTSNLASGVPVRFSGSGSGTSYSLTISRMEAEDAATYYCQQWSTYPWTFGGGTKLEIK

SEQ ID NO: 318 - protein, 106 aa, synthetic construct
QSVLTQSPAIMSASPGEKVTMTCSANSSVSDMYWYQQRPGSSPRLLIYDTSNLASGVPVRFSGSGSGTSYSLTISRMEAEDAATYYCQQWSTYPWTFGGGTKLEIK

SEQ ID NO: 319 - protein, 106 aa, synthetic construct
QIVLTQSPAVMSASPGEKVAMTCSASSSVTYMYWYQQKPGSSPRLLIYDTSNLASGVPVRFSGSGSGTSYSLTISRMEAEDAATYYCQQWSTYPFPFGSGTKLEIK

SEQ ID NO: 320 - protein, 106 aa, synthetic construct
QIVLTQSPAIMSASPGEKVTLTCSATSSVSYMYWYQQKPGSSPRLLIYDTSNLASGVPVRFSGSGSGTSYSLTISRMEAEDDATYYCQQWSNYPFTFGGGTKLEIK

SEQ ID NO: 321 - protein, 107 aa, synthetic construct
DIVMTQSHKFMSTSVGDRVSITCKASQDVGTAVAWYQQKPGQSPKLLIYWASTRHTGVPDRFTGSGSGTDFTLTINNVQSEDLADYFCQQYSSYPWTFGGGTTLEIK

SEQ ID NO: 322 - protein, 107 aa, synthetic construct
DIQMTQTTSSLSASLGDRVTISCRASQDISNYLNWYQQKPDGTVKLLIYYPSRLHSGVPSRFSGSGSGTDYSLTISNLEQEDFATYFCQQGNTLPYTFGGGTKLEIK

SEQ ID NO: 323 - protein, 107 aa, synthetic construct
EIQMTQTTSSLSASLGDRVTISCRASQDISNYLNWYQQKPDGTVKLLIYYPSRLHSGVPSRFSGSGSGTDYSLTISNLEQEDFATYFCQQGNTLPYTFGGGTKLEIK

SEQ ID NO: 324 - protein, 107 aa, synthetic construct
DIQMTQTTSSLSASLGDRVTISCRASQDISNSLNWYQQKPDGTVKLLIYYTSRLHSRVPSRFSGSGSGTDYSLTISNLDQEDIATYFCQQANTLPYTFGGGTKLEIK

SEQ ID NO: 325 - protein, 107 aa, synthetic construct; identical to SEQ ID NO: 323

SEQ ID NO: 326 - protein, 112 aa, synthetic construct
DVLMTQTPLSLPVSLGDQASISCRSSQTIVHSNGDTYLEWYLQKPGQSPNLLIYKVSNRFSGVPDRFSGSGSGTDFTLKISRVEAEDLGIYYCFQGSHVPPTFGGGTKLEIK

SEQ ID NO: 327 - protein, 112 aa, synthetic construct
DVLMTQTPLSLPVSLGDQASISCRSSQTIVHSNGDTYLEWYLQKPGQSPNLLIYKVSNRFSGVPDRFSGSGSGTDFTLKISRVEAEDLGVYYCFQGSHVPPTFGGGTTLEIK

SEQ ID NO: 328 - protein, 112 aa, synthetic construct
DVLMTQTPLSLPVSLGDQASISCRSNQTIVHSNGDTYLEWYLQKPGQSPNLLIYKVSNRFSGVPDRFSGSGSGTDFTLRISRVEAEDLGVYYCFQGSHVPPTFGGGTKLEIK

SEQ ID NO: 329 - protein, 112 aa, synthetic construct
DVLMTQTPLSLPVSLGDQASISCRSSQTIVHSNGDTYLEWYLQKPGQSPNLLIYKVSNRFSGVPDRFSGSGSGTDFTLKISRVEAEDLGVYYCFQGSHVPPTFGGGTKLEIK

SEQ ID NO: 330 - protein, 112 aa, synthetic construct; identical to SEQ ID NO: 329

SEQ ID NO: 331 - protein, 112 aa, synthetic construct
DVLMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPLTFGAGTKLELK

SEQ ID NO: 332 - protein, 107 aa, synthetic construct
DIQMTQSPASLSVSVGETVTITCRASENIYSHLAWFQQKQGKSPRLLVYSATNLPDGVPSRFSGSGSGTQYSLKINILQSEDFGSYYCQHFWGTPWTFGGGTKLEIK

SEQ ID NO: 333 - protein, 106 aa, synthetic construct
QIVLTQSPAIMSASPGEKVTMTCSASSSVSYMYWYQQKPGSSPRLLIYDISNLASGVPVRFSGSGSGTSYSLTISRMEAEDAATYYCQQWDTYPWTFGGGTKLEIK

SEQ ID NO: 334 - protein, 112 aa, synthetic construct
DVLMTQTPLSLPVSLGDQVSISCRSSQTIVHSNGDTYLEWYLQKPGQSPNLLIYKVSNRFSGVPDRFSGSGSGTDFTLKISRVEAEDLGVYYCFQGSHVPPTFGGGTKLEIK

SEQ ID NO: 335 - protein, 108 aa, synthetic construct
EIVLTQSPALMAASPGEKVTITCSVSSSISSSSLHWYRQKSGTSPKPWIYGTSHLASGVPVRFSGSGSGTSYSLTISSMEAEDAATYYCQQWDTYPWTFGGGTKLEIK

SEQ ID NO: 336 - protein, 108 aa, synthetic construct
EIVLTQSPALMAASPGEKVTITCRVSSSISSSSLHWYQQKSGTSPKPWIYGTSNLASGVPVRFSGSRSGTSYSLTISSMEAEDAATYYCQQWSDYPWTFGGGTKLEIK

SEQ ID NO: 337 - protein, 106 aa, synthetic construct
DIQMTQTTSSLSASLGDRVTISCRASQDISNYLNWYQQKPDGTVKLLIYYTSNLHSGVPSRFSGSGSGTDYSLTISNLEQEDIATYFCQQDSKHRTFGGGTKLEIK

SEQ ID NO: 338 - protein, 107 aa, synthetic construct
DIQMTQTTSSLSASLGDRVTISCRASQVISNYLNWYQQKPDGTVKLLIYYTSRLHSGVPSRFSGSGSGTDYSLTISNLEPEDIATYYCQQYSKLPYTFGSGTKLEIK

SEQ ID NO: 339 - protein, 112 aa, synthetic construct
DIQLTQSPSSLSASVGDRVTITCRSSQTIVHSNGDTYLEWYQQKPGKAPNLLIYKVSNRFSGVPSRFSGSGSGTDFTLTISSLQPEDFATYYCFQGSHVPPTFGQGTKVEIK

SEQ ID NO: 340 - protein, 112 aa, synthetic construct
DIQMTQSPSSLSASVGDRVTITCRSSQTIVHSNGDTYLEWYQQKPGKAPNLLIYKVSNRFSGVPSRFSGSGSGTDFTLTISSLQPEDFATYYCFQGSHVPPTFGQGTKVEIK

SEQ ID NO: 341 - protein, 112 aa, synthetic construct
DVVMTQSPLSLPVTLGQPASISCRSSQTIVHSNGDTYLEWFQQRPGQSPNLLIYKVSNRFSGVPDRFSGSGSGTDFTLKISRVEAEDVGVYYCFQGSHVPPTFGGGTKVEIK

SEQ ID NO: 342 - protein, 10 aa, synthetic construct
GFTFSDYYMA

SEQ ID NO: 343 - protein, 10 aa, synthetic construct
GYTFTDYYMN

SEQ ID NO: 344 - protein, 10 aa, synthetic construct
GYTFTDHYMN

SEQ ID NO: 345 - protein, 10 aa, synthetic construct
GYIFRNYGMN

SEQ ID NO: 346 - protein, 10 aa, synthetic construct
GYTFTDYDMD

SEQ ID NO: 347 - protein, 10 aa, synthetic construct
GYTFTDYNID

SEQ ID NO: 348 - protein, 10 aa, synthetic construct
GYSFTGYTMT

SEQ ID NO: 349 - protein, 10 aa, synthetic construct
DFNIKDTYMH

SEQ ID NO: 350 - protein, 10 aa, synthetic construct
GFNIKHTYIH

SEQ ID NO: 351 - protein, 10 aa, synthetic construct
GFNIKHTYMH

SEQ ID NO: 352 - protein, 10 aa, synthetic construct
GFNIKDTYMH

SEQ ID NO: 353 - protein, 10 aa, synthetic construct
GFNIKATYMH

SEQ ID NO: 354 - protein, 10 aa, synthetic construct
GYSFTDYIIL

SEQ ID NO: 355 - protein, 10 aa, synthetic construct
GYTFTSYWMH

SEQ ID NO: 356 - protein, 10 aa, synthetic construct
GYTFTSYWMH

SEQ ID NO: 357 - protein, 10 aa, synthetic construct
GYTFTSYWMH

SEQ ID NO: 358 - protein, 10 aa, synthetic construct
GYTFTSYWMH

SEQ ID NO: 359 - protein, 10 aa, synthetic construct
GYTFTDYYKN

SEQ ID NO: 360 - protein, 10 aa, synthetic construct
GYTFTDYYTN

SEQ ID NO: 361 - protein, 10 aa, synthetic construct
GYTFTDYYKN

SEQ ID NO: 362 - protein, 10 aa, synthetic construct
GYTFTDYYKN

SEQ ID NO: 363 - protein, 10 aa, synthetic construct
GYTFTDFYKN

SEQ ID NO: 364 - protein, 10 aa, synthetic construct
GYTFTDHYMN

SEQ ID NO: 365 - protein, 10 aa, synthetic construct
AYTFTDYYIN

SEQ ID NO: 366 - protein, 10 aa, synthetic construct
GFNIKNTYMH

SEQ ID NO: 367 - protein, 10 aa, synthetic construct
GYTFTSYYKN

SEQ ID NO: 368 - protein, 10 aa, synthetic construct
GFNIKNTYMH

SEQ ID NO: 369 - protein, 10 aa, synthetic construct
GFNIKNTYMH

SEQ ID NO: 370 - protein, 10 aa, synthetic construct
GYTFTGYWIE

SEQ ID NO: 371 - protein, 10 aa, synthetic construct
GFSLTSYGVD

SEQ ID NO: 372 - protein, 16 aa, synthetic construct
INYDGSSTYYLDSLKS

SEQ ID NO: 373 - protein, 16 aa, synthetic construct
INPNNGDPSYNQKFKD

SEQ ID NO: 374 - protein, 16 aa, synthetic construct
INPNNGGTSCNQKFKG

SEQ ID NO: 375 - protein, 16 aa, synthetic construct
INTYTGEPTYADDFKG

SEQ ID NO: 376 - protein, 16 aa, synthetic construct
INPNNGGTIYNQKFKG

SEQ ID NO: 377 - protein, 16 aa, synthetic construct
INPNNGGINYNQKFKG

SEQ ID NO: 378 - protein, 16 aa, synthetic construct
INPYNGGTNYNQKFKG

SEQ ID NO: 379 - protein, 16 aa, synthetic construct
IDPANGNTEFDPKFQG

SEQ ID NO: 380 - protein, 16 aa, synthetic construct
IDPANGNTKYDPKFQG

SEQ ID NO: 381 - protein, 16 aa, synthetic construct
IDPANGNTKYDPKFQG

SEQ ID NO: 382 - protein, 16 aa, synthetic construct
IDPANGYTKDDPKFQG

SEQ ID NO: 383 - protein, 16 aa, synthetic construct
IDPANGHTIYDPQFQG

SEQ ID NO: 384 - protein, 16 aa, synthetic construct
INPYYDYTSYNLKFKG

SEQ ID NO: 385 - protein, 16 aa, synthetic construct
IDPSDSYTNYNQKFKG

SEQ ID NO: 386 - protein, 16 aa, synthetic construct
IDPSDSYTNYNQKFKG

SEQ ID NO: 387 - protein, 16 aa, synthetic construct
IDPSDSYTKYNQKFKD

SEQ ID NO: 388 - protein, 16 aa, synthetic construct
IDPSDSYTYYNQKFKG

SEQ ID NO: 389 - protein, 16 aa, synthetic construct
INPNSGDANYNQKFKG

SEQ ID NO: 390 - protein, 16 aa, synthetic construct
INPNTGDTSYNQKFRV

SEQ ID NO: 391 - protein, 16 aa, synthetic construct
INPNNGDTSYNQKFRG

SEQ ID NO: 392 - protein, 16 aa, synthetic construct
INPNNGDANYNQKFKG

SEQ ID NO: 393 - protein, 16 aa, synthetic construct
INPNNGGTNYNQKFKG

SEQ ID NO: 394 - protein, 16 aa, synthetic construct
INPNNGGTSYNQKFKG

SEQ ID NO: 395 - protein, 16 aa, synthetic construct
IFPGSNSTYSNEKFEV

SEQ ID NO: 396 - protein, 17 aa, synthetic construct
RIDPANGITKYAPNFQG

SEQ ID NO: 397 - protein, 16 aa, synthetic construct
INPNSGDTAYNQKFKG

SEQ ID NO: 398 - protein, 17 aa, synthetic construct
RIDPANGDTTYAPKFQG

SEQ ID NO: 399 - protein, 17 aa, synthetic construct
RIDPANGDTTYAPKFQG

SEQ ID NO: 400 - protein, 17 aa, synthetic construct
ETLPGSDSNNYNEKFKG

SEQ ID NO: 401 - protein, 16 aa, synthetic construct
VIWGVGSTNYNSALKS

SEQ ID NO: 402 - protein, 13 aa, synthetic construct
ARGEDFYLYAMDY

SEQ ID NO: 403 - protein, 9 aa, synthetic construct
AREGPSFAY

SEQ ID NO: 404 - protein, 9 aa, synthetic construct
TREGASFAF

SEQ ID NO: 405 - protein, 9 aa, synthetic construct
VRDGPGFAY

SEQ ID NO: 406 - protein, 7 aa, synthetic construct
GTGDFAY

SEQ ID NO: 407 - protein, 7 aa, synthetic construct
GTGDYAY

SEQ ID NO: 408 - protein, 11 aa, synthetic construct
ARRHYGSSWDY

SEQ ID NO: 409 - protein, 6 aa, synthetic construct
TRAMDY

SEQ ID NO: 410 - protein, 6 aa, synthetic construct
VNAMEY

SEQ ID NO: 411 - protein, 6 aa, synthetic construct
LNAMEY

SEQ ID NO: 412 - protein, 6 aa, synthetic construct
ASAMDY

SEQ ID NO: 413 - protein, 6 aa, synthetic construct
AEAMDY

SEQ ID NO: 414 - protein, 11 aa, synthetic construct
ARSDGYYGGDY

SEQ ID NO: 415 - protein, 11 aa, synthetic construct
GRNGYDGSMDY

SEQ ID NO: 416 - protein, 11 aa, synthetic construct
ARNGYDGSMDY

SEQ ID NO: 417 - protein, 11 aa, synthetic construct
AGNGYDGSMDY

SEQ ID NO: 418 - protein, 11 aa, synthetic construct
ARNGYDGAMDY

SEQ ID NO: 419 - protein, 9 aa, synthetic construct
AREGPSFAY

SEQ ID NO: 420 - protein, 9 aa, synthetic construct
TREGPSFAY

SEQ ID NO: 421 - protein, 9 aa, synthetic construct
AREGPSFAY

SEQ ID NO: 422 - protein, 9 aa, synthetic construct
AREGPSFAY

SEQ ID NO: 423 - protein, 9 aa, synthetic construct
AREGPSFAY

SEQ ID NO: 424 - protein, 9 aa, synthetic construct
TREGASFAF

SEQ ID NO: 425 - protein, 15 aa, synthetic construct
ARLGYFGSSYHALDY

SEQ ID NO: 426 - protein, 6 aa, synthetic construct
TRAMDY

SEQ ID NO: 427 - protein, 9 aa, synthetic construct
AREGPSFAY

SEQ ID NO: 428 - protein, 8 aa, synthetic construct
SLYDYDGY

SEQ ID NO: 429 - protein, 8 aa, synthetic construct
ALYDYDGY

SEQ ID NO: 430 - protein, 12 aa, synthetic construct
ARDYSNYWYFDV

SEQ ID NO: 431 - protein, 13 aa, synthetic construct
ARSYDGSYWYFDV

SEQ ID NO: 432 - protein, 11 aa, synthetic construct
RASENIHSYLA

SEQ ID NO: 433 - protein, 16 aa, synthetic construct
RSSQTIVHSNGDTYLE

SEQ ID NO: 434 - protein, 16 aa, synthetic construct
RSSQSIVHSNGDTYLE

SEQ ID NO: 435 - protein, 16 aa, synthetic construct
RSSLIIEHSDGNTYLE

SEQ ID NO: 436 - protein, 12 aa, synthetic construct
SASSSVSSSYLY

SEQ ID NO: 437 - protein, 12 aa, synthetic construct
SASSSVSSSYLY

SEQ ID NO: 438 - protein, 11 aa, synthetic construct
KASQDINTYLS

SEQ ID NO: 439 - protein, 10 aa, synthetic construct
SAGSSVSYMY

SEQ ID NO: 440 - protein, 10 aa, synthetic construct
SANSSVSDMY

SEQ ID NO: 441 - protein, 10 aa, synthetic construct
SANSSVSDMY

SEQ ID NO: 442 - protein, 10 aa, synthetic construct
SASSSVTYMY

SEQ ID NO: 443 - protein, 10 aa, synthetic construct
SATSSVSYMY

SEQ ID NO: 444 - protein, 11 aa, synthetic construct
KASQDVGTAVA

SEQ ID NO: 445 - protein, 11 aa, synthetic construct
RASQDISNYLN

SEQ ID NO: 446 - protein, 11 aa, synthetic construct
RASQDISNYLN

SEQ ID NO: 447 - protein, 11 aa, synthetic construct
RASQDISNSLN

SEQ ID NO: 448 - protein, 11 aa, synthetic construct
RASQDISNYLN

SEQ ID NO: 449 - protein, 16 aa, synthetic construct
RSSQTIVHSNGDTYLE

SEQ ID NO: 450 - protein, 16 aa, synthetic construct
RSSQTIVHSNGDTYLE

SEQ ID NO: 451 - protein, 16 aa, synthetic construct
RSNQTIVHSNGDTYLE

SEQ ID NO: 452 - protein, 16 aa, synthetic construct
RSSQTIVHSNGDTYLE

SEQ ID NO: 453 - protein, 16 aa, synthetic construct
RSSQTIVHSNGDTYLE

SEQ ID NO: 454 - protein, 16 aa, synthetic construct
RSSQSIVHSNGDTYLE

SEQ ID NO: 455 - protein, 11 aa, synthetic construct
RASENIYSHLA

SEQ ID NO: 456 - protein, 10 aa, synthetic construct
SASSSVSYMY

SEQ ID NO: 457 - protein, 16 aa, synthetic construct
RSSQTIVHSNGDTYLE

SEQ ID NO: 458 - protein, 12 aa, synthetic construct
SVSSSISSSSLH

SEQ ID NO: 459 - protein, 12 aa, synthetic construct
RVSSSISSSSLH

SEQ ID NO: 460 - protein, 11 aa, synthetic construct
RASQDISNYLN

SEQ ID NO: 461 - protein, 11 aa, synthetic construct
RASQVISNYLN

SEQ ID NO: 462 - protein, 7 aa, synthetic construct
NTKTLAE

SEQ ID NO: 463 - protein, 7 aa, synthetic construct
KVSNRFS

SEQ ID NO: 464 - protein, 7 aa, synthetic construct
KVSNRFS

SEQ ID NO: 465 - protein, 7 aa, synthetic construct
KVSNRFS

SEQ ID NO: 466 - protein, 7 aa, synthetic construct
STSNLAS

SEQ ID NO: 467 - protein, 7 aa, synthetic construct
STSNLAS

SEQ ID NO: 468 - protein, 7 aa, synthetic construct
RANRLVD

SEQ ID NO: 469 - protein, 7 aa, synthetic construct
DTSNLAS

SEQ ID NO: 470 - protein, 7 aa, synthetic construct
DTSNLAS

SEQ ID NO: 471 - protein, 7 aa, synthetic construct
DTSNLAS

SEQ ID NO: 472 - protein, 7 aa, synthetic construct
DTSNLAS

SEQ ID NO: 473 - protein, 7 aa, synthetic construct
DTSNLAS

SEQ ID NO: 474 - protein, 7 aa, synthetic construct
WASTRHT

SEQ ID NO: 475 - protein, 7 aa, synthetic construct
YPSRLHS

SEQ ID NO: 476 - protein, 7 aa, synthetic construct
YPSRLHS

SEQ ID NO: 477 - protein, 7 aa, synthetic construct
YTSRLHS

SEQ ID NO: 478 - protein, 7 aa, synthetic construct
YPSRLHS

SEQ ID NO: 479 - protein, 7 aa, synthetic construct
KVSNRFS

SEQ ID NO: 480 - protein, 7 aa, synthetic construct
KVSNRFS

SEQ ID NO: 481 - protein, 7 aa, synthetic construct
KVSNRFS

SEQ ID NO: 482 - protein, 7 aa, synthetic construct
KVSNRFS

SEQ ID NO: 483 - protein, 7 aa, synthetic construct
KVSNRFS

SEQ ID NO: 484 - protein, 7 aa, synthetic construct
KVSNRFS

SEQ ID NO: 485 - protein, 7 aa, synthetic construct
SATNLPD

SEQ ID NO: 486 - protein, 7 aa, synthetic construct
DISNLAS

SEQ ID NO: 487 - protein, 7 aa, synthetic construct
KVSNRFS

SEQ ID NO: 488 - protein, 7 aa, synthetic construct
GTSHLAS

SEQ ID NO: 489 - protein, 7 aa, synthetic construct
GTSNLAS

SEQ ID NO: 490 - protein, 7 aa, synthetic construct
YTSNLHS

SEQ ID NO: 491 - protein, 7 aa, synthetic construct
YTSRLHS

SEQ ID NO: 492 - protein, 9 aa, synthetic construct
QHHYGIPPT

SEQ ID NO: 493 - protein, 9 aa, synthetic construct
FQGSHVPPT

SEQ ID NO: 494 - protein, 9 aa, synthetic construct
FQGSHVPVT

SEQ ID NO: 495 - protein, 9 aa, synthetic construct
FQGSHVPVT

SEQ ID NO: 496 - protein, 9 aa, synthetic construct
HQWSSYPPT

SEQ ID NO: 497 - protein, 9 aa, synthetic construct
HQWTSYPPT

SEQ ID NO: 498 - protein, 9 aa, synthetic construct
LQYDEFPYT

SEQ ID NO: 499 - protein, 9 aa, synthetic construct
QQWSNYPYT

SEQ ID NO: 500 - protein, 9 aa, synthetic construct
QQWSTYPWT

SEQ ID NO: 501 - protein, 9 aa, synthetic construct
QQWSTYPWT

SEQ ID NO: 502 - protein, 9 aa, synthetic construct
QQWSNYPFT

SEQ ID NO: 503 - protein, 9 aa, synthetic construct
QQWSNYPFT

SEQ ID NO: 504 - protein, 9 aa, synthetic construct
QQYSSYPWT

SEQ ID NO: 505 - protein, 9 aa, synthetic construct
QQGNTLPYT

SEQ ID NO: 506 - protein, 9 aa, synthetic construct
QQGNTLPYT

SEQ ID NO: 507 - protein, 9 aa, synthetic construct
QQANTLPYT

SEQ ID NO: 508 - protein, 9 aa, synthetic construct
QQGNTLPYT

SEQ ID NO: 509 - protein, 9 aa, synthetic construct
FQGSHVPPT

SEQ ID NO: 510 - protein, 9 aa, synthetic construct
FQGSHVPPT

SEQ ID NO: 511 - protein, 9 aa, synthetic construct
FQGSHVPPT

SEQ ID NO: 512 - protein, 9 aa, synthetic construct
FQGSHVPPT

SEQ ID NO: 513 - protein, 9 aa, synthetic construct
FQGSHVPPT

SEQ ID NO: 514 - protein, 9 aa, synthetic construct
FQGSHVPLT

SEQ ID NO: 515 - protein, 9 aa, synthetic construct
QHFWGTPWT

SEQ ID NO: 516 - protein, 9 aa, synthetic construct
QQWDTYPWT

SEQ ID NO: 517 - protein, 9 aa, synthetic construct
FQGSHVPPT

SEQ ID NO: 518 - protein, 9 aa, synthetic construct
QQWDTYPWT

SEQ ID NO: 519 - protein, 9 aa, synthetic construct
QQWSDYPWT

SEQ ID NO: 520 - protein, 8 aa, synthetic construct
QQDSKHRT

SEQ ID NO: 521 - protein, 9 aa, synthetic construct
QQYSKLPYT

SEQ ID NO: 522 - protein, 121 aa, synthetic construct
EVQLQQSGPELVKPGASVKISCKASDNSFTGYYMHWVKQSHGNILDWIGYIDPYNGVSSYNQKFKGKATLTVDKSSSTAYMEIRSLTSEDSAVYYCASYYGSSPYWYFDVWGTGTTVTVSS

SEQ ID NO: 523 - protein, 120 aa, synthetic construct; identical to SEQ ID NO: 276

SEQ ID NO: 524 - protein, 116 aa, synthetic construct; identical to SEQ ID NO: 277

SEQ ID NO: 525 - protein, 119 aa, synthetic construct
EVQLQQSGAELVRPGSSVKMSCKTSGKTFTSHGINWVKQRPGQGLEWIGYIYIGNGYNEYNEKFKGKATLTSDTSSSTAYMQLSSLTFEDSAIYFCVRKAYGNYGFDDWGQGTTLTVSS

SEQ ID NO: 526 - protein, 116 aa, synthetic construct; identical to SEQ ID NO: 278

SEQ ID NO: 527 - protein, 113 aa, synthetic construct
EVQLQQSGAELVKPGASVKLSCTASGFKIKDTYMHWVKERPEQGLEWIGRIDPANGNTKYDPKFQGKATITADTSSNTAYLQLSSLTSEDTAVYYCANAMDYWGQGTSVTVSS

SEQ ID NO: 528 - protein, 113 aa, synthetic construct; identical to SEQ ID NO: 527

SEQ ID NO: 529 - protein, 113 aa, synthetic construct
EVQLQQSGPELVKAGASVKMSCTASGFNIKDTYMHWVKQRPEQGLAWIGRIDPANGNIKYDPKFQGKATITADTSSNTAYLQLSSLTSDDTAVYYCTSAMDYWGQGTSVTVSS

SEQ ID NO: 530 - protein, 116 aa, synthetic construct; identical to SEQ ID NO: 279

SEQ ID NO: 531 - protein, 114 aa, synthetic construct; identical to SEQ ID NO: 280

SEQ ID NO: 532 - protein, 117 aa, synthetic construct
EVKLVESGGGLVQPGGSLKLSCAASGFTFSSNTMSWVRQTPEKRLEWVAYITNGGGSTYYPDTVKGRFTISRDNARNTLYLQMSSLKSEDTAMYYCARHDYYTMDYWGQGTSVTVSS

SEQ ID NO: 533 - protein, 113 aa, synthetic construct
EVQLQQSGAELVKPGASVKLSCTASGFKIKDTYMHWVKERPDQGLEWIGRIDPANGNTKYDPKFQGKATITADTSSNTAYLQLSSLTSEDTAVYYCANAMDYWGQGTSVTVSS

SEQ ID NO: 534 - protein, 118 aa, synthetic construct
EVKLVESGGGLVQPGGSLKLSCAASGFTFSSYIMSWVRQTPEKRLEWVAYITNGGGNTYYPDTIKGRFTISRDNAKNTLYLQMSSLKSEDTAMYYCARHGTGYAMDYWGQGTSVTVSS

SEQ ID NO: 535 - protein, 113 aa, synthetic construct
EVQLQQSGAELVKPGASVKLSCTASGFNIKDTYMHWVKQRPEQGLEWIGRIDPANGYTEYDPKFQGKATITADTSSNTAYLQLSSLTSEDSAAYYCTSAMEFWGQGTSVTVSS

SEQ ID NO: 536 - protein, 122 aa, synthetic construct
QVQLQQSGAELAKPGASVKMSCKASGYTFTNFWMHWVKQRPGQGLEWIGFFNPSTAYTEYNQKFKDKATLTADKSSSTAYLHLSSLTSEDSAVYYCARRDYGSSYGWYFDVWGAGTTVTVSS

SEQ ID NO: 537 - protein, 122 aa, synthetic construct
DVQLQESGPDLVKPSQSLSLTCTVTDYSITSGYSWHWIRQFPGNKLEWLGYIHSSGNTNYNPSLKSRFSITRDTSKNQFFLQLNSVTSEDTATYYCALHYYGSSFGWYFDVWGAGTTVTVSS

SEQ ID NO: 538 - protein, 119 aa, synthetic construct
QVQLQQSAAELARPGASVKMSCKASGYTFTSYTVHWVKQRPGQGLEWIGYINPSSGFTDYNQKFKDKTTLTADISSSTAYIQLSSLTSEDSAVYYCARRGVNWSWFAYWGQGTLVTVSA

SEQ ID NO: 539 - protein, 122 aa, synthetic construct
QVQLQQSGAELARPGASVKMSCKASGYTFATYTIHWVKQRPGQGLEWIGYLNLRNDYTHYNQKFRDKAALTADKSSSTAYMQLSSLTSEDSAVYNCAFRLGNDRQGWYFDVWGAGTTVTVSS

SEQ ID NO: 540 - protein, 122 aa, synthetic construct
DVQLQESGPDLVKPSQSLSLTCTVTGYSITSGYSWHWIRQFPGNTLEWMGYIHYSGSTNYNPSLESRISFTRDTSKNQFFLQLNSVTTEDTATYYCALHFYGYNLGWYFDVWGAGTTVTVSS

SEQ ID NO: 541 - protein, 114 aa, synthetic construct; identical to SEQ ID NO: 281

SEQ ID NO: 542 - protein, 117 aa, synthetic construct
QVQLQQSGAELAKPGASVKMSCKASGYTFTNYWMHWVKQRPGQGLEWIGYINPSIGYTEYNQKFKDKATLTADKSSSTAYMQLSSLTSEDSAVFYCATFIYYAMDYWGQGTSVTVSS

SEQ ID NO: 543 - protein, 118 aa, synthetic construct
EVQLQQSGPELVKPGASMKISCTASGYSFTGYTMTWVKQSHGKNLEWIGLINPYNGGTNYNQKFKGKATLTVDKSSSIAYMELLSLTSEDSAVYYCARRHYGSNWDYWGQGTTLTVSS

SEQ ID NO: 544 - protein, 118 aa, synthetic construct; identical to SEQ ID NO: 282

SEQ ID NO: 545 - protein, 123 aa, synthetic construct
EVQLQQSGPELVKPGTSMKISCKASGYSFADYTMNWVKQSHGKSLEWIGLINPYNGGTSYNQKFMGKATLTVDKSSSTAYMELLSLTSEDSAVYYCARWGTYSHNYDYAMDYWGQGTSVTVSS

SEQ ID NO: 546 - protein, 123 aa, synthetic construct
EVQLQQSGPEMVKPGASMKISCKASGYSFADYTLNWVKQSHGKSLEWIGLINPYNGGTSYNQKFMGKATLTVDKSSSTAYMELLSLTSEDSAVYYCTRWGTYSHNYDYAMDYWGQGTSVTVSS

SEQ ID NO: 547 - protein, 113 aa, synthetic construct; identical to SEQ ID NO: 283

SEQ ID NO: 548 - protein, 113 aa, synthetic construct; identical to SEQ ID NO: 284

SEQ ID NO: 549 - protein, 113 aa, synthetic construct; identical to SEQ ID NO: 285

SEQ ID NO: 550 - protein, 113 aa, synthetic construct; identical to SEQ ID NO: 286

SEQ ID NO: 551 - protein, 113 aa, synthetic construct; identical to SEQ ID NO: 287

SEQ ID NO: 552 - protein, 122 aa, synthetic construct
QVQLKESGPGLVAPSQSLSITCTVSGFSLSSYGVQWVRQPPGQGLEWLVVIWRDGSITYNSALKSRLSISKDNSKSQVFLKMNSLQTDDTAMYYCGRTSHYGNYNYYVMDYWGQGTAVTVSS

SEQ ID NO: 553 - protein, 122 aa, synthetic construct
QVQLKESGPGLVAPSQSLSITCTVSGFSLNSYGVQWVRQPPGQGLEWLVVIWRDGTITYNSALKSRLSINKDNSKSQVFLKMNSLQTDDTAMYYCGRTSHYGNFNYYVMDYWGQGTAVTVSS

SEQ ID NO: 554 - protein, 122 aa, synthetic construct
QVQLKESGPGLVAPSQSLSITCTVSGFSLNSYGVQWVRQPPGQGLEWLGVIWRDGSITYNSALKSRLSIRKDNSKSQVFLKMNSLQTDDTAMYYCGRTSHYGNYNYYVMDYWGQGTAVTVSS

SEQ ID NO: 555 - protein, 117 aa, synthetic construct
EVKLVESGGGLVQPGGSLKVSCAASGFTFSTYTMSWVRQTPEKRLEWVAYITNGGGSTYYPDTEKGRFTISRDNAKNTLYLQMSSLKSEDTAMYYCVRHDYYAMDYWGQGTSVTVSS

SEQ ID NO: 556 - protein, 117 aa, synthetic construct
EVKLVESGGGLVQPGGSLKLSCAASGFTFSNYIMSWVRQTPEKRLEWVAYITNGGGATYYPDTVKGQFTISRDNAKNTLYLQMSSLKSEDTAIYYCARHDFYALDFWGQGTSVTVSS

SEQ ID NO: 557 - protein, 117 aa, synthetic construct
EVKLVESGGDLVQPGGSLKLSCAASGFTFSRYIMSWVRLTPEKRLEWVAFITNGGGNTYHPDTVKGRFTISRDNANNTLYLQMSSLKSEDTAIYYCARHDYYALDYWGQGTSVTVSS

SEQ ID NO: 558 - protein, 118 aa, synthetic construct
EVKLVESGGGLVQPGGSLKLSCAASGFTFSTYIMSWVRQTPEKRLEWVAYITSGGSSTYYPDTVKGRFTISRDNAKSTLYLQMSSLKSEDTAMYYCARHAHFYAMDYWGQGTSVTVSS

SEQ ID NO: 559 - protein, 118 aa, synthetic construct
EVKLVESGGGLVQPGGSLKLSCAASGFIFSSYIMSWVRQTPEKRLEWVAYITNGGGSTYYPDTVKGRLTISRDNAKNTLYLQMSSLKSEDTAMYYCVRHAHYYAMDYWGQGTSVTVSS

SEQ ID NO: 560 - protein, 118 aa, synthetic construct
EVKLVESGGGLVQPGGSLKLSCAASGFTFSSYIMSWVRQTPEKRLGWVAYITSGGGSTYYPDTVKGRFTISRDNAKNTLYLQMSSLKSEDTAMYYCARHGTGYAMDYWGQGTSVTVSS

SEQ ID NO: 561 - protein, 122 aa, synthetic construct
QVQLQQSGAELARPGASVKMSCKASGYTFTTYTIHWVKQRPGQGLEWIGYINPSSDFTNYNQNFADKATLTADRSSSTAYMQLSSLTSEESAVYYCAIRLGYDRQGWYFDVWGAGTTVTVSS

SEQ ID NO: 562 - protein, 117 aa, synthetic construct
QVQLQQSAAELARPGASVKMSCKASGYTFTDYTMHWVKQRPGQGLEWIGYINPSGGYTDYNQKFKDKTALTADKSSSTAYMQLSSLTSEDSAVYYCARRRDYWFAYWGQGTLVTVSA

SEQ ID NO: 563 - protein, 118 aa, synthetic construct; identical to SEQ ID NO: 288

SEQ ID NO: 564 - protein, 116 aa, synthetic construct
QVQLLQPGAELVRPGTSVKLSCKASGYTFSNYWINWVKQRPGQGLEWIGNIYPSDSSINYNQKFKDKATLTVDKSSTTAYMQLSSPTSEDSAVYYCTGTDWYFDVWGAGTTVTVSS

SEQ ID NO: 565 - protein, 116 aa, synthetic construct
EVQLQQSGAELVRPGALVKLSCKASGFNIKDYYMHWVKQRPEQGLEWIGWIDPENGHTIYDPRFQGKATITADTSSNTAYLQLSSLTSEDTAVYYCSRGLLGFAYWGQGTLVTVSA

SEQ ID NO: 566 - protein, 118 aa, synthetic construct; identical to SEQ ID NO: 289

SEQ ID NO: 567 - protein, 118 aa, synthetic construct; identical to SEQ ID NO: 290

SEQ ID NO: 568 - protein, 118 aa, synthetic construct; identical to SEQ ID NO: 291

SEQ ID NO: 569 - protein, 118 aa, synthetic construct; identical to SEQ ID NO: 292

SEQ ID NO: 570 - protein, 121 aa, synthetic construct
EVQLQQSGPELVKPGASVKISCKASGYSFTGYYMHWVKQSHGNILDWIGYIDPDNGVSSKNQKFTGKATVTADKSSSTAYMELRSLTSEDSAVYYCAGYYGSSWYWYFDVWGTGTTVTVSS

SEQ ID NO: 571 - protein, 121 aa, synthetic construct
EVQLQQSGPELVKPGASVKISCKASGYSFTDYYMHWVKQSHGNILDWIGYIDPYNGVSSYNQKFKGKATLSVDQSSSTAYMELRSLTSEDSAVYYCSSYYGSSPYWYFDVWGTGTRVTVSS

SEQ ID NO: 572 - protein, 122 aa, synthetic construct
EVQLQQSGPELVKPGASVKISCKASGYSFTAYYMNWVKHSPEKSLEWIGDINPSTGGTTYNQKFKARATLTVDKSSSTAYMQLKSLTSEDSAVYYCATTYYSGNSYVGFAYWGQGTLVTVSA

SEQ ID NO: 573 - protein, 122 aa, synthetic construct
EVQLQQSGPELVKPGASVKISCKASGYSFTAYYMNWVKQSPEKSLEWIGDINPSTGGTTYNQNFKAKATLTVDKSSSTAYMHLKSLTSEDSAVYYCATTYYSGNSYVGFAYWGQGTLVTVSA

SEQ ID NO: 574 - protein, 122 aa, synthetic construct
EVQLQQSGPELVKPGASVKISCKASGYSFTGYYMNWVKQSPEKSLEWIGDINPSTGGTTYNQKFKAKATLTVDKSSSTAYMQLKSLTSEDSAVYYCATPYYYGSSYVGFAYWGQGTLVTVSA

SEQ ID NO: 575 - protein, 116 aa, synthetic construct; identical to SEQ ID NO: 293

SEQ ID NO: 576 - protein, 116 aa, synthetic construct; identical to SEQ ID NO: 294

SEQ ID NO: 577 - protein, 116 aa, synthetic construct; identical to SEQ ID NO: 295

SEQ ID NO: 578 - protein, 116 aa, synthetic construct; identical to SEQ ID NO: 296

SEQ ID NO: 579 - protein, 116 aa, synthetic construct; identical to SEQ ID NO: 297

SEQ ID NO: 580 - protein, 116 aa, synthetic construct; identical to SEQ ID NO: 298

SEQ ID NO: 581 - protein, 119 aa, synthetic construct
EVQLQQSGAELVRPGSSVKMSCKTSGNTFTSYGINWVKQRPGQGLEWIGYIYIGTGYTEYNEKFKGKATLTSDTSSSTAYMQLSSLTSEDSAIYFCVRKAYGNYGFDYWGQGTTLTVSS

SEQ ID NO: 582 - protein, 119 aa, synthetic construct; identical to SEQ ID NO: 525

SEQ ID NO: 583 - protein, 123 aa, synthetic construct
QVQLQQPGAELVRPESSVKLSCKASGYTFTNFWMDWVKQRPGQGLEWIGNIYPSGSETHYNQKFKDKATLTVDKSSTTAYMQLSSLTSEDSAVYYCARSGYYGSRYLYYFDYWGQGTTLTVSS

SEQ ID NO: 584 - protein, 123 aa, synthetic construct
QVQLQQPGAELVRPESSVKLSCKASGYTFTSYWMDWVMQRPGQGLEWIGNIYPSGSETHYNQKFKDKATLTVDKSSTTAYMQLSSLTFEDSAVYYCARSGFIGSRYLYYFDYWGQGTTLTVSS

SEQ ID NO: 585 - protein, 120 aa, synthetic construct
EVQLVESGGGLVQPKGSLKLSCAASGFSFNTYAMNWVRQAPGKGLEWVARIRSKSNNYATYYADSVKDRFTISRDDSESMVYLQMNNLKTEDTAMYYCVRHGYYWYFDVWGTGTTVTVSS

SEQ ID NO: 586 - protein, 122 aa, synthetic construct; identical to SEQ ID NO: 299

SEQ ID NO: 587 - protein, 121 aa, synthetic construct
QVQLQQSGPELVKPGASVKISCKASGYSFTDYYINWVKQRPGQGLEWIGWIFPGSGSTYYNEKFKGKATLTVDKSSSTAYMLLSSLTSEDSAVYFCARTGYYSNLYAVDYWGQGTSVTVSS

SEQ ID NO: 588 - protein, 121 aa, synthetic construct
EVQLVESGGGLLQPKGSLKLSCAASGFTFNTYAMNWVRQAPGKGLEWVARIRSKSSNYATYYADSVKDRFTISRDDSQSMFYLEMNNLKTEDTAMYYCVRDRGYYYVMDYWGQGTSVTVSS

SEQ ID NO: 589 - protein, 121 aa, synthetic construct
EVQLQQSGPEMVKPGASVKMSCQASGYTFTDYYMNWVKQSHGETLEWIGDIYPHNGYTAYNQKFKGKATLTVDKSSSTAYMELRSLTSEDSAVYYCARGGQLRLPAWFAYWGQGTLVTVSA

SEQ ID NO: 590 - protein, 117 aa, synthetic construct
EVQLQQSGPELVKPGASVRMSCKASGYIFTDYSIHWVKQSHGKSLEWIGYINPNNGGTSYNQKFKGKATLTVNKSSTTAYMELRSLTSEDSAVYFCARDTTIVGDYWGQGTTLTVSS

SEQ ID NO: 591 - protein, 113 aa, synthetic construct; identical to SEQ ID NO: 300

SEQ ID NO: 592 - protein, 122 aa, synthetic construct
EVQLVESGGDLVKPGGSLKLSCAASGFTFSGYGMSWVRQIPDKRLEWVAISSRDNSFTYYPDSVKGRFTISRDNAKNTLYLQMSSLKSEDTALYFCTRHPYLPTGGYVMDYWGQGTSVTVSS

SEQ ID NO: 593 - protein, 119 aa, synthetic construct
EVQLQQSGAELVRPGSSVKMSCKTSGYTFTSYGINWVKQRPGQGLEWIGYIYIANGYTENNEKFKGKAKLTSDISSSTAYMQLSSLTSEDSAIYFCARRFDYAGALDYWGQGTSVTVSS

SEQ ID NO: 594 - protein, 116 aa, synthetic construct; identical to SEQ ID NO: 301

SEQ ID NO: 595 - protein, 119 aa, synthetic construct
EVQLQQSGAELVRPGSSVKMSCKTSGNTFTSHGINWVKQRPGQGLEWIGYIYIGNGYNEYNEKFKGKATLTSDTSSSTAYMQLSSLTSEDSAIYFCVRKAYGNYGFDDWGQGTTLTVSS

SEQ ID NO: 596 - protein, 120 aa, synthetic construct
AVQLVESGGGLVQPKGSLKLSCAASGFSFNTYAMNWVRQAPGKGLEWVARIRSKSNNYATYYADSVKDRFTLSRDDSESMLYLQMNNLKTEDTAMYYCVRQGFYWYFDVWGTGTTVTVSS

SEQ ID NO: 597 - protein, 115 aa, synthetic construct; identical to SEQ ID NO: 302

SEQ ID NO: 598 - protein, 119 aa, synthetic construct
EVQLQQSGPVLVKPGASVTMSCKASGYTFTEYYMNWVKQSHGKSLDWIGVINPYSGGTSYKQKFKDKATLTVDKSSSTAYMELNGLTSEDSAVYFCVRGGLRRNYFDYWGQGTTLTVSS

SEQ ID NO: 599 - protein, 121 aa, synthetic construct
EVHLVESGGDLVKPGGSLKLSCAASGFTFSRYGMSWVRQTPDKRLEWVATISSAGSYTYYPDSVKGRFTISRDNAKNTLFLQMSSLKSEDTAMYYCARPLNYYGTSSFDYWGQGTTLTVSS

SEQ ID NO: 600 - protein, 115 aa, synthetic construct; identical to SEQ ID NO: 303

SEQ ID NO: 601 - protein, 122 aa, synthetic construct
QVHLQQSGPELVKPGASVKISCKASGYTFIDYYINWVKQRPGQGLEWIGWIFPGSDSTYYNEKFKGKATLTVDKSSSTAYMLLSSLTSEDSAVYFCARYGYYGSSFYAMDYWGQGTSVTVSS

SEQ ID NO: 602 - protein, 118 aa, synthetic construct
QVQVQQPGAEFVKTGASVKLSCKTSGYTFIDYWIHWVKQRPGHGLEWIGRIDPNTGGSKYYEKFKRKATLTVDKPSRTVYMQLSSLTSEDSAVYYCTREYDYGWFGYWGQGTLVTVSE

SEQ ID NO: 603 - protein, 122 aa, synthetic construct
QVQLQQSGPELVKPGASVKISCKASGYTFTDYYINWVKQRPGQGLEWIGWIFPGSGSTYYNEKFKGKATLTVDKSSSTAYMLLSSLPSEDSAVYFCARYGYYGSSFYAMDYWGQGTSVTVSS

SEQ ID NO: 604 - protein, 119 aa, synthetic construct; identical to SEQ ID NO: 304

SEQ ID NO: 605 - protein, 119 aa, synthetic construct; identical to SEQ ID NO: 305

SEQ ID NO: 606 - protein, 122 aa, synthetic construct
QVQLQQSGPELVKPGASVKISCKASGYTFTDYYINWMKQRPGQGLEWIGWIFPGSDSTYYNEKFKGKATLTVDKSSSTAYMLLSSLTSEDSAVYFCARLGYYSHSYYAMDYWGQGTSVTVSS

SEQ ID NO: 607 - protein, 120 aa, synthetic construct
EVQLVESGGGLVQPKGSLKLSCAASGFSFNTYAMNWVRQAPGKGLEWVARIRSKSNNYATYYADSVKDRFTIFRDDSESMLYLQMNNLKTEDTAMYYCVRHGYYWYFDVWGTGTTVTVSS

SEQ ID NO: 608 - protein, 118 aa, synthetic construct
QVQLQQPGTELVKPGASVKLPCKASGYTFTSYWMQWVKQRPGQGLEWIGEIDPSDTYINYNQKFKGKATLTVDTSSTTAYMQLSSLTSEDSAVYYCARYTTIMASDYWGQGTTLTVSS

SEQ ID NO: 609 - protein, 120 aa, synthetic construct; identical to SEQ ID NO: 585

SEQ ID NO: 610 - protein, 106 aa, synthetic construct
QIVLSQSPAILSASPGEKVTMTCRASSSVSYMHWYQQKPGSSPKPWIYATSNLASGVPARFSGSGSGTSHFLTISRLEAEDAATYFCQQWSSNPYTFGGGTKLEIK

SEQ ID NO: 611 - protein, 107 aa, synthetic construct; identical to SEQ ID NO: 309

SEQ ID NO: 612 - protein, 112 aa, synthetic construct; identical to SEQ ID NO: 310

SEQ ID NO: 613 - protein, 107 aa, synthetic construct
DIQMTQSPTSLSASLGESVSLTCRASQEISGYLNWLQQKPDGSIKRLIYAASTLDSGVPKRFSGSRSGSDYSLTISSLESEDFADYYCLQYANYPFTFGSGTKLEVK

SEQ ID NO: 614 - protein, 112 aa, synthetic construct; identical to SEQ ID NO: 311

SEQ ID NO: 615 - protein, 106 aa, synthetic construct
QIILTQSPAIMSASPGEKVTMTCSARSSVSNMYWYQQKPGSSPRLLIYDTSNLASGVPVRFSGSGSGTSYSLTISRMEAEDAATYYCQQWSSYPWTFGGGTKLEIK

SEQ ID NO: 616 - protein, 106 aa, synthetic construct
QIILTQSPAIMSASPGERVTMTCSASSSVSNMYWYQQKPGSSPRLLIYDTSNLASGVPLRFSGSGSGTSYSLTISRMEAEDAATYYCQQWSSYPWTFGGGTKLEIK

SEQ ID NO: 617 - protein, 106 aa, synthetic construct
QIVLTQSPEIMSASPGEKVTMTCSARSSVSYMYWYQQKPGSSPRLLIYDTSNLASGVPVRFSGSGSGTSYSLTISRMETEDAATYYCQQWSTYPFTFGSGTKLEIK

SEQ ID NO: 618 - protein, 112 aa, synthetic construct; identical to SEQ ID NO: 312

SEQ ID NO: 619 - protein, 108 aa, synthetic construct; identical to SEQ ID NO: 313

SEQ ID NO: 620 - protein, 107 aa, synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLLYTFGGGTKLEIK

SEQ ID NO: 621 - protein, 106 aa, synthetic construct
QIILTQSPAIMSASPGEKVTMTCSASSSVSNMYWYQQKPGSSPRLLIYDTSNLASGVPVRFSGSGSGASYSLTISRMEAEDAATYYCQQWSSYPWTFGGGTKLEIK

SEQ ID NO: 622 - protein, 108 aa, synthetic construct
EIVLTQSPTTMAASPGEKITITCSASSSISSNYLHWYQQKPGFSPKLLIYRTSNLASGVPARFSGSGSGTSYSLTIGTMEAEDVATYYCQQGSSIPLTFGAGTKLELK

SEQ ID NO: 623 - protein, 106 aa, synthetic construct
QIVLTQSPAILSASPGEKVTMTCSASSSVSYIYWYQQKPRSSPRLLIYDTSNLASGVPVRFSGSGSGTSYSLTISRMEAEDAATYYCQQWSSYPFTFGGGTKLEVK

SEQ ID NO: 624 - protein, 106 aa, synthetic construct
DIQMTQSPASLSASVGETVTITCRASGNIHNYLAWYQQKQGKSPQLLVYNAKTLADGVPSRFSGSGSGTQYSLKINSLQPEDFGYYYCQHFWSTPTFGGGTKLEIK

SEQ ID NO: 625 - protein, 107 aa, synthetic construct
DIVMTQSHKFMSTSVGDRVYITCKASQDVGTAVAWYQQTSGQSPKLLIYWASTRHTGVPDRFTGSGSGTDFTLTLSNVQSEDLADYFCQQYTSYPLTFGAGTKLELK

SEQ ID NO: 626 - protein, 112 aa, synthetic construct
NIMMTQSPSSLAVSAGEKVTMSCKSSQSVLYSSNQKNYLAWYQQKPGQSPQLLIYWASTRESGVPERFTGSGSGTDFTLTISSVQAEDLAVYYCHQYLSSWTFGGGTKLEIK

SEQ ID NO: 627 - protein, 107 aa, synthetic construct
DIVMTQAHRFMSTSVGDRVIISCKASQDVGTAVAWYQQTPGQSPKILIYWTSTRHTGVPDRFTGSRSGTDFTLTISNVQSEDLADYFCQQYTTYPLTFGGGTKLEIK

SEQ ID NO: 628 - protein, 107 aa, synthetic construct
DIVMTQSHKFMSTSIGDRVIITCKASQDVGTTVAWYQQRPGQSPKLLIYWASTRHTAVPDRFTGSGSGTDFTLTISNVQSEDLADYFCQQYTSYPLTFGAGTQLELK

SEQ ID NO: 629 - protein, 108 aa, synthetic construct; identical to SEQ ID NO: 314

SEQ ID NO: 630 - protein, 107 aa, synthetic construct
DIQMNQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLEIK

SEQ ID NO: 631 - protein, 107 aa, synthetic construct
DIKLTQSPSSIYTSLGERVTITCKASQDINTYLSWFQQRPGKSPKTLIYRADRLVDGVPSRVRGSGSGQDYSLTISSLEYEDMGIYYCLQYDEFPYTFGGGTKLEIK

SEQ ID NO: 632 - protein, 107 aa, synthetic construct; identical to SEQ ID NO: 315

SEQ ID NO: 633 - protein, 113 aa, synthetic construct
DVVMTQTPLSLPVSLGDQASISCRSSQSLVHSNGNTYLYWYFQKPGQSPKFLIYKVSNRFSGISDRFSGSGSGTDFTLKISRVEAEDLGVYFCSQSTHVPPFTFGSGTKLEIK

SEQ ID NO: 634 - protein, 113 aa, synthetic construct
DVVMTQTPLSLSVSLGDQASISCRSSQSLVHSNGNTYLYWYLQKPGQSPKLLIYKVSNRFSGITDRFSGSGSGTDFTLKISRVEAEDLGVYFCSQSTHVPPFTFGSGTKLEIK

SEQ ID NO: 635 - protein, 106 aa, synthetic construct; identical to SEQ ID NO: 316

SEQ ID NO: 636 - protein, 106 aa, synthetic construct; identical to SEQ ID NO: 317

SEQ ID NO: 637 - protein, 106 aa, synthetic construct; identical to SEQ ID NO: 318

SEQ ID NO: 638 - protein, 106 aa, synthetic construct
QIVLTQSPAVMSASPGEKVAMTCSASSSVTYMYWYQQKPGSSPRLLIYDTSNLASGVPVRFSGSGSGTSYSLTISRMEAEDAATYYCQQWSTYPFTPGSGTKLEIK

SEQ ID NO: 639 - protein, 106 aa, synthetic construct; identical to SEQ ID NO: 320

SEQ ID NO: 640 - protein, 111 aa, synthetic construct
DIVMTQSPASLAVSLGQRATISCRASESVDKYGISFLNWFQQKPGQPPKLLIYAASNQGSGVPARFSGSGSGTDFSLNIHPMEEDDPAMYFCQQGKEVPWTFGGGTKLEIK

SEQ ID NO: 641 - protein, 111 aa, synthetic construct
DIVMTQSPTSLAVSLGQRATISCRASESVDKYGISFLNWFQQKPGQPPRLLIYAASNQGSGVPARFSGSGSGTDFSLNIHPMEEDDPAVYFCQQGKEFPWTFGGGTKLEIK

SEQ ID NO: 642 - protein, 111 aa, synthetic construct
DIVMTQSPASLAVSLGQRATISCRASESVDKYGISFLNWFQQKPGQPPKLLIYAASNQGSGVPARFSGSGSGTDFSLNIHPMEEDDPAIYFCQQGKEVPWTFGGGTKLEIK

SEQ ID NO: 643 - protein, 107 aa, synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLLYTFGGGTTLEIK

SEQ ID NO: 644 - protein, 107 aa, synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLKYDNLLYTFGGGTKLEIK

SEQ ID NO: 645 - protein, 107 aa, synthetic construct; identical to SEQ ID NO: 620

SEQ ID NO: 646 - protein, 107 aa, synthetic construct; identical to SEQ ID NO: 620

SEQ ID NO: 647 - protein, 107 aa, synthetic construct
DIQMTQSPSSLSASLGGKVTITCKASQDINKYITWYQHKPGKGPRLLIHYTSTLQPGIPSRFSGSGSGRDYSFSISNLEPEDIATYYCLQYDNLLYTFGGGTKLEIK

SEQ ID NO: 648 - protein, 107 aa, synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLLYTFGGGTELEIK

SEQ ID NO: 649 - protein, 107 aa, synthetic construct
DIVMTQSHKFLSTSLGDRVSITCKASQDVGSAVAWYQQKPGQSPDLLIYWTFTRHTGVPDRFTGSRSGTDFTLTISNVQSGDLADYFCQQYSNYPLTFGGGTKLEIK

SEQ ID NO: 650 - protein, 112 aa, synthetic construct
DIVMSQSPSSLAVSVGEKVTMSCKSSQSLLYSGNQKNYLAWYQQKPGQSPKLLIYWASTRESGVPDRFTGSGSGTDFTLTISSVKAEDLAVYYCHQYYSYLTFGAGTKLELK

SEQ ID NO: 651 - protein, 107 aa, synthetic construct; identical to SEQ ID NO: 321

SEQ ID NO: 652 - protein, 107 aa, synthetic construct
DIQMTQTTSSLSASLGDRVTISCSGGQGISNYLNWYQQKPDGTFKLLIYYTSTLHSGVPSRFSGSGSGTDYSLTISNLEPEDVATYYCQQYSKLPYTFGGGTKLEIK

SEQ ID NO: 653 - protein, 109 aa, synthetic construct
DIVMTQSQKFMSTSVGDRVSVTCKASQNVGTNVAWYQQKPGQSPKALIYTASYRYSGVPDRFTGSGSGTDFTLTISNVQSEDLAEYFCQQYNSYPHMYTFGGGTKLEIK

SEQ ID NO: 654 - protein, 107 aa, synthetic construct; identical to SEQ ID NO: 322

SEQ ID NO: 655 - protein, 107 aa, synthetic construct; identical to SEQ ID NO: 323

SEQ ID NO: 656 - protein, 107 aa, synthetic construct; identical to SEQ ID NO: 324

SEQ ID NO: 657 - protein, 107 aa, synthetic construct; identical to SEQ ID NO: 323

SEQ ID NO: 658 - protein, 106 aa, synthetic construct
QIVLSQSPAILSASPGEKVTMTCRASSSVSYMHWYQQKPGSSPKPWIYATSNLASGVPARFSGSGSGTSYSLTISRVEAEDAATYYCQQWSSNPYTFGGGTKLEIK

SEQ ID NO: 659 - protein, 106 aa, synthetic construct
QIVLSQSPAILSASPGEKVTMTCRASSSVSYMHWYQQKPGSSPKPWIYATSNLASGVPTRFSGSGSGTSYSLTISKLEAEDAATYYCQQWSSNPYTFGGGTKLEIK

SEQ ID NO: 660 - protein, 107 aa, synthetic construct
DIVMTQSHKFMSTSVGDRVSITCKASQDVSTAVAWYQQKPGQSPKLLIFWTSTRHTGVPDRFTGSGSGTDYTLTISSVQAEDLALYYCQQHYTTPWTFGGGTKLEIK

SEQ ID NO: 661 - protein, 107 aa, synthetic construct
DIVMTQSHKFMSTSVGDRVSITCKASQDVSTAVAWYQQKPGQSPKLLIFWASTRHTGVPDRFTGSGSGTDYTLTISSVQAEDLALYYCQQHYSTPWTFGGGTKLEIK

SEQ ID NO: 662 - protein, 107 aa, synthetic construct
DIVMTQSHKFMSTSVGDRVSITCKASQDVSTAVDWYQQKPGQSPKLLIYWASTRHTGVPDRFTGSGSGTDYTLTISSVQAEDLALYYCQQHYSTPWTFGGGTKLEIK

SEQ ID NO: 663 - protein, 112 aa, synthetic construct; identical to SEQ ID NO: 326

SEQ ID NO: 664 - protein, 112 aa, synthetic construct; identical to SEQ ID NO: 327

SEQ ID NO: 665 - protein, 112 aa, synthetic construct; identical to SEQ ID NO: 328

SEQ ID NO: 666 - protein, 112 aa, synthetic construct; identical to SEQ ID NO: 329

SEQ ID NO: 667 - protein, 112 aa, synthetic construct; identical to SEQ ID NO: 329

SEQ ID NO: 668 - protein, 112 aa, synthetic construct; identical to SEQ ID NO: 331

SEQ ID NO: 669 - protein, 107 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYASYPFTFGSGTKLEIK

SEQ ID NO: 670 - protein, 107 aa, synthetic construct
DIQMTQSPTSLSASLGESVSLTCRASQEISGNLNWLQQKPDGSIKRLIYAASTLDSGVPKRFSGSRSGSDYSLTISSLESEDFADYYCLQYANYPFTFGSGTKLEVK

SEQ ID NO: 671 - protein, 107 aa, synthetic construct
DIVMTQSHKFMSTSVGDRVSITCKASQDVSTAVAWYQQKPGQSPKLLIYSASYRSTGVPDRFTGSGSGTDFTFTISSVQAEDLAVYYCQQHYSTPYTFGGGTKLEIR

SEQ ID NO: 672 - protein, 107 aa, synthetic construct
DIVMTQSHKFMSTSVGDRVTITCKASQDVSNAVAWYQLKPGQSPKLLIYSASYRYTGVPDRFTGSGSGTDFTFTISSVQAADLAVYYCQQHYITPYTFGGGTKLEIK

SEQ ID NO: 673 - protein, 112 aa, synthetic construct
DVLMTQTPLSLPVSLGDQASISCRSSQSIVHSDGNTYLEWYLQKPGQSPKLLIYRVSNRFSGVPDRFSGSGSGTDFTLKISRMEAEDLGVYYCFQGSHVPYTFGGGTKLEIK

SEQ ID NO: 674 - protein, 107 aa, synthetic construct; identical to SEQ ID NO: 332

SEQ ID NO: 675 - protein, 107 aa, synthetic construct
DIQMTQSPASLSVSVGETVTITCRASENIYSNLAWYQQKQGKSPQLLVYVATNLADGVPSRFSGSGSGTQYSLKINSLQSEDFGNYYCQHFWGTPYTFGGGTKLEMR

SEQ ID NO: 676 - protein, 111 aa, synthetic construct
DIVLTQSPASLAVSLGQRATISCRASESVEYYGTSLMQWYQQKPGQPPKLLINAASNVESGVPARFSGSGSGTDFSLNIHPVEEDDIAMYFCQQSRKVPWTFGGGTKLEIK

SEQ ID NO: 677 - protein, 107 aa, synthetic construct
ETTVTQSPASLSMAIGEKVTIRCITSTDIDDDMNWYQQKPGEPPKLLISEGNSLRPGVPSRFSSSGYGTDFVFTIEDMFSEDVADYHCLQSDNLPYTFGGGTKLEIK

SEQ ID NO: 678 - protein, 107 aa, synthetic construct
DIVMTQSQKFMSTSVGDRVSVTCKASQHVGTNVVWYQQKPGQSPKALIYSASYRYSGVPDRFSGSGSGTDFTLTISNVQSEDLAEYFCQQFNSYPLTFGGGTKLEIK

SEQ ID NO: 679 - protein, 106 aa, synthetic construct; identical to SEQ ID NO: 333

SEQ ID NO: 680 - protein, 107 aa, synthetic construct
DIVMTQSHKFMSTSVGDRVSITCKASQDVGTAVAWYQQRPGQSPKLLIYWASTRHTGVPDRFTGSGSGTDFTLTVSNVQSEDLADYFCQQYTSYPLTFGAGTKLELK

SEQ ID NO: 681 - protein, 111 aa, synthetic construct
DIVLTQSPASLAVSLGQRATISCRASESVDSYGNSFMHWYQQKPGQPPKLLIHRASNLESGIPARFSGSGSRTDFTLTINPVEADDVAIYYCQQTNDDPYTFGGGTNLEIK

SEQ ID NO: 682 - protein, 112 aa, synthetic construct; identical to SEQ ID NO: 334

SEQ ID NO: 683 - protein, 107 aa, synthetic construct
DIQMTQSPSSLSASLGERVSLTCRASQEISGYLSWLQQKPDGTIKRLIYAASTLDSGVPKRFSGSSSGSDYSLTISSLESDDFADYYCLQYASYPFTFGSGTKLEIK

SEQ ID NO: 684 - protein, 112 aa, synthetic construct
DVLMTQTPLSLPVSLGDQASISCRSSQTIVHSNGNTYLEWYLQKSGQSPKLLIYNVSNRFSGVPDRFRGSGSGTDFTLKISRVEAEDLGVYYCFQGSHVPYTFGSGTKLEIK

SEQ ID NO: 685 - protein, 108 aa, synthetic construct; identical to SEQ ID NO: 335

SEQ ID NO: 686 - protein, 108 aa, synthetic construct
EIVLTQSPALMTASPGEKVTITCSVSSSISSTNLHWYQQKSGTSPKPWIFGTSNLASGVPVRFSGSGSGTSYSLTISNMEAEDAATYYCQQWNSYPFTFGTGTKLEIK

SEQ ID NO: 687 - protein, 108 aa, synthetic construct
QIVLTQSPAIMSASPGEKVTMTCRASSSVSSTYLHWYQQKPGSSPKLWIYSTSNLASGVPPRFSGSGSGTSYSLTISSVEAEDAATYYCQQYDSSPNTFGAGTKLELK

SEQ ID NO: 688 - protein, 108 aa, synthetic construct; identical to SEQ ID NO: 336

SEQ ID NO: 689 - protein, 107 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPPTFGGGSKLEIK

SEQ ID NO: 690 - protein, 107 aa, synthetic construct
DIQMTQSPASLSASVGETVTITCGASENIYGALNWYQRKQGKSPQLLIYGATNLADGMSSRFSGSGSGRQYSLKISSLHPDDVATYYCQNVLSTPWTFGGGTKLEIK

SEQ ID NO: 691 - protein, 107 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGSPPTFGGGTKLEIK

SEQ ID NO: 692 - protein, 106 aa, synthetic construct; identical to SEQ ID NO: 337

SEQ ID NO: 693 - protein, 107 aa, synthetic construct; identical to SEQ ID NO: 338

SEQ ID NO: 694 - protein, 107 aa, synthetic construct
DIQMTQSPASLSVSTGETVTITCRASENIYSNLAWFQQKQGKSPQLLVYAATNLADGVPSRFSGSGSGTQYSLKITSLQSEDFGSYYCQHFWGTPLTFGAGTKLDLK

SEQ ID NO: 695 - protein, 112 aa, synthetic construct
DVLMTQNPLSLPVSLGDQASISCRSSQSIVHSNGNTYLEWYLQKPGQSPNLLIYNVFNRFSGVPDRFSGSGSGTDFTLKISRVEAEDLGVYYCFQGSHVPYTFGSGTKLEIK

SEQ ID NO: 696 - protein, 107 aa, synthetic construct
DIQMTQTTSSLSVSLGDRVTISCRASQDITNYLNWYQQKPDGTVKLLIYFTSRLHSGVPSRFSGSGSGTDYSLTISNLEPEDIATYYCQQYSKLPWTFGGGTKLEIK

SEQ ID NO: 697 - protein, 112 aa, synthetic construct; identical to SEQ ID NO: 673

SEQ ID NO: 698 - protein, 116 aa, synthetic construct; identical to SEQ ID NO: 306

SEQ ID NO: 699 - protein, 116 aa, synthetic construct
EVQLVESGGGLVQPGGSLRLSCAASGYTFTDYYMNWVRQAPGKGLEWIGDINPVTGDTSYNADFKRRFTFSLDTSKSTAYLQMNSLRAEDTAVYYCTREGPSFAYWGQGTLVTVSS

SEQ ID NO: 700 - protein, 116 aa, synthetic construct
EVQLVESGGGLVQPGGSLRLSCAASGYTFTDYYMNWVRQAPGKGLEWIGDINPITGDTSYNADFKRRFTFSLDTSKSTAYLQMNSLRAEDTAVYYCTREGPSFAYWGQGTLVTVSS

SEQ ID NO: 701 - protein, 116 aa, synthetic construct
EVQLVESGGGLVQPGGSLRLSCAASGYTFTDYYMNWVRQAPGKGLEWIGDINPVTGDTDYNADFKRRFTFSLDTSKSTAYLQMNSLRAEDTAVYYCTREGPSFAYWGQGTLVTVSS

SEQ ID NO: 702 - protein, 116 aa, synthetic construct
EVQLVESGGGLVQPGGSLRLSCAASGYTFTDYYMNWVRQAPGKGLEWIGDINPITGDTDYNADFKRRFTFSLDTSKSTAYLQMNSLRAEDTAVYYCTREGPSFAYWGQGTLVTVSS

SEQ ID NO: 703 - protein, 116 aa, synthetic construct
EVQLVESGGGLVQPGGSLRLSCAASGYTFTDYYINWVRQAPGKGLEWIGDINPITGDTDYNADFKRRFTFSLDTSKSTAYLQMNSLRAEDTAVYYCTREGPSFAYWGQGTLVTVSS

SEQ ID NO: 704 - protein, 116 aa, synthetic construct
EVQLVESGGGLVQPGGSLRLSCAASGYTFTSYYINWVRQAPGKGLEWIGDINPITGDTDYNADFKRRFTFSLDTSKSTAYLQMNSLRAEDTAVYYCTREGPSFAYWGQGTLVTVSS

SEQ ID NO: 705 - protein, 116 aa, synthetic construct
EVQLVESGGGLVQPGGSLRLSCAASGYTFTDYYINWVRQAPGKGLEWIGDINPITGDTDYNADFKRRFTFSLDTSKSTAYLQMSSLRAEDTAVYYCTREGPSFAYWGQGTLVTVSS

SEQ ID NO: 706 - protein, 116 aa, synthetic construct
EVQLVESGGGLVQPGGSLRLSCAASGYTFTSYYINWVRQAPGKGLEWIGDINPITGDTDYNADFKRRFTFSLDTSKSTAYLQMSSLRAEDTAVYYCTREGPSFAYWGQGTLVTVSS

SEQ ID NO: 707 - protein, 112 aa, synthetic construct; identical to SEQ ID NO: 339

SEQ ID NO: 708 - protein, 112 aa, synthetic construct
DIQLTQSPSSLSASVGDRVTITCRSSQTIVHSNGDTYLEWYQQKPGKAPNLLIRKVSNRFSGVPSRFSGSGSGTDFTLTISSLQPEDFATYYCFQGSHVPPTFGQGTKVEIK

SEQ ID NO: 709 - protein, 112 aa, synthetic construct
DIQLTQSPSSLSASVGDRVTITCRSSQTIVHSNGDTYLEWYQQKPGKAPNLLIYKVSNRFSGVPSRFSGSGSGTDFTLTISSLQPEDFATYYCFQGSHVPVTFGQGTKVEIK

SEQ ID NO: 710 - protein, 112 aa, synthetic construct
DIQLTQSPSSLSASVGDRVTITCRSSQTIVHSNGDTYLEWYQQKPGKAPNLLIRKVSNRFSGVPSRFSGSGSGTDFTLTISSLQPEDFATYYCFQGSHVPVTFGQGTKVEIK

SEQ ID NO: 711 - protein, 112 aa, synthetic construct
DIQLTQSPSSLSASVGDRVTITCRSSQTIVHSNGDTYLEWYQQKPGKAPNLLIGKVSNRFSGVPSRFSGSGSGTDFTLTISSLQPEDFATYYCFQGSHVPVTFGQGTKVEIK

SEQ ID NO: 712 - protein, 664 aa, synthetic construct; identical to SEQ ID NO: 118

SEQ ID NO: 713 - protein, 664 aa, synthetic construct; identical to SEQ ID NO: 118

SEQ ID NO: 714 - protein, 664 aa, synthetic construct; identical to SEQ ID NO: 118

SEQ ID NO: 715 - protein, 664 aa, synthetic construct; identical to SEQ ID NO: 122

SEQ ID NO: 716 - protein, 664 aa, synthetic construct; identical to SEQ ID NO: 122

SEQ ID NO: 717 - protein, 664 aa, synthetic construct; identical to SEQ ID NO: 122

SEQ ID NO: 718 - protein, 664 aa, synthetic construct; identical to SEQ ID NO: 170

SEQ ID NO: 719 - protein, 664 aa, synthetic construct; identical to SEQ ID NO: 170

SEQ ID NO: 720 - protein, 664 aa, synthetic construct; identical to SEQ ID NO: 170

SEQ ID NO: 721 - protein, 664 aa, synthetic construct; identical to SEQ ID NO: 128

SEQ ID NO: 722 - protein, 664 aa, synthetic construct; identical to SEQ ID NO: 128

SEQ ID NO: 723 - protein, 664 aa, synthetic construct; identical to SEQ ID NO: 128

SEQ ID NO: 724 - protein, 664 aa, synthetic construct; identical to SEQ ID NO: 134

SEQ ID NO: 725 - protein, 664 aa, synthetic construct; identical to SEQ ID NO: 134

SEQ ID NO: 726 - protein, 664 aa, synthetic construct; identical to SEQ ID NO: 134

SEQ ID NO: 727 - protein, 664 aa, synthetic construct; identical to SEQ ID NO: 266

SEQ ID NO: 728 - protein, 664 aa, synthetic construct; identical to SEQ ID NO: 266

SEQ ID NO: 729 - protein, 664 aa, synthetic construct; identical to SEQ ID NO: 266

SEQ ID NO: 730 - protein, 213 aa, synthetic construct; identical to SEQ ID NO: 28

SEQ ID NO: 731 - protein, 213 aa, synthetic construct; identical to SEQ ID NO: 29

SEQ ID NO: 732 - protein, 213 aa, synthetic construct; identical to SEQ ID NO: 30

SEQ ID NO: 733 - protein, 213 aa, synthetic construct; identical to SEQ ID NO: 28

SEQ ID NO: 734 - protein, 213 aa, synthetic construct; identical to SEQ ID NO: 29

SEQ ID NO: 735 - protein, 213 aa, synthetic construct; identical to SEQ ID NO: 30

SEQ ID NO: 736 - protein, 213 aa, synthetic construct; identical to SEQ ID NO: 28

SEQ ID NO: 737 - protein, 213 aa, synthetic construct; identical to SEQ ID NO: 29

SEQ ID NO: 738 - protein, 213 aa, synthetic construct; identical to SEQ ID NO: 30

SEQ ID NO: 739 - protein, 213 aa, synthetic construct; identical to SEQ ID NO: 28

SEQ ID NO: 740 - protein, 213 aa, synthetic construct; identical to SEQ ID NO: 29

SEQ ID NO: 741 - protein, 213 aa, synthetic construct; identical to SEQ ID NO: 30

SEQ ID NO: 742 - protein, 213 aa, synthetic construct; identical to SEQ ID NO: 28

SEQ ID NO: 743 - protein, 213 aa, synthetic construct; identical to SEQ ID NO: 29

SEQ ID NO: 744 - protein, 213 aa, synthetic construct; identical to SEQ ID NO: 30

SEQ ID NO: 745 - protein, 213 aa, synthetic construct; identical to SEQ ID NO: 28

SEQ ID NO: 746 - protein, 213 aa, synthetic construct; identical to SEQ ID NO: 29

SEQ ID NO: 747 - protein, 213 aa, synthetic construct; identical to SEQ ID NO: 30

SEQ ID NO: 748 - protein, 10 aa, synthetic construct
GGGGSGGGGS

SEQ ID NO: 749 - protein, 11 aa, synthetic construct
SASQDISNYLN

SEQ ID NO: 750 - protein, 7 aa, synthetic construct
FTSSLHS

SEQ ID NO: 751 - protein, 9 aa, synthetic construct
QQYSTVPWT

SEQ ID NO: 752 - protein, 10 aa, synthetic construct
GYTFTNYGMN

SEQ ID NO: 753 - protein, 17 aa, synthetic construct
WINTYTGEPTYAADFKR

SEQ ID NO: 754 - protein, 10 aa, synthetic construct
GYDFTHYGMN

SEQ ID NO: 755 - protein, 14 aa, synthetic construct
YPHYYGSSHWYFDV

SEQ ID NO: 756 - protein, 14 aa, synthetic construct
YPYYYGTSHWYFDV

(19) United States
(12) Patent Application Publication
(10) Pub. No.: US 2023/0250133 A1
(43) Pub. Date: Aug. 10, 2023

(54) METHODS OF PURIFYING A PRODUCT

(71) Applicant: Kodiak Sciences Inc., Palo Alto, CA (US)

(72) Inventors: Andreas Sebastian Zurbriggen, Termen (CH); Carole In-Albon, Lalden (CH)

(21) Appl. No.: 18/166,755

(22) Filed: Feb. 9, 2023

Related U.S. Application Data
(60) Provisional application No. 63/267,810, filed on Feb. 10, 2022.

(51) Int. Cl. C07K 1/22; C07K 16/22

(52) U.S. Cl. CPC C07K 1/22; C07K 16/22; C07K 2317/565

(57) ABSTRACT

Provided herein are embodiments relating to affinity chromatography purification and separation of contaminant species, including HCPs (Host Cell Proteins), from desired molecular and chemical species.

### SEQUENCE LISTING

The present application is being filed along with a Sequence Listing in electronic format. The Sequence Listing is provided as a file entitled KDIAK.141A.xml, created Feb. 8, 2023, which is 843,264 bytes in size. The information in the electronic format of the Sequence Listing is incorporated herein by reference in its entirety.

### INCORPORATION BY REFERENCE TO ANY PRIORITY APPLICATIONS

This application claims priority to U.S. Provisional App. No. 63/267,810, filed Feb. 10, 2022.

Any and all applications for which a foreign or domestic priority claim is identified in the Application Data Sheet as filed with the present application are hereby incorporated by reference under 37 CFR 1.57.

### BACKGROUND

### Field of the Invention

The present disclosure relates to methods of purifying antibodies and other cellular products.

### Background

Unit operations involved in the processing and purification of molecular species derived from cellular cultures involve the preferential separation of desirable and undesirable molecular species. In particular, chromatography, and protein A chromatography allow for preferential separation of certain species based on the interaction of various molecular bindings in comparison to any given wash or flow through buffer solution. Optimization of unit operations at this step may increase overall purity and percent yield of a desirable molecular species, while decreasing impurities.

### SUMMARY

Provided herein are methods of purifying a product by administration of a chaotropic agent.

Some embodiments provided herein allow one to improve product purity and composition by providing new purification methods for use in processing steps involved in product purification. In some embodiments, the product may be a protein product. In some embodiments, the purification involves chromatography. In some embodiments, the chromatography may be protein A, or protein G. In some embodiments, the chaotropic agent may comprise one or more of lithium and lithium salts, magnesium and magnesium salts, calcium and calcium salts and guanidinium and guanidinium salts.

Provided herein are methods of purifying a protein product using affinity chromatography. Some embodiments provided herein allow one to overcome certain limitations in the prior art by providing new methods for purifying a protein product after harvesting a cell culture. In some embodiments, the protein product is an antibody. In some embodiments, the protein product is an anti-VEGF antibody. In some embodiments, the anti-VEGF antibody of the present disclosure may be an anti-VEGF antibody conjugate (e.g., KSI-301, KSI-501), or anti-VEGF protein conjugate, that includes a polymeric moiety that extends the half-life (e.g., ocular half-life, etc.) of the antibody or protein when administered to a subject. In some embodiments, provided herein are compositions of buffer reagents that may be used to purify a protein product.

In some embodiments, provided herein is a method of purifying a product and reducing impurities from a load fluid comprising the protein and one or more impurities by passing the load fluid through an affinity chromatography matrix, followed by at least one wash solution comprising a chaotropic salt, and collecting the protein using an elution solution.

In some embodiments, provided herein is a method for separating impurities in an eluate comprising a protein of interest, the method comprising loading an eluent comprising a protein of interest onto an affinity chromatography matrix; and washing the affinity chromatography matrix with one or more buffer solutions comprising one or more of lithium and lithium salts, magnesium and magnesium salts, calcium and calcium salts and guanidinium and guanidinium salts.

In some embodiments provided herein is a method of producing a product using affinity chromatography. The method comprises loading an eluent containing a protein of interest onto an affinity chromatography matrix, then a first wash of the affinity chromatography matrix with a first buffer comprising sodium phosphate and a salt, and then a second wash of the affinity chromatography matrix with a second buffer comprising a chaotropic agent.

In some embodiments provided herein is a method of producing a product using affinity chromatography. The method comprising loading an eluent containing a protein of interest onto an affinity chromatography matrix, a first wash with a first buffer containing Tris and a salt, and a second wash with a second buffer containing Tris and a chaotropic agent, wherein the second buffer chaotropic agent is not the same salt as contained in the first buffer.

In some embodiments provided herein is a method of producing a product. The method comprising collecting a load fluid, wherein the load fluid comprises a protein of interest, loading the load fluid onto an affinity chromatography matrix, wherein the affinity chromatography matrix binds to the protein of interest, washing the affinity chromatography matrix with a buffer solution comprising a chaotropic salt, eluting the bound protein of interest; and collecting an eluate, wherein the eluate contains the protein of interest.

In some embodiments provided herein is a method of producing a product. The method comprising collecting a load fluid, wherein the load fluid comprises a protein of interest, loading the load fluid onto an affinity chromatography matrix, wherein the affinity chromatography matrix binds to the protein of interest, feeding to the affinity chromatography matrix a buffer solution comprising a chaotropic salt, eluting the bound protein of interest; and collecting an eluate, wherein the eluate contains the protein of interest.

In some embodiments provided herein is a method of producing a product, the method comprising collecting a conjugate protein, wherein the conjugate protein comprises an antibody bound to a conjugate polymer loading the conjugate protein onto an affinity chromatography matrix, wherein the affinity chromatography matrix binds to the conjugate protein, washing the affinity chromatography matrix with a buffer solution comprising a chaotropic salt, eluting the conjugate protein, and collecting an eluate, wherein the eluate contains the conjugate protein.

In some embodiments provided herein is a method of producing a product, the method comprising washing an affinity chromatography matrix bound to a target protein of interest with a buffer comprising a chaotropic salt, then eluting and collecting an eluate, wherein the eluate contains the target protein of interest, and removing viral contaminants from the eluate. In some embodiments of the method provided herein, removing viral contaminants from the eluate comprises one or more of low pH inactivation, detergent inactivation, polishing chromatography steps, viral filtration (VF), ultrafiltration (UF) and/or diafiltration (DF).

In some embodiments of the methods provided herein is provided a method of producing a product, the method comprising washing an affinity chromatography matrix bound to a target protein of interest with a buffer comprising a chaotropic salt, removing the chaotropic salt, and eluting and collecting an eluate, wherein the eluate contains the target protein of interest. In some embodiments of the methods provided herein the eluate is further combined with an acceptable pharmaceutical excipient to form a pharmaceutical composition. In some embodiments of the methods provided herein a buffer solution is added to the pharmaceutical composition. In some embodiments of the methods provided herein a preservative solution is added to the pharmaceutical composition. In some embodiments of the methods provided herein the pharmaceutical composition is further refined for intravitreal injection.

In some embodiments, provided herein is a method for processing a product. The method comprises loading an eluent into an affinity chromatography matrix. The method further comprises washing with a wash buffer comprising a chaotropic salt to collect an eluate, wherein the concentration of the chaotropic salt is increased from a first concentration to a second concentration, wherein the eluate is collected in at least one fraction, wherein the at least one fraction comprises a product of interest.

In some embodiments, provided herein is a method of producing a product, the method comprising collecting a load fluid, wherein the load fluid is comprised of a protein of interest, loading the load fluid into an affinity chromatography matrix, wherein the affinity chromatography matrix binds to the protein of interest, washing the affinity chromatography matrix with a buffer solution comprising a chaotropic salt, eluting and collecting an eluate, wherein eluate contain the target protein of interest, and removing viral contaminants from the eluate.

In some embodiments, provided herein is a method of producing a product, the method comprising loading an eluent into an affinity chromatography matrix, washing with a first wash buffer washing with a second wash buffer comprising a chaotropic salt, washing with a third wash buffer, wherein the third wash buffer removes the chaotropic salt, eluting with an elution buffer, wherein an eluate is collected, wherein the eluate comprises a protein product. In some embodiments, the first wash buffer comprises 50 mM Na-Phosphate. In some embodiments, the first wash buffer further comprises 250 mM NaCl. In some embodiments, provided herein is a method wherein the first wash buffer comprises Tris and a salt. In some embodiments, provided herein is a method further comprising removing viral contaminants from the eluate.

In some embodiments, provided herein is a method wherein removing viral contaminants comprises one or more of low pH inactivation, detergent inactivation, polishing chromatography steps, viral filtration (VF), ultrafiltration (UF), or diafiltration (DF). In some embodiments, the eluent comprises a protein of interest. In some embodiments, the protein of interest is an antibody. In some embodiments, the antibody is further conjugated to a polymer to form an antibody conjugate. In some embodiments, the antibody conjugate comprises a bispecific antibody. In some embodiments, the bispecific antibody comprises anti-VEGF and anti IL-6 binding moieties.

In some embodiments, the antibody conjugate has the structure of Formula (I):

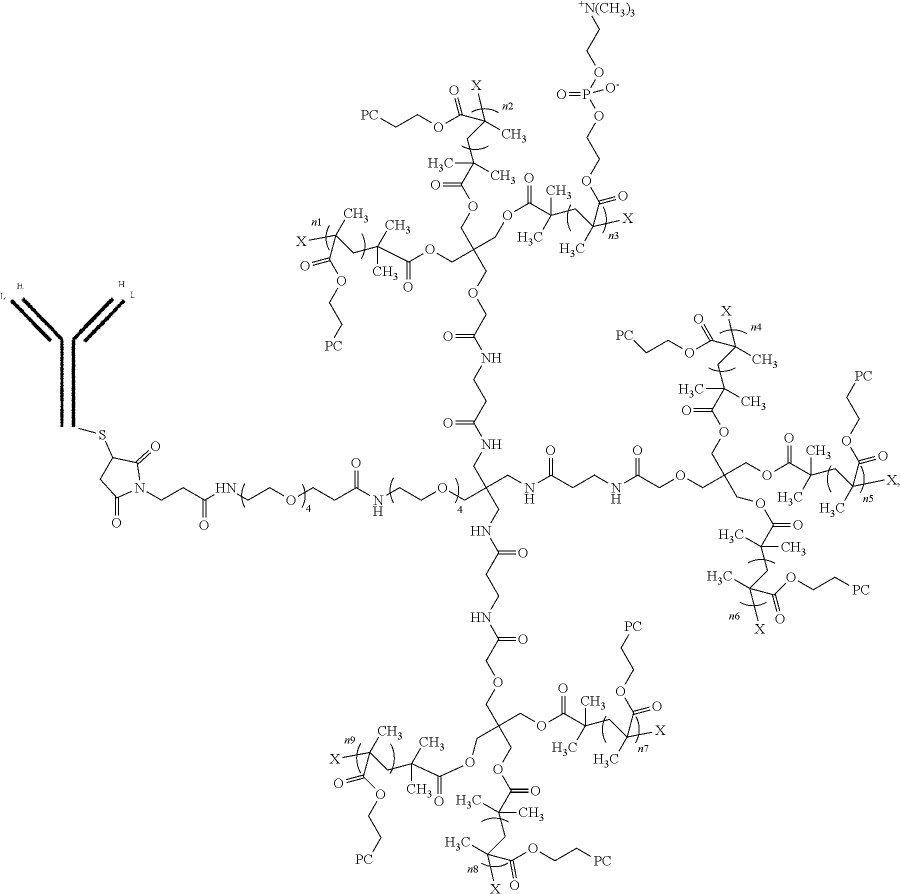

wherein each heavy chain of the anti-VEGF-A antibody is denoted by the letter H, and each light chain of the anti-VEGF-A antibody is denoted by the letter L, and the polymer is bonded to the anti-VEGF-A antibody through the sulfhydryl of C443 (EU numbering), which bond is depicted on one of the heavy chains. For purposes of the above structure, PC refers to a structure having the following:

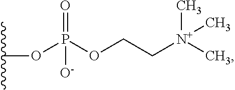 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

In some embodiments, provided herein is a method of producing a product, the method comprising recovering a cell culture supernatant, wherein the cell culture supernatant comprises a protein of interest, then processing the cell culture supernatant into an eluent, wherein the eluent comprises the protein of interest, then loading the eluent into an affinity chromatography matrix and proceeding to wash with a first wash buffer comprising Tris or Sodium Phosphate, then washing with a second wash buffer comprising a chaotropic salt, eluting with an elution buffer, wherein an eluate is collected, wherein the eluate comprises a protein product, then inactivating viral contaminants present in the eluate with a low pH viral buffer to yield a viral inactivated eluate, filtering the viral inactivated eluate, performing at least one round of ion exchange chromatography on the viral inactivated eluate, and filtering the viral inactivated eluate to yield a retentate, wherein the retentate comprises the protein of interest.

In some embodiments, the cell culture supernatant was produced in a bioreactor using animal component free cell culture. In some embodiments, the cell culture supernatant comprises harvesting cell products from a cell culture. In some embodiments, the cell culture is clarified to remove cells and cellular debris. In some embodiments, the eluent comprises the clarified cell culture supernatant.

In some embodiments, provided herein is a method of purifying a protein using affinity chromatography, the method comprising contacting a load fluid with a medium, wherein the medium is an affinity chromatography matrix that binds a protein of interest, then washing the medium with a buffer solution comprising a chaotropic agent, wherein the chaotropic agent is a salt, and contacting the washed medium with an elution solution under conditions suitable for eluting the protein of interest.

In some embodiments, provided herein is a method for producing a product, the method comprising applying the solution containing a protein of interest onto an affinity chromatography matrix, washing the affinity chromatography matrix with a first buffer, then washing the affinity chromatography matrix with a second buffer containing a chaotropic agent, then washing the affinity chromatography matrix with a third buffer to remove the chaotropic agent. Then eluting with an elution buffer, wherein an eluate is collected, wherein the eluate comprises a protein product.

In some embodiments, provided herein is a system for protein purification, comprising a column having a first antigen binding protein bound to the column, a phosphate wash buffer comprising sodium phosphate and a salt, an intermediate wash buffer comprising tris, a second wash buffer comprising magnesium chloride, and an elution buffer comprising sodium formate.

In some embodiments, provided herein is a system for protein purification, comprising: a column having a first antigen binding protein bound to the column; a first tris wash buffer comprising tris and a salt, an intermediate tris wash buffer, a second wash buffer comprising magnesium chloride, and an elution buffer comprising sodium formate. In some embodiments, the column comprises a ligand for affinity chromatography. In some embodiments, the ligand comprises protein A or protein G. In some embodiments, the first wash buffer comprising sodium phosphate and a salt has a pH between 5.5 and 9.5. In some embodiments, the phosphate wash buffer comprising sodium phosphate and a salt comprises about 50 mM sodium phosphate. In some embodiments, the phosphate wash buffer comprising sodium phosphate and a salt comprises about 250 mM NaCl. In some embodiments, the first tris wash buffer comprises about 50 mM Tris. In some embodiments, the first tris wash buffer further comprises about 250 mM NaCl. In some embodiments, the intermediate tris wash buffer comprises about 50 mM Tris. In some embodiments, the pH of the first tris wash buffer is about 7.2. In some embodiments, the pH of the second wash buffer is about 7.8. In some embodiments, the concentration of magnesium chloride in the second wash buffer is about 2.8 M. In some embodiments, the concentration of sodium formate in the elution buffer comprises 10 mM.

In some embodiments, described herein is a system for antibody purification, comprising a column having a protein A resin bound to an antibody, wherein the antibody comprises a light and heavy chain of at least one of SEQ ID NOs: 91-93, 28-30, and at least one of SEQ ID NOs: 7-13, 19-27, 89, 90, 256-262 respectively, a chaotropic wash buffer comprising a chaotropic salt, and an elution buffer comprising sodium formate.

In some embodiments, the protein of interest is a bispecific antibody. In some embodiments, the bispecific antibody is specific for VEGF and IL-6. In some embodiments, the protein of interest is an antibody conjugate. In some embodiments, the affinity chromatography matrix is a protein A chromatography matrix. In some embodiments, the chaotropic agent in the buffer solution is comprised of one or more of lithium and lithium salts, magnesium and magnesium salts, calcium and calcium salts and guanidinium and guanidinium salts. In some embodiments, the concentration of the one or more of lithium and lithium salts, magnesium and magnesium salts, calcium and calcium salts and guanidinium and guanidinium salts is between 0.05-3.5 M. In some embodiments, the buffer solution further comprises tris. In some embodiments, the concentration of tris in the buffer solution is at least 5 mM. In some embodiments, the pH of the buffer solution is greater than 5.5. In some embodiments, the eluate further contains viral impurities.

In some embodiments of the methods described herein, the methods further comprise removing viral impurities. In some embodiments of the methods described herein, the methods further comprise inactivating the viral impurities. In some embodiments of the methods described herein, the methods further comprise the step of washing the affinity chromatography matrix loaded with the load fluid with a prewash buffer solution prior to washing with the buffer solution. In some embodiments of the methods described herein, the methods further comprise the step of washing the affinity chromatography matrix loaded with the eluent with a post-wash buffer solution after washing with buffer solution. In some embodiments, the prewash buffer solution comprises sodium phosphate. In some embodiments, the prewash buffer solution comprises Tris and a salt. In some embodiments, the antibody conjugate has the structure of Formula (I),

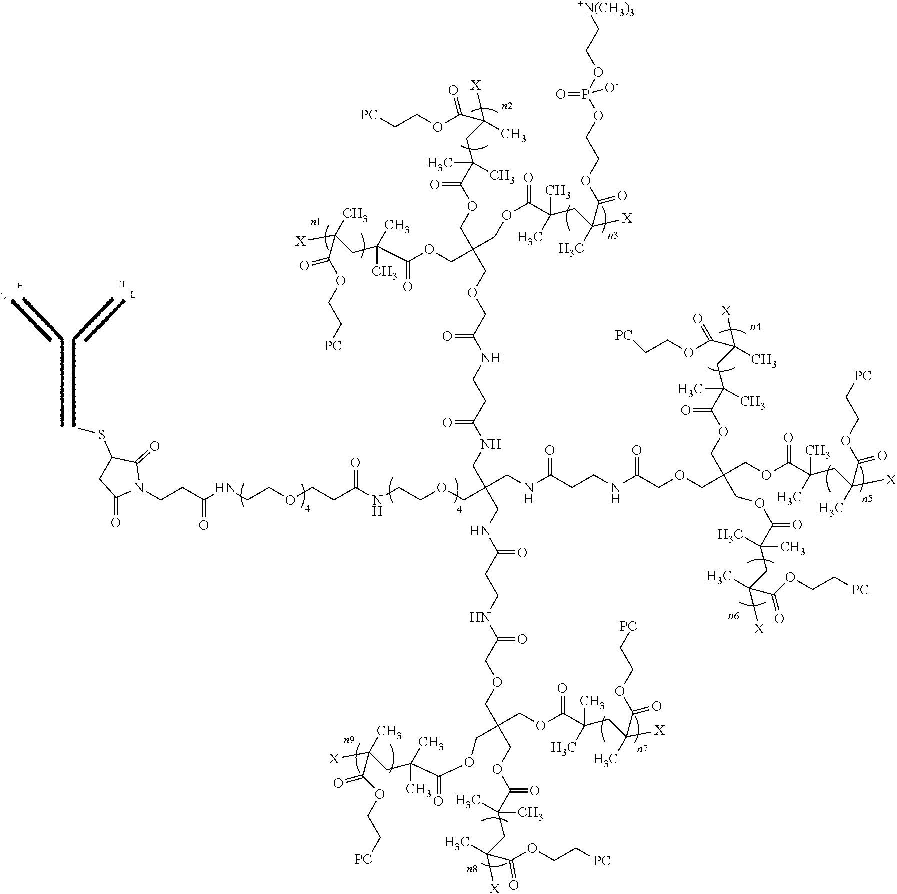

wherein each heavy chain of the anti-VEGF-A antibody is denoted by the letter H, and each light chain of the anti-VEGF-A antibody is denoted by the letter L, and the polymer is bonded to the anti-VEGF-A antibody through the sulfhydryl of C443 (EU numbering), which bond is depicted on one of the heavy chains, and where PC has the following structure:

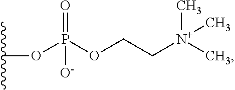 [SMILES: *OP(=O)([O-])OCC[N+](C)(C)C]

wherein the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

In some embodiments, the antibody conjugate comprises an anti-VEGF antibody conjugate comprising an anti-VEGF-A light chain and an anti-VEGF-A heavy chain, wherein the anti-VEGF-A antibody heavy chain comprises CDRH1: that is a CDRH1 in SEQ ID NO: 172, CDRH2: that is a CDRH2 in SEQ ID NO: 173, and CDRH3: that is a CDRH3 in SEQ ID NO: 174, and the anti-VEGF-A antibody light chain comprises CDRL1: that is a CDRL1 in SEQ ID NO: 199, CDRL2: that is a CDRL2 in SEQ ID NO: 200, and CDRL3: that is a CDRL3 in SEQ ID NO: 201.

In some embodiments, the anti-VEGF antibody conjugate comprises: an antibody conjugate comprising an anti-VEGF-A immunoglobulin G (IgG) bonded to a polymer, which polymer comprises MPC monomers, wherein the sequence of the anti-VEGF-A antibody heavy chain is at least one of SEQ ID Nos: 7-13, 19-27, 89, 90, 256-262, and the sequence of the anti-VEGF-A antibody light chain is at least one of SEQ ID Nos: 91-93, 28-30., and wherein the antibody is bonded at C449 to the polymer.

In some embodiments, the target protein of interest is produced by a cell culture. In some embodiments, the cell culture comprises CHO cells. In some embodiments, the methods described herein further comprise the step of washing the affinity chromatography matrix loaded with the eluent with a post-wash buffer solution after washing with buffer solution. In some embodiments, washing the affinity chromatography matrix with the buffer solution removes nucleic acids, endotoxins, antifoam agents, or other small molecules other than the target protein of interest. In some embodiments, washing the affinity chromatography matrix with the buffer solution removes impurities while keeping the target protein of interest bound to the affinity chromatography matrix. In some embodiments, washing the affinity chromatography matrix with the buffer solution removes host cell proteins besides the target protein of interest. In some embodiments, the addition of chaotropic agent in the buffer solution does not elute the target protein of interest. In some embodiments of the methods described herein, the methods further comprise one or more of virus inactivation, tangential flow filtration, diafiltration, ultrafiltration, ion exchange chromatography, or virus reduction filtration. In some embodiments, the eluent was produced in a bioreactor using animal component free cell culture. In some embodiments, the product is a protein of interest. In some embodiments, impurities comprise host cell protein impurities.

### BRIEF DESCRIPTION OF THE DRAWINGS

FIG. 1 describes a general protocol for some embodiments of the collection and purification of a protein of interest.

FIG. 2 describes a general protocol for the collection and purification of a protein of interest.

FIG. 3 describes a general protocol for column chromatography according to an embodiment of the present disclosure.

FIG. 4 describes a set of chromatography profiles run in different wash buffer solutions.

FIG. 5 describes a set of chromatography profiles run in different wash buffer solutions.

FIG. 6 describes a set of chromatography profiles run in different wash buffer solutions.

FIG. 7 describes a set of chromatography profiles run in different wash buffer solutions.

FIG. 8 is an amino acid sequence for some embodiments of an anti-VEGF antibody.

FIG. 9 depicts some embodiments of an IL-6-VEGF Trap fusion protein. The VEGF Trap domains are positioned at either at the N-terminus immediately preceding the variable domain (left) or positioned between the Fab region and the hinge region of the antibody (right).

FIG. 10 depicts the sequence listings of VEGF_trap_variant_1, VEGF_trap_variant_2, and VEGF_trap_variant_3.

FIG. 11 illustrates embodiments of Anti-IL-6 heavy chain variable region sequences. CDRs are underlined.

FIG. 12 illustrates various embodiments of VEGF trap sequences. Section that varies between the sequences are in bold and underlined. FIG. 12 further illustrates some embodiments of linker (GS) sequence embodiments. It can be present as a double repeat Gly-Gly-Gly-Gly-Ser linker (GS).

FIG. 13-14 illustrates some embodiments of heavy chain sequence for Anti-IL-6 molecules. CDRs are underlined.

FIG. 15 illustrates some embodiments of light chain sequences for Anti-IL-6 molecules. CDRs are underlined.

FIG. 16 illustrates some embodiments of heavy chain sequences for Anti-IL-6 molecules. CDRs are underlined.

FIGS. 17A-17B illustrate some embodiments of combinations of CDRs.

FIG. 18 illustrates some embodiments of VEGFR-Fc sequence variants. Section that varies between the sequences are in bold and underlined.

### DETAILED DESCRIPTION

In some embodiments, provided herein are methods and systems for reducing impurities by using chromatography to preferentially separate desirable and undesirable molecular species.

In some embodiments is method of purifying a product using affinity chromatography. In some embodiments, the method compromises: loading an eluent into an affinity chromatography matrix, wherein the affinity chromatography matrix binds to a protein of interest; and washing the affinity chromatography matrix with a buffer solution comprising a chaotropic agent.

In some embodiments is a method of purifying a product and reducing impurities from a load fluid comprising the protein and one or more impurities by passing the load fluid through an affinity chromatography matrix, followed by at least one wash solution comprising a chaotropic salt, and collecting the protein using an elution solution.

In some embodiments is a method for separating impurities in an eluate comprising a protein of interest. In some embodiments, the method comprises loading an eluent comprising a protein of interest onto an affinity chromatography matrix; and washing the affinity chromatography matrix with one or more buffer solutions comprising magnesium or a magnesium salt.

In some embodiments is a method of producing a product using affinity chromatography. In some embodiments, the method comprises loading an eluent containing a protein of interest onto an affinity chromatography matrix, a first wash of the affinity chromatography matrix with a first buffer comprising sodium phosphate and a salt, and a second wash of the affinity chromatography matrix with a second buffer comprising a chaotropic agent.

In some embodiments is a method of producing a product using affinity chromatography. In some embodiments, the method comprises loading an eluent containing a protein of interest onto an affinity chromatography matrix, a first wash with a first buffer containing Tris and a salt, a second wash with a second buffer containing Tris and a chaotropic agent, wherein the second buffer chaotropic agent is not the same salt as contained in the first buffer.

In some embodiments is a method of producing a product. In some embodiments, the method comprises (i) collecting a load fluid, wherein the load fluid comprises a protein of interest, (ii) loading the load fluid onto an affinity chromatography matrix, wherein the affinity chromatography matrix binds to the protein of interest, (iii) washing the affinity chromatography matrix with a buffer solution comprising a chaotropic salt, (iv) eluting the bound protein of interest; and (v) collecting an eluate, wherein the eluate contains the protein of interest.

In some embodiments is a method of producing a product. In some embodiments, the method comprises collecting a load fluid, wherein the load fluid comprises a protein of interest, loading the load fluid onto an affinity chromatography matrix, wherein the affinity chromatography matrix binds to the protein of interest, feeding to the affinity chromatography matrix a buffer solution comprising a chaotropic salt, eluting the bound protein of interest; and collecting an eluate, wherein the eluate contains the protein of interest.

In some embodiments is a method of producing a product. In some embodiments, the method comprises collecting a conjugate protein, wherein the conjugate protein comprises an antibody bound to a conjugate polymer loading the conjugate protein onto an affinity chromatography matrix, wherein the affinity chromatography matrix binds to the conjugate protein, washing the affinity chromatography matrix with a buffer solution comprising a chaotropic salt, eluting the conjugate protein, and collecting an eluate, wherein the eluate contains the conjugate protein.

In some embodiments is a method of producing a product. In some embodiments, the method comprises washing an affinity chromatography matrix bound to a target protein of interest with a buffer comprising a chaotropic salt, eluting and collecting an eluate, wherein the eluate contains the target protein of interest, and removing viral contaminants from the eluate. In some embodiments, removing viral contaminants from the eluate comprises: one or more of low pH inactivation, detergent inactivation, polishing chromatography steps, viral filtration (VF), ultrafiltration (UF) and/or diafiltration (DF).

In some embodiments is a method of producing a product. In some embodiments, the method comprises washing an affinity chromatography matrix bound to a target protein of interest with a buffer comprising a chaotropic salt, removing the chaotropic salt, and eluting and collecting an eluate, wherein the eluate contains the target protein of interest. In some embodiments, the eluate is further combined with an acceptable pharmaceutical excipient to form a pharmaceutical composition. In some embodiments, a buffer solution is added to the pharmaceutical composition. In some embodiments, a preservative solution is added to the pharmaceutical composition. In some embodiments, the pharmaceutical composition is further refined for intravitreal injection.

In some embodiments is a method of producing a product. In some embodiments, the method comprises collecting a load fluid, wherein the load fluid is comprised of a protein of interest, loading the load fluid into an affinity chromatography matrix, wherein the affinity chromatography matrix binds to the protein of interest, washing the affinity chromatography matrix with a buffer solution comprising a chaotropic salt, eluting and collecting an eluate, wherein eluate contain the target protein of interest, and removing viral contaminants from the eluate.

In some embodiments is a method of producing a product. In some embodiments, the method comprises loading an eluent into an affinity chromatography matrix, washing with a first wash buffer, washing with a second wash buffer comprising a chaotropic salt, washing with a third wash buffer, wherein the third wash buffer removes the chaotropic salt, eluting with an elution buffer, wherein an eluate is collected, wherein the eluate comprises a protein product. In some embodiments, the first wash buffer comprises 50 mM Na-Phosphate. In some embodiments, the first wash buffer further comprises 250 mM NaCl. In some embodiments, the first wash buffer comprises Tris and a salt. In some embodiments, the method further comprises removing viral contaminants from the eluate. In some embodiments, removing viral contaminants comprises: one or more of low pH inactivation, detergent inactivation, polishing chromatography steps, viral filtration (VF), ultrafiltration (UF), or diafiltration (DF). In some embodiments, the eluent comprises a protein of interest. In some embodiments, the protein of interest is an antibody. In some embodiments, the antibody is further conjugated to a polymer to form an antibody conjugate. In some embodiments, the antibody conjugate has the structure of Formula (I):

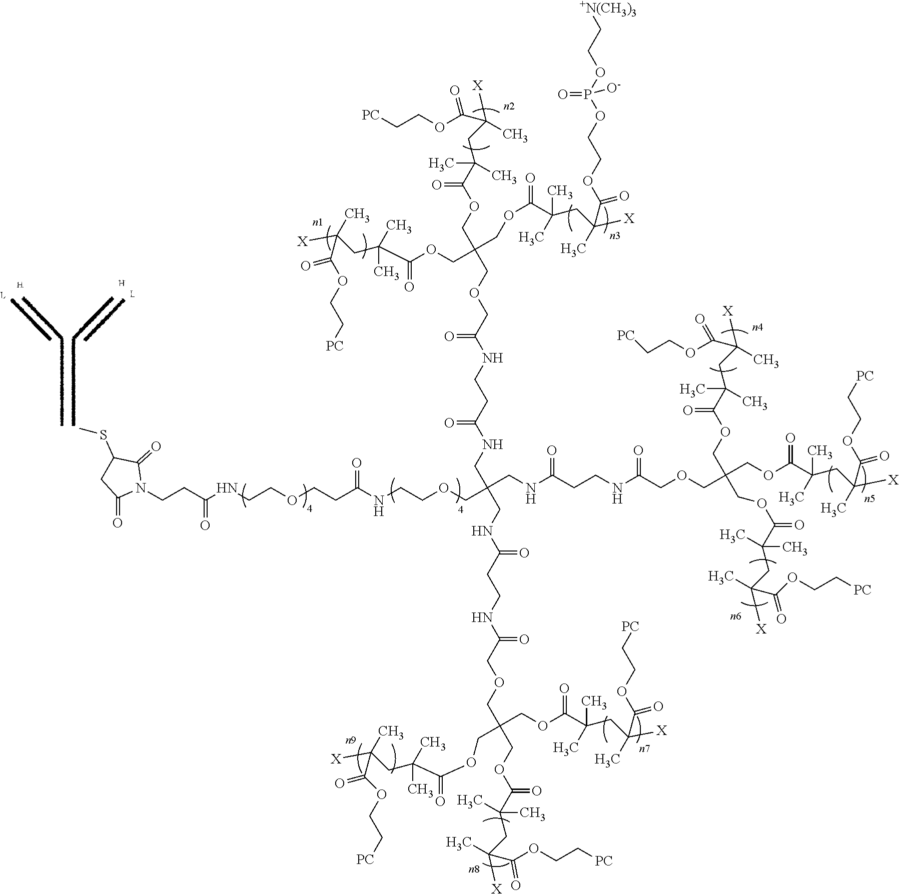

wherein: each heavy chain of the anti-VEGF-A antibody is denoted by the letter H, and each light chain of the anti-VEGF-A antibody is denoted by the letter L; the polymer is bonded to the anti-VEGF-A antibody through the sulfhydryl of C443 (EU numbering), which bond is depicted on one of the heavy chains; PC is:

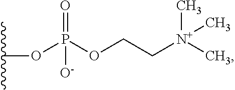 [SMILES: *OP(=O)([O-])OCC[N+](C)(C)C]

wherein the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%. In some embodiments, the antibody conjugate comprises a bispecific antibody. In some embodiments, the bispecific antibody comprises anti-VEGF and anti IL-6 binding moieties.

In some embodiments, the antibody conjugate has the structure of Formula (II):

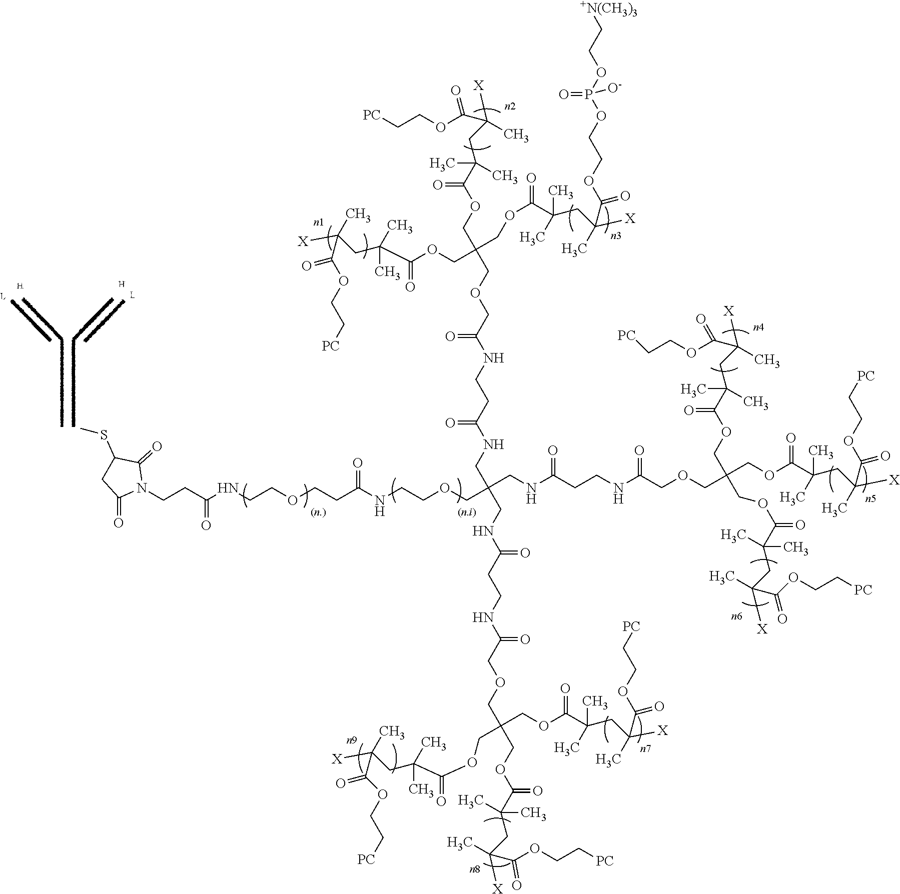

wherein: wherein “n.” is an integer from 1 to 50 and “n.i” is an integer from 1 to 50; each heavy chain of the anti-VEGF-A antibody is denoted by the letter H, and each light chain of the anti-VEGF-A antibody is denoted by the letter L; the polymer is bonded to the anti-VEGF-A antibody through the sulfhydryl of C443 (EU numbering), which bond is depicted on one of the heavy chains; PC is:

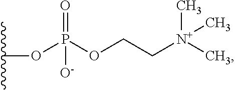 [SMILES: *OP(=O)([O-])OCC[N+](C)(C)C]

wherein the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%. In some embodiments, the antibody conjugate comprises a bispecific antibody. In some embodiments, the bispecific antibody comprises anti-VEGF and anti IL-6 binding moieties.

In some embodiments is a method of producing a product. In some embodiments, the method comprises recovering a cell culture supernatant, wherein the cell culture supernatant comprises a protein of interest, processing the cell culture supernatant into an eluent, wherein the eluent comprises the protein of interest, loading the eluent into an affinity chromatography matrix, washing with a first wash buffer comprising Tris or Sodium Phosphate, washing with a second wash buffer comprising a chaotropic salt, eluting with an elution buffer, wherein an eluate is collected, wherein the eluate comprises a protein product, inactivating viral contaminants present in the eluate with a low pH viral buffer to yield a viral inactivated eluate, filtering the viral inactivated eluate, performing at least one round of ion exchange chromatography on the viral inactivated eluate, and filtering the viral inactivated eluate to yield a retentate, wherein the retentate comprises the protein of interest. In some embodiments, the cell culture supernatant was produced in a bioreactor using animal component free cell culture. In some embodiments, processing the cell culture supernatant comprises harvesting cell products from a cell culture. In some embodiments, the cell culture is clarified to remove cells and cellular debris. In some embodiments, the eluent comprises the clarified cell culture supernatant.

In some embodiments is a method of purifying a protein using affinity chromatography. In some embodiments, the method comprises contacting a load fluid with a medium, wherein the medium is an affinity chromatography matrix that binds a protein of interest, washing the medium with a buffer solution comprising a chaotropic agent, wherein the chaotropic agent is a salt, and contacting the washed medium with an elution solution under conditions suitable for eluting the protein of interest.

In some embodiments is a method of producing a product. In some embodiments, the method comprises applying the solution containing a protein of interest onto an affinity chromatography matrix, washing the affinity chromatography matrix with a first buffer, washing the affinity chromatography matrix with a second buffer containing a chaotropic agent, washing the affinity chromatography matrix with a third buffer to remove the chaotropic agent, eluting with an elution buffer, wherein an eluate is collected, wherein the eluate comprises a protein product.

In some embodiments is a system for protein purification. In some embodiments, the system comprises a column having a first antigen binding protein bound to the column; a phosphate wash buffer comprising sodium phosphate and a salt, an intermediate wash buffer comprising tris, a second wash buffer comprising magnesium chloride, and an elution buffer comprising sodium formate,

In some embodiments is a system for protein purification. In some embodiments, the system comprises a column having a first antigen binding protein bound to the column; a first tris wash buffer comprising tris and a salt, an intermediate tris wash buffer, a second wash buffer comprising magnesium chloride, and an elution buffer comprising sodium formate, In some embodiments, the column comprises a ligand for affinity chromatography. In some embodiments, the ligand comprises protein A or Protein G. In some embodiments, the first wash buffer comprising sodium phosphate and a salt has a pH between 5.5 and 9.5. In some embodiments, the phosphate wash buffer comprising sodium phosphate and a salt comprises about 50 mM sodium phosphate. In some embodiments, the phosphate wash buffer comprising sodium phosphate and a salt comprises about 250 mM NaCl. In some embodiments, the first tris wash buffer comprises about 50 mM Tris. In some embodiments, the first tris wash buffer further comprises about 250 mM NaCl. In some embodiments, the intermediate tris wash buffer comprises about 50 mM Tris. In some embodiments, the pH of the first tris wash buffer is about 7.2. In some embodiments, the pH of the second wash buffer is about 7.8. In some embodiments, the concentration of magnesium chloride in the second wash buffer is about 2.8 M. In some embodiments, the concentration of sodium formate in the elution buffer comprises 10 mM.

In some embodiments is a system for antibody purification. In some embodiments, the system comprises a column having a protein A resin bound to an antibody, wherein the antibody comprises a light and heavy chain of at least one of SEQ ID NOs: 91-93, 28-30., and at least one of SEQ ID NOs: 7-13, 19-27, 89, 90, 256-262, respectively, and; a chaotropic wash buffer comprising a chaotropic salt, and an elution buffer comprising sodium formate. In some embodiments, the protein of interest is a bispecific antibody. In some embodiments, the bispecific antibody is specific for VEGF and IL-6. In some embodiments, the bispecific antibody is OG2072. In some embodiments, the protein of interest is an antibody conjugate. In some embodiments, the affinity chromatography matrix is a protein A chromatography matrix. In some embodiments, the chaotropic agent in the buffer solution is comprised of one or more of a lithium, lithium salt, magnesium, magnesium salt, calcium, calcium salt, guanidinium, and/or guanidinium salt. In some embodiments, the concentration of the one or more of a lithium, lithium salt, magnesium, magnesium salt, calcium, calcium salt, guanidinium, and/or guanidinium salt is between 0.05-3.5 M, respectively. In some embodiments, the buffer solution further comprises tris. In some embodiments, the concentration of tris in the buffer solution is at least 5 mM. In some embodiments, the pH of the buffer solution is greater than 5.5. In some embodiments, the eluate further contains viral impurities. In some embodiments, the method further comprises removing the viral impurities. In some embodiments, the method further comprises inactivating the viral impurities. In some embodiments, the method further comprises the step of washing the affinity chromatography matrix loaded with the load fluid with a prewash buffer solution prior to washing with the buffer solution. In some embodiments, the method further comprises the step of washing the affinity chromatography matrix loaded with the eluent with a postwash buffer solution after washing with buffer solution. In some embodiments, the prewash buffer solution comprises sodium phosphate. In some embodiments, the prewash buffer solution comprises Tris and a salt. In some embodiments, the antibody conjugate has the structure of Formula (I),

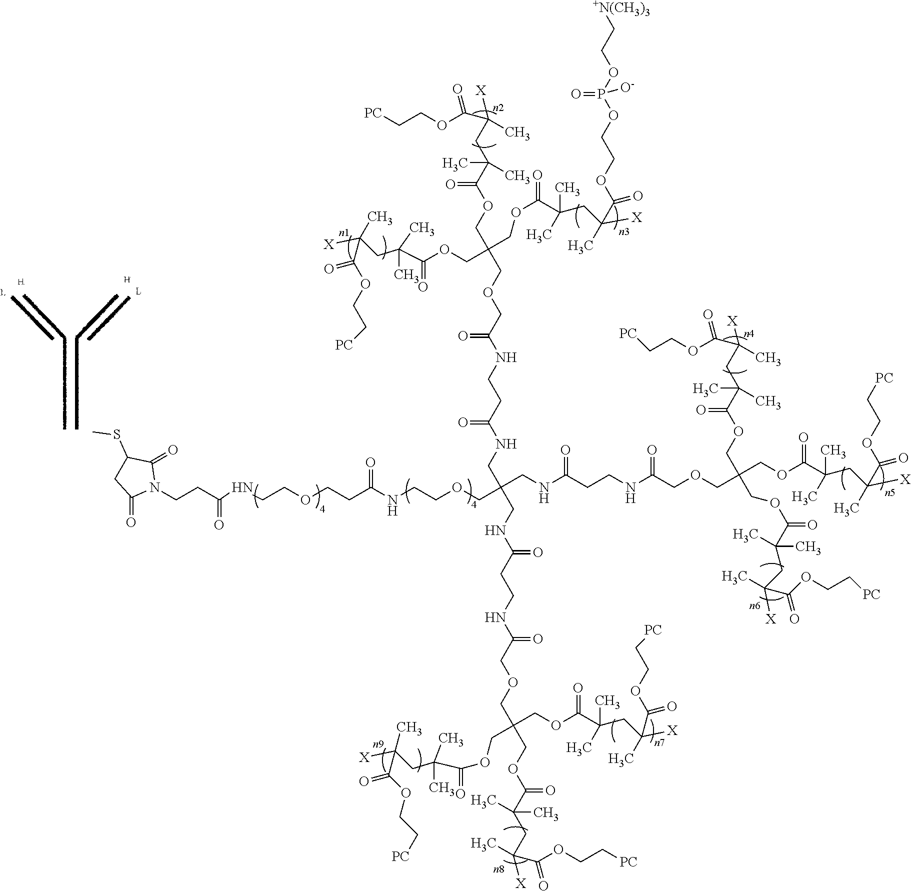

wherein: each heavy chain of the anti-VEGF-A antibody is denoted by the letter H, and each light chain of the anti-VEGF-A antibody is denoted by the letter L; the polymer is bonded to the anti-VEGF-A antibody through the sulfhydryl of C443 (EU numbering), which bond is depicted on one of the heavy chains; PC is:

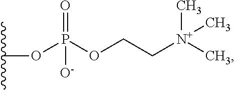 [SMILES: *OP(=O)([O-])OCC[N+](C)(C)C]

wherein the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%. In some embodiments, the antibody conjugate comprises an anti-VEGF antibody conjugate comprising an anti-VEGF-A light chain and an anti-VEGF-A heavy chain, wherein the anti-VEGF-A antibody heavy chain comprises CDRH1: that is a CDRH1 in SEQ ID NO: 172, CDRH2: that is a CDRH2 in SEQ ID NO: 173, and CDRH3: that is a CDRH3 in SEQ ID NO: 174, and the anti-VEGF-A antibody light chain comprises CDRL1: that is a CDRL1 in SEQ ID NO: 199, CDRL2: that is a CDRL2 in SEQ ID NO: 200, and CDRL3: that is a CDRL3 in SEQ ID NO: 201. In some embodiments, the anti-VEGF antibody conjugate comprises: an antibody conjugate comprising an anti-VEGF-A immunoglobulin G (IgG) bonded to a polymer, which polymer comprises MPC monomers, wherein the sequence of the anti-VEGF-A antibody heavy chain is at least one of SEQ ID NOs: 7-13, 19-27, 89, 90, 256-262, and the sequence of the anti-VEGF-A antibody light chain is at least one of SEQ ID NOs: 91-93, 28-30, and wherein the antibody is bonded at C449 to the polymer.

In some embodiments, the target protein of interest is produced by a cell culture. In some embodiments, the cell culture comprises CHO cells. In some embodiments, the method further comprises the step of washing the affinity chromatography matrix loaded with the eluent with a postwash buffer solution after washing with buffer solution. In some embodiments, washing the affinity chromatography matrix with the buffer solution removes nucleic acids, endotoxins, antifoam agents, or other small molecules other than the target protein of interest. In some embodiments, washing the affinity chromatography matrix with the buffer solution removes impurities while keeping the target protein of interest bound to the affinity chromatography matrix. In some embodiments, washing the affinity chromatography matrix with the buffer solution removes host cell proteins besides the target protein of interest. In some embodiments, the addition of chaotropic agent in the buffer solution does not elute the target protein of interest. In some embodiments, the method further comprises one or more of virus inactivation, tangential flow filtration, diafiltration, ultrafiltration, ion exchange chromatography, or virus reduction filtration. In some embodiments, the eluent was produced in a bioreactor using animal component free cell culture. In some embodiments, the product is a protein of interest. In some embodiments, impurities comprise host cell protein impurities. In some embodiments, the first wash buffer comprises 10 mM Na-Phosphate. In some embodiments, the first wash buffer comprises a phosphate-based species. In some embodiments, the first wash buffer further comprises 50 mM NaCl.

In some embodiments is a method for processing a product. In some embodiments, the method comprises loading an eluent into an affinity chromatography matrix; and washing with a wash buffer comprising a chaotropic salt to collect an eluate, wherein the concentration of the chaotropic salt is increased from a first concentration to a second concentration, wherein the eluate is collected in at least one fraction, wherein the at least one fraction comprises a product of interest. In some embodiments, the concentration of the chaotropic salt at the first concentration is 0 M, wherein the concentration of the chaotropic salt at the second concentration is 4.0 M. In some embodiments, the chaotropic salt is one or more of a lithium, lithium salt, magnesium, magnesium salt, calcium, calcium salt, guanidinium, and/or guanidinium salt. In some embodiments, the chaotropic salt is selected from magnesium chloride, calcium chloride, lithium chloride, and guanidinium hydrochloride.

In some embodiments, the buffer solution further comprises one or more of the following: Acetate, Citrate, ACES, BES, Bicine, HEPES, MES, MOPS, MOPSO, TAPS, Tricine, Bis-Tris, Bis-Tris propane, Cacodylate, CAPS, CAPSO, CHES, Glycine, Glycylglycine, Imidazole, PIPES, TEA, or TES.

In some embodiments, unwanted molecules are removed from the solution using a methodology that comprises chaotropic agents to dissolve unspecific interactions. In some embodiments, chaotropic agents are used as part of an affinity chromatography step. In some embodiments, chaotropic agents are used as part of ion exchange chromatography step. In some embodiments, the chaotropic agents used as part of ion exchange chromatography have low conductivity like urea, alcohols and detergents. In some embodiments, unwanted molecules are removed from the solution using a methodology that comprises hydrophobic interaction resins, wherein the protein of interest is bound onto the resin with a kosmotropic agent and eluted while applying a gradient to a chaotropic salts.

When collecting from a cell culture, while a desirable molecular species may be present in significant concentrations, there may also exist other undesirable molecular species, such as host cell proteins, cellular debris, nucleic acids, endotoxins, or other molecules that can compromise the purity of the desirable molecular species. Based on differential properties related to binding, shape, size, charge, and other physical properties, it becomes possible to sort and purify various supernatant compositions. In some embodiments, purification of the desirable molecular species may allow for better patient outcomes and fewer complications as a result of administration of said desirable molecular species. In some embodiments, the present methods may allow for increased efficacy, or lower dosage of the desirable molecular species.

In some embodiments, the desirable molecular species is collected from blood. In some embodiments, the desirable molecular species is collected from plasma. In some embodiments, the desirable molecular species is collected from a cell culture. In some embodiments, the desirable molecular species is collected from animal component free cell culture. In some embodiments, the desirable molecular species is collected from clarified cell culture fluid (CCCF). In some embodiments, the desirable molecular species is collected from cellular supernatant.

In some embodiments, the cell culture is bacterial. In some embodiments, the cell culture is E. coli. In some embodiments, the cell culture is eukaryotic. In some embodiments the cell culture is S. cerevisiae. In some embodiments, the cell culture is mammalian. In some embodiments, the cell culture is from a human cell. In some embodiments, the cell culture is primary tissue. In some embodiments, the cell culture is an established cell line. Non-limiting examples of an established cell line include CHO cells, HeLa cells, mouse 3T3 fibroblasts, HEK293 cells, and KT-3 cells. In some embodiments, the cell culture comprises a mixture of cell types. In some embodiments, the cell culture comprises a single cell type. In some embodiments, the cell culture is an immune cell. In some embodiments, the cell culture is a lymphocyte. In some embodiments, the cell culture is a T cell.

In some embodiments, the desirable molecular species may comprise an anti-VEGF antibody conjugate that includes a polymeric moiety that extends the half-life of the antibody when administered to a subject (e.g. KSI-301, KSI-501). In some embodiments, the undesirable molecular species may comprise host cell proteins (HCPs). In some embodiments, chromatography may be achieved using a protein A substrate, wherein administration of certain chaotropic agents may efficiently elute undesirable molecular species, while keeping desirable molecular species bound to the substrate. Thus, in some embodiments, the methods and systems of the present disclosure may provide for a method of purification of cellular products harvested from cell cultures. In some embodiments, the present methods can achieve a higher purity of desired molecular species with lower rates of loss as cellular processing proceeds. In some embodiments, the present methods can reduce levels of HCPs so as to reduce patient complications when administering a final drug product. In some embodiments, the present methods may allow for decreased costs or time associated with purifying cellular products.

Harvesting and processing cell cultures can involve significant purification and collecting efforts, as often whole cell extracts possess not only desirable molecular species, but also undesirable impurities. Multi-stage processing of collected cell cultures allows for the efficient removal of impurities, including host cell proteins (HCPs), product-related impurities such as high molecular weight (HMW) species and low molecular weight (LMW) species. In the first stage of a multi-stage purification process for a protein of interest (e.g., an antibody), some processes use affinity chromatography, wherein the efficiency and purity of the resultant protein of interest affects all downstream purification procedures. Additionally, affinity chromatography during downstream processing may serve to concentrate the product, allowing for the use of proportionally smaller apparatus in later processing steps, which may serve to decrease costs and time spent during processing. Therefore, there is an advantage in optimizing the removal of impurities during affinity chromatography, without losing or comprising the yield of concentration for subsequent processing steps.

In some embodiments, the method comprises purifying an antibody and/or protein. In some embodiments, the method involves an optimized wash buffer. In some embodiments, the optimized wash buffer is at pH 6.0, pH 6.5, pH 7.0, pH 7.5, pH 8.0, pH 8.5, pH 9.0, or any integer that is between pH 6.0 and pH 9.0. In some embodiments, the optimized wash buffer is at pH 7.0. In some embodiments, the optimized wash buffer is at pH 7.2. In some embodiments, the optimized wash buffer is at pH 6.0.

In some embodiments, the optimized wash buffer comprises 5 mM, 10 mM, 25 mM, 50 mM, 100 mM, 150 mM, 200 mM, or any integer that is between 5 mM and 200 mM of sodium phosphate (Na-Phosphate). In some embodiments, the optimized wash buffer comprises 5 mM Na-Phosphate. In some embodiments, the optimized wash buffer comprises 50 mM Na-Phosphate.

In some embodiments, the optimized wash buffer comprises 5 mM, 10 mM, 25 mM, 50 mM, 100 mM, 150 mM, 200 mM, or any integer that is between 5 mM and 200 mM of Tris. In some embodiments, the optimized wash buffer comprises 5 mM Tris. In some embodiments, the optimized wash buffer comprises 50 mM Tris.

In some embodiments, the optimized wash buffer comprises 5 mM, 10 mM, 25 mM, 50 mM, 100 mM, 150 mM, 200 mM, or any integer that is between 5 mM and 200 mM of Bis-Tris. In some embodiments, the optimized wash buffer comprises 5 mM Bis-Tris. In some embodiments, the optimized wash buffer comprises 50 mM Bis-Tris.

In some embodiments, the optimized wash buffer comprises 50 mM, 100 mM, 250 mM, 500 mM, 750 mM, 1 M, 1.5 M, 2 M, 3 M or any integer that is between 50 mM and 3 M of NaCl. In some embodiments, the optimized wash buffer comprises 2 M NaCl. In some embodiments, the optimized wash buffer comprises 50 mM NaCl.

In some embodiments, the optimized wash buffer comprises 1 M, 1.5 M, 1.65 M, 2 M, 2.8 M, 3 M, 4 M, 5 M or any integer that is between 1 M and 5 M of MgCl₂. In some embodiments, the optimized wash buffer comprises 2.8 M MgCl₂. In some embodiments, the optimized wash buffer comprises 2 M MgCl₂.

In some embodiments, the optimized wash buffer comprises a chaotrop. In some embodiments, the optimized wash buffer comprises a chaotrop salt. In some embodiments, the optimized wash buffer comprises a chaotropic cation. In some embodiments, the optimized wash buffer comprises a chaotropic anion. Non-limiting examples of a chaotropic cations include guanidinium, magnesium, calcium, sodium, and lithium. Non-limiting examples of chaotropic anions include chloride, sulfate, acetate, citrate, nitrate, and nitrite. In some embodiments, the chaotrop salt is CaCl2), guanidinium chloride, Li-Acetate, LiCl, MgCl2, MgSO4, NaNO3, or NaCl. In some embodiments, the optimized wash buffer comprises two or more chaotrop salts. In some embodiments, the chaotrop is present in the optimized wash buffer at 0.05M, 0.1 M, 0.2M, 0.5 M, 1 M, 1.5 M, 1.65M, 2 M, 2.8M, 3 M, 4M, 5 M, 6 M, 7M, 10 M or any integer that is between 0.05 M and 10 M. In some embodiments, the chaotrop is present in the optimized wash buffer at 1 M. In some embodiments, the chaotrop is present in the optimized wash buffer at 2.8 M. In some embodiments, the concentration of the chaotrop present in the optimized wash buffer is optimized for the protein and/or antibody that is being purified.

### Relevance

As described herein, a wash procedure was established that alleviates HCP levels in the eluate of an affinity chromatography step. the ranges for buffer strength, conductivity, and pH within the buffer were also defined. Salts were identified that allowed reducing HCP levels in the eluate. It was found that the salts' efficiency in reducing HCP levels correlate with their chaotropic strength as per the Hofmeister series. It was also found that this approach works for different antibody types and different Fc fusion proteins, and therefore the concept of the invention as disclosed herein can be applied generically to use with any antibody and/or protein. The technique was also shown to work with two different Protein A affinity chromatography resins.

The work described here used different antibodies and antibody-like constructs derived from Chinese Hamster Ovary (CHO) cells. The CHO expression system is the most common system used in the industry for the production biopharmaceuticals due to its ability to produce complex proteins with post-translational modifications similar to those produced in humans. So far, more than 6,000 CHO HCPs have been identified.

Downstream processing of biopharmaceutical products of mammalian cell culture currently accounts for a large fraction of the total production cost. A major challenge in the downstream processing is the removal of Host cell proteins (HCPs). HCPs are process-related impurities that may copurify with biopharmaceutical drug products. Downstream processing typically includes Protein A affinity chromatography step followed by additional polishing steps to remove aggregates, product variants, HCPs, and host cell DNA. Many of the same HCPs are found across the biopharmaceutical industry after the Protein A chromatography step. This is a result of the industry almost universally using CHO cells for the production of antibodies and antibody-like constructs as well as using the Protein A affinity chromatography step with very comparable condition. For the latter the biopharmaceutical is applied to the resin in physiological conditions (neutral pH and physiological salt conditions), washing the resin with physiological conditions as well as high sodium chloride concentration buffer followed by an elution step to collect the product. As a result of this platform approach, the affinity chromatography eluate contains many of the same HCPs and comparable levels. There is some but small variance based on the type of biopharmaceutical product. In a survey, 69% of companies indicated that they had experienced issues with individual HCPs during drug production and are considered one of the biggest challenges in biomanufacturing. Some HCPs are high-risk and can include those that are immunogenic, biologically active, or enzymatically active with the potential to degrade either product molecules or excipients used in formulation. In process development, the need to remove HCPs is easy to recognize, but it is hard but important to alleviate HCP levels as much as possible. Some of these high-risk HCPs are lipases, which are enzymes that break down fats and lipids. Lipases can also degrade polysorbates, which are often used in formulations, thus affecting the stability of the therapeutic product. Similarly, hydrolases were found to be a root cause for polysorbate degradation. Such HCPs may then compromise stability and reduce the shelf life of drug products. Polysorbate degradation can also lead to the formation of particles in formulations, creating safety concerns. Then, proteases like serine proteases, cathepsins, and metalloproteinases can degrade antibodies, antibody-like constructs, and other proteins. In conclusion, various HCP species can impact both active ingredients and excipients in biologic formulations. Importantly, HCPs also have the potential to be immunogenic in humans. An unwanted immune response can be the most serious harm caused by HCPs and can even be lethal. Phospholipase B-like 2 protein was found in lebrikizumab and shown to trigger immune responses in approximately 90% of subjects based on data from clinical studies. An overview of high/risk HCPs and their potential impact is shown on Table 5A. For all these reasons, it is crucial to alleviate HCP levels as much as possible. Some HCPs unspecifically bind to the product and are then co-purified. These HCPs are the most challenging to remove. This work focused on disintegrating these unspecific interactions of product and HCPs and thereby alleviating HCP levels. The characterization of the HCPs types that were removed through this approach was beyond the scope of this work. Here the inventors show that through the application of a wash step that contains a chaotropic salts the level of HCPs can be alleviated significantly. It remains to be seen what type of HCPs are removed.

### Definitions

The term “cell” includes those of prokaryotes and eukaryotes, and may further include bacterial cells, mycobacteria cells, fungal cells, yeast cells, plant cells, insect cells, non-human animal cells, human cells, or cell fusions such as, for example, hybridomas. In some embodiments, the cell is a mammalian cell. In some embodiments, the cell is derived from human, monkey, ape, hamster, rat, or mouse cells. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes.

As used herein, “affinity chromatography” is a method that makes use of the specific, reversible interactions between biomolecules to effect chromatographic separation.

As used herein, “Protein A chromatography” refers to a specific affinity chromatographic method that relies on the affinity of the IgG binding domains of Protein A to the Fc portion of an immunoglobulin molecule. In immunoglobulin, the Fc portion comprises immunoglobulin constant domains CH2 and CH3 or immunoglobulin domains substantially similar to these. Protein A was derived from native protein from the cell wall of Staphylococcus aureus, and Protein A produced by recombinant or synthetic methods, as well as variants of Protein A can retain the ability to bind to an Fc region. Protein A chromatography is often immobilized to a solid support, like those in a protein A column. Protein G and Protein L may also be used in an analogous manner. In some embodiments, the solid support is a matrix which is adhered to protein A.

As used herein, the term “affinity chromatography matrix” or “AC matrix”, is intended to refer to a solid phase medium, typically a gel or resin, that allows for separation of biochemical mixtures based on a highly specific binding interaction between a protein of interest and the AC matrix, such as between a receptor and ligand, enzyme and substrate or antigen and antibody. Thus, the solid phase medium comprises a target to which the protein of interest is capable of reversibly affixing, depending upon the buffer conditions. Non-limiting examples of immobilized or solid phase media that can comprise the AC matrix include a gel matrix, such as agarose beads (such as commercially available Sepharose matrices), and a glass matrix, such as porous glass beads (such as commercially available ProSep matrices).

In some embodiments, the methodology for isolating the protein of interest comprises column chromatography. In this process, an AC matrix is formed into a column, and a biochemical mixture containing a protein of interest is flowed through the column. The protein of interest becomes bound to the AC matrix. This is then followed by washing of the column by flowing through the column a wash solution, followed by elution of the protein of interest from the column by flowing through the column an elution buffer.

In some embodiments, the methodology for isolating the protein of interest comprises membrane chromatography. Membrane chromatography methods rely on the AC matrix formatted to fit on a membranous sheet, wherein a biochemical mixture containing the protein of interest is flowed through the membrane. A non-limiting example of membrane chromatography is Sartorius' Sartobind Rapid A. In some embodiments, wherein the protein of interest is at least about 500 kDa, membrane chromatography is the preferred chromatography method.

Further affinity chromatography systems which can be employed in the invention include, for example Protein G, Protein A/G and Protein L columns, each of which are also immunoglobulin-binding bacterial proteins with binding properties established in the art. Thus, an AC matrix that is a Protein G matrix, a Protein A/G matrix or a Protein L matrix can be used to purify antibodies, antibody fragments, or proteins comprising an Fc region (e.g., Fc fusion proteins).

While the present disclosure is described in particular with respect to purification of antibodies using Protein A, insofar as any protein (including fusion proteins) is known the art to selectively bind to a particular AC matrix, the protein is amenable to purification using the washing methods described herein.

As used herein, “chaotropic agents” are molecules which weaken or otherwise interfere with non-covalent forces and increase entropy within biomolecular systems. In some embodiments, lithium chloride, magnesium chloride, calcium chloride and/or guanidinium chloride are a chaotropic agent. Non-limiting examples of chaotropic agents include butanol, calcium chloride, ethanol, guanidinium chloride, lithium perchlorate, lithium acetate, magnesium chloride, phenol, propanol, sodium dodecyl sulfate, thiourea, and urea. Chaotropic agents include salts that affect the solubility of proteins. The more chaotropic anions include for example chloride, nitrate, bromide, chlorate, iodide, perchlorate, and thiocyanate. The more chaotropic cations include for example lithium, magnesium, calcium, and guanidinium.

As used herein, “eluent,” or “eluant” refers to the carrier portion of the mobile phase in chromatography. In liquid chromatography, an eluent is the liquid solvent entering into the column, while in gas chromatography, it is the carrier gas. In some embodiments, eluent refers to a carrier liquid solvent comprising antibodies, host cell proteins (HCPs), and other molecules of interest.

As used herein, “eluate” refers to the analyte material that emerges from a chromatographic step, and includes both analytes and solutes passing through a solid phase. In some embodiments, eluate refers specifically to the analyte material that is collected for further processing. In some embodiments, eluate refers to antibodies collected from harvested cells, otherwise known as the “product eluate.” In some embodiments, eluate refers to HCPs collected from harvested cells, otherwise known as the “wash eluate.”

A “neovascular disorder” is a disorder or disease state characterized by altered, dysregulated or unregulated angiogenesis. Examples of neovascular disorders include neoplastic transformation (e.g. cancer) and ocular neovascular disorders including diabetic retinopathy, age-related macular degeneration, and retinal vein occlusion.

An “ocular neovascular” disorder is a disorder characterized by altered, dysregulated or unregulated angiogenesis in the eye of a patient. Such disorders include retinal vein occlusion, optic disc neovascularization, iris neovascularization, retinal neovascularization, choroidal neovascularization, corneal neovascularization, vitreal neovascularization, glaucoma, pannus, pterygium, macular edema, diabetic retinopathy, diabetic macular edema, vascular retinopathy, retinal degeneration, uveitis, inflammatory diseases of the retina, and proliferative vitreoretinopathy.

The term antibody includes intact antibodies and binding fragments thereof. A binding fragment refers to a molecule other than an intact antibody that comprises a portion of an intact antibody that binds the antigen to which the intact antibody binds. Examples of binding fragments include Fv, Fab′, Fab′-SH, F(ab′)2; diabodies; linear antibodies; single-chain antibody molecules (e.g. scFv); and multispecific antibodies formed from antibody fragments. scFv antibodies are described in Houston J S. 1991. Methods in Enzymol. 203:46-96. In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

Specific binding of an antibody to its target antigen(s) means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody or fusion protein binds one and only one target.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once the antibodies or fusion proteins have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcR binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2^{nd} ed. Raven Press, N.Y., 1989), Ch. 7) (incorporated by reference in its entirety for all purposes).

The mature variable regions of each light/heavy chain pair form the antibody binding site. Thus, an intact antibody has two binding sites, i.e., is divalent. In natural antibodies, the binding sites are the same. However, bispecific antibodies can be made in which the two binding sites are different (see, e.g., Songsivilai S, Lachmann P C. 1990. Bispecific antibody: a tool for diagnosis and treatment of disease. Clin Exp Immunol. 79:315-321; Kostelny S A, Cole M S, Tso J Y. 1992. Formation of bispecific antibody by the use of leucine zippers. J Immunol. 148: 1547-1553). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. For convenience, the variable heavy CDRs can be referred to as CDR_{H}1, CDR_{H}2 and CDR_{H}3; the variable light chain CDRs can be referred to as CDR_{L}1, CDR_{L}2 and CDR_{L}3. The assignment of amino acids to each domain is in accordance with the definitions of Kabat E A, et al. 1987 and 1991. Sequences of Proteins of Immunological Interest (National Institutes of Health, Bethesda, Md.) or Chothia C, Lesk A M. 1987. Canonical Structures for the Hypervariable Regions of Immunoglobulins. J Mol Biol 196:901-917; Chothia C, et al. 1989. Conformations of Immunoglobulin Hypervariable Regions. Nature 342:877-883. Kabat also provides a widely used numbering convention (Kabat numbering) in which corresponding residues between different heavy chain variable regions or between different light chain variable regions are assigned the same number. Although Kabat numbering can be used for antibody constant regions, EU numbering is more commonly used, as is the case in this application. Although specific sequences are provided for exemplary antibodies disclosed herein, it will be appreciated that after expression of protein chains one to several amino acids at the amino or carboxy terminus of the light and/or heavy chain, particularly a heavy chain C-terminal lysine residue, may be missing or derivatized in a proportion or all of the molecules.

The term “epitope” refers to a site on an antigen to which an antibody or extracellular trap segment binds. An epitope on a protein can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody (or Fab fragment) bound to its antigen to identify contact residues.

Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50: 1495, 1990). A test antibody competes with a reference antibody if an excess of a test antibody (e.g., at least 2×, 5×, 10×, 20× or 100×) inhibits binding of the reference antibody by at least 50%. In some embodiments the test antibody inhibits binding of the reference antibody by 75%, 90%, or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “patient” includes human and other mammalian subjects that receive either prophylactic or therapeutic treatment.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. Sequence identities of other sequences can be determined by aligning sequences using algorithms, such as BESTFIT, FASTA, and TFASTA in the Wisconsin Genetics Software Package Release 7.0, Genetics Computer Group, 575 Science Dr., Madison, Wis., using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occur in both sequences to yield the number of matched positions, dividing the number of matched positions by the total number of positions in the window of comparison (i.e., the window size), and multiplying the result by 100 to yield the percentage of sequence identity.

Compositions or methods “comprising” one or more recited elements may include other elements not specifically recited. For example, a composition that comprises antibody may contain the antibody alone or in combination with other ingredients.

The term “antibody-dependent cellular cytotoxicity”, or ADCC, is a mechanism for inducing cell death that depends upon the interaction of antibody-coated target cells (i.e., cells with bound antibody) with immune cells possessing lytic activity (also referred to as effector cells). Such effector cells include natural killer cells, monocytes/macrophages and neutrophils. ADCC is triggered by interactions between the Fc region of an antibody bound to a cell and Fcy receptors, particularly FcγRI and FcγRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term opsonization also known as “antibody-dependent cellular phagocytosis”, or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component Clq which in turn activates the complement cascade leading to target cell death. Activation of complement may also result in deposition of complement components on the target cell surface that facilitate ADCC by binding complement receptors (e.g., CR3) on leukocytes.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. Nos. 5,859,205, 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly, a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly, a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (which can be as defined by Kabat) from a mouse antibody, they can also be made with less than all CDRs (e.g., at least 3, 4, or 5 CDRs from a mouse antibody) (e.g., De Pascalis R, Iwahashi M, Tamura M, et al. 2002. Grafting “Abbreviated” Complementary-Determining Regions Containing Specificity-Determining Residues Essential for Ligand Contact to Engineer a Less Immunogenic Humanized Monoclonal Antibody. J Immunol. 169:3076-3084; Vajdos F F, Adams C W, Breece T N, Presta L G, de Vos A M, Sidhu, S S. 2002. Comprehensive functional maps of the antigen-binding site of an anti-ErbB2 antibody obtained with shotgun scanning mutagenesis. J Mol Biol. 320: 415-428; Iwahashi M, Milenic D E, Padlan E A, et al. 1999. CDR substitutions of a humanized monoclonal antibody (CC49): Contributions of individual CDRs to antigen binding and immunogenicity. Mol Immunol. 36:1079-1091; Tamura M, Milenic D E, Iwahashi M, et al. 2000. Structural correlates of an anticarcinoma antibody: Identification of specificity-determining regions (SDRs) and development of a minimally immunogenic antibody variant by retention of SDRs only. J Immunol. 164:1432-1441).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan E A. 1991. A possible procedure for reducing the immunogenicity of antibody variable domains while preserving their ligand-binding properties. Mol Immunol. 28:489-98) with residues from the corresponding positions of a human antibody sequence. The result is an antibody in which the CDRs are entirely or substantially from a non-human antibody and the variable region frameworks of the non-human antibody are made more human-like by the substitutions. A human antibody can be isolated from a human, or otherwise result from expression of human immunoglobulin genes (e.g., in a transgenic mouse, in vitro or by phage display). Methods for producing human antibodies include the trioma method of Ostberg L, Pursch E. 1983. Human×(mouse×human) hybridomas stably producing human antibodies. Hybridoma 2:361-367; Ostberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991) and phage display methods (see, .e.g. Dower et al., WO 91/17271 and McCafferty et al., WO 92/01047, U.S. Pat. Nos. 5,877,218, 5,871,907, 5,858,657, 5,837,242, 5,733,743 and 5,565,332.

“Polymer” refers to a series of monomer groups linked together. A polymer is composed of multiple units of a single monomer (a homopolymer) or different monomers (a heteropolymer). High MW polymers are prepared from monomers that include, but are not limited to, acrylates, methacrylates, acrylamides, methacrylamides, styrenes, vinyl-pyridine, vinyl-pyrrolidone and vinyl esters such as vinyl acetate. Additional monomers are useful in high MW polymers. When two different monomers are used, the two monomers are called “comonomers,” meaning that the different monomers are copolymerized to form a single polymer. The polymer can be linear or branched. When the polymer is branched, each polymer chain is referred to as a “polymer arm.” The end of the polymer arm linked to the initiator moiety is the proximal end, and the growing-chain end of the polymer arm is the distal end. On the growing chain-end of the polymer arm, the polymer arm end group can be the radical scavenger, or another group.

“Initiator” refers to a compound capable of initiating a polymerization using monomers or comonomers. The polymerization can be a conventional free radical polymerization or a controlled/“living” radical polymerization, such as Atom Transfer Radical Polymerization (ATRP), Reversible Addition-Fragmentation-Termination (RAFT) polymerization or nitroxide mediated polymerization (NMP). The polymerization can be a “pseudo” controlled polymerization, such as degenerative transfer. When the initiator is suitable for ATRP, it contains a labile bond which can be homolytically cleaved to form an initiator fragment, I, being a radical capable of initiating a radical polymerization, and a radical scavenger, I′, which reacts with the radical of the growing polymer chain to reversibly terminate the polymerization. The radical scavenger I′ is typically a halogen, but can also be an organic moiety, such as a nitrile. In some embodiments, the initiator contains one of more 2-bromoisobutyrate groups as sites for polymerization via ATRP.

A “chemical linker” refers to a chemical moiety that links two groups together, such as a half-life extending moiety and a protein. The linker can be cleavable or non-cleavable. Cleavable linkers can be hydrolyzable, enzymatically cleavable, pH sensitive, photolabile, or disulfide linkers, among others. Other linkers include homobifunctional and heterobifunctional linkers. A “linking group” is a functional group capable of forming a covalent linkage consisting of one or more bonds to a bioactive agent. Non-limiting examples include those illustrated in Table 1 of WO2013059137 (incorporated by reference).

The term “reactive group” refers to a group that is capable of reacting with another chemical group to form a covalent bond, i.e. is covalently reactive under suitable reaction conditions, and generally represents a point of attachment for another substance. The reactive group is a moiety, such as maleimide or succinimidyl ester, is capable of chemically reacting with a functional group on a different moiety to form a covalent linkage. Reactive groups generally include nucleophiles, electrophiles and photoactivatable groups.

“Phosphorylcholine,” also denoted as “PC,” refers to the following:

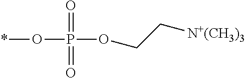

where * denotes the point of attachment. The phosphorylcholine is a zwitterionic group and includes salts (such as inner salts), and protonated and deprotonated forms thereof.

“Phosphorylcholine containing polymer” is a polymer that contains phosphorylcholine. “Zwitterion containing polymer” refers to a polymer that contains a zwitterion.

Poly(acryloyloxyethyl phosphorylcholine) containing polymer refers to a polymer containing 2-(acryloyloxy)ethyl-2-(trimethylammonium)ethyl phosphate (HEA-PC shown below in Example 6) as monomer.

Poly(methacryloyloxyethyl phosphorylcholine) containing polymer refers to a polymer containing 2-(methacryloyloxy)ethyl-2-(trimethylammonium)ethyl phosphate (HEMA-PC or MPC) as monomer (see below):

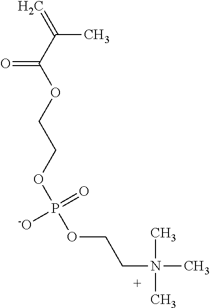

As used herein, “MPC” and “HEMA-PC” are interchangeable.

“Molecular weight” in the context of the polymer can be expressed as either a number average molecular weight, or a weight average molecular weight or a peak molecular weight. Unless otherwise indicated, all references to molecular weight herein refer to the peak molecular weight. These molecular weight determinations, number average (Mn), weight average (Mw) and peak (Mp), can be measured using size exclusion chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight, or the use of light scattering techniques, ultracentrifugation or viscometry to determine weight average molecular weight. In some embodiments, the molecular weight is measured by SEC-MALS (size exclusion chromatography-multi angle light scattering). In some embodiments, the polymeric reagents are typically polydisperse (i.e., number average molecular weight and weight average molecular weight of the polymers are not equal), and can possess low polydispersity values of, for example, less than about 1.5, as judged, for example, by the PDI value derived from the SEC-MALS measurement. In some embodiments, the polydispersities (PDI) are in the range of about 1.4 to about 1.2. In some embodiments the PDI is less than about 1.15, 1.10, 1.05, or 1.03.

The phrase “a” or “an” entity refers to one or more of that entity; for example, a compound refers to one or more compounds or at least one compound. As such, the terms “a” (or “an”), “one or more”, and “at least one” can be used interchangeably herein.

“About” means variation one might see in measurements taken among different instruments, samples, and sample preparations.

“Protected,” “protected form,” “protecting group” and “protective group” refer to the presence of a group (i.e., the protecting group) that prevents or blocks reaction of a particular chemically reactive functional group in a molecule under certain reaction conditions. Protecting groups vary depending upon the type of chemically reactive group being protected as well as the reaction conditions to be employed and the presence of additional reactive or protecting groups in the molecule, if any. Suitable protecting groups include those such as found in the treatise by Greene et al., “Protective Groups In Organic Synthesis,” 3^{rd} Edition, John Wiley and Sons, Inc., New York, 1999.

“Alkyl” refers to a straight or branched, saturated, aliphatic radical having the number of carbon atoms indicated. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together.

The term “lower” referred to above and hereinafter in connection with organic radicals or compounds respectively defines a compound or radical which can be branched or unbranched with up to and including 7 or up to and including 4 and (as unbranched) one or two carbon atoms.

“Alkylene” refers to an alkyl group, as defined above, linking at least two other groups, i.e., a divalent hydrocarbon radical. The two moieties linked to the alkylene can be linked to the same atom or different atoms of the alkylene. For instance, a straight chain alkylene can be the bivalent radical of —(CH₂)ₙ, where n is 1, 2, 3, 4, 5 or 6. Alkylene groups include, but are not limited to, methylene, ethylene, propylene, isopropylene, butylene, isobutylene, sec-butylene, pentylene and hexylene.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. The term “alkyl” includes groups such as haloalkyl (e.g., —CF₃ and —CH₂CF₃) and acyl (e.g., —C(O)CH₃, —C(O)CF₃, —C(O)CH₂OCH₃, and the like). In some embodiments, the substituted alkyl and heteroalkyl groups have from 1 to 4 substituents. In some embodiments, the substituted akyl and heteroalkyl groups have 1, 2 or 3 substituents. Exceptions are those perhalo alkyl groups (e.g., pentafluoroethyl and the like).

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″ ″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

“Alkoxy” refers to alkyl group having an oxygen atom that either connects the alkoxy group to the point of attachment or is linked to two carbons of the alkoxy group. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

“Carboxyalkyl” means an alkyl group (as defined herein) substituted with a carboxy group. The term “carboxycycloalkyl” means a cycloalkyl group (as defined herein) substituted with a carboxy group. The term alkoxyalkyl means an alkyl group (as defined herein) substituted with an alkoxy group. The term “carboxy” employed herein refers to carboxylic acids and their esters.

“Haloalkyl” refers to alkyl as defined above where some or all of the hydrogen atoms are substituted with halogen atoms. Halogen (halo) represents chloro or fluoro, but may also be bromo or iodo. For example, haloalkyl includes trifluoromethyl, fluoromethyl, 1,2,3,4,5-pentafluoro-phenyl, etc. The term “perfluoro” defines a compound or radical which has all available hydrogens that are replaced with fluorine. For example, perfluorophenyl refers to 1,2,3,4,5-pentafluorophenyl, perfluoromethyl refers to 1,1,1-trifluoromethyl, and perfluoromethoxy refers to 1,1,1-trifluoromethoxy.

“Fluoro-substituted alkyl” refers to an alkyl group where one, some, or all hydrogen atoms have been replaced by fluorine.

“Cytokine” is a member of a group of protein signaling molecules that may participate in cell-cell communication in immune and inflammatory responses. Cytokines are typically small, water-soluble glycoproteins that have a mass of about 8-35 kDa.

“Cycloalkyl” refers to a cyclic hydrocarbon group that contains from about 3 to 12, from 3 to 10, or from 3 to 7 endocyclic carbon atoms. Cycloalkyl groups include fused, bridged and spiro ring structures.

“Endocyclic” refers to an atom or group of atoms which comprise part of a cyclic ring structure.

“Exocyclic” refers to an atom or group of atoms which are attached but do not define the cyclic ring structure.

“Cyclic alkyl ether” refers to a 4 or 5 member cyclic alkyl group having 3 or 4 endocyclic carbon atoms and 1 endocyclic oxygen or sulfur atom (e.g., oxetane, thietane, tetrahydrofuran, tetrahydrothiophene); or a 6 to 7 member cyclic alkyl group having 1 or 2 endocyclic oxygen or sulfur atoms (e.g., tetrahydropyran, 1,3-dioxane, 1,4-dioxane, tetrahydrothiopyran, 1,3-dithiane, 1,4-dithiane, 1,4-oxathiane).

“Alkenyl” refers to either a straight chain or branched hydrocarbon of 2 to 6 carbon atoms, having at least one double bond. Examples of alkenyl groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can also have from 2 to 3, 2 to 4, 2 to 5, 3 to 4, 3 to 5, 3 to 6, 4 to 5, 4 to 6 and 5 to 6 carbons. The alkenyl group is typically monovalent, but can be divalent, such as when the alkenyl group links two moieties together.

“Alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene.

“Alkynyl” refers to either a straight chain or branched hydrocarbon of 2 to 6 carbon atoms, having at least one triple bond. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can also have from 2 to 3, 2 to 4, 2 to 5, 3 to 4, 3 to 5, 3 to 6, 4 to 5, 4 to 6 and 5 to 6 carbons. The alkynyl group is typically monovalent, but can be divalent, such as when the alkynyl group links two moieties together.

“Alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, butynylene, sec-butynylene, pentynylene and hexynylene.

“Cycloalkyl” refers to a saturated or partially unsaturated, monocyclic, fused bicyclic or bridged polycyclic ring assembly containing from 3 to 12 ring atoms, or the number of atoms indicated. Monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C₃₋₈cycloalkyl includes cyclopropyl, cyclobutyl, cyclopentyl, cyclohexyl, cyclooctyl, and norbornane.

“Cycloalkylene” refers to a cycloalkyl group, as defined above, linking at least two other groups, i.e., a divalent hydrocarbon radical. The two moieties linked to the cycloalkylene can be linked to the same atom or different atoms of the cycloalkylene. Cycloalkylene groups include, but are not limited to, cyclopropylene, cyclobutylene, cyclopentylene, cyclohexylene, and cyclooctylene.

“Heterocycloalkyl” refers to a ring system having from 3 ring members to about 20 ring members and from 1 to about 5 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heterocycle includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl and 1,4-dioxa-8-aza-spiro[4.5]dec-8-yl.

“Heterocycloalkylene” refers to a heterocyclalkyl group, as defined above, linking at least two other groups. The two moieties linked to the heterocycloalkylene can be linked to the same atom or different atoms of the heterocycloalkylene.

“Aryl” refers to a monocyclic or fused bicyclic, tricyclic or greater, aromatic ring assembly containing 6 to 16 ring carbon atoms. For example, aryl may be phenyl, benzyl or naphthyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g. methylenedioxy or ethylenedioxy. Oxy-C₂-C₃-alkylene is also a divalent substituent attached to two adjacent carbon atoms of phenyl, e.g. oxyethylene or oxypropylene. An example for oxy-C₂-C₃-alkylene-phenyl is 2,3-dihydrobenzofuran-5-yl.

In some embodiments the aryl is naphthyl, phenyl or phenyl mono- or disubstituted by alkoxy, phenyl, halogen, alkyl or trifluoromethyl, especially phenyl or phenyl-mono- or disubstituted by alkoxy, halogen or trifluoromethyl, and in particular phenyl.

Examples of substituted phenyl groups as R are, e.g. 4-chlorophen-1-yl, 3,4-dichlorophen-1-yl, 4-methoxyphen-1-yl, 4-methylphen-1-yl, 4-aminomethylphen-1-yl, 4-methoxyethylaminomethylphen-1-yl, 4-hydroxyethylaminomethylphen-1-yl, 4-hydroxyethyl-(methyl)-aminomethylphen-1-yl, 3-aminomethylphen-1-yl, 4-N-acetylaminomethylphen-1-yl, 4-aminophen-1-yl, 3-aminophen-1-yl, 2-aminophen-1-yl, 4-phenyl-phen-1-yl, 4-(imidazol-1-yl)-phenyl, 4-(imidazol-1-ylmethyl)-phen-1-yl, 4-(morpholin-1-yl)-phen-1-yl, 4-(morpholin-1-ylmethyl)-phen-1-yl, 4-(2-methoxyethylaminomethyl)-phen-1-yl and 4-(pyrrolidin-1-ylmethyl)-phen-1-yl, 4-(thiophenyl)-phen-1-yl, 4-(3-thiophenyl)-phen-1-yl, 4-(4-methylpiperazin-1-yl)-phen-1-yl, and 4-(piperidinyl)-phenyl and 4-(pyridinyl)-phenyl optionally substituted in the heterocyclic ring.

“Arylene” refers to an aryl group, as defined above, linking at least two other groups. The two moieties linked to the arylene are linked to different atoms of the arylene. Arylene groups include, but are not limited to, phenylene.

“Arylene-oxy” refers to an arylene group, as defined above, where one of the moieties linked to the arylene is linked through an oxygen atom. Arylene-oxy groups include, but are not limited to, phenylene-oxy.

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

“Heteroaryl” refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. In some embodiments, quinolinyl represents 2-, 3- or 4-quinolinyl. In some embodiments, isoquinolinyl represents 1-, 3- or 4-isoquinolinyl. In some embodiments, benzopyranyl, benzothiopyranyl can represent 3-benzopyranyl or 3-benzothiopyranyl, respectively. In some embodiments, thiazolyl can represent 2- or 4-thiazolyl. In some embodiments, triazolyl can be 1-, 2- or 5-(1,2,4-triazolyl). In some embodiments, tetrazolyl can be 5-tetrazolyl.

In some embodiments, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

The term “heteroalkyl” refers to an alkyl group having from 1 to 3 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heteroalkyl can include ethers, thioethers, alkyl-amines and alkyl-thiols.

The term “heteroalkylene” refers to a heteroalkyl group, as defined above, linking at least two other groups. The two moieties linked to the heteroalkylene can be linked to the same atom or different atoms of the heteroalkylene.

“Electrophile” refers to an ion or atom or collection of atoms, which may be ionic, having an electrophilic center, i.e., a center that is electron seeking, capable of reacting with a nucleophile. An electrophile (or electrophilic reagent) is a reagent that forms a bond to its reaction partner (the nucleophile) by accepting both bonding electrons from that reaction partner.

“Nucleophile” refers to an ion or atom or collection of atoms, which may be ionic, having a nucleophilic center, i.e., a center that is seeking an electrophilic center or capable of reacting with an electrophile. A nucleophile (or nucleophilic reagent) is a reagent that forms a bond to its reaction partner (the electrophile) by donating both bonding electrons. A “nucleophilic group” refers to a nucleophile after it has reacted with a reactive group. Non limiting examples include amino, hydroxyl, alkoxy, haloalkoxy and the like.

“Maleimido” refers to a pyrrole-2,5-dione-1-yl group having the structure:

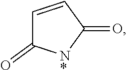

which upon reaction with a sulfhydryl (e.g., a thio alkyl) forms an —S-maleimido group having the structure:

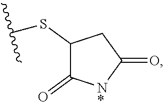

where “⋅” indicates the point of attachment for the maleimido group and “ [symbol] ” indicates the point of attachment of the sulfur atom the thiol to the remainder of the original sulfhydryl bearing group.

For the purpose of this disclosure, “naturally occurring amino acids” found in proteins and polypeptides are L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyrosine, and or L-valine. “Non-naturally occurring amino acids” found in proteins are any amino acid other than those recited as naturally occurring amino acids. Non-naturally occurring amino acids include, without limitation, the D isomers of the naturally occurring amino acids, and mixtures of D and L isomers of the naturally occurring amino acids. Other amino acids, such as N-alpha-methyl amino acids (e.g. sarcosine), 4-hydroxyproline, desmosine, isodesmosine, 5-hydroxylysine, epsilon-N-methyllysine, 3-methylhistidine, although found in naturally occurring proteins, are considered to be non-naturally occurring amino acids found in proteins for the purpose of this disclosure as they are generally introduced by means other than ribosomal translation of mRNA.

“Linear” in reference to the geometry, architecture or overall structure of a polymer, refers to polymer having a single polymer arm.

“Branched,” in reference to the geometry, architecture or overall structure of a polymer, refers to a polymer having 2 or more polymer “arms” extending from a core structure contained within an initiator. The initiator may be employed in an atom transfer radical polymerization (ATRP) reaction. A branched polymer may possess 2 polymer chains (arms), 3 polymer arms, 4 polymer arms, 5 polymer arms, 6 polymer arms, 7 polymer arms, 8 polymer arms, 9 polymer arms or more. Each polymer arm extends from a polymer initiation site. Each polymer initiation site is capable of being a site for the growth of a polymer chain by the addition of monomers. For example and not by way of limitation, using ATRP, the site of polymer initiation on an initiator is typically an organic halide undergoing a reversible redox process catalyzed by a transition metal compound such as cuprous halide. In some embodiments, the halide is a bromine.

“Pharmaceutically acceptable excipient” refers to an excipient that can be included in compositions and that causes no significant adverse toxicological effect on the patient and is approved or approvable by the FDA for therapeutic use, particularly in humans. Non-limiting examples of pharmaceutically acceptable excipients include water, NaCl, normal saline solutions, lactated Ringer's, normal sucrose, normal glucose and the like.

Therapeutic proteins are administered in an effective regime meaning a dosage, route of administration and frequency of administration that delays the onset, reduces the severity, inhibits further deterioration, and/or ameliorates at least one sign or symptom of a disorder. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

The “biological half-life” of a substance is a pharmacokinetic parameter which specifies the time required for one half of the substance to be removed from a tissue or an organism following introduction of the substance.

“OG1786” is a 9-arm initiator used for polymer synthesis, which depicts that salt form of OG1786 with trifluororacetic acid. OG1786 may be used as other salts are used or as the free base.

“OG1801” is an approximately (+/−15%) 750 kDa polymer (either by Mn or Mp) made using OG1786 as an initiator for ATRP synthesis using the monomer HEMA-PC.

“OG1802” is OG1801 with a maleimide functionality added wherein each of n₁, n₂, n₃, n₄, n₅, n₆, n₇, n₈ and n₉ is an integer (positive) (from 0 up to about 3000) such that the total molecular weight of the polymer is (Mw) 750,000±15% Daltons.

Multi-angle light scattering (MALS) is a technique of analyzing macromolecules where the laser light impinges on the molecule, the oscillating electric field of the light induces an oscillating dipole within it. This oscillating dipole will re-radiate light and can be measured using a MALS detector such as Wyatt miniDawn TREOS. The intensity of the radiated light depends on the magnitude of the dipole induced in the macromolecule which in turn is proportional to the polarizability of the macromolecule, the larger the induced dipole, and hence, the greater the intensity of the scattered light. Therefore, in order to analyze the scattering from a solution of such macromolecules, one should know their polarizability relative to the surrounding medium (e.g., the solvent). This may be determined from a measurement of the change, Δn, of the solution's refractive index n with the molecular concentration change, Δc, by measuring the dn/dc (=Δn/Δc) value using a Wyatt Optilab T-rEX differential refractometer. Two molar weight parameters that MALS determination employ are number average molecular weight (Mn) and weight average molecular weight (Mw) where the polydispersity index (PDI) equals Mw divided by Mn. SEC also allows another average molecular weight determination of the peak molecular weight Mp which is defined as the molecular weight of the highest peak at the SEC.

The PDI is used as a measure of the broadness of a molecular weight distribution of a polymer and bioconjugate which is derived from conjugation of a discrete protein (e.g. OG1950) to a polydisperse biopolymer (e.g., OG1802). For a protein sample, its polydispersity is close to 1.0 due to the fact that it is a product of translation where every protein molecule in a solution is expected to have almost the same length and molar mass. In contrast, due to the polydisperse nature of the biopolymer where the various length of polymer chains are synthesized during the polymerization process, it is very important to determine the PDI of the sample as one of its quality attribute for narrow distribution of molecular weight.

Size exclusion chromatography (SEC) is a chromatography technique in which molecules in solution are separated by their size. Typically an aqueous solution is applied to transport the sample through the column which is packed with resins of various pore sizes. The resin is expected to be inert to the analyte when passing through the column and the analytes separate from each other based on their unique size and the pore size characteristics of the selected column.

Coupling the SEC with MALS or SEC/MALS provides accurate distribution of molar mass and size (root mean square radius) as opposed to relying on a set of SEC calibration standards. This type of arrangement has many advantages over traditional column calibration methods. Since the light scattering and concentration are measured for each eluting fraction, the molar mass and size can be determined independently of the elution position. This is particularly relevant for species with non-globular shaped macromolecules such as the biopolymers (OG1802) or bioconjugates (e.g., KSI-301, KSI-501); such species typically do not elute in a manner that might be described by a set of column calibration standards.

In some embodiments, a SEC/MALS analysis includes a Waters HPLC system with Alliance 2695 solvent delivery module and Waters 2996 Photodiole Array Detector equipped with a Shodex SEC-HPLC column (7.8×300 mm). This is connected online with a Wyatt miniDawn TREOS and Wyatt Optilab T-rEX differential refractometer. The Empower software from Waters can be used to control the Waters HPLC system and the ASTRA V 6.1.7.16 software from Wyatt can be used to acquire the MALS data from the Wyatt miniDawn TREOS, dn/dc data from the T-rEX detector and the mass recovery data using the A280 absorbance signal from the Waters 2996 Photodiole Array detector. SEC can be carried out at 1 ml/min in 1×PBS pH 7.4, upon sample injection, the MALS and RI signals can be analyzed by the ASTRA software for determination of absolute molar mass (Mp, Mw, Mn) and polydisperse index (PDI). In addition, the calculation also involves the input dn/dc values for polymer and protein as 0.142 and 0.183, respectively. For KSI-301 dn/dc value, the dn/dc is calculated based on the weighted MW of the polymer and the protein to be about 0.148 using the formula below:

Conjugate dn/dc=0.142×[Mwpolymer/(Mwpolymer+Mwprotein)]+0.183×[Mwprotein/(Mwpolymer+Mwprotein)]
where Mwpolymer for OG1802 is 800 kDa and the Mwprotein for OG1950 is 146 kDa.

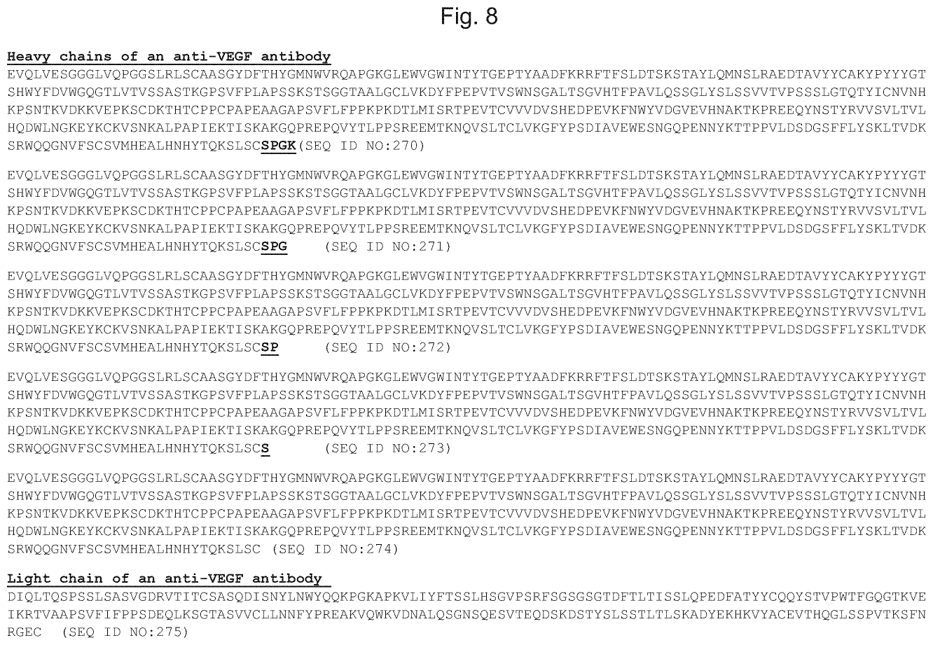

“KSI-301” is a bioconjugate of a recombinant, mammalian cell expressed full-length humanized anti-VEGF monoclonal antibody which is covalently conjugated to a branched high molecular weight phosphorylcholine based biopolymer. In some embodiments, KSI-301 is supplied as a preservative free, sterile, aqueous solution in a single-use glass vial at a concentration of 50 mg/mL (based on antibody mass). FIG. 8 displays the amino acid sequence of the antibody portion of KSI-301. KSI-301 is an anti-vascular endothelial growth factor (VEGF) biopharmaceutical with an extended ocular half-life. KSI-301 is a bioconjugate of two intermediates: (1) OG1950 antibody intermediate, a recombinant, full-length humanized, anti-huVEGF A monoclonal antibody, and (2) OG1802 biopolymer intermediate, a phosphorylcholine biopolymer. The addition of OG1802, an inert biopolymer, increases the size of the biologic, thereby extending the ocular pharmacokinetics (PK) of KSI-301 beyond that of currently approved anti-huVEGF-A therapeutics. Nonclinical studies with KSI-301 indicate that it appropriately binds with high affinity to huVEGF-A whose binding to huVEGF Receptors 1 and 2 (huVEGFR) is then inhibited. This in turn abrogates huVEGF-A mediated function.

In some embodiments, the molecule to be administered in any one or more of the methods provided herein is any one of the molecules disclosed in U.S. Pat. Pub. No. 2017/0190766, herein incorporated by reference in its entirety.

FIG. 8 displays the amino acid sequence of the antibody portion of KSI-301 with a terminal lysine removed. Terminal lysine removal is a post-translational modification in antibodies. The lysine residues at the heavy chain C-terminus of recombinant IgGs are removed (often to a large extent) during cell culture by carboxypeptidases that are endogenous to CHO host cells. For all antibody sequences recited herein, although specific sequences (which may be longer) are provided for exemplary antibodies disclosed herein, it will be appreciated that after expression of protein chains one to several amino acids at the amino or carboxy terminus of the light and/or heavy chain, particularly a heavy chain C-terminal lysine residue, may be missing or derivatized in a proportion or all of the molecules. Thus, in some embodiments, any of the antibody sequences provided herein may be modified by this lysine clip, which can include the modified version as shown in FIG. 8 (with one or more of the underlined residues being removed). In some embodiments, the lysine in the heavy chain of FIG. 8 is removed. In some embodiments, the GK is removed. In some embodiments, the PGK is removed. In some embodiments, the SPGK is removed. As will be appreciated by those in the art, across a population of molecules, the lysine clip may vary and not be complete. Thus, for example, 90, 95, 98, 99, or 100% of the molecules may have one or more of the clip versions, while 10, 5, 2, 1, or down to 0% may be full length (including any range defined between any two of the preceding values). Thus, as used herein, KSI-301 includes any one of and all options for the heavy chain variations outlined in FIG. 8.

As used herein, any time “anti-VEGF antibody” or “anti-VEGF antibody conjugate” is referenced, an anti-VEGF protein, such as an anti-VEGF fusion protein, e.g., aflibercept, is also contemplated. Thus, as disclosed herein, any time “anti-VEGF antibody conjugate” is referenced, an anti-VEGF protein, e.g., aflibercept, covalently bonded to a phosphorylcholine containing biopolymer (e.g., OG1802) as disclosed herein, is also contemplated. In the various embodiments disclosed herein, any reference to an anti-VEGF antibody conjugate therapy, also contemplates an anti-VEGF protein, e.g., aflibercept, conjugate therapy. In the various embodiments of methods of treating an eye disorder, disclosed herein, any reference to an anti-VEGF antibody conjugate, also contemplates an aflibercept biopolymer conjugate.

### Methods of Purification

In some embodiments, a method of purifying a product is provided. The method comprises washing a bound protein product with a chaotropic agent, and later collecting the bound protein. The method can further include additional upstream and downstream purification processes.

In some embodiments, a method of purifying a product using affinity chromatography is provided. The method comprises loading an eluent into an affinity chromatography matrix, wherein the affinity chromatography matrix binds to a protein of interest; and washing the affinity chromatography matrix with a buffer solution comprising a chaotropic agent.

In some embodiments, the target protein of interest is produced by a cell culture. In some embodiments, the cell culture are CHO cells. In some embodiments, the protein of interest is a bispecific antibody. In some embodiments, the bispecific antibody is specific for VEGF and IL-6. In some embodiments, the bispecific antibody is OG2072. In some embodiments, the protein of interest is an antibody conjugate. In some embodiments, the affinity chromatography matrix is a protein A chromatography matrix.

In some embodiments, the chaotropic agent in the buffer solution is comprised of one or more of lithium and lithium salts, magnesium and magnesium salts, calcium and calcium salts and guanidinium and guanidinium salts.

In some embodiments, the concentration of magnesium salt is between 2-3.5 M. In some embodiments, the concentration is about 2.8M of MgCl₂.

In some embodiments, the concentration of magnesium salt is: 2, 2.1, 2.2, 2.3, 2.4. 2.5, 2.6, 2.7, 2.8, 2.9, 3, 3.1, 3.2, 3.3, 3.4, or 3.5 M, or any value between the aforementioned range.

In some embodiments, the concentration of calcium salt is between 1-3 M. In some embodiments, the concentration is about 2.0M of CaCl₂).

In some embodiments, the concentration of calcium salt is: 1, 1.5, 1.8, 1.9, 2, 2.1, 2.2, 2.3, 2.4. 2.5, 2.7, or 3 M, or any value between the aforementioned range.

In some embodiments, the concentration of guanidinium salt is between 0.05-3 M. In some embodiments, the concentration is about 1.0M of guanidinium hydrochloride.

In some embodiments, the concentration of guanidinium salt is: 0.05, 0.075, 0.1, 0.2, 0.25, 0.5, 0.75, 1, 1.5, 1.75, 2, 2.5, 2.75, or 3 M, or any value between the aforementioned range.

In some embodiments, the buffer solution further comprises tris.

In some embodiments, the concentration of tris in the buffer solution is at least 5 mM.

In some embodiments, the concentration of tris in the buffer solution is at least 10 mM.

In some embodiments, the concentration of tris in the buffer solution is at least: 25 mM, 30 mM, 35 mM, 40 mM, 50 mM or a value greater than 50 mM.

In some embodiments, the pH of the buffer solution is greater than 5.5.

In some embodiments, a method of purifying a product and reducing impurities from a load fluid comprising the protein and one or more impurities is provided herein. The method comprises passing the load fluid through an affinity chromatography matrix, followed by at least one wash solution comprising a chaotropic salt, and collecting the protein using an elution solution.

In some embodiments, provided herein is a method for separating impurities in an eluate comprising a protein of interest. The method comprising loading an eluent comprising a protein of interest onto an affinity chromatography matrix; and washing the affinity chromatography matrix with one or more buffer solutions comprising one or more of lithium and lithium salts, magnesium and magnesium salts, calcium and calcium salts and guanidinium and guanidinium salts.

In some embodiments, provided herein is a method for separating impurities in an eluate comprising a protein of interest. The method comprising loading an eluent comprising a protein of interest onto an affinity chromatography matrix; and washing the affinity chromatography matrix with one or more buffer solutions comprising one or more of lithium and lithium salts, magnesium and magnesium salts, calcium and calcium salts and guanidinium and guanidinium salts. In some embodiments, the method further comprises the step of washing the affinity chromatography matrix loaded with the eluent with a postwash buffer solution after washing with buffer solution. In some embodiments, washing the affinity chromatography matrix with the buffer solution removes nucleic acids, endotoxins, antifoam agents, other molecules, or other small molecules other than the target protein of interest. In some embodiments, washing the affinity chromatography matrix with the buffer solution removes impurities while keeping the target protein of interest bound to the affinity chromatography matrix. In some embodiments, washing the affinity chromatography matrix with the buffer solution removes host cell proteins besides the target protein of interest. In some embodiments, the addition of chaotropic agent in the buffer solution does not elute the target protein of interest. In some embodiments, the method further comprises one or more of virus inactivation, tangential flow filtration, diafiltration, ultrafiltration, ion exchange chromatography, or virus reduction filtration. In some embodiments, the eluent was produced in a bioreactor using animal component free cell culture. In some embodiments, the product is a purified protein of interest. In some embodiments, the impurities comprise host cell protein (HCP) impurities. In some embodiments, the eluate further comprises viral impurities. In some embodiments, the method further comprises removing viral impurities. In some embodiments, the method further comprises the step of washing the affinity chromatography matrix loaded with the load fluid with a prewash buffer solution prior to washing with the buffer solution. In some embodiments, the method further comprises washing the affinity chromatography matrix loaded with the eluent with a post-wash buffer solution after washing with buffer solution. In some embodiments, the prewash buffer solution comprises sodium phosphate. In some embodiments, the prewash buffer solution comprises tris and a salt. In some embodiments of the method, the antibody conjugate comprises an anti-VEGF antibody conjugate comprising an anti-VEGF-A light chain and an anti-VEGF-A heavy chain, wherein the anti-VEGF-A antibody heavy chain comprises CDRH1: that is a CDRH1 in SEQ ID NO: 172, CDRH2: that is a CDRH2 in SEQ ID NO: 173, and CDRH3: that is a CDRH3 in SEQ ID NO: 174, and the anti-VEGF-A antibody light chain comprises CDRL1: that is a CDRL1 in SEQ ID NO: 199, CDRL2: that is a CDRL2 in SEQ ID NO: 200, and CDRL3: that is a CDRL3 in SEQ ID NO: 201. In some embodiments of the method provided herein, the anti-VEGF antibody conjugate comprises: an antibody conjugate comprising an anti-VEGF-A immunoglobulin G (IgG) bonded to a polymer, which polymer comprises MPC monomers, wherein the sequence of the anti-VEGF-A antibody heavy chain is at least one of SEQ ID Nos: 7-13, 19-27, 89, 90, 256-262, and the sequence of the anti-VEGF-A antibody light chain is at least one of SEQ ID Nos: 91-93, 28-30, and wherein the antibody is bonded at C449 to the polymer.

In some embodiments, a method of producing a product using affinity chromatography is provided. The method comprises loading an eluent comprising a protein of interest onto an affinity chromatography matrix; and washing the affinity chromatography matrix with one or more buffer solutions comprising one or more of lithium and lithium salts, magnesium and magnesium salts, calcium and calcium salts and guanidinium and guanidinium salts.

In some embodiments, a method for processing a product is provided. The method comprises loading an eluent into an affinity chromatography matrix. In some embodiments, the method further comprises washing with a wash buffer comprising a chaotropic salt to collect an eluate, wherein the concentration of the chaotropic salt is increased from a first concentration to a second concentration, wherein the eluate is collected in at least one fraction, wherein the at least one fraction comprises a product of interest. In some embodiments, the method further comprises wherein the concentration of the chaotropic salt at the first concentration is 0 M, and wherein the concentration of the chaotropic salt at the second concentration is 4.0 M. In some embodiments, the chaotropic salt is magnesium based. In some embodiments, the chaotropic salt is MgCl2.

In some embodiments, a method of producing a product using affinity chromatography is provided. The method comprises loading an eluent containing a protein of interest onto an affinity chromatography matrix, a first wash of the affinity chromatography matrix with a first buffer comprising sodium phosphate and a salt, and a second wash of the affinity chromatography matrix with a second buffer comprising a chaotropic agent.

In some embodiments, method of producing a product using affinity chromatography is provided. The method comprises loading an eluent containing a protein of interest onto an affinity chromatography matrix, a first wash with a first buffer containing Tris and a salt, a second wash with a second buffer containing Tris and a chaotropic agent, wherein the second buffer chaotropic agent is not the same salt as contained in the first buffer.

In some embodiments, a method of producing a product is provided. The method comprises collecting a load fluid, wherein the load fluid comprises a protein of interest, loading the load fluid onto an affinity chromatography matrix, wherein the affinity chromatography matrix binds to the protein of interest, then washing the affinity chromatography matrix with a buffer solution comprising a chaotropic salt, eluting the bound protein of interest; and collecting an eluate, wherein the eluate contains the protein of interest.

In some embodiments, a method of producing a product is provided. The method comprises collecting a load fluid, wherein the load fluid comprises a protein of interest, loading the load fluid onto an affinity chromatography matrix, wherein the affinity chromatography matrix binds to the protein of interest, feeding to the affinity chromatography matrix a buffer solution comprising a chaotropic salt, eluting the bound protein of interest; and collecting an eluate, wherein the eluate contains the protein of interest.

In some embodiments, a method of producing a product is provided. The method comprises collecting a conjugate protein, wherein the conjugate protein comprises an antibody bound to a conjugate polymer loading the conjugate protein onto an affinity chromatography matrix, wherein the affinity chromatography matrix binds to the conjugate protein, washing the affinity chromatography matrix with a buffer solution comprising a chaotropic salt, eluting the conjugate protein, and collecting an eluate, wherein the eluate contains the conjugate protein.

In some embodiments, a method of producing a product is provided. The method comprises washing an affinity chromatography matrix bound to a target protein of interest with a buffer comprising a chaotropic salt, eluting and collecting an eluate, wherein the eluate contains the target protein of interest, and removing viral contaminants from the eluate.

In some embodiments, removing viral contaminants from the eluate comprises one or more of low pH inactivation, detergent inactivation, polishing chromatography steps, viral filtration, ultrafiltration, and/or diafiltration.

In some embodiments, a method of producing a product is provided. The method comprises washing an affinity chromatography matrix bound to a target protein of interest with a buffer comprising a chaotropic salt, removing the chaotropic salt, and eluting and collecting an eluate, wherein the eluate contains the target protein of interest.

In some embodiments, the eluate is further combined with an acceptable pharmaceutical excipient to form a pharmaceutical composition. In some embodiments, a buffer solution is added to the pharmaceutical composition. In some embodiments, a preservative solution is added to the pharmaceutical composition. In some embodiments, the pharmaceutical composition is further refined for intravitreal injection.

In some embodiments, a method of producing a product is provided. The method comprises collecting a load fluid, wherein the load fluid is comprised of a protein of interest, loading the load fluid into an affinity chromatography matrix, wherein the affinity chromatography matrix binds to the protein of interest, washing the affinity chromatography matrix with a buffer solution comprising a chaotropic salt, eluting and collecting an eluate, wherein eluate contain the target protein of interest, and removing viral contaminants from the eluate.

In some embodiments, a method of producing a product is provided. The method comprises collecting a load fluid, wherein the load fluid comprises a protein of interest, loading the load fluid onto an affinity chromatography matrix, wherein the affinity chromatography matrix binds to the protein of interest, washing the affinity chromatography matrix with a buffer solution comprising a chaotropic salt, eluting the bound protein of interest; and collecting an eluate, wherein the eluate contains the protein of interest.

In some embodiments, a method of producing a product is provided. The method comprises collecting a load fluid, wherein the load fluid comprises a protein of interest, loading the load fluid onto an affinity chromatography matrix, wherein the affinity chromatography matrix binds to the protein of interest, feeding to the affinity chromatography matrix a buffer solution comprising a chaotropic salt, eluting the bound protein of interest; and collecting an eluate, wherein the eluate contains the protein of interest.

In some embodiments, a method of producing a product is provided. The method comprises collecting a conjugate protein, wherein the conjugate protein comprises an antibody bound to a conjugate polymer loading the conjugate protein onto an affinity chromatography matrix, wherein the affinity chromatography matrix binds to the conjugate protein, washing the affinity chromatography matrix with a buffer solution comprising a chaotropic salt, eluting the conjugate protein, and collecting an eluate, wherein the eluate contains the conjugate protein.

In some embodiments, a method of producing a product is provided. The method comprises washing an affinity chromatography matrix bound to a target protein of interest with a buffer comprising a chaotropic salt, eluting and collecting an eluate, wherein the eluate contains the target protein of interest, and removing viral contaminants from the eluate.

The method may further include wherein removing viral contaminants from the eluate comprises one or more of low pH inactivation, detergent inactivation, polishing chromatography steps, viral filtration (VF), ultrafiltration (UF) and/or diafiltration (DF). The method may further include wherein the eluate is further combined with an acceptable pharmaceutical excipient to form a pharmaceutical composition. The method may further include wherein a buffer solution is added to the pharmaceutical composition. The method may further include wherein a preservative solution is added to the pharmaceutical composition. The method may further include wherein the pharmaceutical composition is further refined for intravitreal injection.

In some embodiments, a method of producing a product is provided. The method comprises washing an affinity chromatography matrix bound to a target protein of interest with a buffer comprising a chaotropic salt, removing the chaotropic salt, and eluting and collecting an eluate, wherein the eluate contains the target protein of interest.

In some embodiments, a method of producing a product is provided. The method comprises collecting a load fluid, wherein the load fluid is comprised of a protein of interest, loading the load fluid into an affinity chromatography matrix, wherein the affinity chromatography matrix binds to the protein of interest, washing the affinity chromatography matrix with a buffer solution comprising a chaotropic salt, eluting and collecting an eluate, wherein eluate contain the target protein of interest, and removing viral contaminants from the eluate.

In some embodiments, a method of producing a product is provided. The method comprises loading an eluent into an affinity chromatography matrix, washing with a first wash buffer washing with a second wash buffer comprising a chaotropic salt, washing with a third wash buffer, wherein the third wash buffer removes the chaotropic salt, eluting with an elution buffer, wherein an eluate is collected, wherein the eluate comprises a protein product.

The method may further include wherein the first wash buffer comprises 50 mM Na-Phosphate. The method may further include wherein the first wash buffer further comprises 250 mM NaCl. The method may further include wherein the first wash buffer comprises Tris and a salt. The method may further include removing viral contaminants from the eluate. The method may further include wherein removing viral contaminants comprises: one or more of low pH inactivation, detergent inactivation, polishing chromatography steps, viral filtration (VF), ultrafiltration (UF), or diafiltration (DF). The method may further include wherein the eluent comprises a protein of interest. The method may further include wherein the protein of interest is an antibody. The method may further include wherein the antibody is further conjugated to a polymer to form an antibody conjugate. The method may further include wherein the antibody conjugate comprises a bispecific antibody. The method may further include wherein the bispecific antibody comprises anti-VEGF and anti-IL-6 binding moieties.

The method may further include wherein the first wash buffer comprises 10, 50, 100, 150, 200 mM, or any integer that is between 10 and 200 mM Na-Phosphate. The method may further include wherein the first wash buffer comprises a phosphate-based species. The method may further include wherein the first wash buffer further comprises 25, 50, 100, 150, 200, or any integer that is between 25 and 200 mM NaCl. The method may further include wherein the concentration of the chaotropic salt at the first concentration is 0 M, wherein the concentration of the chaotropic salt at the second concentration is 4.0 M. The method may further include wherein the chaotropic salt is Magnesium based. The method may further include wherein the chaotropic salt is MgCl2.

In some embodiments, the antibody conjugate comprises a protein construct comprising an antagonist IL-6 antibody fused to a VEGF trap, wherein the antibody comprises an isolated antagonistic IL-6 antibody or fragment thereof. In some embodiments, the bispecific antibody comprises a VEGF-anti-IL-6 dual inhibitor, wherein the VEGFR-anti-IL-6 dual inhibitor comprises a trap antibody fusion of an anti-IL 6 antibody or fragment thereof and an anti-VEGF trap (VEGFR1/2), wherein the dual inhibitor includes at least one point mutation with a VEGFR sequence to reduce cleavage of the VEGFR protein wherein the antibody comprises a fragment antigen binding (Fab) region, a hinge region, and a fragment crystallizable (Fc) region, wherein the anti-VEGF trap is positioned either at an N-terminal end of a heavy chain of the antibody, wherein the heavy chain comprises IL-6 VH, or between the Fab and hinge regions. In some embodiments, the antibody conjugate comprises an antibody conjugate comprising (1) an anti-VEGF-A antibody and (2) a phosphorylcholine containing polymer, wherein the polymer is covalently bonded to the antibody at a cysteine outside a variable region of the antibody wherein said cysteine has been added via recombinant DNA technology. In some embodiments, the antibody conjugate comprises an isolated antagonist antibody that specifically binds to complement factor D (CFD) and directly inhibits a proteolytic activity of CFD.

In some embodiments, the antibody conjugate has the structure of Formula (I):

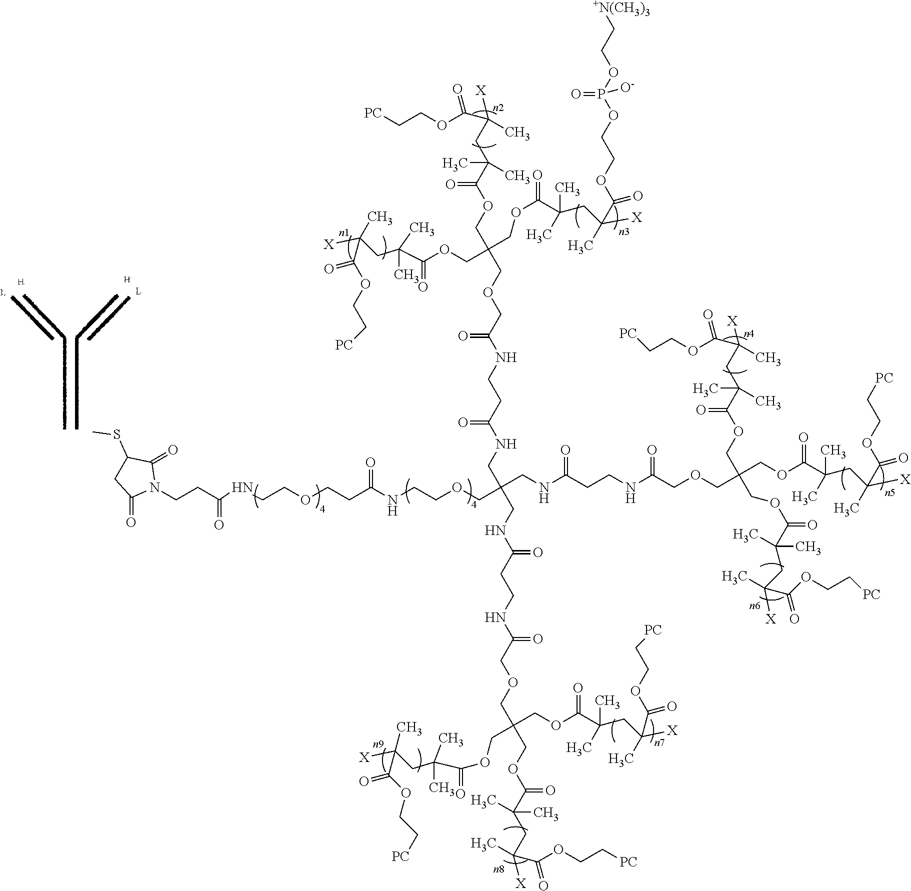

wherein each heavy chain of the antibody is denoted by the letter H, and each light chain of the anti-CFD antibody is denoted by the letter L; the polymer is bonded to the antibody through the sulfhydryl of C443 (EU numbering), which bond is depicted on one of the heavy chains; PC is

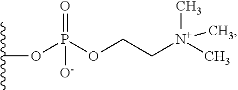 [SMILES: *OP(=O)([O-])OCC[N+](C)(C)C]

where the curvy line indicates the point of attachment to the rest of the polymer, where X=a) OR where R═H, methyl, ethyl, propyl, isopropyl, b) H, or c) any halide, including Br; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%. In some embodiments, the sum of n1, n2, n3, n4, n5, n6, n7, n8 and n9 is about 1500 to about 3500 plus or minus about 10% to about 20%.

Some embodiments provide any of the following, or compositions (including pharmaceutical compositions) comprising an anti-CFD antibody having a partial light chain sequence and a partial heavy chain sequence as found in Tables 1A, 1B, 1C, 1D and/or 1E, or variants thereof. In Table 1A, the underlined sequences are some embodiments of CDR sequences as provided herein. In some embodiments, a composition as disclosed herein comprises an antibody having a partial or complete light chain sequence and a partial or complete heavy chain sequence from any of the options provided in Tables 1A, 1B, 1C, 1D and/or 1E, or variants thereof. In some embodiments, the antibody (or binding fragment thereof) can include any one or more of the CDRs provided in Tables: 1A, 1B, 1C, 1D and/or 1E. In some embodiments, the antibody (or binding fragment thereof) can include any three or more of the CDRs provided in Tables: 1A, 1B, 1C, 1D and/or 1E. In some embodiments, the antibody (or binding fragment thereof) can include any all six of the CDRs provided in Tables 1A, 1B, 1C, 1D and/or 1E. CDR sequences for various constructs are also found in Tables 1A, 1B, 1C, 1D and/or 1E.

TABLE 1A
Variable Regions Sequences of anti-CFD Antagonist monoclonal Antibodies (CDRs
are underlined).

| mAb | Heavy Chain | Light Chain |
|---|---|---|
| KCD002 | EVKLVESEGGLVQPGSSMKLSCTASGFTFSDYY | DIQMTQSPASLSASVGETVTITCRASENIHSY |
| | MAWVRQVPEKGLEWVGNINYDGSSTYYLDSLK | LAWYQQKQGKSPQLIVYNTKTLAEGVPSRF |
| | SRFIISRDSAKNILYLQMSSLKSEDTATYFCARGE | SGSGSGTQFSLKINSLQPEDFGSYYCQHHYGI |
| | DFYLYAMDYWGQGTSVTVSS (SEQ ID NO: 276) | PPTFGGGTKLEIK (SEQ ID NO: 309) |
| KCD003 | EVQLQQSRPELVKPGASVKIFCKASGYTFTDYY | DVLMTQTPLSLPVSLGEQASISCRSSQTIVHS |
| | MNWMRQRHGETLEWIGDINPNNGDPSYNQKFK | NGDTYLEWYLQKPGQSPNLLIYKVSNRFSG |
| | DKATLTVDKSSSTASMELRSLTSDDSAVYYCAR | VPDRFSGSGSGTDFTLKISRVEAEDLGVYYC |
| | EGPSFAYWGQGTLVTVSA (SEQ ID NO: 277) | FQGSHVPPTFGGGTKLEIK (SEQ ID NO: 310) |
| KCD005 | EVQLQQSGPELVKPGASVKISCKASGYTFTDHY | DVLMTQTPLSLPVSLGDQASISCRSSQSIVHS |
| | MNWVKQSHGKSLEWIGDINPNNGGTSCNQKFK | NGDTYLEWYLQKPGQSPKLLIYKVSNRFSG |
| | GKATLTVDKSSSTAYMELRSLTSGDSAVYYCTR | VPDRFSGSGSGTDFTLKISRVEAEDLGVYYC |
| | EGASFAFWGQGTLVTVSA (SEQ ID NO: 278) | FQGSHVPVTFGAGTKLELK (SEQ ID NO: 311) |
| KCD009 | QIQLVQSGPELKKPGETVKISCKASGYIFRNYGM | DVLMTQTPLSLPVSLGDQASISCRSSLIIEHSD |
| | NWVKQGPGKGLKWMGWINTYTGEPTYADDFK | GNTYLEWYLQKPGQSPKLLIYKVSNRFSGVP |
| | GRFAFSLETSASTAYLQISNLKNEDTATYFCVRD | DRFSGSGSGTDFTLKISRVEADDLGVYYCFQ |
| | GPGFAYWGQGTLVTVSA (SEQ ID NO: 279) | GSHVPVTFGAGTNLELK (SEQ ID NO: 312) |
| KCD010 | EVLLQQSGPELVKPGASVKIPCKASGYTFTDYD | QIVLTQSPAIMSVSPGEKVTLTCSASSSVSSS |
| | MDWVKQSHGKSLEWIGHINPNNGGTIYNQKFK | YLYWYQKKPGSSPKLWIYSTSNLASGVPAR |
| | GKATLTVDKSSSTAYMELRSLTSEDTAVYYCGT | FSGSGSGTSYSLTISSMEAEDAASYFCHQWS |
| | GDFAYWGHGTLVTVSA (SEQ ID NO: 280) | SYPPTFGAGTKLELK (SEQ ID NO: 313) |
| KCD023 | EVLLQQSGPELVKPGASVKIPCKASGYTFTDYNI | QIVLTQSPAFMSASPGEKVTLTCSASSSVSSS |
| | DWVKQSHGKSLEWIGDINPNNGGINYNQKFKG | YLYWYQQKPGSSPKLWIYSTSNLASGVPGR |
| | KATLTVDKSSSTAYMELRSLTSEDTAVYYCGTG | FSGSGSGTSYSLTISSMEAEDAASYFCHQWT |
| | DYAYWGQGTLVTVSA (SEQ ID NO: 281) | SYPPTFGAGTKLELK (SEQ ID NO: 314) |
| KCD036 | EVQLQQSGPELVKPGASMKISCKASGYSFTGYT | DIKLTQSPSSMYASLGERVTITCKASQDINTY |
| | MTWVKQSHGKNLEWIGLINPYNGGTNYNQKFK | LSWFQQKPGKSPKTLIYRANRLVDGVPSRFS |
| | GKATFTVDKSSSTAYMELLSLTSEDSAVYYCAR | GSGSGQDYSLTISSLEYEEMGIYYCLQYDEFP |
| | RHYGSSWDYWGQGTTLTVSS (SEQ ID NO: 282) | YTFGGGTKLEIK (SEQ ID NO: 315) |
| KCD040 | EVQLQQSGAELVKPGASVKLSCTASDFNIKDTY | QIVLTQSPAIMSASPGEKVTMTCSAGSSVSY |
| | MHWVMQRPEQGLEWIGKIDPANGNTEFDPKFQ | MYWYQQKPGSSPRVLIYDTSNLASGVPVRF |
| | GKATITADTSSNTAYLQLTSLTSEDTAVYYCTR | SGSGSGTSYSLTISRMEAEDAATYYCQQWS |
| | AMDYWGQGTSVTVSS (SEQ ID NO: 283) | NYPYTFGGGTKLEIK (SEQ ID NO: 316) |
| KCD042 | EVQLQQSGAELVKPGASVRLSCTASGFNIKHTYI | QSVLTQSPAIMSASPGEKVTMTCSANSSVSD |
| | HWVSQRPEQGLEWIGKIDPANGNTKYDPKFQG | MYWFQQRPGSSPRLLIYDTSNLASGVPVRFS |
| | KATITADTSSNTAYLQLSSLTSEDTAVYYCVNA | GSGSGTSYSLTISRMEAEDAATYYCQQWST |
| | MEYWGQGTSVTVSS (SEQ ID NO: 284) | YPWTFGGGTKLEIK (SEQ ID NO: 317) |
| KCD044 | EVQLQQSGAELVKPGASVRLSCTASGFNIKHTY | QSVLTQSPAIMSASPGEKVTMTCSANSSVSD |
| | MHWVSQRPERGLEWIGKIDPANGNTKYDPKFQ | MYWYQQRPGSSPRLLIYDTSNLASGVPVRFS |
| | GKATITADTSSNTVYLQLSSLTSEDTAVYYCLN | GSGSGTSYSLTISRMEAEDAATYYCQQWST |
| | AMEYWGQGTSVTVSS (SEQ ID NO: 285) | YPWTFGGGTKLEIK (SEQ ID NO: 318) |
| KCD047 | EVQLQQSGAEFVKPGASVRLSCTASGFNIKDTY | QIVLTQSPAVMSASPGEKVAMTCSASSSVTY |
| | MHWVKQRPEQGLEWIGRIDPANGYTKDDPKFQ | MYWYQQKPGSSPRLLIYDTSNLASGVPVRFS |
| | GKATITADTSSNTAYLQLSSLTSEDTAVYYCASA | GSGSGTSYSLTISRMEAEDAATYYCQQWST |
| | MDYWGQGTSVTVSS (SEQ ID NO: 286) | YPFPFGSGTKLEIK (SEQ ID NO: 319) |
| KCD048 | EVQLQQSGADLVKPGASVKLSCTASGFNIKATY | QIVLTQSPAIMSASPGEKVTLTCSATSSVSYM |
| | MHWVRQRPEKGLEWIGRIDPANGHTIYDPQFQG | YWYQQKPGSSPRLLIYDTSNLASGVPVRFSG |
| | KATITSDTSSNTAYLQLNSLTSEDTAVYYCAEA | SGSGTSYSLTISRMEAEDDATYYCQQWSNY |
| | MDYWGQGTSVTVSS (SEQ ID NO: 287) | PFTFGGGTKLEIK (SEQ ID NO: 320) |
| KCD070 | EIQLQQTGPELVKPGASVKISCKASGYSFTDYIIL | DIVMTQSHKFMSTSVGDRVSITCKASQDVGT |
| | WVKQSHGKSLEWIGNINPYYDYTSYNLKFKGK | AVAWYQQKPGQSPKLLIYWASTRHTGVPDR |
| | ATLTVDKSSSTAYMQLNSLTSEDSAVYYCARSD | FTGSGSGTDFTLTINNVQSEDLADYFCQQYS |
| | GYYGGDYWGQGTSVTVSS (SEQ ID NO: 2788) | SYPWTFGGGTTLEIK (SEQ ID NO: 321) |
| KCD101 | QVQLQQPGAELVRPGTSVKLSCKASGYTFTSY | DIQMTQTTSSLSASLGDRVTISCRASQDISNY |
| | WMHWVKQRPGQGLEWIGVIDPSDSYTNYNQKF | LNWYQQKPDGTVKLLIYYPSRLHSGVPSRFS |
| | KGKATLTVDTSSSTAYMQLSSLTSEDSAVYYCG | GSGSGTDYSLTISNLEQEDFATYFCQQGNTL |
| | RNGYDGSMDYWGQGTSVTVSS (SEQ ID NO: | PYTFGGGTKLEIK (SEQ ID NO: 322) |
| | 289) | |
| KCD102 | QVQLQQPGAELVRPGTSVKLSCKASGYTFTSY | EIQMTQTTSSLSASLGDRVTISCRASQDISNY |
| | WMHWVKQRPGQGLEWIGVIDPSDSYTNYNQKF | LNWYQQKPDGTVKLLIYYPSRLHSGVPSRFS |
| | KGKATLTVDTSSSTAYMQLSSLTSEDSAVYYCA | GSGSGTDYSLTISNLEQEDFATYFCQQGNTL |
| | RNGYDGSMDYWGQGTSVTVSS (SEQ ID NO: | PYTFGGGTKLEIK (SEQ ID NO: 323) |
| | 290) | |
| KCD103 | QVQLQQPGAELVRPGTSVKLSCKASGYTFTSY | DIQMTQTTSSLSASLGDRVTISCRASQDISNS |
| | WMHWVKQRPGQGLEWIGVIDPSDSYTKYNQKF | LNWYQQKPDGTVKLLIYYTSRLHSRVPSRFS |
| | KDKATLTVETSSSTAYMQLSSLTSEDSAVYYCA | GSGSGTDYSLTISNLDQEDIATYFCQQANTLP |
| | GNGYDGSMDYWGQGTSVTVSS (SEQ ID NO: | YTFGGGTKLEIK (SEQ ID NO: 324) |
| | 291) | |
| KCD104 | QVQLQQPGAELVRPGTSVKLSCKASGYTFTSY | EIQMTQTTSSLSASLGDRVTISCRASQDISNY |
| | WMHWVKQRPGQGLEWIGVIDPSDSYTYYNQKF | LNWYQQKPDGTVKLLIYYPSRLHSGVPSRFS |
| | KGKATLTVDTSSSTAYMQLSSLTSEDSAVYYCA | GSGSGTDYSLTISNLEQEDFATYFCQQGNTL |
| | RNGYDGAMDYWGQGTSVTVSS (SEQ ID NO: | PYTFGGGTKLEIK (SEQ ID NO: 325) |
| | 292) | |
| KCD118 | EVQLQQSGPELVKPGASVKISCKAFGYTFTDYY | DVLMTQTPLSLPVSLGDQASISCRSSQTIVHS |
| | KNWMRQRHGESLEWIGDINPNSGDANYNQKFK | NGDTYLEWYLQKPGQSPNLLIYKVSNRFSG |
| | GKATLTVDKSSSTAYMELRSLTSEDSAVYYCAR | VPDRFSGSGSGTDFTLKISRVEAEDLGIYYCF |
| | EGPSFAYWGHGTLVTVSA (SEQ ID NO: 293) | QGSHVPPTFGGGTKLEIK (SEQ ID NO: 326) |
| KCD119 | EVQLQQSGPELVKPGASVKISCKASGYTFTDYY | DVLMTQTPLSLPVSLGDQASISCRSSQTIVHS |
| | TNWMRQRHGESLEWIGDINPNTGDTSYNQKFR | NGDTYLEWYLQKPGQSPNLLIYKVSNRFSG |
| | VKATLTVDKSSGTAYMGLRSLTSEDSAVYYCT | VPDRFSGSGSGTDFTLKISRVEAEDLGVYYC |
| | REGPSFAYWGQGTLVTVSA (SEQ ID NO: 294) | FQGSHVPPTFGGGTTLEIK (SEQ ID NO: 327) |
| KCD121 | EVQLQQSGPELVKPGASVKISCKASGYTFTDYY | DVLMTQTPLSLPVSLGDQASISCRSNQTIVHS |
| | KNWMRQRHGESLEWIGDINPNNGDTSYNQKFR | NGDTYLEWYLQKPGQSPNLLIYKVSNRFSG |
| | GKATLTVDKSSSTAFMELRSLTSEDSAVYYCAR | VPDRFSGSGSGTDFTLRISRVEAEDLGVYYC |
| | EGPSFAYWGQGTLVTVSA (SEQ ID NO: 295) | FQGSHVPPTFGGGTKLEIK (SEQ ID NO: 328) |
| KCD122 | EVQLQQSGPELVKPGASVKISCKASGYTFTDYY | DVLMTQTPLSLPVSLGDQASISCRSSQTIVHS |
| | KNWMRQRHGESLEWIGDINPNNGDANYNQKFK | NGDTYLEWYLQKPGQSPNLLIYKVSNRFSG |
| | GKATLTVDKSSSTAYMELRSLTSEDSAVYFCAR | VPDRFSGSGSGTDFTLKISRVEAEDLGVYYC |
| | EGPSFAYWGHGTLVTVSA (SEQ ID NO: 296) | FQGSHVPPTFGGGTKLEIK (SEQ ID NO: 329) |
| KCD123 | EVHLQQSGPELVKPGASVKISCKASGYTFTDFY | DVLMTQTPLSLPVSLGDQASISCRSSQTIVHS |
| | KNWMRQRHGESLEWIGDINPNNGGTNYNQKFK | NGDTYLEWYLQKPGQSPNLLIYKVSNRFSG |
| | GKATLTVDKSSSTAYMELRSLTSEDSAVYYCAR | VPDRFSGSGSGTDFTLKISRVEAEDLGVYYC |
| | EGPSFAYWGQGTLVTVSA (SEQ ID NO: 297) | FQGSHVPPTFGGGTKLEIK (SEQ ID NO: 330) |
| KCD124 | EVQLQQSGPELVKPGASVKISCKASGYTFTDHY | DVLMTQTPLSLPVSLGDQASISCRSSQSIVHS |
| | MNWVKQSHGKSLEWIGDINPNNGGTSYNQKFK | NGDTYLEWYLQKPGQSPKLLIYKVSNRFSG |
| | GKATLTVDKSSSTAYMELRSLTSGDSAVYYCTR | VPDRFSGSGSGTDFTLKISRVEAEDLGVYYC |
| | EGASFAFWGQGTLVTVSA (SEQ ID NO: 298) | FQGSHVPLTFGAGTKLELK (SEQ ID NO: 331) |
| KCD131 | QVQLQQSGPELVKPGASVKISCKASAYTFTDYY | DIQMTQSPASLSVSVGETVTITCRASENIYSH |
| | INWVKQRPGQGPEWIGWIFPGSNSTYSNEKFEV | LAWFQQKQGKSPRLLVYSATNLPDGVPSRF |
| | KATLTVDESSSTAYMLLSSLTSEDSAVYFCARL | SGSGSGTQYSLKINILQSEDFGSYYCQHFWG |
| | GYFGSSYHALDYWGQGTSVTVSS (SEQ ID NO: | TPWTFGGGTKLEIK (SEQ ID NO: 332) |
| | 299) | |
| KCD136 | EVQLQQSVAELVRPGASVKLSCSASGFNIKNTY | QIVLTQSPAIMSASPGEKVTMTCSASSSVSY |
| | MHWVNQRPEQGLEWIGRIDPANGITKYAPNFQ | MYWYQQKPGSSPRLLIYDISNLASGVPVRFS |
| | GKATITADTSSNTAYLQLSNLTSEDTAIYYCTRA | GSGSGTSYSLTISRMEAEDAATYYCQQWDT |
| | MDYWGQGTSVTVSS (SEQ ID NO: 300) | YPWTFGGGTKLEIK (SEQ ID NO: 333) |
| KCD200 | EVQLQQSGPELVKPGASVKISCKASGYTFTSYY | DVLMTQTPLSLPVSLGDQVSISCRSSQTIVHS |
| | KNWMRQRHGESLEWIGDINPNSGDTAYNQKFK | NGDTYLEWYLQKPGQSPNLLIYKVSNRFSG |
| | GKATLTVDRSSSTAYMELRSLTSEDSAVYYCAR | VPDRFSGSGSGTDFTLKISRVEAEDLGVYYC |
| | EGPSFAYWGQGTLVTVSA (SEQ ID NO: 301) | FQGSHVPPTFGGGTKLEIK (SEQ ID NO: 334) |
| KCD208 | EVQLQQSVAELVRPGASVKLSCTVSGFNIKNTY | EIVLTQSPALMAASPGEKVTITCSVSSSISSSS |
| | MHWVKQRPEQGLEWIGRIDPANGDTTYAPKFQ | LHWYRQKSGTSPKPWIYGTSHLASGVPVRFS |
| | GKATITADTSSNSAYLHLSRLTSEDTAIYYCSLY | GSGSGTSYSLTISSMEAEDAATYYCQQWDT |
| | DYDGYWGQGTTLTVSS (SEQ ID NO: 302) | YPWTFGGGTKLEIK (SEQ ID NO: 335) |
| KCD214 | EVQLQQSVAEFVRPGASVKLSCTASGFNIKNTY | EIVLTQSPALMAASPGEKVTITCRVSSSISSSS |
| | MHWVKQRPEQGLEWIGRIDPANGNTEYAPKFQ | LHWYQQKSGTSPKPWIYGTSNLASGVPVRF |
| | GKATITADTSSNTAYLQLSSLTSEDTAIYYCALY | SGSRSGTSYSLTISSMEAEDAATYYCQQWSD |
| | DYDGYWGQGTTLTVSS (SEQ ID NO: 303) | YPWTFGGGTKLEIK (SEQ ID NO: 336) |
| KCD220 | QVQLQQSGAELMEPGASVKLSCKATGYTFTGY | DIQMTQTTSSLSASLGDRVTISCRASQDISNY |
| | WIEWVKQRPGHGLEWIGETLPGSDSNNYNEKF | LNWYQQKPDGTVKLLIYYTSNLHSGVPSRFS |
| | KGKATFTADTSSNTAYMQLSSLTTEDSAIYYCA | GSGSGTDYSLTISNLEQEDIATYFCQQDSKH |
| | RDYSNYWYFDVWGTGTTVTVSS (SEQ ID NO: | RTFGGGTKLEIK (SEQ ID NO: 337) |
| | 304) | |
| KCD224 | QVQLKESGPGLVAPSQSLSITCTVSGFSLTSYGV | DIQMTQTTSSLSASLGDRVTISCRASQVISNY |
| | DWIRQSPGKGLEWLGVIWGVGSTNYNSALKSR | LNWYQQKPDGTVKLLIYYTSRLHSGVPSRFS |
| | LSISKDNSKSQVFLKMNSLQTDDTAMYYCARSY | GSGSGTDYSLTISNLEPEDIATYYCQQYSKLP |
| | DGSYWYFDVWGTGTTVTVSS (SEQ ID NO: 305) | YTFGSGTKLEIK (SEQ ID NO: 338) |
| 119_ | EVOLVESGGGLVQPGGSLRLSCAASGYTFTDYY | DIQLTQSPSSLSASVGDRVTITCRSSQTIVHSN |
| TAF | MNWVRQAPGKGLEWIGDINPNTGDTSYNADFK | GDTYLEWYQQKPGKAPNLLIYKVSNRFSGV |
| | RRFTFSLDTSKSTAYLQMNSLRAEDTAVYYCTR | PSRFSGSGSGTDFTLTISSLQPEDFATYYCFQ |
| | EGPSFAYWGQGTLVTVSS (SEQ ID NO: 306) | GSHVPPTFGQGTKVEIK (SEQ ID NO: 339) |
| 119_ | EVQLVESGGGLVQPGGSLRLSCAASGYTFTDYY | DIQMTQSPSSLSASVGDRVTITCRSSQTIVHS |
| TAF Germ | MSWVRQAPGKGLEWIGDINPNTGDTSYNADSV | NGDTYLEWYQQKPGKAPNLLIYKVSNRFSG |
| | KGRFTISRDNSKNTLYLQMNSLRAEDTAVYYCT | VPSRFSGSGSGTDFTLTISSLQPEDFATYYCF |
| | REGPSFAYWGQGTLVTVSS (SEQ ID NO: 307) | QGSHVPPTFGQGTKVEIK (SEQ ID NO: 340) |
| 119_ | EVKKPGASVKVSCKASGYTFTDYYMHWVRQA | DVVMTQSPLSLPVTLGQPASISCRSSQTIVHS |
| Human Germ | PGQGLEWIGDINPNTGDTSYNQKFQGRVTSTRD | NGDTYLEWFQQRPGQSPNLLIYKVSNRFSGV |
| | TSISTAYMELSRLRSDDTVVYYCTREGPSFAYW | PDRFSGSGSGTDFTLKISRVEAEDVGVYYCF |
| | GQGTLVTVSS (SEQ ID NO: 308) | QGSHVPPTFGGGTKVEIK (SEQ ID NO: 341) |

TABLE 1B
CDRs of anti-CFD Antagonist monoclonal Antibodies based on a broad CDR
definition

| | H1 | H2 | H3 | L1 | L2 | L3 |
|---|---|---|---|---|---|---|
| KCD002 | GFTFSDYYM | INYDGSSTYY | ARGEDFYLY | RASENIHSYL | NTKTLAE | QHHYGIPPT |
| | A (SEQ ID NO: | LDSLKS (SEQ | AMDY (SEQ | A (SEQ ID NO: | (SEQ ID | (SEQ ID NO: |
| | 342) | ID NO: 372) | ID NO: 402) | 432) | NO: 462) | 492) |
| KCD003 | GYTFTDYYM | INPNNGDPSY | AREGPSFAY | RSSQTIVHSN | KVSNRFS | FQGSHVPPT |
| | N (SEQ ID NO: | NQKFKD (SEQ | (SEQ ID NO: | GDTYLE (SEQ | (SEQ ID | (SEQ ID NO: |
| | 343) | ID NO: 373) | 403) | ID NO: 433) | NO: 463) | 493) |
| KCD005 | GYTFTDHYM | INPNNGGTSC | TREGASFAF | RSSQSIVHSN | KVSNRFS | FQGSHVPVT |
| | N (SEQ ID NO: | NQKFKG (SEQ | (SEQ ID NO: | GDTYLE (SEQ | (SEQ ID | (SEQ ID NO: |
| | 344) | ID NO: 374) | 404) | ID NO: 434) | NO: 464) | 494) |
| KCD009 | GYIFRNYGM | INTYTGEPTY | VRDGPGFAY | RSSLIIEHSDG | KVSNRFS | FQGSHVPVT |
| | N (SEQ ID NO: | ADDFKG (SEQ | (SEQ ID NO: | NTYLE (SEQ | (SEQ ID | (SEQ ID NO: |
| | 345) | ID NO: 375) | 405) | ID NO: 435) | NO: 465) | 495) |
| KCD010 | GYTFTDYDM | INPNNGGTIY | GTGDFAY | SASSSVSSSY | STSNLAS | HQWSSYPPT |
| | D (SEQ ID NO: | NQKFKG (SEQ | (SEQ ID NO: | LY (SEQ ID | (SEQ ID | (SEQ ID NO: |
| | 346) | ID NO: 376) | 406) | NO: 436) | NO: 466) | 496) |
| KCD023 | GYTFTDYNID | INPNNGGINY | GTGDYAY | SASSSVSSSY | STSNLAS | HQWTSYPPT |
| | (SEQ ID NO: | NQKFKG (SEQ | (SEQ ID NO: | LY (SEQ ID | (SEQ ID | (SEQ ID NO: |
| | 347) | ID NO: 377) | 407) | NO: 437) | NO: 467) | 497) |
| KCD036 | GYSFTGYTM | INPYNGGTNY | ARRHYGSSW | KASQDINTYL | RANRLVD | LQYDEFPYT |
| | T (SEQ ID NO: | NQKFKG (SEQ | DY (SEQ ID | S (SEQ ID NO: | (SEQ ID | (SEQ ID NO: |
| | 348) | ID NO: 378) | NO: 408) | 438) | NO: 468) | 498) |
| KCD040 | DFNIKDTYM | IDPANGNTEF | TRAMDY | SAGSSVSYM | DTSNLAS | QQWSNYPYT |
| | H (SEQ ID NO: | DPKFQG (SEQ | (SEQ ID NO: | Y (SEQ ID NO: | (SEQ ID | (SEQ ID NO: |
| | 349) | ID NO: 379) | 409) | 439) | NO: 469) | 499) |
| KCD042 | GFNIKHTYIH | IDPANGNTKY | VNAMEY | SANSSVSDM | DTSNLAS | QQWSTYPW |
| | (SEQ ID NO: | DPKFQG (SEQ | (SEQ ID NO: | Y (SEQ ID NO: | (SEQ ID | T (SEQ ID NO: |
| | 350) | ID NO: 380) | 410) | 440) | NO: 470) | 500) |
| KCD044 | GFNIKHTYM | IDPANGNTKY | LNAMEY | SANSSVSDM | DTSNLAS | QQWSTYPW |
| | H (SEQ ID NO: | DPKFQG (SEQ | (SEQ ID NO: | Y (SEQ ID NO: | (SEQ ID | T (SEQ ID NO: |
| | 351) | ID NO: 381) | 411) | 441) | NO: 471) | 501) |
| KCD047 | GFNIKDTYM | IDPANGYTKD | ASAMDY | SASSSVTYMY | DTSNLAS | QQWSNYPFT |
| | H (SEQ ID NO: | DPKFQG (SEQ | (SEQ ID NO: | (SEQ ID NO: | (SEQ ID | (SEQ ID NO: |
| | 352) | ID NO: 382) | 412) | 442) | NO: 472) | 502) |
| KCD048 | GFNIKATYM | IDPANGHTIY | AEAMDY | SATSSVSYMY | DTSNLAS | QQWSNYPFT |
| | H (SEQ ID NO: | DPQFQG (SEQ | (SEQ ID NO: | (SEQ ID NO: | (SEQ ID | (SEQ ID NO: |
| | 353) | ID NO: 383) | 413) | 443) | NO: 473) | 503) |
| KCD070 | GYSFTDYIIL | INPYYDYTSY | ARSDGYYGG | KASQDVGTA | WASTRHT | QQYSSYPWT |
| | (SEQ ID NO: | NLKFKG (SEQ | DY (SEQ ID | VA (SEQ ID | (SEQ ID | (SEQ ID NO: |
| | 354) | ID NO: 384) | NO: 414) | NO: 444) | NO: 474) | 504) |
| KCD101 | GYTFTSYWM | IDPSDSYTNY | GRNGYDGSM | RASQDISNYL | YPSRLHS | QQGNTLPYT |
| | H (SEQ ID NO: | NQKFKG (SEQ | DY (SEQ ID | N (SEQ ID NO: | (SEQ ID | (SEQ ID NO: |
| | 355) | ID NO: 385) | NO: 415) | 445) | NO: 475) | 505) |
| KCD102 | GYTFTSYWM | IDPSDSYTNY | ARNGYDGSM | RASQDISNYL | YPSRLHS | QQGNTLPYT |
| | H (SEQ ID NO: | NQKFKG (SEQ | DY (SEQ ID | N (SEQ ID NO: | (SEQ ID | (SEQ ID NO: |
| | 356) | ID NO: 386) | NO: 416) | 446) | NO: 476) | 506) |
| KCD103 | GYTFTSYWM | IDPSDSYTKY | AGNGYDGSM | RASQDISNSL | YTSRLHS | QQANTLPYT |
| | H (SEQ ID NO: | NQKFKD (SEQ | DY (SEQ ID | N (SEQ ID NO: | (SEQ ID | (SEQ ID NO: |
| | 357) | ID NO: 387) | NO: 417) | 447) | NO: 477) | 507) |
| KCD104 | GYTFTSYWM | IDPSDSYTYY | ARNGYDGAM | RASQDISNYL | YPSRLHS | QQGNTLPYT |
| | H (SEQ ID NO: | NQKFKG (SEQ | DY (SEQ ID | N (SEQ ID NO: | (SEQ ID | (SEQ ID NO: |
| | 358) | ID NO: 388) | NO: 418) | 448) | NO: 478) | 508) |
| KCD118 | GYTFTDYYK | INPNSGDANY | AREGPSFAY | RSSQTIVHSN | KVSNRFS | FQGSHVPPT |
| | N (SEQ ID NO: | NQKFKG (SEQ | (SEQ ID NO: | GDTYLE (SEQ | (SEQ ID | (SEQ ID NO: |
| | 359) | ID NO: 389) | 419) | ID NO: 449) | NO: 479) | 509) |
| KCD119 | GYTFTDYYT | INPNTGDTSY | TREGPSFAY | RSSQTIVHSN | KVSNRFS | FQGSHVPPT |
| | N (SEQ ID NO: | NQKFRV (SEQ | (SEQ ID NO: | GDTYLE (SEQ | (SEQ ID | (SEQ ID NO: |
| | 360) | ID NO: 390) | 420) | ID NO: 450) | NO: 480) | 510) |
| KCD121 | GYTFTDYYK | INPNNGDTSY | AREGPSFAY | RSNQTIVHSN | KVSNRFS | FQGSHVPPT |
| | N (SEQ ID NO: | NOKFRG (SEQ | (SEQ ID NO: | GDTYLE (SEQ | (SEQ ID | (SEQ ID NO: |
| | 361) | ID NO: 391) | 421) | ID NO: 451) | NO: 481) | 511) |
| KCD122 | GYTFTDYYK | INPNNGDANY | AREGPSFAY | RSSQTIVHSN | KVSNRFS | FQGSHVPPT |
| | N (SEQ ID NO: | NQKFKG (SEQ | (SEQ ID NO: | GDTYLE (SEQ | (SEQ ID | (SEQ ID NO: |
| | 362) | ID NO: 392) | 422) | ID NO: 452) | NO: 482) | 512) |
| KCD123 | GYTFTDFYKN | INPNNGGTNY | AREGPSFAY | RSSQTIVHSN | KVSNRFS | FQGSHVPPT |
| | (SEQ ID NO: | NQKFKG (SEQ | (SEQ ID NO: | GDTYLE (SEQ | (SEQ ID | (SEQ ID NO: |
| | 363) | ID NO: 393) | 423) | ID NO: 453) | NO: 483) | 513) |
| KCD124 | GYTFTDHYM | INPNNGGTSY | TREGASFAF | RSSQSIVHSN | KVSNRFS | FQGSHVPLT |
| | N (SEQ ID NO: | NQKFKG (SEQ | (SEQ ID NO: | GDTYLE (SEQ | (SEQ ID | (SEQ ID NO: |
| | 364) | ID NO: 394) | 424) | ID NO: 454) | NO: 484) | 514) |
| KCD131 | AYTFTDYYIN | IFPGSNSTYSN | ARLGYFGSSY | RASENIYSHL | SATNLPD | QHFWGTPW |
| | (SEQ ID NO: | EKFEV (SEQ | HALDY (SEQ | A (SEQ ID NO: | (SEQ ID | T (SEQ ID NO: |
| | 365) | ID NO: 395) | ID NO: 425) | 455) | NO: 485) | 515) |
| KCD136 | GFNIKNTYM | RIDPANGITK | TRAMDY | SASSSVSYMY | DISNLAS | QQWDTYPW |
| | H (SEQ ID NO: | YAPNFQG | (SEQ ID NO: | (SEQ ID NO: | (SEQ ID | T (SEQ ID NO: |
| | 366) | (SEQ ID NO: | 426) | 456) | NO: 486) | 516) |
| | | 396) | | | | |
| KCD200 | GYTFTSYYKN | INPNSGDTAY | AREGPSFAY | RSSQTIVHSN | KVSNRFS | FQGSHVPPT |
| | (SEQ ID NO: | NQKFKG (SEQ | (SEQ ID NO: | GDTYLE (SEQ | (SEQ ID | (SEQ ID NO: |
| | 367) | ID NO: 397) | 427) | ID NO: 457) | NO: 487) | 517) |
| KCD208 | GFNIKNTYM | RIDPANGDTT | SLYDYDGY | SVSSSISSSSL | GTSHLAS | QQWDTYPW |
| | H (SEQ ID NO: | YAPKFQG | (SEQ ID NO: | H (SEQ ID NO: | (SEQ ID | T (SEQ ID NO: |
| | 368) | (SEQ ID NO: | 428) | 458) | NO: 488) | 518) |
| | | 398) | | | | |
| KCD214 | GFNIKNTYM | RIDPANGDTT | ALYDYDGY | RVSSSISSSSL | GTSNLAS | QQWSDYPW |
| | H (SEQ ID NO: | YAPKFQG | (SEQ ID NO: | H (SEQ ID NO: | (SEQ ID | T (SEQ ID NO: |
| | 369) | (SEQ ID NO: | 429) | 459) | NO: 489) | 519) |
| | | 399) | | | | |
| KCD220 | GYTFTGYWIE | ETLPGSDSNN | ARDYSNYWY | RASQDISNYL | YTSNLHS | QQDSKHRT |
| | (SEQ ID NO: | YNEKFKG | FDV (SEQ ID | N (SEQ ID NO: | (SEQ ID | (SEQ ID NO: |
| | 370) | (SEQ ID NO: | NO: 430) | 460) | NO: 490) | 520) |
| | | 400) | | | | |
| KCD224 | GFSLTSYGVD | VIWGVGSTN | ARSYDGSYW | RASQVISNYL | YTSRLHS | QQYSKLPYT |
| | (SEQ ID NO: | YNSALKS | YFDV (SEQ ID | N (SEQ ID NO: | (SEQ ID | (SEQ ID NO: |
| | 371) | (SEQ ID NO: | NO: 431) | 461) | NO: 491) | 521) |
| | | 401) | | | | |

TABLE 1C
Heavy and Light Chain Variable Sequences of 88 mouse anti-CFD antibodies with
CDRs underlined.

| | Heavy Chain Variable | ID | Light Chain Variable |
|---|---|---|---|
| ID | Region AA Sequence | | Region AA Sequence |
| FKCD001 | EVQLQQSGPELVKPGASVKISCKASDN | KCD001 | QIVLSQSPAILSASPGEKVTMTCRASSSV |
| | SFTGYYMHWVKQSHGNILDWIGYIDP | | SYMHWYQQKPGSSPKPWIYATSNLASG |
| | YNGVSSYNQKFKGKATLTVDKSSSTA | | VPARFSGSGSGTSHFLTISRLEAEDAAT |
| | YMEIRSLTSEDSAVYYCASYYGSSPYW | | YFCQQWSSNPYTFGGGTKLEIK (SEQ ID |
| | YFDVWGTGTTVTVSS (SEQ ID NO: 522) | | NO: 610) |
| KCD002 | EVKLVESEGGLVQPGSSMKLSCTASGF | KCD002 | DIQMTQSPASLSASVGETVTITCRASENI |
| | TFSDYYMAWVRQVPEKGLEWVGNINY | | HSYLAWYQQKQGKSPQLIVYNTKTLAE |
| | DGSSTYYLDSLKSRFIISRDSAKNILYLQ | | GVPSRFSGSGSGTQFSLKINSLQPEDFGS |
| | MSSLKSEDTATYFCARGEDFYLYAMD | | YYCQHHYGIPPTFGGGTKLEIK (SEQ ID |
| | YWGQGTSVTVSS (SEQ ID NO: 523) | | NO: 611) |
| KCD003 | EVQLQQSRPELVKPGASVKIFCKASGY | KCD003 | DVLMTQTPLSLPVSLGEQASISCRSSQTI |
| | TFTDYYMNWMRQRHGETLEWIGDINP | | VHSNGDTYLEWYLQKPGQSPNLLIYKV |
| | NNGDPSYNQKFKDKATLTVDKSSSTAS | | SNRFSGVPDRFSGSGSGTDFTLKISRVEA |
| | MELRSLTSDDSAVYYCAREGPSFAYW | | EDLGVYYCFQGSHVPPTFGGGTKLEIK |
| | GQGTLVTVSA (SEQ ID NO: 524) | | (SEQ ID NO: 612) |
| KCD004 | EVQLQQSGAELVRPGSSVKMSCKTSG | KCD004 | DIQMTQSPTSLSASLGESVSLTCRASQEI |
| | KTFTSHGINWVKQRPGQGLEWIGYIYI | | SGYLNWLQQKPDGSIKRLIYAASTLDSG |
| | GNGYNEYNEKFKGKATLTSDTSSSTAY | | VPKRFSGSRSGSDYSLTISSLESEDFADY |
| | MQLSSLTFEDSAIYFCVRKAYGNYGFD | | YCLQYANYPFTFGSGTKLEVK (SEQ ID |
| | DWGQGTTLTVSS (SEQ ID NO: 525) | | NO: 613) |
| KCD005 | EVQLQQSGPELVKPGASVKISCKASGY | KCD005 | DVLMTQTPLSLPVSLGDQASISCRSSQSI |
| | TFTDHYMNWVKQSHGKSLEWIGDINP | | VHSNGDTYLEWYLQKPGQSPKLLIYKV |
| | NNGGTSCNQKFKGKATLTVDKSSSTA | | SNRFSGVPDRFSGSGSGTDFTLKISRVEA |
| | YMELRSLTSGDSAVYYCTREGASFAF | | EDLGVYYCFQGSHVPVTFGAGTKLELK |
| | WGQGTLVTVSA (SEQ ID NO: 526) | | (SEQ ID NO: 614) |
| KCD006 | EVQLQQSGAELVKPGASVKLSCTASGF | KCD006 | QIILTQSPAIMSASPGEKVTMTCSARSSV |
| | KIKDTYMHWVKERPEQGLEWIGRIDPA | | SNMYWYQQKPGSSPRLLIYDTSNLASG |
| | NGNTKYDPKFQGKATITADTSSNTAYL | | VPVRFSGSGSGTSYSLTISRMEAEDAAT |
| | QLSSLTSEDTAVYYCANAMDYWGQGT | | YYCQQWSSYPWTFGGGTKLEIK (SEQ |
| | SVTVSS (SEQ ID NO: 527) | | ID NO: 615) |
| KCD007 | EVQLQQSGAELVKPGASVKLSCTASGF | KCD007 | QIILTQSPAIMSASPGERVTMTCSASSSV |
| | KIKDTYMHWVKERPEQGLEWIGRIDPA | | SNMYWYQQKPGSSPRLLIYDTSNLASG |
| | NGNTKYDPKFQGKATITADTSSNTAYL | | VPLRFSGSGSGTSYSLTISRMEAEDAAT |
| | QLSSLTSEDTAVYYCANAMDYWGQGT | | YYCQQWSSYPWTFGGGTKLEIK (SEQ |
| | SVTVSS (SEQ ID NO: 528) | | ID NO: 616) |
| KCD008 | EVQLQQSGPELVKAGASVKMSCTASG | KCD008 | QIVLTQSPEIMSASPGEKVTMTCSARSS |
| | FNIKDTYMHWVKQRPEQGLAWIGRIDP | | VSYMYWYQQKPGSSPRLLIYDTSNLAS |
| | ANGNIKYDPKFQGKATITADTSSNTAY | | GVPVRFSGSGSGTSYSLTISRMETEDAA |
| | LQLSSLTSDDTAVYYCTSAMDYWGQG | | TYYCQQWSTYPFTFGSGTKLEIK (SEQ |
| | TSVTVSS (SEQ ID NO: 529) | | ID NO: 617) |
| KCD009 | QIQLVQSGPELKKPGETVKISCKASGYI | KCD009 | DVLMTQTPLSLPVSLGDQASISCRSSLII |
| | FRNYGMNWVKQGPGKGLKWMGWIN | | EHSDGNTYLEWYLQKPGQSPKLLIYKV |
| | TYTGEPTYADDFKGRFAFSLETSASTA | | SNRFSGVPDRFSGSGSGTDFTLKISRVEA |
| | YLQISNLKNEDTATYFCVRDGPGFAYW | | DDLGVYYCFQGSHVPVTFGAGTNLELK |
| | GQGTLVTVSA (SEQ ID NO: 530) | | (SEQ ID NO: 618) |
| KCD010 | EVLLQQSGPELVKPGASVKIPCKASGY | KCD010 | QIVLTQSPAIMSVSPGEKVTLTCSASSSV |
| | TFTDYDMDWVKQSHGKSLEWIGHINP | | SSSYLYWYQKKPGSSPKLWIYSTSNLAS |
| | NNGGTIYNQKFKGKATLTVDKSSSTAY | | GVPARFSGSGSGTSYSLTISSMEAEDAA |
| | MELRSLTSEDTAVYYCGTGDFAYWGH | | SYFCHQWSSYPPTFGAGTKLELK (SEQ |
| | GTLVTVSA (SEQ ID NO: 531) | | ID NO: 619) |
| KCD011 | EVKLVESGGGLVQPGGSLKLSCAASGF | KCD011 | DIQMTQSPSSLSASLGGKVTITCKASQDI |
| | TFSSNTMSWVRQTPEKRLEWVAYITNG | | NKYIAWYQHKPGKGPRLLIHYTSTLQP |
| | GGSTYYPDTVKGRFTISRDNARNTLYL | | GIPSRFSGSGSGRDYSFSISNLEPEDIATY |
| | QMSSLKSEDTAMYYCARHDYYTMDY | | YCLQYDNLLYTFGGGTKLEIK (SEQ ID |
| | WGQGTSVTVSS (SEQ ID NO: 532) | | NO: 620) |
| KCD013 | EVQLQQSGAELVKPGASVKLSCTASGF | KCD013 | QIILTQSPAIMSASPGEKVTMTCSASSSV |
| | KIKDTYMHWVKERPDQGLEWIGRIDP | | SNMYWYQQKPGSSPRLLIYDTSNLASG |
| | ANGNTKYDPKFQGKATITADTSSNTAY | | VPVRFSGSGSGASYSLTISRMEAEDAAT |
| | LQLSSLTSEDTAVYYCANAMDYWGQG | | YYCQQWSSYPWTFGGGTKLEIK (SEQ |
| | TSVTVSS (SEQ ID NO: 533) | | ID NO: 621) |
| KCD014 | EVKLVESGGGLVQPGGSLKLSCAASGF | KCD014 | EIVLTQSPTTMAASPGEKITITCSASSSIS |
| | TFSSYIMSWVRQTPEKRLEWVAYITNG | | SNYLHWYQQKPGFSPKLLIYRTSNLASG |
| | GGNTYYPDTIKGRFTISRDNAKNTLYL | | VPARFSGSGSGTSYSLTIGTMEAEDVAT |
| | QMSSLKSEDTAMYYCARHGTGYAMD | | YYCQQGSSIPLTFGAGTKLELK (SEQ ID |
| | YWGQGTSVTVSS (SEQ ID NO: 534) | | NO: 622) |
| KCD015 | EVQLQQSGAELVKPGASVKLSCTASGF | KCD015 | QIVLTQSPAILSASPGEKVTMTCSASSSV |
| | NIKDTYMHWVKQRPEQGLEWIGRIDP | | SYIYWYQQKPRSSPRLLIYDTSNLASGV |
| | ANGYTEYDPKFQGKATITADTSSNTAY | | PVRFSGSGSGTSYSLTISRMEAEDAATY |
| | LQLSSLTSEDSAAYYCTSAMEFWGQGT | | YCQQWSSYPFTFGGGTKLEVK (SEQ ID |
| | SVTVSS (SEQ ID NO: 535) | | NO: 623) |
| KCD016 | QVQLQQSGAELAKPGASVKMSCKASG | KCD016 | DIQMTQSPASLSASVGETVTITCRASGNI |
| | YTFTNFWMHWVKQRPGQGLEWIGFFN | | HNYLAWYQQKQGKSPQLLVYNAKTLA |
| | PSTAYTEYNQKFKDKATLTADKSSSTA | | DGVPSRFSGSGSGTQYSLKINSLQPEDF |
| | YLHLSSLTSEDSAVYYCARRDYGSSYG | | GYYYCQHFWSTPTFGGGTKLEIK (SEQ |
| | WYFDVWGAGTTVTVSS (SEQ ID NO: | | ID NO: 624) |
| | 536) | | |
| KCD017 | DVQLQESGPDLVKPSQSLSLTCTVTDY | KCD017 | DIVMTQSHKFMSTSVGDRVYITCKASQ |
| | SITSGYSWHWIRQFPGNKLEWLGYIHS | | DVGTAVAWYQQTSGQSPKLLIYWASTR |
| | SGNTNYNPSLKSRFSITRDTSKNQFFLQ | | HTGVPDRFTGSGSGTDFTLTLSNVQSED |
| | LNSVTSEDTATYYCALHYYGSSFGWY | | LADYFCQQYTSYPLTFGAGTKLELK |
| | FDVWGAGTTVTVSS (SEQ ID NO: 537) | | (SEQ ID NO: 625) |
| KCD018 | QVQLQQSAAELARPGASVKMSCKASG | KCD018 | NIMMTQSPSSLAVSAGEKVTMSCKSSQ |
| | YTFTSYTVHWVKQRPGQGLEWIGYINP | | SVLYSSNQKNYLAWYQQKPGQSPQLLI |
| | SSGFTDYNQKFKDKTTLTADISSSTAYI | | YWASTRESGVPERFTGSGSGTDFTLTISS |
| | QLSSLTSEDSAVYYCARRGVNWSWFA | | VQAEDLAVYYCHQYLSSWTFGGGTKL |
| | YWGQGTLVTVSA (SEQ ID NO: 538) | | EIK (SEQ ID NO: 626) |
| KCD019 | QVQLQQSGAELARPGASVKMSCKASG | KCD019 | DIVMTQAHRFMSTSVGDRVIISCKASQD |
| | YTFATYTIHWVKQRPGQGLEWIGYLNL | | VGTAVAWYQQTPGQSPKILIYWTSTRH |
| | RNDYTHYNQKFRDKAALTADKSSSTA | | TGVPDRFTGSRSGTDFTLTISNVQSEDL |
| | YMQLSSLTSEDSAVYNCAFRLGNDRQ | | ADYFCQQYTTYPLTFGGGTKLEIK (SEQ |
| | GWYFDVWGAGTTVTVSS (SEQ ID NO: | | ID NO: 627) |
| | 539) | | |
| KCD022 | DVQLQESGPDLVKPSQSLSLTCTVTGY | KCD022 | DIVMTQSHKFMSTSIGDRVIITCKASQD |
| | SITSGYSWHWIRQFPGNTLEWMGYIHY | | VGTTVAWYQQRPGQSPKLLIYWASTRH |
| | SGSTNYNPSLESRISFTRDTSKNQFFLQ | | TAVPDRFTGSGSGTDFTLTISNVQSEDL |
| | LNSVTTEDTATYYCALHFYGYNLGWY | | ADYFCQQYTSYPLTFGAGTQLELK |
| | FDVWGAGTTVTVSS (SEQ ID NO: 540) | | (SEQ ID NO: 628) |
| KCD023 | EVLLQQSGPELVKPGASVKIPCKASGY | KCD023 | QIVLTQSPAFMSASPGEKVTLTCSASSS |
| | TFTDYNIDWVKQSHGKSLEWIGDINPN | | VSSSYLYWYQQKPGSSPKLWIYSTSNL |
| | NGGINYNQKFKGKATLTVDKSSSTAY | | ASGVPGRFSGSGSGTSYSLTISSMEAED |
| | MELRSLTSEDTAVYYCGTGDYAYWGQ | | AASYFCHQWTSYPPTFGAGTKLELK |
| | GTLVTVSA (SEQ ID NO: 541) | | (SEQ ID NO: 629) |
| KCD030 | QVQLQQSGAELAKPGASVKMSCKASG | KCD030 | DIQMNQSPSSLSASLGDTITITCHASQNI |
| | YTFTNYWMHWVKQRPGQGLEWIGYIN | | NVWLSWYQQKPGNIPKLLIYKASNLHT |
| | PSIGYTEYNQKFKDKATLTADKSSSTA | | GVPSRFSGSGSGTGFTLTISSLQPEDIAT |
| | YMQLSSLTSEDSAVFYCATFIYYAMDY | | YYCQQGQSYPYTFGGGTKLEIK (SEQ |
| | WGQGTSVTVSS (SEQ ID NO: 542) | | ID NO: 630) |
| KCD033 | EVQLQQSGPELVKPGASMKISCTASGY | KCD033 | DIKLTQSPSSIYTSLGERVTITCKASQDIN |
| | SFTGYTMTWVKQSHGKNLEWIGLINPY | | TYLSWFQQRPGKSPKTLIYRADRLVDG |
| | NGGTNYNQKFKGKATLTVDKSSSIAY | | VPSRVRGSGSGQDYSLTISSLEYEDMGI |
| | MELLSLTSEDSAVYYCARRHYGSNWD | | YYCLQYDEFPYTFGGGTKLEIK (SEQ ID |
| | YWGQGTTLTVSS (SEQ ID NO: 543) | | NO: 631) |
| KCD036 | EVQLQQSGPELVKPGASMKISCKASGY | KCD036 | DIKLTQSPSSMYASLGERVTITCKASQDI |
| | SFTGYTMTWVKQSHGKNLEWIGLINPY | | NTYLSWFQQKPGKSPKTLIYRANRLVD |
| | NGGTNYNQKFKGKATFTVDKSSSTAY | | GVPSRFSGSGSGQDYSLTISSLEYEEMGI |
| | MELLSLTSEDSAVYYCARRHYGSSWD | | YYCLQYDEFPYTFGGGTKLEIK (SEQ ID |
| | YWGQGTTLTVSS (SEQ ID NO: 544) | | NO: 632) |
| KCD038 | EVQLQQSGPELVKPGTSMKISCKASGY | KCD038 | DVVMTQTPLSLPVSLGDQASISCRSSQS |
| | SFADYTMNWVKQSHGKSLEWIGLINP | | LVHSNGNTYLYWYFQKPGQSPKFLIYK |
| | YNGGTSYNQKFMGKATLTVDKSSSTA | | VSNRFSGISDRFSGSGSGTDFTLKISRVE |
| | YMELLSLTSEDSAVYYCARWGTYSHN | | AEDLGVYFCSQSTHVPPFTFGSGTKLEI |
| | YDYAMDYWGQGTSVTVSS (SEQ ID | | K (SEQ ID NO: 633) |
| | NO: 545) | | |
| KCD039 | EVQLQQSGPEMVKPGASMKISCKASG | KCD039 | DVVMTQTPLSLSVSLGDQASISCRSSQS |
| | YSFADYTLNWVKQSHGKSLEWIGLINP | | LVHSNGNTYLYWYLQKPGQSPKLLIYK |
| | YNGGTSYNQKFMGKATLTVDKSSSTA | | VSNRFSGITDRFSGSGSGTDFTLKISRVE |
| | YMELLSLTSEDSAVYYCTRWGTYSHN | | AEDLGVYFCSQSTHVPPFTFGSGTKLEI |
| | YDYAMDYWGQGTSVTVSS (SEQ ID | | K (SEQ ID NO: 634) |
| | NO: 546) | | |
| KCD040 | EVQLQQSGAELVKPGASVKLSCTASDF | KCD040 | QIVLTQSPAIMSASPGEKVTMTCSAGSS |
| | NIKDTYMHWVMQRPEQGLEWIGKIDP | | VSYMYWYQQKPGSSPRVLIYDTSNLAS |
| | ANGNTEFDPKFQGKATITADTSSNTAY | | GVPVRFSGSGSGTSYSLTISRMEAEDAA |
| | LQLTSLTSEDTAVYYCTRAMDYWGQG | | TYYCQQWSNYPYTFGGGTKLEIK (SEQ |
| | TSVTVSS (SEQ ID NO: 547) | | ID NO: 635) |
| KCD042 | EVQLQQSGAELVKPGASVRLSCTASGF | KCD042 | QSVLTQSPAIMSASPGEKVTMTCSANSS |
| | NIKHTYIHWVSQRPEQGLEWIGKIDPA | | VSDMYWFQQRPGSSPRLLIYDTSNLAS |
| | NGNTKYDPKFQGKATITADTSSNTAYL | | GVPVRFSGSGSGTSYSLTISRMEAEDAA |
| | QLSSLTSEDTAVYYCVNAMEYWGQGT | | TYYCQQWSTYPWTFGGGTKLEIK (SEQ |
| | SVTVSS (SEQ ID NO: 548) | | ID NO: 636) |
| KCD044 | EVQLQQSGAELVKPGASVRLSCTASGF | KCD044 | QSVLTQSPAIMSASPGEKVTMTCSANSS |
| | NIKHTYMHWVSQRPERGLEWIGKIDPA | | VSDMYWYQQRPGSSPRLLIYDTSNLAS |
| | NGNTKYDPKFQGKATITADTSSNTVYL | | GVPVRFSGSGSGTSYSLTISRMEAEDAA |
| | QLSSLTSEDTAVYYCLNAMEYWGQGT | | TYYCQQWSTYPWTFGGGTKLEIK (SEQ |
| | SVTVSS (SEQ ID NO: 549) | | ID NO: 637) |
| KCD047 | EVQLQQSGAEFVKPGASVRLSCTASGF | KCD047 | QIVLTQSPAVMSASPGEKVAMTCSASSS |
| | NIKDTYMHWVKQRPEQGLEWIGRIDP | | VTYMYWYQQKPGSSPRLLIYDTSNLAS |
| | ANGYTKDDPKFQGKATITADTSSNTAY | | GVPVRFSGSGSGTSYSLTISRMEAEDAA |
| | LQLSSLTSEDTAVYYCASAMDYWGQG | | TYYCQQWSTYPFTPGSGTKLEIK (SEQ |
| | TSVTVSS (SEQ ID NO: 550) | | ID NO: 638) |
| KCD048 | EVQLQQSGADLVKPGASVKLSCTASGF | KCD048 | QIVLTQSPAIMSASPGEKVTLTCSATSSV |
| | NIKATYMHWVRQRPEKGLEWIGRIDPA | | SYMYWYQQKPGSSPRLLIYDTSNLASG |
| | NGHTIYDPQFQGKATITSDTSSNTAYLQ | | VPVRFSGSGSGTSYSLTISRMEAEDDAT |
| | LNSLTSEDTAVYYCAEAMDYWGQGTS | | YYCQQWSNYPFTFGGGTKLEIK (SEQ ID |
| | VTVSS (SEQ ID NO: 551) | | NO: 639) |
| KCD049 | QVQLKESGPGLVAPSQSLSITCTVSGFS | KCD049 | DIVMTQSPASLAVSLGQRATISCRASES |
| | LSSYGVQWVRQPPGQGLEWLVVIWRD | | VDKYGISFLNWFQQKPGQPPKLLIYAAS |
| | GSITYNSALKSRLSISKDNSKSQVFLKM | | NQGSGVPARFSGSGSGTDFSLNIHPMEE |
| | NSLQTDDTAMYYCGRTSHYGNYNYY | | DDPAMYFCQQGKEVPWTFGGGTKLEIK |
| | VMDYWGQGTAVTVSS (SEQ ID NO: | | (SEQ ID NO: 640) |
| | 552) | | |
| KCD050 | QVQLKESGPGLVAPSQSLSITCTVSGFS | KCD050 | DIVMTQSPTSLAVSLGQRATISCRASES |
| | LNSYGVQWVRQPPGQGLEWLVVIWRD | | VDKYGISFELNWFQQKPGQPPRLLIYAAS |
| | GTITYNSALKSRLSINKDNSKSQVFLK | | NQGSGVPARFSGSGSGTDFSLNIHPMEE |
| | MNSLQTDDTAMYYCGRTSHYGNFNY | | DDPAVYFCQQGKEFPWTFGGGTKLEIK |
| | YVMDYWGQGTAVTVSS (SEQ ID NO: | | (SEQ ID NO: 641) |
| | 553) | | |
| KCD052 | QVQLKESGPGLVAPSQSLSITCTVSGFS | KCD052 | DIVMTQSPASLAVSLGQRATISCRASES |
| | LNSYGVQWVRQPPGQGLEWLGVIWRD | | VDKYGISFLNWFQQKPGQPPKLLIYAAS |
| | GSITYNSALKSRLSIRKDNSKSQVFLKM | | NQGSGVPARFSGSGSGTDFSLNIHPMEE |
| | NSLQTDDTAMYYCGRTSHYGNYNYY | | DDPAIYFCQQGKEVPWTFGGGTKLEIK |
| | VMDYWGQGTAVTVSS (SEQ ID NO: | | (SEQ ID NO: 642) |
| | 554) | | |
| KCD056 | EVKLVESGGGLVQPGGSLKVSCAASGF | KCD056 | DIQMTQSPSSLSASLGGKVTITCKASQDI |
| | TESTYTMSWVRQTPEKRLEWVAYITN | | NKYIAWYQHKPGKGPRLLIHYTSTLQP |
| | GGGSTYYPDTEKGRFTISRDNAKNTLY | | GIPSRFSGSGSGRDYSFSISNLEPEDIATY |
| | LQMSSLKSEDTAMYYCVRHDYYAMD | | YCLQYDNLLYTFGGGTTLEIK (SEQ ID |
| | YWGQGTSVTVSS (SEQ ID NO: 555) | | NO: 643) |
| KCD057 | EVKLVESGGGLVQPGGSLKLSCAASGF | KCD057 | DIQMTQSPSSLSASLGGKVTITCKASQDI |
| | TFSNYIMSWVRQTPEKRLEWVAYITNG | | NKYIAWYQHKPGKGPRLLIHYTSTLQP |
| | GGATYYPDTVKGQFTISRDNAKNTLYL | | GIPSRFSGSGSGRDYSFSISNLEPEDIATY |
| | QMSSLKSEDTAIYYCARHDFYALDFW | | YCLKYDNLLYTFGGGTKLEIK (SEQ ID |
| | GQGTSVTVSS (SEQ ID NO: 556) | | NO: 644) |
| KCD058 | EVKLVESGGDLVQPGGSLKLSCAASGF | KCD058 | DIQMTQSPSSLSASLGGKVTITCKASQDI |
| | TFSRYIMSWVRLTPEKRLEWVAFITNG | | NKYIAWYQHKPGKGPRLLIHYTSTLQP |
| | GGNTYHPDTVKGRFTISRDNANNTLYL | | GIPSRFSGSGSGRDYSFSISNLEPEDIATY |
| | QMSSLKSEDTAIYYCARHDYYALDYW | | YCLQYDNLLYTFGGGTKLEIK (SEQ ID |
| | GQGTSVTVSS (SEQ ID NO: 557) | | NO: 645) |
| KCD062 | EVKLVESGGGLVQPGGSLKLSCAASGF | KCD062 | DIQMTQSPSSLSASLGGKVTITCKASQDI |
| | TESTYIMSWVRQTPEKRLEWVAYITSG | | NKYIAWYQHKPGKGPRLLIHYTSTLQP |
| | GSSTYYPDTVKGRFTISRDNAKSTLYL | | GIPSRFSGSGSGRDYSFSISNLEPEDIATY |
| | QMSSLKSEDTAMYYCARHAHFYAMD | | YCLQYDNLLYTFGGGTKLEIK (SEQ ID |
| | YWGQGTSVTVSS (SEQ ID NO: 558) | | NO: 646) |
| KCD063 | EVKLVESGGGLVQPGGSLKLSCAASGF | KCD063 | DIQMTQSPSSLSASLGGKVTITCKASQDI |
| | IFSSYIMSWVRQTPEKRLEWVAYITNG | | NKYITWYQHKPGKGPRLLIHYTSTLQPG |
| | GGSTYYPDTVKGRLTISRDNAKNTLYL | | IPSRFSGSGSGRDYSFSISNLEPEDIATYY |
| | QMSSLKSEDTAMYYCVRHAHYYAMD | | CLQYDNLLYTFGGGTKLEIK (SEQ ID |
| | YWGQGTSVTVSS (SEQ ID NO: 559) | | NO: 647) |
| KCD064 | EVKLVESGGGLVQPGGSLKLSCAASGF | KCD064 | DIQMTQSPSSLSASLGGKVTITCKASQDI |
| | TFSSYIMSWVRQTPEKRLGWVAYITSG | | NKYIAWYQHKPGKGPRLLIHYTSTLQP |
| | GGSTYYPDTVKGRFTISRDNAKNTLYL | | GIPSRFSGSGSGRDYSFSISNLEPEDIATY |
| | QMSSLKSEDTAMYYCARHGTGYAMD | | YCLQYDNLLYTFGGGTELEIK (SEQ ID |
| | YWGQGTSVTVSS (SEQ ID NO: 560) | | NO: 648) |
| KCD065 | QVQLQQSGAELARPGASVKMSCKASG | KCD065 | DIVMTQSHKFLSTSLGDRVSITCKASQD |
| | YTFTTYTIHWVKQRPGQGLEWIGYINP | | VGSAVAWYQQKPGQSPDLLIYWTFTRH |
| | SSDFTNYNQNFADKATLTADRSSSTAY | | TGVPDRFTGSRSGTDFTLTISNVQSGDL |
| | MQLSSLTSEESAVYYCAIRLGYDRQG | | ADYFCQQYSNYPLTFGGGTKLEIK (SEQ |
| | WYFDVWGAGTTVTVSS (SEQ ID NO: | | ID NO: 649) |
| | 561) | | |
| KCD066 | QVQLQQSAAELARPGASVKMSCKASG | KCD066 | DIVMSQSPSSLAVSVGEKVTMSCKSSQS |
| | YTFTDYTMHWVKQRPGQGLEWIGYIN | | LLYSGNQKNYLAWYQQKPGQSPKLLIY |
| | PSGGYTDYNQKFKDKTALTADKSSSTA | | WASTRESGVPDRFTGSGSGTDFTLTISS |
| | YMQLSSLTSEDSAVYYCARRRDYWFA | | VKAEDLAVYYCHQYYSYLTFGAGTKL |
| | YWGQGTLVTVSA (SEQ ID NO: 562) | | ELK (SEQ ID NO: 650) |
| KCD070 | EIQLQQTGPELVKPGASVKISCKASGYS | KCD070 | DIVMTQSHKFMSTSVGDRVSITCKASQ |
| | FTDYIILWVKQSHGKSLEWIGNINPYYD | | DVGTAVAWYQQKPGQSPKLLIYWAST |
| | YTSYNLKFKGKATLTVDKSSSTAYMQ | | RHTGVPDRFTGSGSGTDFTLTINNVQSE |
| | LNSLTSEDSAVYYCARSDGYYGGDYW | | DLADYFCQQYSSYPWTFGGGTTLEIK |
| | GQGTSVTVSS (SEQ ID NO: 563) | | (SEQ ID NO: 651) |
| KCD075 | QVQLLQPGAELVRPGTSVKLSCKASGY | KCD075 | DIQMTQTTSSLSASLGDRVTISCSGGQGI |
| | TFSNYWINWVKQRPGQGLEWIGNIYPS | | SNYLNWYQQKPDGTFKLLIYYTSTLHS |
| | DSSINYNQKFKDKATLTVDKSSTTAYM | | GVPSRFSGSGSGTDYSLTISNLEPEDVAT |
| | QLSSPTSEDSAVYYCTGTDWYFDVWG | | YYCQQYSKLPYTFGGGTKLEIK (SEQ ID |
| | AGTTVTVSS (SEQ ID NO: 564) | | NO: 652) |
| KCD077 | EVQLQQSGAELVRPGALVKLSCKASGF | KCD077 | DIVMTQSQKFMSTSVGDRVSVTCKASQ |
| | NIKDYYMHWVKQRPEQGLEWIGWIDP | | NVGTNVAWYQQKPGQSPKALIYTASYR |
| | ENGHTIYDPRFQGKATITADTSSNTAYL | | YSGVPDRFTGSGSGTDFTLTISNVQSED |
| | QLSSLTSEDTAVYYCSRGLLGFAYWG | | LAEYFCQQYNSYPHMYTFGGGTKLEIK |
| | QGTLVTVSA (SEQ ID NO: 565) | | (SEQ ID NO: 653) |
| KCD101 | QVQLQQPGAELVRPGTSVKLSCKASG | KCD101 | DIQMTQTTSSLSASLGDRVTISCRASQDI |
| | YTFTSYWMHWVKQRPGQGLEWIGVID | | SNYLNWYQQKPDGTVKLLIYYPSRLHS |
| | PSDSYTNYNQKFKGKATLTVDTSSSTA | | GVPSRFSGSGSGTDYSLTISNLEQEDFAT |
| | YMQLSSLTSEDSAVYYCGRNGYDGSM | | YFCQQGNTLPYTFGGGTKLEIK (SEQ ID |
| | DYWGQGTSVTVSS (SEQ ID NO: 566) | | NO: 654) |
| KCD102 | QVQLQQPGAELVRPGTSVKLSCKASG | KCD102 | EIQMTQTTSSLSASLGDRVTISCRASQDI |
| | YTFTSYWMHWVKQRPGQGLEWIGVID | | SNYLNWYQQKPDGTVKLLIYYPSRLHS |
| | PSDSYTNYNQKFKGKATLTVDTSSSTA | | GVPSRFSGSGSGTDYSLTISNLEQEDFAT |
| | YMQLSSLTSEDSAVYYCARNGYDGSM | | YFCQQGNTLPYTFGGGTKLEIK (SEQ ID |
| | DYWGQGTSVTVSS (SEQ ID NO: 567) | | NO: 655) |
| KCD103 | QVQLQQPGAELVRPGTSVKLSCKASG | KCD103 | DIQMTQTTSSLSASLGDRVTISCRASQDI |
| | YTFTSYWMHWVKQRPGQGLEWIGVID | | SNSLNWYQQKPDGTVKLLIYYTSRLHS |
| | PSDSYTKYNQKFKDKATLTVETSSSTA | | RVPSRFSGSGSGTDYSLTISNLDQEDIAT |
| | YMQLSSLTSEDSAVYYCAGNGYDGSM | | YFCQQANTLPYTFGGGTKLEIK (SEQ ID |
| | DYWGQGTSVTVSS (SEQ ID NO: 568) | | NO: 656) |
| KCD104 | QVQLQQPGAELVRPGTSVKLSCKASG | KCD104 | EIQMTQTTSSLSASLGDRVTISCRASQDI |
| | YTFTSYWMHWVKQRPGQGLEWIGVID | | SNYLNWYQQKPDGTVKLLIYYPSRLHS |
| | PSDSYTYYNQKFKGKATLTVDTSSSTA | | GVPSRFSGSGSGTDYSLTISNLEQEDFAT |
| | YMQLSSLTSEDSAVYYCARNGYDGAM | | YFCQQGNTLPYTFGGGTKLEIK (SEQ ID |
| | DYWGQGTSVTVSS (SEQ ID NO: 569) | | NO: 657) |
| KCD110 | EVQLQQSGPELVKPGASVKISCKASGY | KCD110 | QIVLSQSPAILSASPGEKVTMTCRASSSV |
| | SFTGYYMHWVKQSHGNILDWIGYIDP | | SYMHWYQQKPGSSPKPWIYATSNLASG |
| | DNGVSSKNQKFTGKATVTADKSSSTA | | VPARFSGSGSGTSYSLTISRVEAEDAAT |
| | YMELRSLTSEDSAVYYCAGYYGSSWY | | YYCQQWSSNPYTFGGGTKLEIK (SEQ |
| | WYFDVWGTGTTVTVSS (SEQ ID NO: | | ID NO: 658) |
| | 570) | | |
| KCD111 | EVQLQQSGPELVKPGASVKISCKASGY | KCD111 | QIVLSQSPAILSASPGEKVTMTCRASSSV |
| | SFTDYYMHWVKQSHGNILDWIGYIDP | | SYMHWYQQKPGSSPKPWIYATSNLASG |
| | YNGVSSYNQKFKGKATLSVDQSSSTA | | VPTRFSGSGSGTSYSLTISKLEAEDAATY |
| | YMELRSLTSEDSAVYYCSSYYGSSPYW | | YCQQWSSNPYTFGGGTKLEIK (SEQ ID |
| | YFDVWGTGTRVTVSS (SEQ ID NO: | | NO: 659) |
| | 571) | | |
| KCD112 | EVQLQQSGPELVKPGASVKISCKASGY | KCD112 | DIVMTQSHKFMSTSVGDRVSITCKASQ |
| | SFTAYYMNWVKHSPEKSLEWIGDINPS | | DVSTAVAWYQQKPGQSPKLLIFWTSTR |
| | TGGTTYNQKFKARATLTVDKSSSTAY | | HTGVPDRFTGSGSGTDYTLTISSVQAED |
| | MQLKSLTSEDSAVYYCATTYYSGNSY | | LALYYCQQHYTTPWTFGGGTKLEIK |
| | VGFAYWGQGTLVTVSA (SEQ ID NO: | | (SEQ ID NO: 660) |
| | 572) | | |
| KCD114 | EVQLQQSGPELVKPGASVKISCKASGY | KCD114 | DIVMTQSHKFMSTSVGDRVSITCKASQ |
| | SFTAYYMNWVKQSPEKSLEWIGDINPS | | DVSTAVAWYQQKPGQSPKLLIFWASTR |
| | TGGTTYNQNFKAKATLTVDKSSSTAY | | HTGVPDRFTGSGSGTDYTLTISSVQAED |
| | MHLKSLTSEDSAVYYCATTYYSGNSY | | LALYYCQQHYSTPWTFGGGTKLEIK |
| | VGFAYWGQGTLVTVSA (SEQ ID NO: | | (SEQ ID NO: 661) |
| | 573) | | |
| KCD115 | EVQLQQSGPELVKPGASVKISCKASGY | KCD115 | DIVMTQSHKFMSTSVGDRVSITCKASQ |
| | SFTGYYMNWVKQSPEKSLEWIGDINPS | | DVSTAVDWYQQKPGQSPKLLIYWASTR |
| | TGGTTYNQKFKAKATLTVDKSSSTAY | | HTGVPDRFTGSGSGTDYTLTISSVQAED |
| | MQLKSLTSEDSAVYYCATPYYYGSSY | | LALYYCQQHYSTPWTFGGGTKLEIK |
| | VGFAYWGQGTLVTVSA (SEQ ID NO: | | (SEQ ID NO: 662) |
| | 574) | | |
| KCD118 | EVQLQQSGPELVKPGASVKISCKAFGY | KCD118 | DVLMTQTPLSLPVSLGDQASISCRSSQTI |
| | TFTDYYKNWMRQRHGESLEWIGDINP | | VHSNGDTYLEWYLQKPGQSPNLLIYKV |
| | NSGDANYNQKFKGKATLTVDKSSSTA | | SNRFSGVPDRFSGSGSGTDFTLKISRVEA |
| | YMELRSLTSEDSAVYYCAREGPSFAY | | EDLGIYYCFQGSHVPPTFGGGTKLEIK |
| | WGHGTLVTVSA (SEQ ID NO: 575) | | (SEQ ID NO: 663) |
| KCD119 | EVQLQQSGPELVKPGASVKISCKASGY | KCD119 | DVLMTQTPLSLPVSLGDQASISCRSSQTI |
| | TFTDYYTNWMRQRHGESLEWIGDINP | | VHSNGDTYLEWYLQKPGQSPNLLIYKV |
| | NTGDTSYNQKFRVKATLTVDKSSGTA | | SNRFSGVPDRFSGSGSGTDFTLKISRVEA |
| | YMGLRSLTSEDSAVYYCTREGPSFAY | | EDLGVYYCFQGSHVPPTFGGGTTLEIK |
| | WGQGTLVTVSA (SEQ ID NO: 576) | | (SEQ ID NO: 664) |
| KCD121 | EVQLQQSGPELVKPGASVKISCKASGY | KCD121 | DVLMTQTPLSLPVSLGDQASISCRSNQTI |
| | TFTDYYKNWMRQRHGESLEWIGDINP | | VHSNGDTYLEWYLQKPGQSPNLLIYKV |
| | NNGDTSYNQKFRGKATLTVDKSSSTAF | | SNRFSGVPDRFSGSGSGTDFTLRISRVEA |
| | MELRSLTSEDSAVYYCAREGPSFAYW | | EDLGVYYCFQGSHVPPTFGGGTKLEIK |
| | GQGTLVTVSA (SEQ ID NO: 577) | | (SEQ ID NO: 665) |
| KCD122 | EVQLQQSGPELVKPGASVKISCKASGY | KCD122 | DVLMTQTPLSLPVSLGDQASISCRSSQTI |
| | TFTDYYKNWMRQRHGESLEWIGDINP | | VHSNGDTYLEWYLQKPGQSPNLLIYKV |
| | NNGDANYNQKFKGKATLTVDKSSSTA | | SNRFSGVPDRFSGSGSGTDFTLKISRVEA |
| | YMELRSLTSEDSAVYFCAREGPSFAYW | | EDLGVYYCFQGSHVPPTFGGGTKLEIK |
| | GHGTLVTVSA (SEQ ID NO: 578) | | (SEQ ID NO: 666) |
| KCD123 | EVHLQQSGPELVKPGASVKISCKASGY | KCD123 | DVLMTQTPLSLPVSLGDQASISCRSSQTI |
| | TFTDFYKNWMRQRHGESLEWIGDINPN | | VHSNGDTYLEWYLQKPGQSPNLLIYKV |
| | NGGTNYNQKFKGKATLTVDKSSSTAY | | SNRFSGVPDRFSGSGSGTDFTLKISRVEA |
| | MELRSLTSEDSAVYYCAREGPSFAYW | | EDLGVYYCFQGSHVPPTFGGGTKLEIK |
| | GQGTLVTVSA (SEQ ID NO: 579) | | (SEQ ID NO: 667) |
| KCD124 | EVQLQQSGPELVKPGASVKISCKASGY | KCD124 | DVLMTQTPLSLPVSLGDQASISCRSSQSI |
| | TFTDHYMNWVKQSHGKSLEWIGDINP | | VHSNGDTYLEWYLQKPGQSPKLLIYKV |
| | NNGGTSYNQKFKGKATLTVDKSSSTA | | SNRFSGVPDRFSGSGSGTDFTLKISRVEA |
| | YMELRSLTSGDSAVYYCTREGASFAF | | EDLGVYYCFQGSHVPLTFGAGTKLELK |
| | WGQGTLVTVSA (SEQ ID NO: 580) | | (SEQ ID NO: 668) |
| KCD125 | EVQLQQSGAELVRPGSSVKMSCKTSG | KCD125 | DIQMTQSPSSLSASLGERVSLTCRASQEI |
| | NTFTSYGINWVKQRPGQGLEWIGYIYI | | SGYLSWLQQKPDGTIKRLIYAASTLDSG |
| | GTGYTEYNEKFKGKATLTSDTSSSTAY | | VPKRFSGSRSGSDYSLTISSLESEDFADY |
| | MQLSSLTSEDSAIYFCVRKAYGNYGFD | | YCLQYASYPFTFGSGTKLEIK (SEQ ID |
| | YWGQGTTLTVSS (SEQ ID NO: 581) | | NO: 669) |
| KCD126 | EVQLQQSGAELVRPGSSVKMSCKTSG | KCD126 | DIQMTQSPTSLSASLGESVSLTCRASQEI |
| | KTFTSHGINWVKQRPGQGLEWIGYIYI | | SGNLNWLQQKPDGSIKRLIYAASTLDSG |
| | GNGYNEYNEKFKGKATLTSDTSSSTAY | | VPKRFSGSRSGSDYSLTISSLESEDFADY |
| | MQLSSLTFEDSAIYFCVRKAYGNYGFD | | YCLQYANYPFTFGSGTKLEVK (SEQ ID |
| | DWGQGTTLTVSS (SEQ ID NO: 582) | | NO: 670) |
| KCD127 | QVQLQQPGAELVRPESSVKLSCKASGY | KCD127 | DIVMTQSHKFMSTSVGDRVSITCKASQ |
| | TFTNFWMDWVKQRPGQGLEWIGNIYP | | DVSTAVAWYQQKPGQSPKLLIYSASYR |
| | SGSETHYNQKFKDKATLTVDKSSTTAY | | STGVPDRFTGSGSGTDFTFTISSVQAEDL |
| | MQLSSLTSEDSAVYYCARSGYYGSRYL | | AVYYCQQHYSTPYTFGGGTKLEIR |
| | YYFDYWGQGTTLTVSS (SEQ ID NO: | | (SEQ ID NO: 671) |
| | 583) | | |
| KCD128 | QVQLQQPGAELVRPESSVKLSCKASGY | KCD128 | DIVMTQSHKFMSTSVGDRVTITCKASQ |
| | TFTSYWMDWVMQRPGQGLEWIGNIYP | | DVSNAVAWYQLKPGQSPKLLIYSASYR |
| | SGSETHYNQKFKDKATLTVDKSSTTAY | | YTGVPDRFTGSGSGTDFTFTISSVQAAD |
| | MQLSSLTFEDSAVYYCARSGFIGSRYL | | LAVYYCQQHYITPYTFGGGTKLEIK |
| | YYFDYWGQGTTLTVSS (SEQ ID NO: | | (SEQ ID NO: 672) |
| | 584) | | |
| KCD129 | EVOLVESGGGLVQPKGSLKLSCAASGF | KCD129 | DVLMTQTPLSLPVSLGDQASISCRSSQSI |
| | SFNTYAMNWVRQAPGKGLEWVARIRS | | VHSDGNTYLEWYLQKPGQSPKLLIYRV |
| | KSNNYATYYADSVKDRFTISRDDSESM | | SNRFSGVPDRFSGSGSGTDFTLKISRME |
| | VYLQMNNLKTEDTAMYYCVRHGYYW | | AEDLGVYYCFQGSHVPYTFGGGTKLEI |
| | YFDVWGTGTTVTVSS (SEQ ID NO: | | K (SEQ ID NO: 673) |
| | 585) | | |
| KCD131 | QVQLQQSGPELVKPGASVKISCKASAY | KCD131 | DIQMTQSPASLSVSVGETVTITCRASENI |
| | TFTDYYINWVKQRPGQGPEWIGWIFPG | | YSHLAWFQQKQGKSPRLLVYSATNLPD |
| | SNSTYSNEKFEVKATLTVDESSSTAYM | | GVPSRFSGSGSGTQYSLKINILQSEDFGS |
| | LLSSLTSEDSAVYFCARLGYFGSSYHA | | YYCQHFWGTPWTFGGGTKLEIK (SEQ |
| | LDYWGQGTSVTVSS (SEQ ID NO: 586) | | ID NO: 674) |
| KCD132 | QVQLQQSGPELVKPGASVKISCKASGY | KCD132 | DIQMTQSPASLSVSVGETVTITCRASENI |
| | SFTDYYINWVKQRPGQGLEWIGWIFPG | | YSNLAWYQQKQGKSPQLLVYVATNLA |
| | SGSTYYNEKFKGKATLTVDKSSSTAY | | DGVPSRFSGSGSGTQYSLKINSLQSEDF |
| | MLLSSLTSEDSAVYFCARTGYYSNLYA | | GNYYCQHFWGTPYTFGGGTKLEMR |
| | VDYWGQGTSVTVSS (SEQ ID NO: 587) | | (SEQ ID NO: 675) |
| KCD133 | EVQLVESGGGLLQPKGSLKLSCAASGF | KCD133 | DIVLTQSPASLAVSLGQRATISCRASESV |
| | TENTYAMNWVRQAPGKGLEWVARIRS | | EYYGTSLMQWYQQKPGQPPKLLINAAS |
| | KSSNYATYYADSVKDRFTISRDDSQSM | | NVESGVPARFSGSGSGTDFSLNIHPVEE |
| | FYLEMNNLKTEDTAMYYCVRDRGYY | | DDIAMYFCQQSRKVPWTFGGGTKLEIK |
| | YVMDYWGQGTSVTVSS (SEQ ID NO: | | (SEQ ID NO: 676) |
| | 588) | | |
| KCD134 | EVQLQQSGPEMVKPGASVKMSCQASG | KCD134 | ETTVTQSPASLSMAIGEKVTIRCITSTDI |
| | YTFTDYYMNWVKQSHGETLEWIGDIY | | DDDMNWYQQKPGEPPKLLISEGNSLRP |
| | PHNGYTAYNQKFKGKATLTVDKSSST | | GVPSRFSSSGYGTDFVFTIEDMFSEDVA |
| | AYMELRSLTSEDSAVYYCARGGQLRL | | DYHCLQSDNLPYTFGGGTKLEIK (SEQ |
| | PAWFAYWGQGTLVTVSA (SEQ ID NO: | | ID NO: 677) |
| | 589) | | |
| KCD135 | EVQLQQSGPELVKPGASVRMSCKASG | KCD135 | DIVMTQSQKFMSTSVGDRVSVTCKASQ |
| | YIFTDYSIHWVKQSHGKSLEWIGYINPN | | HVGTNVVWYQQKPGQSPKALIYSASYR |
| | NGGTSYNQKFKGKATLTVNKSSTTAY | | YSGVPDRFSGSGSGTDFTLTISNVQSED |
| | MELRSLTSEDSAVYFCARDTTIVGDYW | | LAEYFCQQFNSYPLTFGGGTKLEIK |
| | GQGTTLTVSS (SEQ ID NO: 590) | | (SEQ ID NO: 678) |
| KCD136 | EVQLQQSVAELVRPGASVKLSCSASGF | KCD136 | QIVLTQSPAIMSASPGEKVTMTCSASSS |
| | NIKNTYMHWVNQRPEQGLEWIGRIDP | | VSYMYWYQQKPGSSPRLLIYDISNLASG |
| | ANGITKYAPNFQGKATITADTSSNTAY | | VPVRFSGSGSGTSYSLTISRMEAEDAAT |
| | LQLSNLTSEDTAIYYCTRAMDYWGQG | | YYCQQWDTYPWTFGGGTKLEIK (SEQ |
| | TSVTVSS (SEQ ID NO: 591) | | ID NO: 679) |
| KCD137 | EVQLVESGGDLVKPGGSLKLSCAASGF | KCD137 | DIVMTQSHKFMSTSVGDRVSITCKASQ |
| | TFSGYGMSWVRQIPDKRLEWVAISSRD | | DVGTAVAWYQQRPGQSPKLLIYWAST |
| | NSFTYYPDSVKGRFTISRDNAKNTLYL | | RHTGVPDRFTGSGSGTDFTLTVSNVQSE |
| | QMSSLKSEDTALYFCTRHPYLPTGGYV | | DLADYFCQQYTSYPLTFGAGTKLELK |
| | MDYWGQGTSVTVSS (SEQ ID NO: 592) | | (SEQ ID NO: 680) |
| KCD139 | EVQLQQSGAELVRPGSSVKMSCKTSG | KCD139 | DIVLTQSPASLAVSLGQRATISCRASESV |
| | YTFTSYGINWVKQRPGQGLEWIGYIYI | | DSYGNSFMHWYQQKPGQPPKLLIHRAS |
| | ANGYTENNEKFKGKAKLTSDISSSTAY | | NLESGIPARFSGSGSRTDFTLTINPVEAD |
| | MQLSSLTSEDSAIYFCARRFDYAGALD | | DVAIYYCQQTNDDPYTFGGGTNLEIK |
| | YWGQGTSVTVSS (SEQ ID NO: 593) | | (SEQ ID NO: 681) |
| KCD200 | EVQLQQSGPELVKPGASVKISCKASGY | KCD200 | DVLMTQTPLSLPVSLGDQVSISCRSSQTI |
| | TFTSYYKNWMRQRHGESLEWIGDINPN | | VHSNGDTYLEWYLQKPGQSPNLLIYKV |
| | SGDTAYNQKFKGKATLTVDRSSSTAY | | SNRFSGVPDRFSGSGSGTDFTLKISRVEA |
| | MELRSLTSEDSAVYYCAREGPSFAYW | | EDLGVYYCFQGSHVPPTFGGGTKLEIK |
| | GQGTLVTVSA (SEQ ID NO: 594) | | (SEQ ID NO: 682) |
| KCD205 | EVQLQQSGAELVRPGSSVKMSCKTSG | KCD205 | DIQMTQSPSSLSASLGERVSLTCRASQEI |
| | NTFTSHGINWVKQRPGQGLEWIGYIYI | | SGYLSWLQQKPDGTIKRLIYAASTLDSG |
| | GNGYNEYNEKFKGKATLTSDTSSSTAY | | VPKRFSGSSSGSDYSLTISSLESDDFADY |
| | MQLSSLTSEDSAIYFCVRKAYGNYGFD | | YCLQYASYPFTFGSGTKLEIK (SEQ ID |
| | DWGQGTTLTVSS (SEQ ID NO: 595) | | NO: 683) |
| KCD207 | AVQLVESGGGLVQPKGSLKLSCAASGF | KCD207 | DVLMTQTPLSLPVSLGDQASISCRSSQTI |
| | SENTYAMNWVRQAPGKGLEWVARIRS | | VHSNGNTYLEWYLQKSGQSPKLLIYNV |
| | KSNNYATYYADSVKDRFTLSRDDSES | | SNRFSGVPDRFRGSGSGTDFTLKISRVE |
| | MLYLQMNNLKTEDTAMYYCVRQGFY | | AEDLGVYYCFQGSHVPYTFGSGTKLEIK |
| | WYFDVWGTGTTVTVSS (SEQ ID NO: | | (SEQ ID NO: 684) |
| | 596) | | |
| KCD208 | EVQLQQSVAELVRPGASVKLSCTVSGF | KCD208 | EIVLTQSPALMAASPGEKVTITCSVSSSI |
| | NIKNTYMHWVKQRPEQGLEWIGRIDP | | SSSSLHWYRQKSGTSPKPWIYGTSHLAS |
| | ANGDTTYAPKFQGKATITADTSSNSAY | | GVPVRFSGSGSGTSYSLTISSMEAEDAA |
| | LHLSRLTSEDTAIYYCSLYDYDGYWGQ | | TYYCQQWDTYPWTFGGGTKLEIK (SEQ |
| | GTTLTVSS (SEQ ID NO: 597) | | ID NO: 685) |
| KCD210 | EVQLQQSGPVLVKPGASVTMSCKASG | KCD210 | EIVLTQSPALMTASPGEKVTITCSVSSSIS |
| | YTFTEYYMNWVKQSHGKSLDWIGVIN | | STNLHWYQQKSGTSPKPWIFGTSNLAS |
| | PYSGGTSYKQKFKDKATLTVDKSSSTA | | GVPVRFSGSGSGTSYSLTISNMEAEDAA |
| | YMELNGLTSEDSAVYFCVRGGLRRNY | | TYYCQQWNSYPFTFGTGTKLEIK (SEQ |
| | FDYWGQGTTLTVSS (SEQ ID NO: 598) | | ID NO: 686) |
| KCD212 | EVHLVESGGDLVKPGGSLKLSCAASGF | KCD212 | QIVLTQSPAIMSASPGEKVTMTCRASSS |
| | TFSRYGMSWVRQTPDKRLEWVATISS | | VSSTYLHWYQQKPGSSPKLWIYSTSNL |
| | AGSYTYYPDSVKGRFTISRDNAKNTLF | | ASGVPPRFSGSGSGTSYSLTISSVEAEDA |
| | LQMSSLKSEDTAMYYCARPLNYYGTS | | ATYYCQQYDSSPNTFGAGTKLELK |
| | SFDYWGQGTTLTVSS (SEQ ID NO: 599) | | (SEQ ID NO: 687) |
| KCD214 | EVQLQQSVAEFVRPGASVKLSCTASGF | KCD214 | EIVLTQSPALMAASPGEKVTITCRVSSSI |
| | NIKNTYMHWVKQRPEQGLEWIGRIDP | | SSSSLHWYQQKSGTSPKPWIYGTSNLAS |
| | ANGNTEYAPKFQGKATITADTSSNTAY | | GVPVRFSGSRSGTSYSLTISSMEAEDAA |
| | LQLSSLTSEDTAIYYCALYDYDGYWGQ | | TYYCQQWSDYPWTFGGGTKLEIK (SEQ |
| | GTTLTVSS (SEQ ID NO: 600) | | ID NO: 688) |
| KCD216 | QVHLQQSGPELVKPGASVKISCKASGY | KCD216 | DIQMTQSPASLSVSVGETVTITCRASENI |
| | TFIDYYINWVKQRPGQGLEWIGWIFPG | | YSNLAWYQQKQGKSPQLLVYAATNLA |
| | SDSTYYNEKFKGKATLTVDKSSSTAY | | DGVPSRFSGSGSGTQYSLKINSLQSEDF |
| | MLLSSLTSEDSAVYFCARYGYYGSSFY | | GSYYCQHFWGTPPTFGGGSKLEIK (SEQ |
| | AMDYWGQGTSVTVSS (SEQ ID NO: | | ID NO: 689) |
| | 601) | | |
| KCD217 | QVQVQQPGAEFVKTGASVKLSCKTSG | KCD217 | DIQMTQSPASLSASVGETVTITCGASENI |
| | YTFIDYWIHWVKQRPGHGLEWIGRIDP | | YGALNWYQRKQGKSPQLLIYGATNLA |
| | NTGGSKYYEKFKRKATLTVDKPSRTV | | DGMSSRFSGSGSGRQYSLKISSLHPDDV |
| | YMQLSSLTSEDSAVYYCTREYDYGWF | | ATYYCQNVLSTPWTFGGGTKLEIK |
| | GYWGQGTLVTVSE (SEQ ID NO: 602) | | (SEQ ID NO: 690) |
| KCD219 | QVQLQQSGPELVKPGASVKISCKASGY | KCD219 | DIQMTQSPASLSVSVGETVTITCRASENI |
| | TFTDYYINWVKQRPGQGLEWIGWIFPG | | YSNLAWYQQKQGKSPQLLVYAATNLA |
| | SGSTYYNEKFKGKATLTVDKSSSTAY | | DGVPSRFSGSGSGTQYSLKINSLQSEDF |
| | MLLSSLPSEDSAVYFCARYGYYGSSFY | | GSYYCQHFWGSPPTFGGGTKLEIK (SEQ |
| | AMDYWGQGTSVTVSS (SEQ ID NO: | | ID NO: 691) |
| | 603) | | |
| KCD220 | QVQLQQSGAELMEPGASVKLSCKATG | KCD220 | DIQMTQTTSSLSASLGDRVTISCRASQDI |
| | YTFTGYWIEWVKQRPGHGLEWIGETLP | | SNYLNWYQQKPDGTVKLLIYYTSNLHS |
| | GSDSNNYNEKFKGKATFTADTSSNTAY | | GVPSRFSGSGSGTDYSLTISNLEQEDIAT |
| | MQLSSLTTEDSAIYYCARDYSNYWYF | | YFCQQDSKHRTFGGGTKLEIK (SEQ ID |
| | DVWGTGTTVTVSS (SEQ ID NO: 604) | | NO: 692) |
| KCD224 | QVQLKESGPGLVAPSQSLSITCTVSGFS | KCD224 | DIQMTQTTSSLSASLGDRVTISCRASQVI |
| | LTSYGVDWIRQSPGKGLEWLGVIWGV | | SNYLNWYQQKPDGTVKLLIYYTSRLHS |
| | GSTNYNSALKSRLSISKDNSKSQVFLK | | GVPSRFSGSGSGTDYSLTISNLEPEDIAT |
| | MNSLQTDDTAMYYCARSYDGSYWYF | | YYCQQYSKLPYTFGSGTKLEIK (SEQ ID |
| | DVWGTGTTVTVSS (SEQ ID NO: 605) | | NO: 693) |
| KCD225 | QVQLQQSGPELVKPGASVKISCKASGY | KCD225 | DIQMTQSPASLSVSTGETVTITCRASENI |
| | TFTDYYINWMKQRPGQGLEWIGWIFPG | | YSNLAWFQQKQGKSPQLLVYAATNLA |
| | SDSTYYNEKFKGKATLTVDKSSSTAY | | DGVPSRFSGSGSGTQYSLKITSLQSEDFG |
| | MLLSSLTSEDSAVYFCARLGYYSHSYY | | SYYCQHFWGTPLTFGAGTKLDLK (SEQ |
| | AMDYWGQGTSVTVSS (SEQ ID NO: | | ID NO: 694) |
| | 606) | | |
| KCD229 | EVQLVESGGGLVQPKGSLKLSCAASGF | KCD229 | DVLMTQNPLSLPVSLGDQASISCRSSQSI |
| | SFNTYAMNWVRQAPGKGLEWVARIRS | | VHSNGNTYLEWYLQKPGQSPNLLIYNV |
| | KSNNYATYYADSVKDRFTIFRDDSESM | | FNRFSGVPDRFSGSGSGTDFTLKISRVEA |
| | LYLQMNNLKTEDTAMYYCVRHGYYW | | EDLGVYYCFQGSHVPYTFGSGTKLEIK |
| | YFDVWGTGTTVTVSS (SEQ ID NO: 607) | | (SEQ ID NO: 695) |
| KCD230 | QVQLQQPGTELVKPGASVKLPCKASG | KCD230 | DIQMTQTTSSLSVSLGDRVTISCRASQDI |
| | YTFTSYWMQWVKQRPGQGLEWIGEID | | TNYLNWYQQKPDGTVKLLIYFTSRLHS |
| | PSDTYINYNQKFKGKATLTVDTSSTTA | | GVPSRFSGSGSGTDYSLTISNLEPEDIAT |
| | YMQLSSLTSEDSAVYYCARYTTIMASD | | YYCQQYSKLPWTFGGGTKLEIK (SEQ |
| | YWGQGTTLTVSS (SEQ ID NO: 608) | | ID NO: 696) |
| KCD232 | EVQLVESGGGLVQPKGSLKLSCAASGF | KCD232 | DVLMTQTPLSLPVSLGDQASISCRSSQSI |
| | SFNTYAMNWVRQAPGKGLEWVARIRS | | VHSDGNTYLEWYLQKPGQSPKLLIYRV |
| | KSNNYATYYADSVKDRFTISRDDSESM | | SNRFSGVPDRFSGSGSGTDFTLKISRME |
| | VYLQMNNLKTEDTAMYYCVRHGYYW | | AEDLGVYYCFQGSHVPYTFGGGTKLEI |
| | YFDVWGTGTTVTVSS (SEQ ID NO: 609) | | K (SEQ ID NO: 697) |

TABLE 1D
Variable Regions Sequences of Variant Humanized CFD Antagonist Antibodies
based on a CDR definition

| | CDR1 | CDR2 | CDR3 |
|---|---|---|---|
| Light chain | QX₁X₂X₃HX₄NX₅X₆X₇YX₈E, | X₁X₂LX₃X₄KX₅X₆X₇RX₈; | X₁QGSX₂X₃PX₄T, wherein |
| | wherein X₁ is T, D, G, H, I, N, Q, R, | wherein X₁ is N, D, E, R, S, | X₁ is F or M; X₂ is H, A, E, |
| | V, or W; X₂ is I, A, or V; X₃ is V, A, | V, or Y; X₂ is L or I; X₃ is I | F, G, L, N, Q, T, V, W, or Y; |
| | D, E, F, I, K, L, Q, R, S, W, or Y; X₄ is | or T; X₄ is Y, D, E, F, G, L, | X₃ is V, W, Q or N; and X₄ is |
| | S, A, D, F, G, I, R, T, V, or W; X₅ is | R, S, T, or V; X₅ is V, A, or | P or V |
| | G, E, F, or S; X₆ is D or E; X₇ is T, S, | I; X₆ is S, A, F, G, K, L, Q, | |
| | or V; X₈ is L or I | R, T, or Y; X₇ is N, E, G, H, | |
| | | I, L, Q, R, T, or Y; X₈ is F, | |
| | | E, G, I, L, R or W | |
| Heavy chain | GYX₁FTX₂X₃X₄X₅, wherein X₁ is T, | WIGDX₁X₂X₃X₄X₅X₆X₇X₈X_{9,}X₁₀, wherein | X₁REGPX₂FX₃X₄, wherein |
| | D, E, F, H, I, K, P, R, S, W, or Y; X₂ is | X₁ is I, L, OR V; X₂ is N, A, | X₁ is T, A, or Q; X₂ is S or A; |
| | D, A, F, G, S, V, or Y; X₃ is Y or F; X₄ | or G; X₃ is P, or T; X₄ is N, | X₃ is A or R; and X₄ is Y, A, |
| | is Y or P; X₅ is M, H, or I | A, D, G, H, I, L, R, T,V, W, | H, F, V, Y, or L |
| | | OR Y; X₅ is T, I, K, L, Q, R, | |
| | | S, or V X₆ is G or V; X₇ is | |
| | | any amino acid; X₈ is any | |
| | | amino acid except I; X₉ is | |
| | | any amino acid except K or | |
| | | R; X₁₀ is any amino acid | |
| | | except I or W. | |

CDRs (streamlined CDR defined sequence)

| | CDR1 | CDR2 | CDR3 |
|---|---|---|---|
| Light Chain | QX₁X₂X₃HX₄NX₅X₆X₇Y, wherein X₁ | KX₁X₂; wherein X₁ is V, A, | X₁QGSX₂X₃PX₄T, wherein |
| | is T, D, G, H, I, N, Q, R, V, or W; X₂ | or I; X₂ is S, A, F, G, K, L, | X₁ is F or M; X₂ is H, A, E, |
| | is I, A, or V; X₃ is V, A, D, E, F, I, K, | Q, R, T, or Y | F, G, L, N, Q, T, V, W, or Y; |
| | L, Q, R, S, W, or Y; X₄ is S, A, D, F, | | X₃ is V, W, Q or N; and X₄ is |
| | G, I, R, T, V, or W; X₅ is G, E, F, or | | P or V |
| | S; X₆ is D or E; X₇ is T, S, or V | | |
| Heavy | X₁X₂X₃, wherein X₁ is D, A, F, G, S, | X₁X₂X₃X₄X₅X₆X₇X₈, | EGPX₁FX₂X₃, wherein X₁ is |
| Chain | V, or Y; X₂ is Y or F; X₃ is Y or P | wherein X₁ is I, L, OR V; | S or A; X₂ is A or R; and X₃ |
| | | X₂ is N, A, or G; X₃ is P, or | is Y, A, H, F, V, Y, or L |
| | | T; X₄ is N, A, D, G, H, I, L, | |
| | | R, T, V, W, OR Y; X₅ is T, | |
| | | I, K, L, Q, R, S, or V X₆ is | |
| | | G or V; X₇ is any amino | |
| | | acid; X₈ is any amino acid | |
| | | except I | |

TABLE 1E
Amino acid sequences of final heavy and light chain candidates and their variants
(KCD119 Variant Sequences; a CDR embodiment is underlined.)
Heavy Chain

| | |
|---|---|
| KCD119 | EVQLVESGGGLVQPGGSLRLSCAASGYTFTDYYMNWVRQAPGKGLEWIGDINPNTGDT |
| TAF | SYNADFKRRFTFSLDTSKSTAYLQMNSLRAEDTAVYYCTREGPSFAYWGQGTLVTVSS |
| | (SEQ ID NO: 698) |
| 54V | EVQLVESGGGLVQPGGSLRLSCAASGYTFTDYYMNWVRQAPGKGLEWIGDINPVTGDT |
| | SYNADFKRRFTFSLDTSKSTAYLQMNSLRAEDTAVYYCTREGPSFAYWGQGTLVTVSS |
| | (SEQ ID NO: 699) |
| 54I | EVQLVESGGGLVQPGGSLRLSCAASGYTFTDYYMNWVRQAPGKGLEWIGDINPITGDTS |
| | YNADFKRRFTFSLDTSKSTAYLQMNSLRAEDTAVYYCTREGPSFAYWGQGTLVTVSS |
| | (SEQ ID NO: 700) |
| 54V59D | EVQLVESGGGLVQPGGSLRLSCAASGYTFTDYYMNWVRQAPGKGLEWIGDINPVTGDT |
| | DYNADFKRRFTFSLDTSKSTAYLQMNSLRAEDTAVYYCTREGPSFAYWGQGTLVTVSS |
| | (SEQ ID NO: 701) |
| 54I59D | EVQLVESGGGLVQPGGSLRLSCAASGYTFTDYYMNWVRQAPGKGLEWIGDINPITGDTD |
| | YNADFKRRFTFSLDTSKSTAYLQMNSLRAEDTAVYYCTREGPSFAYWGQGTLVTVSS |
| | (SEQ ID NO: 702) |
| 34154159D | EVQLVESGGGLVQPGGSLRLSCAASGYTFTDYYINWVRQAPGKGLEWIGDINPITGDTD |
| | YNADFKRRFTFSLDTSKSTAYLQMNSLRAEDTAVYYCTREGPSFAYWGQGTLVTVSS |
| | (SEQ ID NO: 703) |
| 31S34I5 | EVQLVESGGGLVQPGGSLRLSCAASGYTFTSYYINWVRQAPGKGLEWIGDINPITGDTDY |
| 4159D | NADFKRRFTFSLDTSKSTAYLQMNSLRAEDTAVYYCTREGPSFAYWGQGTLVTVSS |
| | (SEQ ID NO: 704) |
| 3415415 | EVQLVESGGGLVQPGGSLRLSCAASGYTFTDYYINWVRQAPGKGLEWIGDINPITGDTD |
| 9D84S | YNADFKRRFTFSLDTSKSTAYLQMSSLRAEDTAVYYCTREGPSFAYWGQGTLVTVSS |
| | (SEQ ID NO: 705) |
| 31S3415 | EVQLVESGGGLVQPGGSLRLSCAASGYTFTSYYINWVRQAPGKGLEWIGDINPITGDTDY |
| 4I59D84S | NADFKRRFTFSLDTSKSTAYLQMSSLRAEDTAVYYCTREGPSFAYWGQGTLVTVSS |
| | (SEQ ID NO: 706) |

Light Chain

| | |
|---|---|
| KCD119 | DIQLTQSPSSLSASVGDRVTITCRSSQTIVHSNGDTYLEWYQQKPGKAPNLLIYKVSNRFS |
| TAF | GVPSRFSGSGSGTDFTLTISSLQPEDFATYYCFQGSHVPPTFGQGTKVEIK (SEQ ID NO: 707) |
| 54R | DIQLTQSPSSLSASVGDRVTITCRSSQTIVHSNGDTYLEWYQQKPGKAPNLLIRKVSNRFS |
| | GVPSRFSGSGSGTDFTLTISSLQPEDFATYYCFQGSHVPPTFGQGTKVEIK (SEQ ID NO: 708) |
| 101V | DIQLTQSPSSLSASVGDRVTITCRSSQTIVHSNGDTYLEWYQQKPGKAPNLLIYKVSNRFS |
| | GVPSRFSGSGSGTDFTLTISSLQPEDFATYYCFQGSHVPVTFGQGTKVEIK (SEQ ID NO: 709) |
| 54R101V | DIQLTQSPSSLSASVGDRVTITCRSSQTIVHSNGDTYLEWYQQKPGKAPNLLIRKVSNRFS |
| | GVPSRFSGSGSGTDFTLTISSLQPEDFATYYCFQGSHVPVTFGQGTKVEIK (SEQ ID NO: 710) |
| 54G101V | DIQLTQSPSSLSASVGDRVTITCRSSQTIVHSNGDTYLEWYQQKPGKAPNLLIGKVSNRFS |
| | GVPSRFSGSGSGTDFTLTISSLQPEDFATYYCFQGSHVPVTFGQGTKVEIK (SEQ ID NO: 711) |

The methods described herein may further include wherein the anti-VEGF-A antibody conjugate has the following structure:

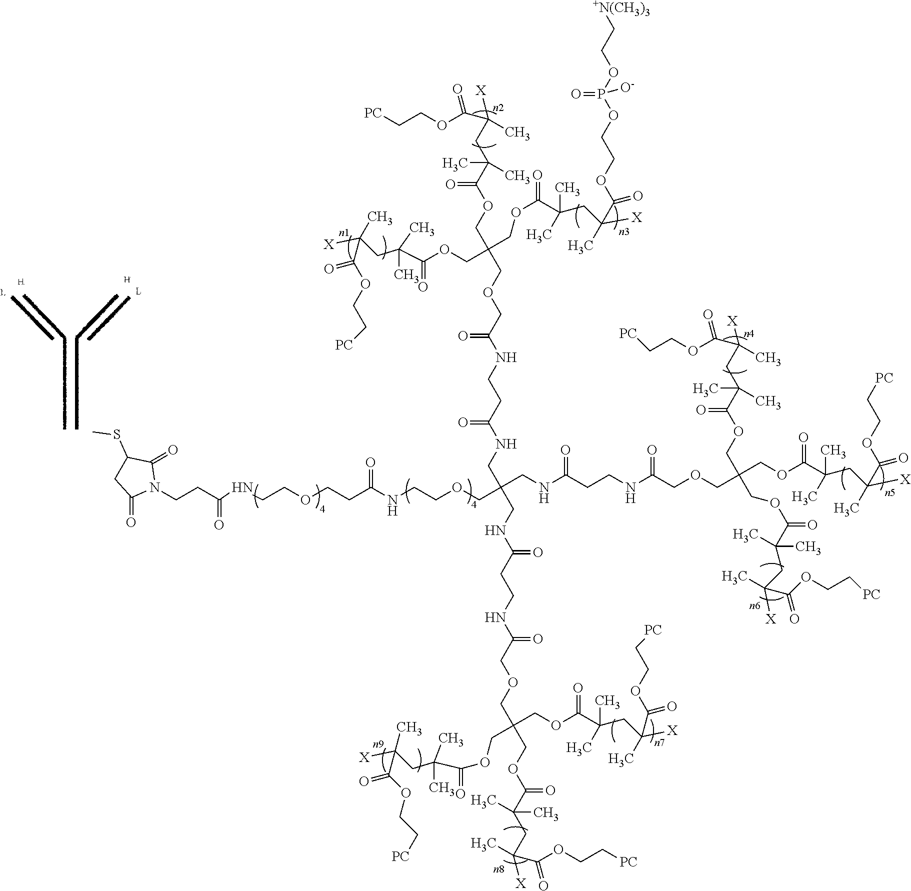

wherein: each heavy chain of the anti-VEGF-A antibody is denoted by the letter H, and each light chain of the anti-VEGF-A antibody is denoted by the letter L; the polymer is bonded to the anti-VEGF-A antibody through the sulfhydryl of C443 (EU numbering), which bond is depicted on one of the heavy chains; PC is

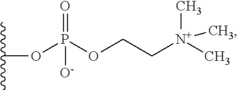 [SMILES: *OP(=O)([O-])OCC[N+](C)(C)C]

where the curvy line indicates the point of attachment to the rest of the polymer, where X=a) OR where R═H, methyl, ethyl, propyl, isopropyl, b) H, or c) any halide, including Br; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

In some embodiments, the anti VEGF A heavy chain comprises CDRH1: that is a CDRH1 in SEQ ID NO: 172, CDRH2: that is a CDRH2 in SEQ ID NO: 173, and CDRH3: that is a CDRH3 in SEQ ID NO: 174, and position 221 is T, and the anti VEGF-A light chain comprises CDRL1: that is a CDRL1 in SEQ ID NO: 199, CDRL2: that is a CDRL2 in SEQ ID NO: 200, and CDRL3: that is a CDRL3 in SEQ ID NO: 201, and Kabat position 4 is L. Optionally, the anti-VEGF A heavy chain isotype is human IgG1. Optionally, the sequence of the anti-VEGF-A heavy chain is at least one of SEQ ID NOs: 7-13, 19-27, 89, 90, 256-262, and the sequence of the anti-VEGF-A antibody light chain is at least one of SEQ ID NOs: 91-93, 28-30. In some embodiments, the antibody that binds to VEGF-A comprises a CDRH1 that is a CDRH1 in SEQ ID NO: 172; a CDRH2 that is a CDRH2 in SEQ ID NO: 173; a CDRH3 that is a CDRH3 in SEQ ID NO: 174: a CDRL1 that is a CDRL1 in SEQ ID NO: 199; a CDRL2 that is a CDRL2 in SEQ ID NO: 200; a CDRL3 that is a CDRL3 in SEQ ID NO: 201; at least one of the following mutations (EU numbering): L234A, L235A, and G237A; and at least one of the following mutations (EU numbering): Q347C or L443C.

The methods described herein may further include wherein the anti-IL-6 antibody conjugate has the following structure:

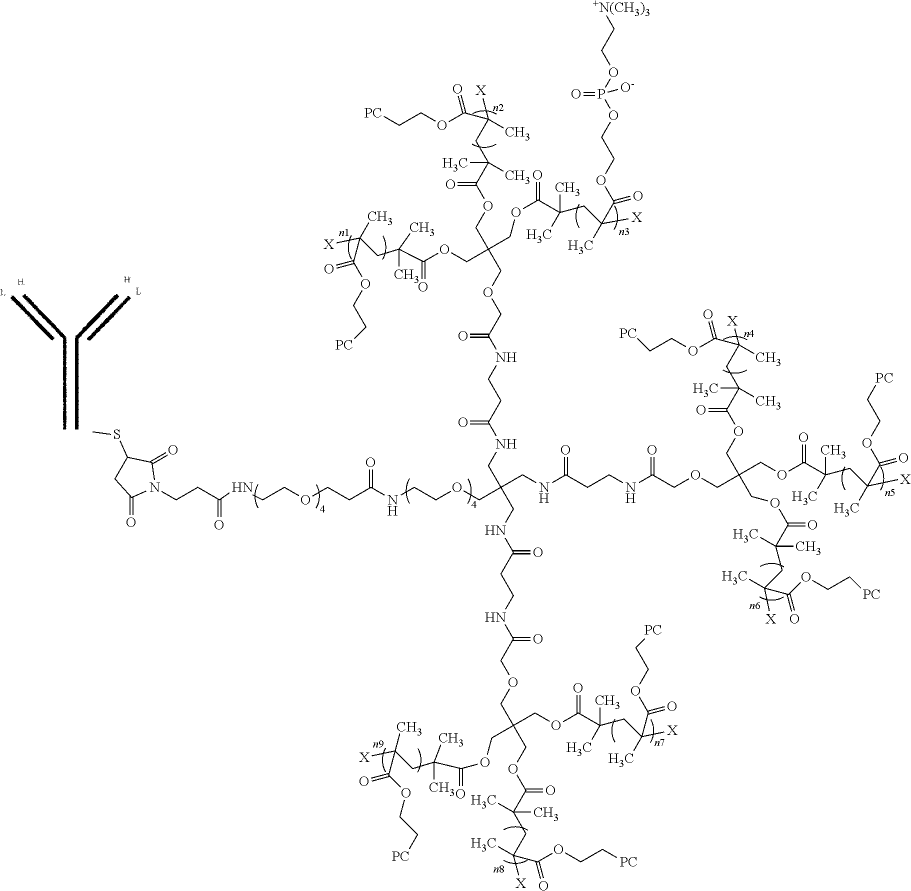

wherein: each heavy chain of the anti-IL-6 antibody is denoted by the letter H, and each light chain of the anti-IL-6 antibody is denoted by the letter L, the polymer is bonded to the anti-IL-6 antibody (and/or Ab-Trap) through the sulfhydryl of C443 (EU numbering), which bond is depicted on one of the heavy chains; PC is,

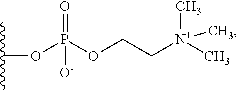 [SMILES: *OP(=O)([O-])OCC[N+](C)(C)C]

where the curvy line indicates the point of attachment to the rest of the polymer; wherein X=a) OR where R═H, Methyl, ethyl, propyl, isopropyl, b) H, or c) any halide, including Br; and n1, n2, n3, n4, n5, n6, n7, n8 and n9 are the same or different such that the sum of n1, n2, n3, n4, n5, n6, n7, n8 and n9 is 2500 plus or minus 15%. In some embodiments, n1, n2, n3, n4, n5, n6, n7, n8 and n9 are the same or different and are integers from 0 to 3000. In some embodiments, n1, n2, n3, n4, n5, n6, n7, n8 and n9 are the same or different and are integers from 0 to 500. In some embodiments, X═OR, where R is a sugar, an aminoalkyl, mono-substituted, poly-substituted or unsubstituted variants of the following residues: saturated C₁-C₂₄ alkyl, unsaturated C₂-C₂₄ alkenyl or C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, amino, aminocarbonyl, aminocarboyloxy, nitro, azido, phenyl, hydroxy, alkylthio, arylthio, oxysulfonyl, carboxy, cyano, and halogenated alkyl including polyhalogenated alkyl, —CO—O—R₇, carbonyl —CCO—R₇, —CO—NR₈R₉, —(CH₂)ₙ—COOR₇, —CO—(CH)ₙ—COOR₇, —(CH₂)ₙ—NR₈R₉, ester, alkoxycarbonyl, aryloxycarbonyl, wherein n is an integer from 1 to 6, wherein each R₇, R8 and R₉ is separately selected from the group consisting of a hydrogen atom, halogen atom, mono-substituted, poly-substituted or unsubstituted variants of the following residues: saturated C₁-C₂₄ alkyl, unsaturated C₂-C₂₄ alkenyl or C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, amino, aminocarbonyl, aminocarboyloxy, nitro, azido, phenyl, hydroxy, alkylthio, arylthio, oxysulfonyl, carboxy, cyano, and halogenated alkyl including polyhalogenated alkyl, a 5-membered ring, and a 6-membered ring.

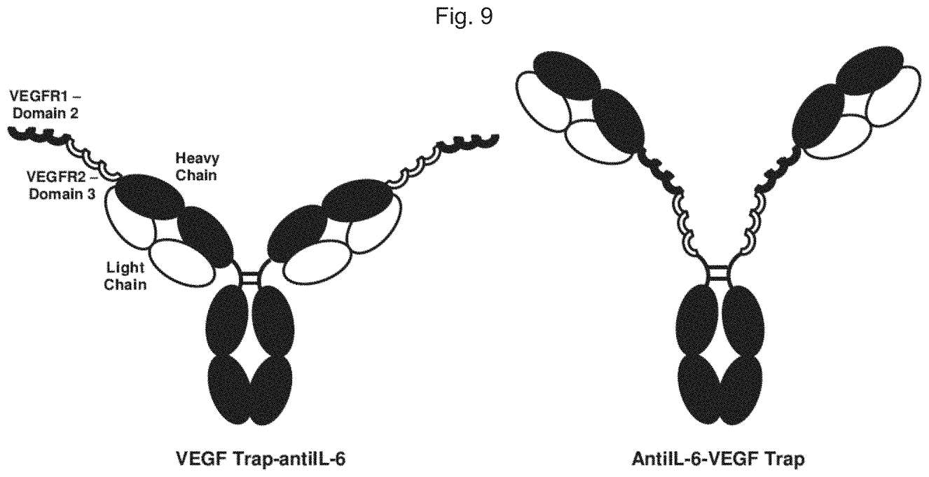

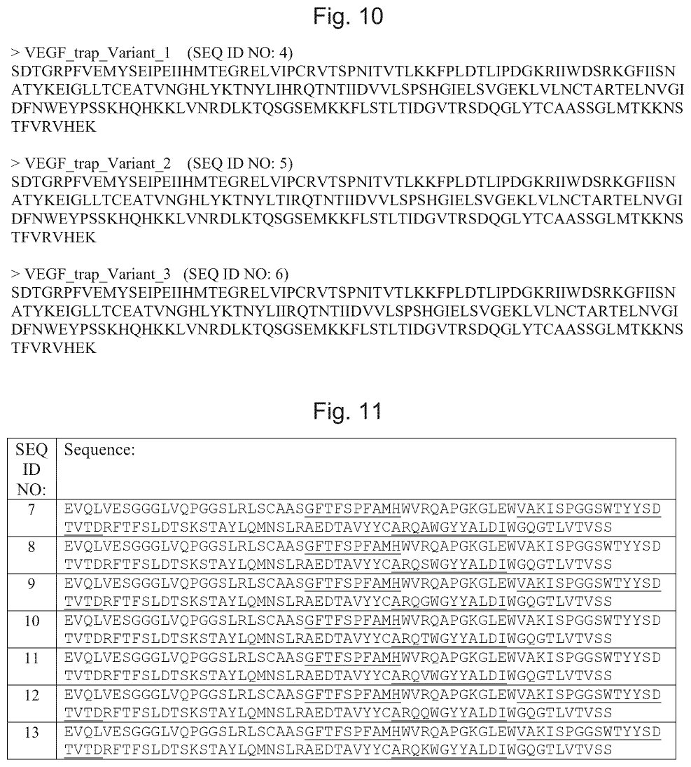

In some embodiments, the anti-IL-6 antibody, or as used otherwise herein, “IL-6 antibody-VEGF Trap fusion”, “IL-6 antibody-VEGF Trap”, “Ab IL-6-VEGF Trap”, “AntiIL-6-VEGF Trap”, “VEGFR-AntiIL6”, “VEGFR-AntiIL-6”, “VEGF Trap-anti-IL6 Antibody Fusion (TAF)”, “VEGF Trap-IL6”, “VEGFR IL-6”, “IL6-VEGFR” or similar term or inverse terms (e.g. “VEGF Trap-IL-6 Ab,” “VEGF Trap-IL-6 antibody fusion,” etc.) denote the fusion between the IL-6 antibody and the VEGF Trap. Embodiments are depicted in FIG. 9. When used generically, the order of the two terms can be swapped. When used specifically, the order of the two terms denotes the relative position of the components in the construct. the term “Ab-Trap”, “IL-6 Ab-VEGF Trap” “Ab IL-6 VEGF Trap” or “Ab IL-6-Trap” or “antiIL-6 VEGF Trap”, “Trap-Ab”, AntiIL-6-VEGFR, AntiIL6-VEGFR or other similar term or inverse terms (e.g. “VEGF Trap-IL-6 Ab,” “VEGF Trap-IL-6 antibody fusion,” etc.) denotes the arrangement of the Ab fused to the relevant domains of a VEGF binding protein so as to provide a VEGF trap. As noted above, this section of the VEGF binding protein is one that prevents VEGF from binding to VEGF receptors. As described herein, the arrangement (ordering) of the Trap and antibody sections can be varied. Thus, unless denoted otherwise explicitly or by context, the phrases used herein regarding Ab-Trap (or Il-6/VEGF Trap, etc.) fusions, denote all disclosed embodiments for the positioning of the antibody and the Trap. Thus, unless explained otherwise, the phrase Ab-Trap (or Il-6/VEGF Trap, etc.), denotes the left embodiment in FIG. 9, and the right embodiment in FIG. 9, and both embodiments in FIG. 9. Thus, the general language is denoted as disclosing all three options for convenience. If the orientation is specifically denoted, it can be denoted, for example, by stating that the “arrangement” can be one of: Trap-Ab, Trap IL-6 Ab, VEGF Trap Ab IL-6, VEGF Trap Ab IL6. Similarly, it will be appreciated that the context of some of the present Examples specific orientations or arrangements of the molecules, which are denoted by the context of the Example. Both arrangements (in the alternative and combined) are explicitly contemplated for all discussions of fusion proteins provided herein. In addition, due to the ordering, it is appreciated that the phrase IL-6 Ab, when used in the context of the fusion protein, includes both the option where the antibody is contiguous, FIG. 9, left-hand side, and where the TRAP is positioned “within” the Ab (FIG. 9 right-hand side). Again, the term “Ab” or “antibody”, when used in the fusion protein context (or other similar term), encompasses all three options (left-hand side of FIG. 9, right-hand side of FIG. 9, and both options), unless otherwise noted. In some embodiments, the VEGF Trap is fused to IL-6 in one of the following manners: to an N-terminal end of a heavy chain comprising IL-6 VH; or between a hinge region and after a CH1 domain of a heavy chain comprising IL-6 VH. There is no difference between the designations of Ab, antibody, “anti” or other similar term when used in a name to designate and antibody or fragment thereof. There is no difference between the designations of “Il-6” or “IL6” or “IL-6”. As used herein, when referencing a fusion construct with IL-6, the terms “VEGF”, “VEGFR”, “VEGF Trap”, “VEGFR Trap” are used interchangeably. The terms can have different meanings when used separately from the IL-6 fusion arrangement, which will depend upon the context of the term in question.

In some embodiments, the antibody can be linked or fused to a VEGF Trap sequence. In some embodiments, this Trap sequence can be as shown in Table 2A. In some embodiments, the sequence is at least 80% identical to that shown in Table 2A, e.g., at least 80, 85, 90, 95, 96, 97, 98, 99% identical to that shown in 2A. In some embodiments, any of the VEGF Trap molecules in U.S. Pub. No. 20150376271 can be employed herein. In some embodiments, the VEGF Trap sequence is fused to IL-6 in one of the following manners: to an N-terminal end of a heavy chain comprising IL-6 VH (FIG. 9 left), or between a hinge region and after a CH1 domain of a heavy chain comprising IL-6 VH (FIG. 9 right). Unless designated otherwise, both options in the alternative and together are contemplated for the embodiments provided herein wherein any Ab-Trap fusion is discussed. In some embodiments, the term “Trap” refers to a full length extracellular region or any portion thereof, or combination of portions from different VEGF receptors that can antagonize signaling between at least one VEGF and VEGFR. Preferably, the extracellular trap segment includes at least one domain from one of VEGFR-1, -2 or -3, and more preferably at least two contiguous domains, such as D2 and D3. Optionally, an extracellular domain includes at least one domain from at least two different VEGFRs. A preferred extracellular domain comprises or consists essentially of D2 of VEGFR-1 and D3 of VEGFR-2.

TABLE 2A
VEGFR1, Domain 2 and VEGFR2, Domain 3 Fusion
sequence.

SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKN
STFVRVHEK (SEQ ID NO: 114)

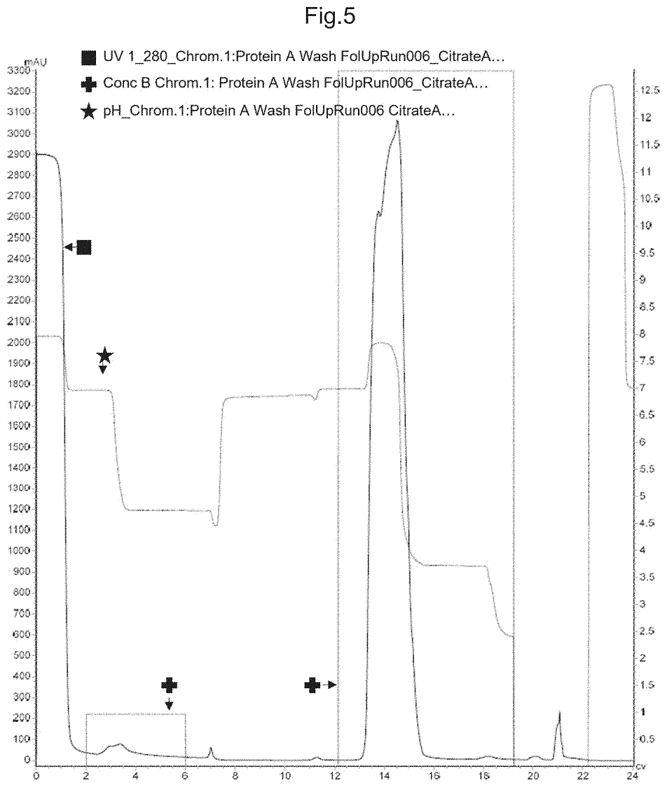

In some embodiments, the IL-6 Ab VEGF Trap construct can have any of the sequences provided in TABLE 2B or 2B-1. In some embodiments, the construct can be at least identical to the sequences in Table 2B or 2B-1, e.g., 80, 85, 90, 95, 96, 97, 98, 99 or higher. In some embodiments, the fusion protein can be in line with those percentages, with the exception that the antibody IL-6 domain does not contain one or more of the CDRs in Table 1E. In some embodiments, the fusion protein is one that contains one or more of the identified sequences in FIG. 5, e.g., one of more of the CDRS (including 2, 3, 4, 5 or 6 of the boxed CDRs) and/or the entire heavy and light chain variable regions, along with a VEGF Trap sequence (e.g., Table 2C). In some embodiments, the sequences can be directly fused to one another. In some embodiments, one or more flexible linking sequences or sections can be used. The linking sequence can be positioned between the Ab sequence and the VEGF Trap sequence. These sequences can be 5 to 30 amino acids in length. In some embodiments, the linking sequence can include G and S in a ratio of about 4:1. In some embodiments, the linker includes the following sequence: GGGGSGGGGS (SEQ ID NO: 748). In some embodiments, any flexible linker can be employed. In some embodiments, the Fc portion of the IL-6 Ab is IgG1.

TABLE 2B
Heavy and light chain sequences for dual inhibitor molecules. CDRs are underlined
in the heavy and light chains, VEGF trap sequence is bolded in black, Gly-Ser linker is
italicized.

| ID | Heavy chain | Light chain |
|---|---|---|
| A | SDTGRPFVEMYSEIPEIIHMTEGRELVIPCRVTSPNI | DIQLTQSPSSLSASVGDRVTITCSASISVSYLYW |
| | TVTLKKFPLDTLIPDGKRIIWDSRKGFIISNATYKEI | YQQKPGKAPKLLIYDDSSLASGVPSRFSGSGSG |
| | GLLTCEATVNGHLYKTNYLTHRQTNTIIDVVLSPS | TDFTLTISSLQPEDFATYYCQQWSGYPYTFGQG |
| | HGIELSVGEKLVLNCTARTELNVGIDENWEYPSSK | TKVEIKRTVAAPSVFIFPPSDEQLKSGTASVVCL |
| | HQHKKLVNRDLKTQSGSEMKKFLSTLTIDGVTRS | LNNFYPREAKVQWKVDNALQSGNSQESVTEQ |
| | DQGLYTCAASSGLMTKKNSTFVRVHEKGGGGSG | DSKDSTYSLSSTLTLSKADYEKHKVYACEVTH |
| | GGGSEVQLVESGGGLVQPGGSLRLSCAASGFTFSP | QGLSSPVTKSFNRGEC (SEQ ID NO: 730) |
| | FAISWVRQAPGKGLEWVAKISPGGSWTYYSDTVT | |
| | DRFTFSLDTSKSTAYLQMNSLRAEDTAVYYCARQ | |
| | LWGYYALDVWGQGTLVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKKVEPKSCDKTHTCPPCPAPEAA | |
| | GAPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTISK | |
| | AKGQPREPQVYTLPPSREEMTKNQVSLTCLVKGF | |
| | YPSDIAVEWESNGQPENNYKTTPPVLDSDGSFFLY | |
| | SKLTVDKSRWQQGNVFSCSVMHEALHNHYTQKS | |
| | LSCSPGK (SEQ ID NO: 712) | |
| B | SDTGRPFVEMYSEIPEIIHMTEGRELVIPCRVTSPNI | DIQLTQSPSSLSASVGDRVTITCSASISVSYLYW |
| | TVTLKKFPLDTLIPDGKRIIWDSRKGFIISNATYKEI | YQQKPGKAPKLLIYDASSLASGVPSRFSGSGSG |
| | GLLTCEATVNGHLYKTNYLTHRQTNTIIDVVLSPS | TDFTLTISSLQPEDFATYYCQQWSGYPYTFGQG |
| | HGIELSVGEKLVLNCTARTELNVGIDFNWEYPSSK | TKVEIKRTVAAPSVFIFPPSDEQLKSGTASVVCL |
| | HQHKKLVNRDLKTQSGSEMKKFLSTLTIDGVTRS | LNNFYPREAKVQWKVDNALQSGNSQESVTEQ |
| | DQGLYTCAASSGLMTKKNSTFVRVHEKGGGGSG | DSKDSTYSLSSTLTLSKADYEKHKVYACEVTH |
| | GGGSEVQLVESGGGLVQPGGSLRLSCAASGFTFSP | QGLSSPVTKSFNRGEC (SEQ ID NO: 731) |
| | FAISWVRQAPGKGLEWVAKISPGGSWTYYSDTVT | |
| | DRFTFSLDTSKSTAYLQMNSLRAEDTAVYYCARQ | |
| | LWGYYALDVWGQGTLVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKKVEPKSCDKTHTCPPCPAPEAA | |
| | GAPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTISK | |
| | AKGQPREPQVYTLPPSREEMTKNQVSLTCLVKGF | |
| | YPSDIAVEWESNGQPENNYKTTPPVLDSDGSFFLY | |
| | SKLTVDKSRWQQGNVFSCSVMHEALHNHYTQKS | |
| | LSCSPGK (SEQ ID NO: 713) | |
| C | SDTGRPFVEMYSEIPEIIHMTEGRELVIPCRVTSPNI | DIQLTQSPSSLSASVGDRVTITCSASISVSYLYW |
| | TVTLKKFPLDTLIPDGKRIIWDSRKGFIISNATYKEI | YQQKPGKAPKLLIYDDSNLASGVPSRFSGSGSG |
| | GLLTCEATVNGHLYKTNYLTHRQTNTIIDVVLSPS | TDFTLTISSLQPEDFATYYCQQWSGYPYTFGQG |
| | HGIELSVGEKLVLNCTARTELNVGIDFNWEYPSSK | TKVEIKRTVAAPSVFIFPPSDEQLKSGTASVVCL |
| | HQHKKLVNRDLKTQSGSEMKKFLSTLTIDGVTRS | LNNFYPREAKVQWKVDNALQSGNSQESVTEQ |
| | DQGLYTCAASSGLMTKKNSTFVRVHEKGGGGSG | DSKDSTYSLSSTLTLSKADYEKHKVYACEVTH |
| | GGGSEVQLVESGGGLVQPGGSLRLSCAASGFTFSP | QGLSSPVTKSFNRGEC (SEQ ID NO: 732) |
| | FAISWVRQAPGKGLEWVAKISPGGSWTYYSDTVT | |
| | DRFTFSLDTSKSTAYLQMNSLRAEDTAVYYCARQ | |
| | LWGYYALDVWGQGTLVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYICN | |
| | VNHKPSNTKVDKKVEPKSCDKTHTCPPCPAPEAA | |
| | GAPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTISK | |
| | AKGQPREPQVYTLPPSREEMTKNQVSLTCLVKGF | |
| | YPSDIAVEWESNGQPENNYKTTPPVLDSDGSFFLY | |
| | SKLTVDKSRWQQGNVFSCSVMHEALHNHYTQKS | |
| | LSCSPGK (SEQ ID NO: 714) | |
| D | SDTGRPFVEMYSEIPEIIHMTEGRELVIPCRVTSPNI | DIQLTQSPSSLSASVGDRVTITCSASISVSYLYW |
| | TVTLKKFPLDTLIPDGKRIIWDSRKGFIISNATYKEI | YQQKPGKAPKLLIYDDSSLASGVPSRFSGSGSG |
| | GLLTCEATVNGHLYKTNYLTHRQTNTIIDVVLSPS | TDFTLTISSLQPEDFATYYCQQWSGYPYTFGQG |
| | HGIELSVGEKLVLNCTARTELNVGIDFNWEYPSSK | TKVEIKRTVAAPSVFIFPPSDEQLKSGTASVVCL |
| | HQHKKLVNRDLKTQSGSEMKKFLSTLTIDGVTRS | LNNFYPREAKVQWKVDNALQSGNSQESVTEQ |
| | DQGLYTCAASSGLMTKKNSTFVRVHEKGGGGSG | DSKDSTYSLSSTLTLSKADYEKHKVYACEVTH |
| | GGGSEVQLVESGGGLVQPGGSLRLSCAASGFTFSP | QGLSSPVTKSFNRGEC (SEQ ID NO: 733) |
| | FAWSWVRQAPGKGLEWVAKISPGGSWTYYSDTV | |
| | TDRFTFSLDTSKSTAYLQMNSLRAEDTAVYYCAR | |
| | QLWGYYALDVWGQGTLVTVSSASTKGPSVFPLAP | |
| | SSKSTSGGTAALGCLVKDYFPEPVTVSWNSGALTS | |
| | GVHTFPAVLQSSGLYSLSSVVTVPSSSLGTQTYICN | |
| | VNHKPSNTKVDKKVEPKSCDKTHTCPPCPAPEAA | |
| | GAPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTISK | |
| | AKGQPREPQVYTLPPSREEMTKNQVSLTCLVKGF | |
| | YPSDIAVEWESNGQPENNYKTTPPVLDSDGSFFLY | |
| | SKLTVDKSRWQQGNVFSCSVMHEALHNHYTQKS | |
| | LSCSPGK (SEQ ID NO: 715) | |
| E | SDTGRPFVEMYSEIPEIIHMTEGRELVIPCRVTSPNI | DIQLTQSPSSLSASVGDRVTITCSASISVSYLYW |
| | TVTLKKFPLDTLIPDGKRIIWDSRKGFIISNATYKEI | YQQKPGKAPKLLIYDASSLASGVPSRFSGSGSG |
| | GLLTCEATVNGHLYKTNYLTHRQTNTIIDVVLSPS | TDFTLTISSLQPEDFATYYCQQWSGYPYTFGQG |
| | HGIELSVGEKLVLNCTARTELNVGIDFNWEYPSSK | TKVEIKRTVAAPSVFIFPPSDEQLKSGTASVVCL |
| | HQHKKLVNRDLKTQSGSEMKKFLSTLTIDGVTRS | LNNFYPREAKVQWKVDNALQSGNSQESVTEQ |
| | DQGLYTCAASSGLMTKKNSTFVRVHEKGGGGSG | DSKDSTYSLSSTLTLSKADYEKHKVYACEVTH |
| | GGGSEVQLVESGGGLVQPGGSLRLSCAASGFTFSP | QGLSSPVTKSFNRGEC (SEQ ID NO: 734) |
| | FAWSWVRQAPGKGLEWVAKISPGGSWTYYSDTV | |
| | TDRFTFSLDTSKSTAYLQMNSLRAEDTAVYYCAR | |
| | QLWGYYALDVWGQGTLVTVSSASTKGPSVFPLAP | |
| | SSKSTSGGTAALGCLVKDYFPEPVTVSWNSGALTS | |
| | GVHTFPAVLQSSGLYSLSSVVTVPSSSLGTQTYICN | |
| | VNHKPSNTKVDKKVEPKSCDKTHTCPPCPAPEAA | |
| | GAPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTISK | |
| | AKGQPREPQVYTLPPSREEMTKNQVSLTCLVKGF | |
| | YPSDIAVEWESNGQPENNYKTTPPVLDSDGSFFLY | |
| | SKLTVDKSRWQQGNVFSCSVMHEALHNHYTQKS | |
| | LSCSPGK (SEQ ID NO: 716) | |
| F | SDTGRPFVEMYSEIPEIIHMTEGRELVIPCRVTSPNI | DIQLTQSPSSLSASVGDRVTITCSASISVSYLYW |
| | TVTLKKFPLDTLIPDGKRIIWDSRKGFIISNATYKEI | YQQKPGKAPKLLIYDDSNLASGVPSRFSGSGSG |
| | GLLTCEATVNGHLYKTNYLTHRQTNTIIDVVLSPS | TDFTLTISSLQPEDFATYYCQQWSGYPYTFGQG |
| | HGIELSVGEKLVLNCTARTELNVGIDFNWEYPSSK | TKVEIKRTVAAPSVFIFPPSDEQLKSGTASVVCL |
| | HQHKKLVNRDLKTQSGSEMKKFLSTLTIDGVTRS | LNNFYPREAKVQWKVDNALQSGNSQESVTEQ |
| | DQGLYTCAASSGLMTKKNSTFVRVHEKGGGGSG | DSKDSTYSLSSTLTLSKADYEKHKVYACEVTH |
| | GGGSEVQLVESGGGLVQPGGSLRLSCAASGFTFSP | QGLSSPVTKSFNRGEC (SEQ ID NO: 735) |
| | FAWSWVRQAPGKGLEWVAKISPGGSWTYYSDTV | |
| | TDRFTFSLDTSKSTAYLQMNSLRAEDTAVYYCAR | |
| | QLWGYYALDVWGQGTLVTVSSASTKGPSVFPLAP | |
| | SSKSTSGGTAALGCLVKDYFPEPVTVSWNSGALTS | |
| | GVHTFPAVLQSSGLYSLSSVVTVPSSSLGTQTYICN | |
| | VNHKPSNTKVDKKVEPKSCDKTHTCPPCPAPEAA | |
| | GAPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTISK | |
| | AKGQPREPQVYTLPPSREEMTKNQVSLTCLVKGF | |
| | YPSDIAVEWESNGQPENNYKTTPPVLDSDGSFFLY | |
| | SKLTVDKSRWQQGNVFSCSVMHEALHNHYTQKS | |
| | LSCSPGK (SEQ ID NO: 717) | |
| G | SDTGRPFVEMYSEIPEIIHMTEGRELVIPCRVTSPNI | DIQLTQSPSSLSASVGDRVTITCSASISVSYLYW |
| | TVTLKKFPLDTLIPDGKRIIWDSRKGFIISNATYKEI | YQQKPGKAPKLLIYDDSSLASGVPSRFSGSGSG |
| | GLLTCEATVNGHLYKTNYLTHRQTNTIIDVVLSPS | TDFTLTISSLQPEDFATYYCQQWSGYPYTFGQG |
| | HGIELSVGEKLVLNCTARTELNVGIDFNWEYPSSK | TKVEIKRTVAAPSVFIFPPSDEQLKSGTASVVCL |
| | HQHKKLVNRDLKTQSGSEMKKFLSTLTIDGVTRS | LNNFYPREAKVQWKVDNALQSGNSQESVTEQ |
| | DQGLYTCAASSGLMTKKNSTFVRVHEKGGGGSG | DSKDSTYSLSSTLTLSKADYEKHKVYACEVTH |
| | GGGSEVQLVESGGGLVQPGGSLRLSCAASGFTFSP | QGLSSPVTKSFNRGEC (SEQ ID NO: 736) |
| | FAMHWVRQAPGKGLEWVAKISPGGSWTYYSDTV | |
| | TDRFTFSLDTSKSTAYLQMNSLRAEDTAVYYCAR | |
| | QAWGYYALDIWGQGTLVTVSSASTKGPSVFPLAP | |
| | SSKSTSGGTAALGCLVKDYFPEPVTVSWNSGALTS | |
| | GVHTFPAVLQSSGLYSLSSVVTVPSSSLGTQTYICN | |
| | VNHKPSNTKVDKKVEPKSCDKTHTCPPCPAPEAA | |
| | GAPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTISK | |
| | AKGQPREPQVYTLPPSREEMTKNQVSLTCLVKGF | |
| | YPSDIAVEWESNGQPENNYKTTPPVLDSDGSFFLY | |
| | SKLTVDKSRWQQGNVFSCSVMHEALHNHYTQKS | |
| | LSCSPGK (SEQ ID NO: 718) | |
| H | SDTGRPFVEMYSEIPEIIHMTEGRELVIPCRVTSPNI | DIQLTQSPSSLSASVGDRVTITCSASISVSYLYW |
| | TVTLKKFPLDTLIPDGKRIIWDSRKGFIISNATYKEI | YQQKPGKAPKLLIYDASSLASGVPSRFSGSGSG |
| | GLLTCEATVNGHLYKTNYLTHRQTNTIIDVVLSPS | TDFTLTISSLQPEDFATYYCQQWSGYPYTFGQG |
| | HGIELSVGEKLVLNCTARTELNVGIDFNWEYPSSK | TKVEIKRTVAAPSVFIFPPSDEQLKSGTASVVCL |
| | HQHKKLVNRDLKTQSGSEMKKFLSTLTIDGVTRS | LNNFYPREAKVQWKVDNALQSGNSQESVTEQ |
| | DQGLYTCAASSGLMTKKNSTFVRVHEKGGGGSG | DSKDSTYSLSSTLTLSKADYEKHKVYACEVTH |
| | GGGSEVQLVESGGGLVQPGGSLRLSCAASGFTFSP | QGLSSPVTKSFNRGEC (SEQ ID NO: 737) |
| | FAMHWVRQAPGKGLEWVAKISPGGSWTYYSDTV | |
| | TDRFTFSLDTSKSTAYLQMNSLRAEDTAVYYCAR | |
| | QAWGYYALDIWGQGTLVTVSSASTKGPSVFPLAP | |
| | SSKSTSGGTAALGCLVKDYFPEPVTVSWNSGALTS | |
| | GVHTFPAVLQSSGLYSLSSVVTVPSSSLGTQTYICN | |
| | VNHKPSNTKVDKKVEPKSCDKTHTCPPCPAPEAA | |
| | GAPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTISK | |
| | AKGQPREPQVYTLPPSREEMTKNQVSLTCLVKGF | |
| | YPSDIAVEWESNGQPENNYKTTPPVLDSDGSFFLY | |
| | SKLTVDKSRWQQGNVFSCSVMHEALHNHYTQKS | |
| | LSCSPGK (SEQ ID NO: 719) | |
| I | SDTGRPFVEMYSEIPEIIHMTEGRELVIPCRVTSPNI | DIQLTQSPSSLSASVGDRVTITCSASISVSYLYW |
| | TVTLKKFPLDTLIPDGKRIIWDSRKGFIISNATYKEI | YQQKPGKAPKLLIYDDSNLASGVPSRFSGSGSG |
| | GLLTCEATVNGHLYKTNYLTHRQTNTIIDVVLSPS | TDFTLTISSLQPEDFATYYCQQWSGYPYTFGQG |
| | HGIELSVGEKLVLNCTARTELNVGIDFNWEYPSSK | TKVEIKRTVAAPSVFIFPPSDEQLKSGTASVVCL |
| | HQHKKLVNRDLKTQSGSEMKKFLSTLTIDGVTRS | LNNFYPREAKVQWKVDNALQSGNSQESVTEQ |
| | DQGLYTCAASSGLMTKKNSTFVRVHEKGGGGSG | DSKDSTYSLSSTLTLSKADYEKHKVYACEVTH |
| | GGGSEVQLVESGGGLVQPGGSLRLSCAASGFTFSP | QGLSSPVTKSFNRGEC (SEQ ID NO: 738) |
| | FAMHWVRQAPGKGLEWVAKISPGGSWTYYSDTV | |
| | TDRFTFSLDTSKSTAYLQMNSLRAEDTAVYYCAR | |
| | QAWGYYALDIWGQGTLVTVSSASTKGPSVFPLAP | |
| | SSKSTSGGTAALGCLVKDYFPEPVTVSWNSGALTS | |
| | GVHTFPAVLQSSGLYSLSSVVTVPSSSLGTQTYICN | |
| | VNHKPSNTKVDKKVEPKSCDKTHTCPPCPAPEAA | |
| | GAPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTISK | |
| | AKGQPREPQVYTLPPSREEMTKNQVSLTCLVKGF | |
| | YPSDIAVEWESNGQPENNYKTTPPVLDSDGSFFLY | |
| | SKLTVDKSRWQQGNVFSCSVMHEALHNHYTQKS | |
| | LSCSPGK (SEQ ID NO: 720) | |
| J | EVQLVESGGGLVQPGGSLRLSCAASGFTFSPFAIS | DIQLTQSPSSLSASVGDRVTITCSASISVSYLYW |
| | WVRQAPGKGLEWVAKISPGGSWTYYSDTVTDRF | YQQKPGKAPKLLIYDDSSLASGVPSRFSGSGSG |
| | TFSLDTSKSTAYLQMNSLRAEDTAVYYCARQLWG | TDFTLTISSLQPEDFATYYCQQWSGYPYTFGQG |
| | YYALDVWGQGTLVTVSSASTKGPSVFPLAPSSKST | TKVEIKRTVAAPSVFIFPPSDEQLKSGTASVVCL |
| | SGGTAALGCLVKDYFPEPVTVSWNSGALTSGVHT | LNNFYPREAKVQWKVDNALQSGNSQESVTEQ |
| | FPAVLQSSGLYSLSSVVTVPSSSLGTQTYICNVNHK | DSKDSTYSLSSTLTLSKADYEKHKVYACEVTH |
| | PSNTKVDKKVEPKSCGGGGSGGGGSSDTGRPFVE | QGLSSPVTKSFNRGEC (SEQ ID NO: 739) |
| | MYSEIPEIIHMTEGRELVIPCRVTSPNITVTLKKFPL | |
| | DTLIPDGKRIIWDSRKGFIISNATYKEIGLLTCEATV | |
| | NGHLYKTNYLTHRQTNTIIDVVLSPSHGIELSVGEK | |
| | LVLNCTARTELNVGIDFNWEYPSSKHQHKKLVNR | |
| | DLKTQSGSEMKKFLSTLTIDGVTRSDQGLYTCAAS | |
| | SGLMTKKNSTFVRVHEKDKTHTCPPCPAPEAAGA | |
| | PSVFLFPPKPKDTLMISRTPEVTCVVVDVSHEDPEV | |
| | KFNWYVDGVEVHNAKTKPREEQYNSTYRVVSVL | |
| | TVLHQDWLNGKEYKCKVSNKALPAPIEKTISKAK | |
| | GQPREPQVYTLPPSREEMTKNQVSLTCLVKGFYPS | |
| | DIAVEWESNGQPENNYKTTPPVLDSDGSFFLYSKL | |
| | TVDKSRWQQGNVFSCSVMHEALHNHYTQKSLSC | |
| | SPGK (SEQ ID NO: 721) | |
| K | EVQLVESGGGLVQPGGSLRLSCAASGFTFSPFAIS | DIQLTQSPSSLSASVGDRVTITCSASISVSYLYW |
| | WVRQAPGKGLEWVAKISPGGSWTYYSDTVTDRF | YQQKPGKAPKLLIYDASSLASGVPSRFSGSGSG |
| | TFSLDTSKSTAYLQMNSLRAEDTAVYYCARQLWG | TDFTLTISSLQPEDFATYYCQQWSGYPYTFGQG |
| | YYALDVWGQGTLVTVSSASTKGPSVFPLAPSSKST | TKVEIKRTVAAPSVFIFPPSDEQLKSGTASVVCL |
| | SGGTAALGCLVKDYFPEPVTVSWNSGALTSGVHT | LNNFYPREAKVQWKVDNALQSGNSQESVTEQ |
| | FPAVLQSSGLYSLSSVVTVPSSSLGTQTYICNVNHK | DSKDSTYSLSSTLTLSKADYEKHKVYACEVTH |
| | PSNTKVDKKVEPKSCGGGGSGGGGSSDTGRPFVE | QGLSSPVTKSFNRGEC (SEQ ID NO: 740) |
| | MYSEIPEIIHMTEGRELVIPCRVTSPNITVTLKKFPL | |
| | DTLIPDGKRIIWDSRKGFIISNATYKEIGLLTCEATV | |
| | NGHLYKTNYLTHRQTNTIIDVVLSPSHGIELSVGEK | |
| | LVLNCTARTELNVGIDENWEYPSSKHQHKKLVNR | |
| | DLKTQSGSEMKKFLSTLTIDGVTRSDQGLYTCAAS | |
| | SGLMTKKNSTFVRVHEKDKTHTCPPCPAPEAAGA | |
| | PSVFLFPPKPKDTLMISRTPEVTCVVVDVSHEDPEV | |
| | KFNWYVDGVEVHNAKTKPREEQYNSTYRVVSVL | |
| | TVLHQDWLNGKEYKCKVSNKALPAPIEKTISKAK | |
| | GQPREPQVYTLPPSREEMTKNQVSLTCLVKGFYPS | |
| | DIAVEWESNGQPENNYKTTPPVLDSDGSFFLYSKL | |
| | TVDKSRWQQGNVFSCSVMHEALHNHYTQKSLSC | |
| | SPGK (SEQ ID NO: 722) | |
| L | EVQLVESGGGLVQPGGSLRLSCAASGFTFSPFAIS | DIQLTQSPSSLSASVGDRVTITCSASISVSYLYW |
| | WVRQAPGKGLEWVAKISPGGSWTYYSDTVTDRF | YQQKPGKAPKLLIYDDSNLASGVPSRFSGSGSG |
| | TFSLDTSKSTAYLQMNSLRAEDTAVYYCARQLWG | TDFTLTISSLQPEDFATYYCQQWSGYPYTFGQG |
| | YYALDVWGQGTLVTVSSASTKGPSVFPLAPSSKST | TKVEIKRTVAAPSVFIFPPSDEQLKSGTASVVCL |
| | SGGTAALGCLVKDYFPEPVTVSWNSGALTSGVHT | LNNFYPREAKVQWKVDNALQSGNSQESVTEQ |
| | FPAVLQSSGLYSLSSVVTVPSSSLGTQTYICNVNHK | DSKDSTYSLSSTLTLSKADYEKHKVYACEVTH |
| | PSNTKVDKKVEPKSCGGGGSGGGGSSDTGRPFVE | QGLSSPVTKSFNRGEC (SEQ ID NO: 741) |
| | MYSEIPEIIHMTEGRELVIPCRVTSPNITVTLKKFPL | |
| | DTLIPDGKRIIWDSRKGFIISNATYKEIGLLTCEATV | |
| | NGHLYKTNYLTHRQTNTIIDVVLSPSHGIELSVGEK | |
| | LVLNCTARTELNVGIDFNWEYPSSKHQHKKLVNR | |
| | DLKTQSGSEMKKFLSTLTIDGVTRSDQGLYTCAAS | |
| | SGLMTKKNSTFVRVHEKDKTHTCPPCPAPEAAGA | |
| | PSVFLFPPKPKDTLMISRTPEVTCVVVDVSHEDPEV | |
| | KFNWYVDGVEVHNAKTKPREEQYNSTYRVVSVL | |
| | TVLHQDWLNGKEYKCKVSNKALPAPIEKTISKAK | |
| | GQPREPQVYTLPPSREEMTKNQVSLTCLVKGFYPS | |
| | DIAVEWESNGQPENNYKTTPPVLDSDGSFFLYSKL | |
| | TVDKSRWQQGNVFSCSVMHEALHNHYTQKSLSC | |
| | SPGK (SEQ ID NO: 723) | |
| M | EVQLVESGGGLVQPGGSLRLSCAASGFTFSPFAWS | DIQLTQSPSSLSASVGDRVTITCSASISVSYLYW |
| | WVRQAPGKGLEWVAKISPGGSWTYYSDTVTDRF | YQQKPGKAPKLLIYDDSSLASGVPSRFSGSGSG |
| | TFSLDTSKSTAYLQMNSLRAEDTAVYYCARQLWG | TDFTLTISSLQPEDFATYYCQQWSGYPYTFGQG |
| | YYALDVWGQGTLVTVSSASTKGPSVFPLAPSSKST | TKVEIKRTVAAPSVFIFPPSDEQLKSGTASVVCL |
| | SGGTAALGCLVKDYFPEPVTVSWNSGALTSGVHT | LNNFYPREAKVQWKVDNALQSGNSQESVTEQ |
| | FPAVLQSSGLYSLSSVVTVPSSSLGTQTYICNVNHK | DSKDSTYSLSSTLTLSKADYEKHKVYACEVTH |
| | PSNTKVDKKVEPKSCGGGGSGGGGSSDTGRPFVE | QGLSSPVTKSFNRGEC (SEQ ID NO: 742) |
| | MYSEIPEIIHMTEGRELVIPCRVTSPNITVTLKKFPL | |
| | DTLIPDGKRIIWDSRKGFIISNATYKEIGLLTCEATV | |
| | NGHLYKTNYLTHRQTNTIIDVVLSPSHGIELSVGEK | |
| | LVLNCTARTELNVGIDENWEYPSSKHQHKKLVNR | |
| | DLKTQSGSEMKKFLSTLTIDGVTRSDQGLYTCAAS | |
| | SGLMTKKNSTFVRVHEKDKTHTCPPCPAPEAAGA | |
| | PSVFLFPPKPKDTLMISRTPEVTCVVVDVSHEDPEV | |
| | KFNWYVDGVEVHNAKTKPREEQYNSTYRVVSVL | |
| | TVLHQDWLNGKEYKCKVSNKALPAPIEKTISKAK | |
| | GQPREPQVYTLPPSREEMTKNQVSLTCLVKGFYPS | |
| | DIAVEWESNGQPENNYKTTPPVLDSDGSFFLYSKL | |
| | TVDKSRWQQGNVFSCSVMHEALHNHYTQKSLSC | |
| | SPGK (SEQ ID NO: 724) | |
| N | EVQLVESGGGLVQPGGSLRLSCAASGFTFSPFAWS | DIQLTQSPSSLSASVGDRVTITCSASISVSYLYW |
| | WVRQAPGKGLEWVAKISPGGSWTYYSDTVTDRF | YQQKPGKAPKLLIYDASSLASGVPSRFSGSGSG |
| | TFSLDTSKSTAYLQMNSLRAEDTAVYYCARQLWG | TDFTLTISSLQPEDFATYYCQQWSGYPYTFGQG |
| | YYALDVWGQGTLVTVSSASTKGPSVFPLAPSSKST | TKVEIKRTVAAPSVFIFPPSDEQLKSGTASVVCL |
| | SGGTAALGCLVKDYFPEPVTVSWNSGALTSGVHT | LNNFYPREAKVQWKVDNALQSGNSQESVTEQ |
| | FPAVLQSSGLYSLSSVVTVPSSSLGTQTYICNVNHK | DSKDSTYSLSSTLTLSKADYEKHKVYACEVTH |
| | PSNTKVDKKVEPKSCGGGGSGGGGSSDTGRPFVE | QGLSSPVTKSFNRGEC (SEQ ID NO: 743) |
| | MYSEIPEIIHMTEGRELVIPCRVTSPNITVTLKKFPL | |
| | DTLIPDGKRIIWDSRKGFIISNATYKEIGLLTCEATV | |
| | NGHLYKTNYLTHRQTNTIIDVVLSPSHGIELSVGEK | |
| | LVLNCTARTELNVGIDFNWEYPSSKHQHKKLVNR | |
| | DLKTQSGSEMKKFLSTLTIDGVTRSDQGLYTCAAS | |
| | SGLMTKKNSTFVRVHEKDKTHTCPPCPAPEAAGA | |
| | PSVFLFPPKPKDTLMISRTPEVTCVVVDVSHEDPEV | |
| | KFNWYVDGVEVHNAKTKPREEQYNSTYRVVSVL | |
| | TVLHQDWLNGKEYKCKVSNKALPAPIEKTISKAK | |
| | GQPREPQVYTLPPSREEMTKNQVSLTCLVKGFYPS | |
| | DIAVEWESNGQPENNYKTTPPVLDSDGSFFLYSKL | |
| | TVDKSRWQQGNVFSCSVMHEALHNHYTQKSLSC | |
| | SPGK (SEQ ID NO: 725) | |
| O | EVQLVESGGGLVQPGGSLRLSCAASGFTFSPFAWS | DIQLTQSPSSLSASVGDRVTITCSASISVSYLYW |
| | WVRQAPGKGLEWVAKISPGGSWTYYSDTVTDRF | YQQKPGKAPKLLIYDDSNLASGVPSRFSGSGSG |
| | TFSLDTSKSTAYLQMNSLRAEDTAVYYCARQLWG | TDFTLTISSLQPEDFATYYCQQWSGYPYTFGQG |
| | YYALDVWGQGTLVTVSSASTKGPSVFPLAPSSKST | TKVEIKRTVAAPSVFIFPPSDEQLKSGTASVVCL |
| | SGGTAALGCLVKDYFPEPVTVSWNSGALTSGVHT | LNNFYPREAKVQWKVDNALQSGNSQESVTEQ |
| | FPAVLQSSGLYSLSSVVTVPSSSLGTQTYICNVNHK | DSKDSTYSLSSTLTLSKADYEKHKVYACEVTH |
| | PSNTKVDKKVEPKSCGGGGSGGGGSSDTGRPFVE | QGLSSPVTKSFNRGEC (SEQ ID NO: 744) |
| | MYSEIPEIIHMTEGRELVIPCRVTSPNITVTLKKFPL | |
| | DTLIPDGKRIIWDSRKGFIISNATYKEIGLLTCEATV | |
| | NGHLYKTNYLTHRQTNTIIDVVLSPSHGIELSVGEK | |
| | LVLNCTARTELNVGIDFNWEYPSSKHQHKKLVNR | |
| | DLKTQSGSEMKKFLSTLTIDGVTRSDQGLYTCAAS | |
| | SGLMTKKNSTFVRVHEKDKTHTCPPCPAPEAAGA | |
| | PSVFLFPPKPKDTLMISRTPEVTCVVVDVSHEDPEV | |
| | KFNWYVDGVEVHNAKTKPREEQYNSTYRVVSVL | |
| | TVLHQDWLNGKEYKCKVSNKALPAPIEKTISKAK | |
| | GQPREPQVYTLPPSREEMTKNQVSLTCLVKGFYPS | |
| | DIAVEWESNGQPENNYKTTPPVLDSDGSFFLYSKL | |
| | TVDKSRWQQGNVFSCSVMHEALHNHYTQKSLSC | |
| | SPGK (SEQ ID NO: 726) | |
| P | EVQLVESGGGLVQPGGSLRLSCAASGFTFSPFAMH | DIQLTQSPSSLSASVGDRVTITCSASISVSYLYW |
| | WVRQAPGKGLEWVAKISPGGSWTYYSDTVTDRF | YQQKPGKAPKLLIYDDSSLASGVPSRFSGSGSG |
| | TFSLDTSKSTAYLQMNSLRAEDTAVYYCARQAW | TDFTLTISSLQPEDFATYYCQQWSGYPYTFGQG |
| | GYYALDIWGQGTLVTVSSASTKGPSVFPLAPSSKS | TKVEIKRTVAAPSVFIFPPSDEQLKSGTASVVCL |
| | TSGGTAALGCLVKDYFPEPVTVSWNSGALTSGVH | LNNFYPREAKVQWKVDNALQSGNSQESVTEQ |
| | TFPAVLQSSGLYSLSSVVTVPSSSLGTQTYICNVNH | DSKDSTYSLSSTLTLSKADYEKHKVYACEVTH |
| | KPSNTKVDKKVEPKSCGGGGSGGGGSSDTGRPFV | QGLSSPVTKSFNRGEC (SEQ ID NO: 745) |
| | EMYSEIPEIIHMTEGRELVIPCRVTSPNITVTLKKFP | |
| | LDTLIPDGKRIIWDSRKGFIISNATYKEIGLLTCEAT | |
| | VNGHLYKTNYLTHRQTNTIIDVVLSPSHGIELSVGE | |
| | KLVLNCTARTELNVGIDFNWEYPSSKHQHKKLVN | |
| | RDLKTQSGSEMKKFLSTLTIDGVTRSDQGLYTCAA | |
| | SSGLMTKKNSTFVRVHEKDKTHTCPPCPAPEAAG | |
| | APSVFLFPPKPKDTLMISRTPEVTCVVVDVSHEDPE | |
| | VKFNWYVDGVEVHNAKTKPREEQYNSTYRVVSV | |
| | LTVLHQDWLNGKEYKCKVSNKALPAPIEKTISKA | |
| | KGQPREPQVYTLPPSREEMTKNQVSLTCLVKGFYP | |
| | SDIAVEWESNGQPENNYKTTPPVLDSDGSFFLYSK | |
| | LTVDKSRWQQGNVFSCSVMHEALHNHYTQKSLS | |
| | CSPGK (SEQ ID NO: 727) | |
| Q | EVQLVESGGGLVQPGGSLRLSCAASGFTFSPFAMH | DIQLTQSPSSLSASVGDRVTITCSASISVSYLYW |
| | WVRQAPGKGLEWVAKISPGGSWTYYSDTVTDRF | YQQKPGKAPKLLIYDASSLASGVPSRFSGSGSG |
| | TFSLDTSKSTAYLQMNSLRAEDTAVYYCARQAW | TDFTLTISSLQPEDFATYYCQQWSGYPYTFGQG |
| | GYYALDIWGQGTLVTVSSASTKGPSVFPLAPSSKS | TKVEIKRTVAAPSVFIFPPSDEQLKSGTASVVCL |
| | TSGGTAALGCLVKDYFPEPVTVSWNSGALTSGVH | LNNFYPREAKVQWKVDNALQSGNSQESVTEQ |
| | TFPAVLQSSGLYSLSSVVTVPSSSLGTQTYICNVNH | DSKDSTYSLSSTLTLSKADYEKHKVYACEVTH |
| | KPSNTKVDKKVEPKSCGGGGSGGGGSSDTGRPFV | QGLSSPVTKSFNRGEC (SEQ ID NO: 746) |
| | EMYSEIPEIIHMTEGRELVIPCRVTSPNITVTLKKFP | |
| | LDTLIPDGKRIIWDSRKGFIISNATYKEIGLLTCEAT | |
| | VNGHLYKTNYLTHRQTNTIIDVVLSPSHGIELSVGE | |
| | KLVLNCTARTELNVGIDFNWEYPSSKHQHKKLVN | |
| | RDLKTQSGSEMKKFLSTLTIDGVTRSDQGLYTCAA | |
| | SSGLMTKKNSTFVRVHEKDKTHTCPPCPAPEAAG | |
| | APSVFLFPPKPKDTLMISRTPEVTCVVVDVSHEDPE | |
| | VKFNWYVDGVEVHNAKTKPREEQYNSTYRVVSV | |
| | LTVLHQDWLNGKEYKCKVSNKALPAPIEKTISKA | |
| | KGQPREPQVYTLPPSREEMTKNQVSLTCLVKGFYP | |
| | SDIAVEWESNGQPENNYKTTPPVLDSDGSFFLYSK | |
| | LTVDKSRWQQGNVFSCSVMHEALHNHYTQKSLS | |
| | CSPGK (SEQ ID NO: 728) | |
| R | EVQLVESGGGLVQPGGSLRLSCAASGFTFSPFAMH | DIQLTQSPSSLSASVGDRVTITCSASISVSYLYW |
| | WVRQAPGKGLEWVAKISPGGSWTYYSDTVTDRF | YQQKPGKAPKLLIYDDSNLASGVPSRFSGSGSG |
| | TFSLDTSKSTAYLQMNSLRAEDTAVYYCARQAW | TDFTLTISSLQPEDFATYYCQQWSGYPYTFGQG |
| | GYYALDIWGQGTLVTVSSASTKGPSVFPLAPSSKS | TKVEIKRTVAAPSVFIFPPSDEQLKSGTASVVCL |
| | TSGGTAALGCLVKDYFPEPVTVSWNSGALTSGVH | LNNFYPREAKVQWKVDNALQSGNSQESVTEQ |
| | TFPAVLQSSGLYSLSSVVTVPSSSLGTQTYICNVNH | DSKDSTYSLSSTLTLSKADYEKHKVYACEVTH |
| | KPSNTKVDKKVEPKSCGGGGSGGGGSSDTGRPFV | QGLSSPVTKSFNRGEC (SEQ ID NO: 747) |
| | EMYSEIPEIIHMTEGRELVIPCRVTSPNITVTLKKFP | |
| | LDTLIPDGKRIIWDSRKGFIISNATYKEIGLLTCEAT | |
| | VNGHLYKTNYLTHRQTNTIIDVVLSPSHGIELSVGE | |
| | KLVLNCTARTELNVGIDFNWEYPSSKHQHKKLVN | |
| | RDLKTQSGSEMKKFLSTLTIDGVTRSDQGLYTCAA | |
| | SSGLMTKKNSTFVRVHEKDKTHTCPPCPAPEAAG | |
| | APSVFLFPPKPKDTLMISRTPEVTCVVVDVSHEDPE | |
| | VKFNWYVDGVEVHNAKTKPREEQYNSTYRVVSV | |
| | LTVLHQDWLNGKEYKCKVSNKALPAPIEKTISKA | |
| | KGQPREPQVYTLPPSREEMTKNQVSLTCLVKGFYP | |
| | SDIAVEWESNGQPENNYKTTPPVLDSDGSFFLYSK | |
| | LTVDKSRWQQGNVFSCSVMHEALHNHYTQKSLS | |
| | CSPGK (SEQ ID NO: 729) | |

TABLE 2B-1

| SEQ ID NO: | SEQUENCE |
|---|---|
| 1 | VPPGEDSKDVAAPHRQPLTSSERIDKQIRYILDGISALRKETCNKSNMCESSKEALAENN |
| | LNLPKMAEKDGCFQSGFNEETCLVKIITGLLEFEVYLEYLQNRFESSEEQARAVQMSTKV |
| | LIQFLQKKAKNLDAITTPDPTTNASLLTKLQAQNQWLQDMTTHLILRSFKEFLQSSLRAL |
| | RQM |
| 2 | EVQLVESGGGLVQPGGSLRLSCAASGFTFSPFAISWVRQAPGKGLEWVAKISPGGSWTYY |
| | SDTVTDRFTFSLDTSKSTAYLQMNSLRAEDTAVYYCARQLWGYYALDVWGQGTLVTVSS |
| 3 | DIQLTQSPSSLSASVGDRVTITCSASISVSYLYWYQQKPGKAPKLLIYDDSSLASGVPSR |
| | FSGSGSGTDFTLTISSLQPEDFATYYCQQWSGYPYTFGQGTKVEIK |

TABLE 2C
Anti-IL-6 heavy chain variable region sequences. CDRs are underlined.

| ID | Sequence |
|---|---|
| I | EVQLVESGGGLVQPGGSLRLSCAASGFTFSPFAISWVRQAPGKGLEWVAKISPGGSWTYYSDTVTDRFTFS |
| | LDTSKSTAYLQMNSLRAEDTAVYYCARQLWGYYALDVWGQGTLVTVSS (SEQ ID NO: 89) |
| II | EVQLVESGGGLVQPGGSLRLSCAASGFTFSPFAWSWVRQAPGKGLEWVAKISPGGSWTYYSDTVTDRFTF |
| | SLDTSKSTAYLQMNSLRAEDTAVYYCARQLWGYYALDVWGQGTLVTVSS (SEQ ID NO: 90) |
| IIa | EVQLVESGGGLVQPGGSLRLSCAASGFTFSPFAMHWVRQAPGKGLEWVAKISPGGSWTYYSDTVTDRFTF |
| | SLDTSKSTAYLQMNSLRAEDTAVYYCARQAWGYYALDIWGQGTLVTVSS (SEQ ID NO: 256) |
| IIb | EVQLVESGGGLVQPGGSLRLSCAASGFTFSPFAMHWVRQAPGKGLEWVAKISPGGSWTYYSDTVTDRFTF |
| | SLDTSKSTAYLQMNSLRAEDTAVYYCARQSWGYYALDIWGQGTLVTVSS (SEQ ID NO: 257) |
| IIc | EVQLVESGGGLVQPGGSLRLSCAASGFTFSPFAMHWVRQAPGKGLEWVAKISPGGSWTYYSDTVTDRFTF |
| | SLDTSKSTAYLQMNSLRAEDTAVYYCARQGWGYYALDIWGQGTLVTVSS (SEQ ID NO: 258) |
| IId | EVQLVESGGGLVQPGGSLRLSCAASGFTFSPFAMHWVRQAPGKGLEWVAKISPGGSWTYYSDTVTDRFTF |
| | SLDTSKSTAYLQMNSLRAEDTAVYYCARQTWGYYALDIWGQGTLVTVSS (SEQ ID NO: 259) |
| Ile | EVQLVESGGGLVQPGGSLRLSCAASGFTFSPFAMHWVRQAPGKGLEWVAKISPGGSWTYYSDTVTDRFTF |
| | SLDTSKSTAYLQMNSLRAEDTAVYYCARQVWGYYALDIWGQGTLVTVSS (SEQ ID NO: 260) |
| IIf | EVQLVESGGGLVQPGGSLRLSCAASGFTFSPFAMHWVRQAPGKGLEWVAKISPGGSWTYYSDTVTDRFTF |
| | SLDTSKSTAYLQMNSLRAEDTAVYYCARQQWGYYALDIWGQGTLVTVSS (SEQ ID NO: 261) |
| IIg | EVQLVESGGGLVQPGGSLRLSCAASGFTFSPFAMHWVRQAPGKGLEWVAKISPGGSWTYYSDTVTDRFTF |
| | SLDTSKSTAYLQMNSLRAEDTAVYYCARQKWGYYALDIWGQGTLVTVSS (SEQ ID NO: 262) |

TABLE 2D
Anti-IL-6 light chain variable region sequences. CDRs are underlined.

| ID | Sequence |
|---|---|
| III | DIQLTQSPSSLSASVGDRVTITCSASISVSYLYWYQQKPGKAPKLLIYDDSSLASGVPSRFSGSGSGTDFTLTI |
| | SSLQPEDFATYYCQQWSGYPYTFGQGTKVEIK (SEQ ID NO: 91) |
| IV | DIQLTQSPSSLSASVGDRVTITCSASISVSYLYWYQQKPGKAPKLLIYDASSLASGVPSRFSGSGSGTDFTLTI |
| | SSLQPEDFATYYCQQWSGYPYTFGQGTKVEIK (SEQ ID NO: 92) |
| V | DIQLTQSPSSLSASVGDRVTITCSASISVSYLYWYQQKPGKAPKLLIYDDSNLASGVPSRFSGSGSGTDFTLTI |
| | SSLQPEDFATYYCQQWSGYPYTFGQGTKVEIK (SEQ ID NO: 93) |

In some embodiments, the antibody does have one or more (or any) of the following CDRs, Table 2E (which includes Table 2E1, Table 2E2, and/or Table 2E3).

TABLE 2E1

| | | Sequence |
|---|---|---|
| Heavy | CDR1 | GFTFSPFAMS (SEQ ID NO: 94) |
| chain | CDR3 | KISPGGSWTYYSDTVTG (SEQ ID NO: 95) |
| | CDR2 | QLWGYYALDI (SEQ ID NO: 171) |
| Light | CDR1 | SASISVSYMY (SEQ ID NO: 96) |
| chain | CDR3 | DMSNLAS (SEQ ID NO: 97) |
| | CDR2 | MQWSGYPYT (SEQ ID NO: 98) |

TABLE 2E2

| | | Sequence |
|---|---|---|
| Heavy | CDR1 | PFAMS (SEQ ID NO: 244) |
| chain | CDR2 | KISPGGSWTYYSDTVTG (SEQ ID NO: 245) |
| | CDR3 | QLWGYYALDI (SEQ ID NO: 246) |
| Light | CDR1 | SASISVSYMY (SEQ ID NO: 247) |
| chain | CDR2 | DMSNLAS (SEQ ID NO: 248) |
| | CDR3 | MQWSGYPYT (SEQ ID NO: 249) |

TABLE 2E3

| | | Sequence |
|---|---|---|
| Heavy | CDR1 | GFTFSPFAMS (SEQ ID NO: 250) |
| chain | CDR2 | WVAKISPGGSWTYYSDTVTG (SEQ ID NO: 251) |
| | CDR3 | ARQLWGYYALDI (SEQ ID NO: 252) |
| Light | CDR1 | SASISVSYMY (SEQ ID NO: 253) |
| chain | CDR2 | LLIYDMSNLAS (SEQ ID NO: 254) |
| | CDR3 | MQWSGYPYT (SEQ ID NO: 255) |

In some embodiments, an IL-6 antagonist antibody comprises three CDRs of any one of the heavy chain variable regions shown in Tables 2C, 21D, 2E1, 2E2 and/or 2E3. In some embodiments, the antibody comprises three CDRs of any one of the light chain variable regions shown in Tables 2C, 21D, 2E1, 2E2 and/or 2E3. In some embodiments, the antibody comprises three CDRs of any one of the heavy chain variable regions shown in Tables 2C, and three CDRs of any one of the light chain variable regions shown in Tables 2D. In some embodiments, the CDRs are one or more of those designated in Table 3 (which includes Table 3A and/or 3B) or Table 4 (which includes Table 4A and/or 4B) below:

TABLE 3A
ANTI IL-6 HEAVY CHAIN CDR SEQUENCES.

| ID | CDR1 | CDR2 | CDR3 |
|---|---|---|---|
| I | GFTFSPFAIS (SEQ ID NO: | VAKISPGGSWTYYSDTVTD | ARQLWGYYALDV (SEQ ID |
| | 99) | (SEQ ID NO: 100) | NO: 101) |
| II | GFTFSPFAWS (SEQ ID NO: | VAKISPGGSWTYYSDTVTD | ARQLWGYYALDV (SEQ ID |
| | 102) | (SEQ ID NO: 103) | NO: 104) |
| IIa | GFTFSPFAMH (SEQ ID | VAKISPGGSWTYYSDTVTD | ARQAWGYYALDI (SEQ ID |
| | NO: 49) | (SEQ ID NO: 50) | NO: 51) |
| IIb | GFTFSPFAMH (SEQ ID | VAKISPGGSWTYYSDTVTD | ARQSWGYYALDI (SEQ ID |
| | NO:52) | (SEQ ID NO:53) | NO:54) |
| IIc | GFTFSPFAMH (SEQ ID | VAKISPGGSWTYYSDTVTD | ARQGWGYYALDI (SEQ ID |
| | NO:55) | (SEQ ID NO: 56) | NO: 57) |
| IId | GFTFSPFAMH (SEQ ID | VAKISPGGSWTYYSDTVTD | ARQTWGYYALDI (SEQ ID |
| | NO: 58) | (SEQ ID NO: 59) | NO: 60) |
| IIe | GFTFSPFAMH (SEQ ID | VAKISPGGSWTYYSDTVTD | ARQVWGYYALDI (SEQ ID |
| | NO: 61) | (SEQ ID NO: 62) | NO: 63) |
| IIf | GFTFSPFAMH (SEQ ID | VAKISPGGSWTYYSDTVTD | ARQQWGYYALDI (SEQ ID |
| | NO: 64) | (SEQ ID NO: 65) | NO: 66) |
| IIg | GFTFSPFAMH (SEQ ID | VAKISPGGSWTYYSDTVTD | ARQKWGYYALDI (SEQ ID |
| | NO: 67) | (SEQ ID NO: 68) | NO: 69) |

TABLE 3B
ANTI IL-6 HEAVY CHAIN CDR SEQUENCES.

| CDR1 | CDR2 | CDR3 |
|---|---|---|
| PFAMH (SEQ ID NO. 172) | KISPGGSWTYYSDTVTD (SEQ ID | QAWGYYALDI (SEQ ID NO. |
| | NO. 173) | 174) |
| PFAMH (SEQ ID NO. 175) | KISPGGSWTYYSDTVTD (SEQ ID | QSWGYYALDI (SEQ ID NO. |
| | NO. 176) | 177) |
| PFAMH (SEQ ID NO. 178) | KISPGGSWTYYSDTVTD (SEQ ID | QGWGYYALDI (SEQ ID NO. |
| | NO. 179) | 180) |
| PFAMH (SEQ ID NO. 181) | KISPGGSWTYYSDTVTD (SEQ ID | QTWGYYALDI (SEQ ID NO. |
| | NO. 182) | 183) |
| PFAMH (SEQ ID NO. 184) | KISPGGSWTYYSDTVTD (SEQ ID | QVWGYYALDI (SEQ ID NO. |
| | NO. 185) | 186) |
| PFAMH (SEQ ID NO. 187) | KISPGGSWTYYSDTVTD (SEQ ID | QQWGYYALDI (SEQ ID NO. |
| | NO. 188) | 189) |
| PFAMH (SEQ ID NO. 190) | KISPGGSWTYYSDTVTD (SEQ ID | QKWGYYALDI (SEQ ID NO. |
| | NO. 191) | 192) |
| PFAIS (SEQ ID NO. 193) | KISPGGSWTYYSDTVTD (SEQ ID | QLWGYYALDV (SEQ ID NO. |
| | NO. 194) | 195) |
| PFAWS (SEQ ID NO. 196) | KISPGGSWTYYSDTVTD (SEQ ID | QLWGYYALDV (SEQ ID NO. |
| | NO. 197) | 198) |

TABLE 4A
ANTI IL-6 LIGHT CHAIN CDR SEQUENCES.

| ID | CDR1 | CDR2 | CDR3 |
|---|---|---|---|
| III | SASISVSYLY (SEQ ID NO: | LLIYDDSSLAS (SEQ ID NO: | QQWSGYPYT (SEQ ID NO: |
| | 105) | 106) | 107) |
| IV | SASISVSYLY (SEQ ID NO: | LLIYDASSLAS (SEQ ID NO: | QQWSGYPYT (SEQ ID NO: |
| | 108) | 109) | 110) |
| V | SASISVSYLY (SEQ ID NO: | LLIYDDSNLAS (SEQ ID NO: | QQWSGYPYT (SEQ ID NO: |
| | 111) | 112) | 113) |

TABLE 4B
ANTI IL-6 LIGHT CHAIN CDR SEQUENCES.

| CDR1 | CDR2 | CDR3 |
|---|---|---|
| SASISVSYLY SEQ ID NO. 199) | DDSSLAS SEQ ID NO. 200) | QQWSGYPYT SEQ ID NO. 201) |
| SASISVSYLY SEQ ID NO. 202) | DASSLAS SEQ ID NO. 203) | QQWSGYPYT SEQ ID NO. 204) |
| SASISVSYLY SEQ ID NO. 205) | DDSNLAS SEQ ID NO. 206) | QQWSGYPYT SEQ ID NO. 207) |

In some embodiments, the antibody used for binding to IL-6 can be one that includes one or more of the sequences in Tables 2C, 2D, 2E1, 2E2 2E3, 3A, 3B, 4A, and/or 4B. In some embodiments, the antibody used for binding to IL-6 can be one that includes three or more of the sequences in Tables 2C, 2D, 2E1, 2E2 2E3, 3A, 3B, 4A, and/or 4B. In some embodiments, the antibody used for binding to IL-6 can be one that includes six of the sequences in any one of Tables 2C, 2D, 2E1, 2E2 2E3, 3A, 3B, 4A, and/or 4B. In some embodiments, the antibody that binds to IL-6 can be one that competes for binding with an antibody that includes 6 of the specified CDRs in any one of Tables 2C, 2D, 2E1, 2E2 2E3, 3A, 3B, 4A, and/or 4B.

The methods described herein may further include wherein the antibody conjugate has the structure of Formula (I),

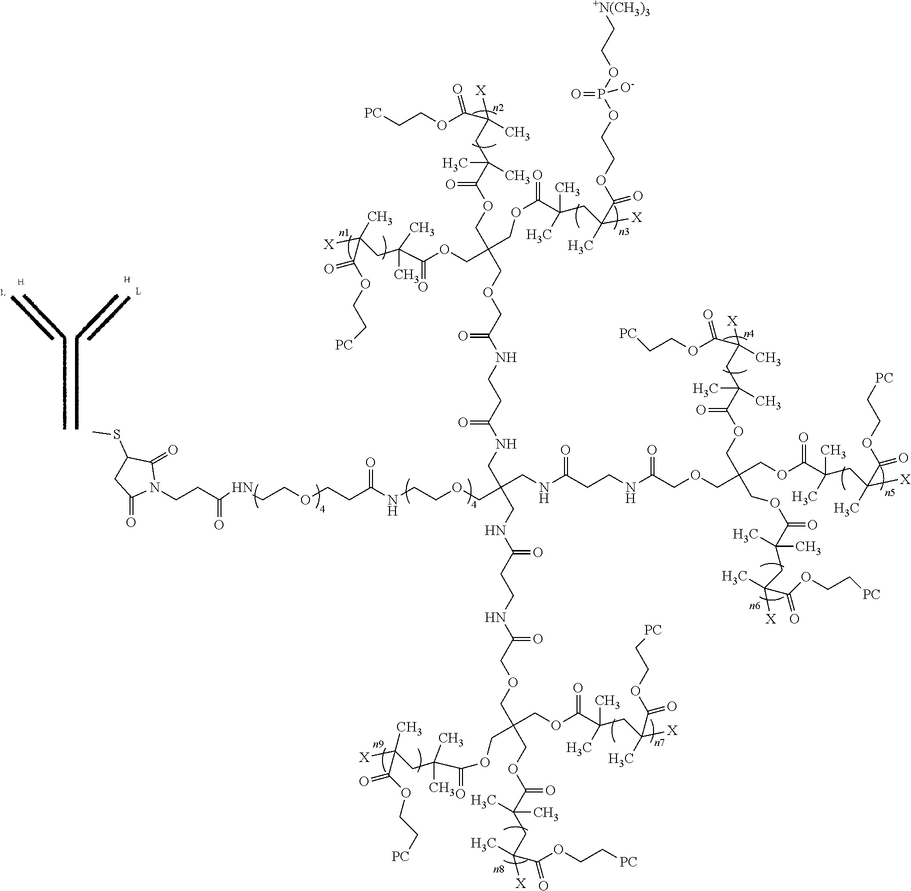

wherein each heavy chain of the anti-VEGF-A antibody is denoted by the letter H, and each light chain of the anti-VEGF-A antibody is denoted by the letter L; the polymer is bonded to the anti-VEGF-A antibody through the sulfhydryl of C443 (EU numbering), which bond is depicted on one of the heavy chains; PC is:

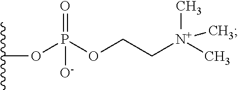 [SMILES: *OP(=O)([O-])OCC[N+](C)(C)C]

wherein the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

In some embodiments, a method of producing a product is provided. The method comprises recovering a cell culture supernatant, wherein the cell culture supernatant comprises a protein of interest, processing the cell culture supernatant into an eluent, wherein the eluent comprises the protein of interest, loading the eluent into an affinity chromatography matrix, washing with a first wash buffer comprising Tris or Sodium Phosphate, washing with a second wash buffer comprising a chaotropic salt, eluting with an elution buffer, wherein an eluate is collected, wherein the eluate comprises a protein product, inactivating viral contaminants present in the eluate with a low pH viral buffer to yield a viral inactivated eluate, filtering the viral inactivated eluate, performing at least one round of ion exchange chromatography on the viral inactivated eluate, and filtering the viral inactivated eluate to yield a retentate, wherein the retentate comprises the protein of interest.

In some embodiments, the method may further include wherein the cell culture supernatant was produced in a bioreactor using animal component free cell culture. The method may further include wherein processing the cell culture supernatant comprises harvesting cell products from a cell culture. The method may further include wherein the cell culture is clarified to remove cells and cellular debris. The method may further include wherein the eluent comprises the clarified cell culture supernatant. A method of purifying a protein using affinity chromatography, the method comprising contacting a load fluid with a medium, wherein the medium is an affinity chromatography matrix that binds a protein of interest, washing the medium with a buffer solution comprising a chaotropic agent, wherein the chaotropic agent is a salt, and contacting the washed medium with an elution solution under conditions suitable for eluting the protein of interest.

In some embodiments, a method of producing a product is provided. The method comprises applying the solution containing a protein of interest onto an affinity chromatography matrix, washing the affinity chromatography matrix with a first buffer, washing the affinity chromatography matrix with a second buffer containing a chaotropic agent, washing the affinity chromatography matrix with a third buffer to remove the chaotropic agent, eluting with an elution buffer, wherein an eluate is collected, wherein the eluate comprises a protein product.

In some embodiments, a system for protein purification is provided. The system comprises a column having a first antigen binding protein bound to the column; a phosphate wash buffer comprising sodium phosphate and a salt, an intermediate wash buffer comprising tris, a second wash buffer comprising magnesium chloride, and an elution buffer comprising sodium formate.

In some embodiments, a system for protein purification is provided. The system comprises a column having a first antigen binding protein bound to the column; a first tris wash buffer comprising tris and a salt, an intermediate tris wash buffer, a second wash buffer comprising magnesium chloride, and an elution buffer comprising sodium formate,

In some embodiments, the system may further include wherein the column comprises a ligand for affinity chromatography. The system may further include wherein the ligand comprises protein A or Protein G. The system may further include wherein the first wash buffer comprising sodium phosphate and a salt has a pH between 5.5 and 9.5. The system may further include wherein the phosphate wash buffer comprising sodium phosphate and a salt comprises about 50 mM sodium phosphate. The system may further include wherein the phosphate wash buffer comprising sodium phosphate and a salt comprises about 250 mM NaCl. The system may further include wherein the first tris wash buffer comprises about 50 mM Tris. The system may further include wherein the first tris wash buffer further comprises about 250 mM NaCl. The system may further include wherein the intermediate tris wash buffer comprises about 50 mM Tris. The system may further include wherein the pH of the first tris wash buffer is about 7.2. The system may further include wherein the pH of the second wash buffer is about 7.8. The system may further include wherein the concentration of magnesium chloride in the second wash buffer is about 2.8 M. The system may further include wherein the concentration of sodium formate in the elution buffer comprises 10 mM.

In some embodiments, a system for protein purification is provided. The system comprises a column having a protein A resin bound to an antibody, wherein the antibody comprises a light and heavy chain of SEQ ID: 2, and SEQ ID 1, respectively, a chaotropic wash buffer comprising a chaotropic salt, and an elution buffer comprising sodium formate.

The methods described herein may further include wherein the protein of interest is a bispecific antibody. The methods described herein may further include wherein the bispecific antibody is specific for VEGF and IL-6. The methods described herein may further include wherein the protein of interest is an antibody conjugate. The methods described herein may further include wherein the affinity chromatography matrix is a protein A chromatography matrix. The methods described herein may further include wherein the chaotropic agent in the buffer solution is comprised of a magnesium salt. The methods described herein may further include wherein the concentration of magnesium salt is between 1.5-3.5 M. The methods described herein may further include wherein the chaotropic agent in the buffer solution is comprised of a calcium salt. The methods described herein may further include wherein the concentration of the calcium salt is between 1-3 M. The methods described herein may further include wherein the chaotropic agent in the buffer solution is comprised of a guanidinium salt. The methods described herein may further include wherein the concentration of the guanidinium salt is between 0.05-3 M.

The methods described herein may further include wherein the buffer solution further comprises tris. The methods described herein may further include wherein the concentration of tris in the buffer solution is at least 5 mM. The methods described herein may further include wherein the pH of the buffer solution is greater than 5.5. The methods described herein may further include wherein the eluate further contains viral impurities. The methods described herein may further include removing the viral impurities. The methods described herein may further include inactivating the viral impurities. The methods described herein may further include washing the affinity chromatography matrix loaded with the load fluid with a prewash buffer solution prior to washing with the buffer solution. The methods described herein may further comprise the step of washing the affinity chromatography matrix loaded with the eluent with a post-wash buffer solution after washing with buffer solution. The methods described herein may further include wherein the prewash buffer solution comprises sodium phosphate. The methods described herein may further include wherein the prewash buffer solution comprises Tris and a salt.

The methods described herein may further include wherein the antibody conjugate has the structure of Formula (I),

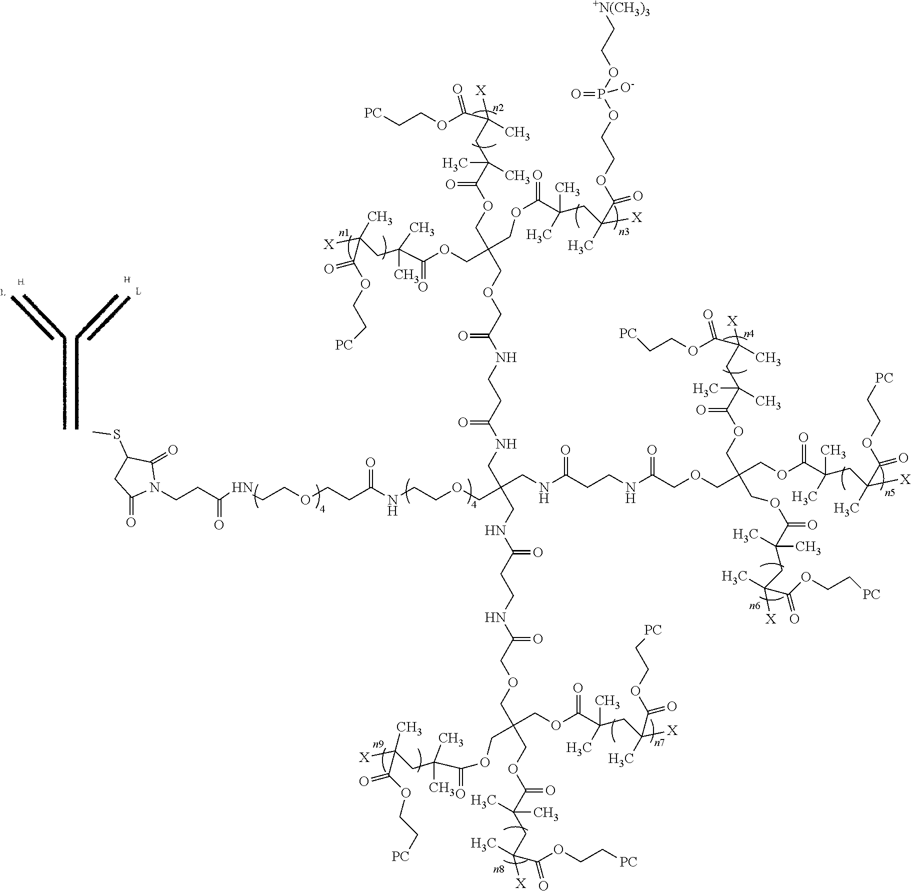

wherein: each heavy chain of the anti-VEGF-A antibody is denoted by the letter H, and each light chain of the anti-VEGF-A antibody is denoted by the letter L; the polymer is bonded to the anti-VEGF-A antibody through the sulfhydryl of C443 (EU numbering), which bond is depicted on one of the heavy chains; PC is:

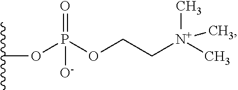 [SMILES: *OP(=O)([O-])OCC[N+](C)(C)C]

wherein the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

The methods described herein may further include wherein the antibody conjugate comprises an anti-VEGF antibody conjugate comprising an anti-VEGF-A light chain and an anti-VEGF-A heavy chain, wherein the anti-VEGF-A antibody heavy chain comprises CDRH1: that is a CDRH1 in SEQ ID NO: 172 CDRH2: that is a CDRH2 in SEQ ID NO: 173, and CDRH3: that is a CDRH3 in SEQ ID NO: 174, and the anti-VEGF-A antibody light chain comprises CDRL1: that is a CDRL1 in SEQ ID NO: 199, CDRL2: that is a CDRL2 in SEQ ID NO: 200, and CDRL3: that is a CDRL3 in SEQ ID NO: 201. The methods described herein may further include wherein the anti-VEGF antibody conjugate comprises: an antibody conjugate comprising an anti-VEGF-A immunoglobulin G (IgG) bonded to a polymer, which polymer comprises MPC monomers, wherein the sequence of the anti-VEGF-A antibody heavy chain is at least one of SEQ ID NOs: 7-13, 19-27, 89, 90, 256-262, and the sequence of the anti-VEGF-A antibody light chain is at least one of SEQ ID NOs: 91-93, 28-30, and wherein the antibody is bonded at C449 in to the polymer. The methods described herein may further include wherein the target protein of interest is produced by a cell culture. The methods described herein may further include wherein the cell culture comprises CHO cells. The methods described herein may further include the step of washing the affinity chromatography matrix loaded with the eluent with a post-wash buffer solution after washing with buffer solution. The methods described herein may further include washing the affinity chromatography matrix with the buffer solution removes nucleic acids, endotoxins, antifoam agents, or other small molecules other than the target protein of interest. The methods described herein may further include washing the affinity chromatography matrix with the buffer solution removes impurities while keeping the target protein of interest bound to the affinity chromatography matrix. The methods described herein may further include wherein washing the affinity chromatography matrix with the buffer solution removes host cell proteins besides the target protein of interest. The methods described herein may further include wherein the addition of chaotropic agent in the buffer solution does not elute the target protein of interest. The methods described herein may further include one or more of virus inactivation, tangential flow filtration, diafiltration, ultrafiltration, ion exchange chromatography, or virus reduction filtration. The methods described herein may further include wherein the eluent was produced in a bioreactor using animal component free cell culture. The methods described herein may further include wherein the product is a protein of interest. The methods described herein may further include wherein impurities comprise host cell protein impurities.

As disclosed herein, the purification method of the present disclosure is effective in removing impurities, like host cell proteins (HCPs). As described in detail in the Examples, the method is effective in reducing HCPs in the wash eluate, while achieving a high percent yield of the protein of interest and a high concentration of the protein of interest. For example, in various embodiments, the method described herein results in a percent yield of the protein of interest that is greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99%.

As disclosed herein, the purification method of the present disclosure is effective in removing impurities from affinity chromatography-based purification methods. In some embodiments, chromatography-based purification methods include methods based on specific macromolecular binding interactions between analytes in a mobile phase and ligands in a stationary phase. In some embodiments, the method is effective in reducing impurities in the wash eluate, while achieving a high percent yield of the species of interest. In some embodiments, the method is effective in reducing impurities in the wash eluate, while achieving a high concentration of the species of interest. For example, in various embodiments, the method described herein results in a percent yield of the species of interest that is greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99%.

As disclosed herein, the purification method of the present disclosure is effective in removing impurities from samples comprising monoclonal antibodies (mAbs). In some embodiments, residues comprising the mAb may be altered or engineered to change overall antibody form or structure. In some embodiments, residues comprising the mAb may be altered or engineered to modulate binding efficacy to a binding partner. In some embodiments, residues comprising the mAb may be altered or engineered to modulate binding efficacy in the antigen binding site. In some embodiments, residues comprising the mAb may be altered or engineered to modulate conjugation efficacy. In some embodiments, the altered residue may be an engineered cysteine. In some embodiments, the monoclonal antibodies comprise specific binding domains, including domains specific for VEGF. In some embodiments, the specific binding domains comprise one or more extracellular components of one or more VEGF receptors. In some embodiments, the binding domains further comprise an Fc portion.

Table 5 lists common species of high-risk HCPs that may be present and removable by the present methods. The table further includes possible downstream impacts on formulated drug products. Without the present techniques, some HCP species become difficult to remove due to physiochemical properties that approach other desirable molecular species. In some embodiments, any one or more of the embodiments provided herein can be used to remove any one or more of the species of Table 5. In some embodiments, the product of interest is selected from one or more of the species of Table 5. In some embodiments, the protein of interest is selected from one or more of the species of Table 5. HCPs that are difficult to remove can be classified as proteins with a molecular weight larger than 15 kDa and/or with a pI between 7.3 and 9.3.

TABLE 5
Common SpeciesA of HCPs

| | MW | | | | Type of | |
|---|---|---|---|---|---|---|
| Protein name | (Da) | pI | Function | Impact | impact | References |
| 78 kDa glucose | 72,379 | 5.07 | Protein folding and | Drug quality | Aggregation | Farrell et al., 2015; |
| regulated protein | | | quality control in | | of drugs | Liu et al., 2019; |
| (GRP78; BiP) | | | the endoplasmic | | | Valente et al., 2015 |
| | | | reticulum lumen | | | |
| Alpha-enolase | 47,141 | 6.37 | Catalyzes the | Drug quality | Modification | Valente et al., 2015; |
| | | | dehydration of 2- | | of drug | Zhang et al., 2014 |
| | | | phosphoglycerate to | | | |
| | | | phosphoenolpyruvate | | | |
| Annexin A5 | 35,752 | 4.82 | Binds with high | Immunogenicity | Immunogenic | Fukuda et al., 2019; |
| (ANXA5) | | | affinity to | | response | Gilgunn & Bones, 2018 |
| | | | phospholipids and | | | |
| | | | serves as a marker | | | |
| | | | for apoptosis | | | |
| C-X-C motif | 10,686 | 8.74 | Cytokine with | Biological | Immunogenic | Gilgunn & Bones, 2018; |
| chemokine 3 | | | potential oncogenic | function in | response | Gilgunn et al., 2019 |
| (CXCL3) | | | properties | | | |
| Carboxyesterase | 60,175 | 5.51 | Catalyzes the | Formulation | Degradation | McShan et al., 2016; |
| (CEB)a | | | cleavage of ester- or | | of | Zhang et al., 2020 |
| | | | amide-containing | | polysorbates | |
| | | | substrates into | | | |
| | | | alcohol and | | | |
| | | | carboxylic acid | | | |
| Carboxypeptidase | 152,406 | 5.77 | Serine exopeptidase | Drug quality | Modification | Hu et al., 2016; |
| D (Cpd) | | | that release C- | | of drug | Dick et al., 2008; |
| | | | terminal amino acids | | | Park, et al., 2017 |
| Cathepsins (B, L, | 37,280 | 5.57 | Cysteine Protease | Drug quality | Fragmentation | Luo et al., 2019; |
| Z)a | | | responsible for | | of drug | Migani et al., 2017; |
| | | | intracellular | | | Park, et al., 2017; |
| | | | proteolysis | | | Vanderlaan et al., 2018 |
| Cathepsin (D, E)a | 43,021 | 4.67 | Aspartyl protease | Drug quality | Fragmentation | Bee et al., 2017; |
| | | | with activity in both | | of drug | Lima et al., 2018; |
| | | | acidic and neutral pH | | | Robert et al., 2009; |
| | | | | | | Yang et al., 2019 |
| Clusterin (CLU) | 51,656 | 5.45 | Multifunctional | Immunogenicity | Immunogenic | Aboulaich et al., 2014; |
| | | | disulfide-linked | | response | Gilgunn & Bones, 2018; |
| | | | heterodimeric | | | Goey et al., 2018 |
| | | | glycoprotein | | | |
| | | | associated with | | | |
| | | | clearance of cellular | | | |
| | | | debris and apoptosis | | | |
| Glutathione-S- | 25,608 | 9.04 | Conjugates reduced | Immunogenicity | Immunogenic | Goey et al., 2018; |
| transferase | | | glutathione to | | response | Jawa et al., 2016 |
| (GST)a | | | exogenous and | | | |
| | | | endogenous | | | |
| | | | hydrophobic | | | |
| | | | electrophiles | | | |
| Lipoprotein | 53,109 | 7.95 | Catalyzes the | Formulation | Degradation | Chiu et al., 2017; |
| lipase (LPL) | | | hydrolysis of | | of | Levy et al., 2014; |
| | | | triacylglycerol of | | polysorbates | Park et al., 2017 |
| | | | LDL and regulates | | | |
| | | | the plasma | | | |
| | | | concentrations of | | | |
| | | | triglycerides and | | | |
| | | | HDL | | | |
| Lysosomal acid | 45,325 | 8.15 | Hydrolyzes | Formulation | Degradation | Levy et al., 2014; |
| lipase (LAL) | | | cholesteryl esters and | | of | McShan et al., 2016 |
| | | | triglycerides | | | |
| Lysosomal | 47,307 | 6.02 | Cleaves the acyl | Formulation | Degradation | Hall et al., 2016; |
| phospholipase A2 | | | ester bonds of | | of | McShan et al., 2016; |
| (LPLA2) | | | glycerophospholipids | | polysorbates | Shayman et al., 2011 |
| | | | to produce free fatty | | | |
| | | | acids | | | |
| Matrix | 70,460 | 9.25 | Endopeptidases | Drug quality | Degradation | Gilgunn & Bones, 2018; |
| metalloproteinase | | | responsible for the | | of drug | Gilgunn et al., 2019 |
| (MMP)a | | | degradation of | | | |
| | | | extracellular matrix | | | |
| | | | proteins | | | |
| Monocyte | 16,326 | 9.81 | Acts as a ligand for | Biological | Cytokine | Leister et al., 2019; |
| chemoattractant | | | C-C chemokine | function in | release | Vanderlaan et al., 2018; |
| Protein-1 (MCP-1) | | | receptor | humans | | Yoshimura & Leonard, 1989 |
| Peptidyl-prolyl | 18,012 | 7.68 | Catalyzes the cis- | Drug quality | Aggregation | Jawa et al., 2013; |
| cis-trans | | | trans isomerization | | of drugs | Zhang et al., 2016 |
| isomerase A | | | of peptide bonds N- | | | |
| (PPIA) | | | terminal to proline | | | |
| | | | residues | | | |
| Peroxiredoxin | 22,263 | 8.22 | Regulates the | Immunogenicity | Immunogenic | Albrecht et al., 2018; |
| (PRDX)a | | | intracellular | | response | Jawa et al., 2016; |
| | | | concentration of | | | Joucla et al., 2013; |
| | | | hydrogen peroxide | | | Park et al., 2017 |
| Phospholipase B- | 66,289 | 5.76 | Potentially catalyzes | Immunogenicity | Immunogenic | Fischer et al., 2017; |
| like 2 (PLBL2) | | | the hydrolysis of | | response | Jawa et al., 2016; |
| | | | phospholipids into | | | Tran et al., 2016; |
| | | | fatty acids | | | Zhang et al., 2020 |
| Procollagen- | 83,595 | 6.08 | Catalyzes | Immunogenicity | Immunogenic | Hogwood et al., 2016; |
| lysine 2- | | | hydroxylation of | | response | Jawa et al., 2016 |
| oxoglutarate 5- | | | lysine residues in | | | |
| deoxygenase_1 | | | collagen alpha chains | | | |
| (PLOD1) | | | | | | |
| Protein disulfide | 57,010 | 4.78 | Catalyzes the | Drug quality | Aggregation | Aboulaich et al., 2014; |
| isomerase (PDI) | | | formation and | | of drugs | Gilgunn &Bones, 2018; |
| | | | breakage of disulfide | | | Goey et al., 2018; |
| | | | bonds | | | Maeda et al., 2007 |
| Protein S100a | 11,241 | 5.28 | Regulates calcium | Immunogenicity | Immunogenic | Gilgunn & Bones, 2018; |
| | | | balance, cell | | response | Gilgunn et al., 2019 |
| | | | apoptosis, migration, | | | |
| | | | proliferation, | | | |
| | | | differentiation, | | | |
| | | | energy metabolism, | | | |
| | | | and inflammation | | | |
| Pyruvate kinase | 62089.32 | 6.19 | Catalyzes the | Immunogenicity | Immunogenic | Goey et al., 2018; |
| (PK) | 58628.38 | 6.07 | conversion of | | response | Jawa et al., 2016; |
| | | | phosphoenolpyruvate | | | Park et al., 2017 |
| | | | to pyruvate and plays | | | |
| | | | a role in regulating | | | |
| | | | cell metabolism | | | |
| Serine protease | 51,214 | 7.82 | Degrades | Drug quality | Modification | Dorai et al., 2011; |
| (HTRA1) | | | proteoglycans and | | of drug | Gilgunn & Bones, 2018; |
| | | | potentially clip N- | | | Goey et al., 2018 |
| | | | terminus | | | |
| Sialate o- | 60,775 | 6.32 | Hydrolyzes | Formulation | Degradation | Schauer et al., 1988 |
| acetylesterase | | | carboxylic acid | | of | |
| (SIAE) | | | ester bonds | | polysorbates | |
| Transforming | 44,310 | 8.90 | Maintains immune | Biological | Cytokine | Beatson et al., 2011; |
| growth factor-β1 | | | homeostasis and | Function in | release | Vanderlaan et al., 2018 |
| (TGF-β1) | | | immune suppression | Humans | | |

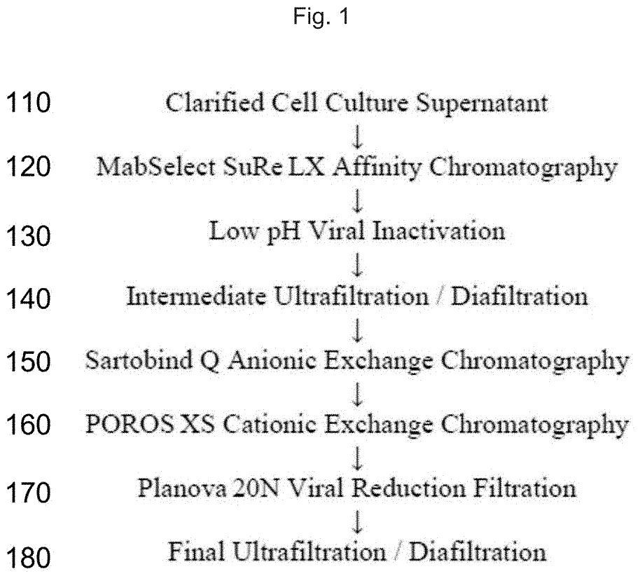

With regards to FIG. 1, a process 100 for the purification of OG2072 antibody from an animal component free cell culture process is disclosed. The process includes three chromatography steps, and two TFF (tangential flow filtration) steps, as well as low pH viral inactivation. First, a clarified cell supernatant is collected 110. Next, Affinity chromatography 120 is performed on the clarified cell supernatant. Low pH Viral inactivation 130, followed by intermediate filtration 140 are then run on an output eluate collected during Affinity Chromatography. Further rounds of chromatography, including Anionic Exchange Chromatography 150, then Cationic Exchange Chromatography 160 are then performed. Viral reduction filtration 170, and a final ultrafiltration/diafiltration 180 follow.

A low pH viral inactivation may include holding a solution at pH 3.5 for 240 minutes followed by neutralization to pH 7. A low pH viral inactivation may alternatively include holding at pH 3.5 for 60 minutes followed by stepwise neutralization to pH 5.5, 6, or 6.5. MabSelect SuRe LX affinity chromatography (MSS LX) can be followed by a virus inactivation/neutralization step and then a first TFF (TFF1) to condition the antibody for Sartobind Q anionic exchange chromatography (AEX chromatography). POROS XS cationic exchange chromatography (CEX chromatography), and a viral reduction filtration (Planova 20N) were then run. POROS XS comprises binding at 10 mM sodium phosphate, pH 5, 40 mM NaCl, plus acetate as supplement (<15 mS/cm), followed by a gradient elution for 10 CVs from 50 mM Na-Acetate, pH 6, 10 mM NaCl to 50 mM Na-Acetate, pH 6, 300 mM NaCl. An additional wash can be performed during POROS XS chromatography comprising 50 mM Na-Acetate, pH 5, and 150 mM NaCl followed by incremental increase of NaCl to 165, 180, 195, or 210 mM). A gradient may be applied to the POROS XS column step so that a gradient from 150 mM NaCl to 400 mM NaCl at pH 5 in 10 CVs with 30 cm bed height, or a gradient from 50 mM NaCl to 350 mM NaCl at pH 6 in 12 CVs with 30 cm bed height are provided. Before gradient elution, wash steps can be performed, the wash steps comprising: 2 CV of 50 mM Na-Acetate, pH 5.0, 10 mM NaCl, followed by 5 CV of 18.8 mM Sodium Phosphate, pH 7.0, 22.5 mM NaCl, followed by 3 CV of 50 mM Na-Acetate, pH 5.0, 10 mM NaCl, followed by 2 CV of 50 mM Sodium Acetate, pH 6.0, 10 mM NaCl. Last, a second TFF (TFF2) was run to formulate the antibody and obtain an antibody intermediate.

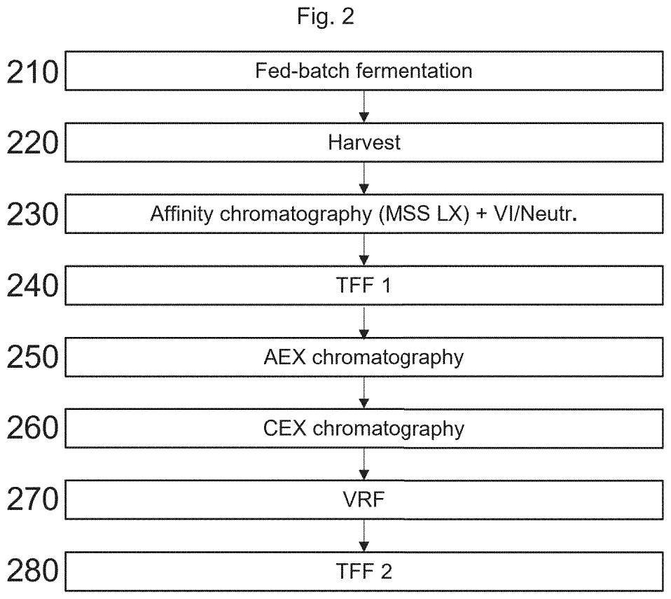

FIG. 2 illustrates a process 200 for purification of a biomolecule. First a cell culture is grown and cultured using a fermentation process 210. The cell culture is then collected at harvest 220 to yield a clarified cell concentrate. The clarified cell concentrate is then purified using affinity chromatography and Viral inactivation/neutralization 230, the output of the process is a product retentate. A tangential flow filtration 240 is then run on the product retentate collected, whereupon an anion exchange chromatography 250 and a cation exchange chromatography 260 are run sequentially. The processed product retentate is then subject to a viral reduction filtration 270, wherein a second tangential flow filtration 280 is then run to yield a purified product.

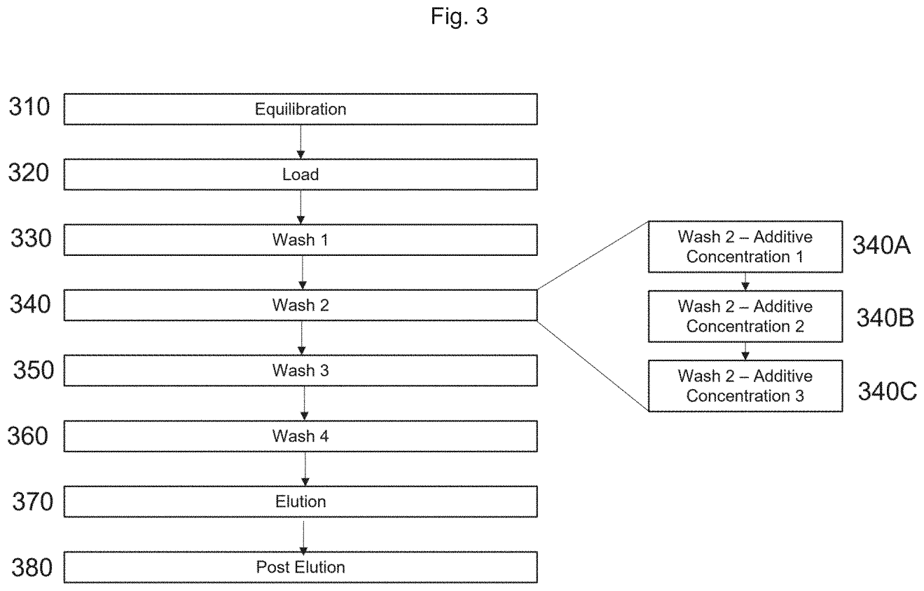

With respect to FIG. 3, a protocol to assess fold-reduction of measurable HCP species was developed to assess yield and purity of a desirable molecular species. A process 300 for the column purification of OG2072 antibody from an animal component free cell culture process is disclosed. The process comprises an equilibration 310, followed by loading 320 the column with clarified cell culture fluid (CCCF). The column is then treated with a series of washes comprising Wash 1 330, followed by Wash 2 340, Wash 3 350, and Wash 4 360. Wash 2 340 may comprise subwashes 340A, 340B, and 340C. Following Wash 4 360, elution 370 is performed, whereby post-elution buffers 380 are run on the column to regenerate the resin. The process includes equilibration 310 of a protein A column with 50 mM Na-phosphate and 250 mM NaCl at pH 7, then a load 320 step, wherein the column is loaded with Clarified cell culture fluid (CCCF), followed by a series of washes 330-360, including a first wash 330 with 50 mM Na/phosphate and 250 mM NaCl at pH 7. Wash 2 340 may comprise various wash buffers and conditions as described in Table 6C. Wash 3 350 comprises a wash with 50 mM Na-phosphate and 2 M NaCl, at pH 7, while Wash 4 360 comprises 50 mM Na-phosphate, 250 mM NaCl, at pH 7. Elution 370 was accomplished with 10 mM Na-Formate at pH 3.5, with post elution run through the column at 100 mM Citric Acid at pH 2.1.

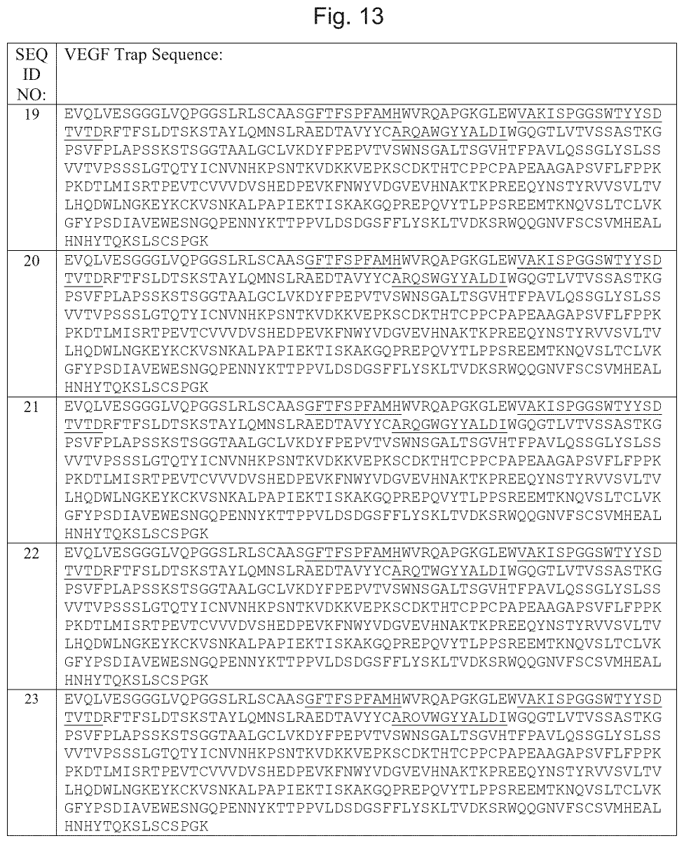

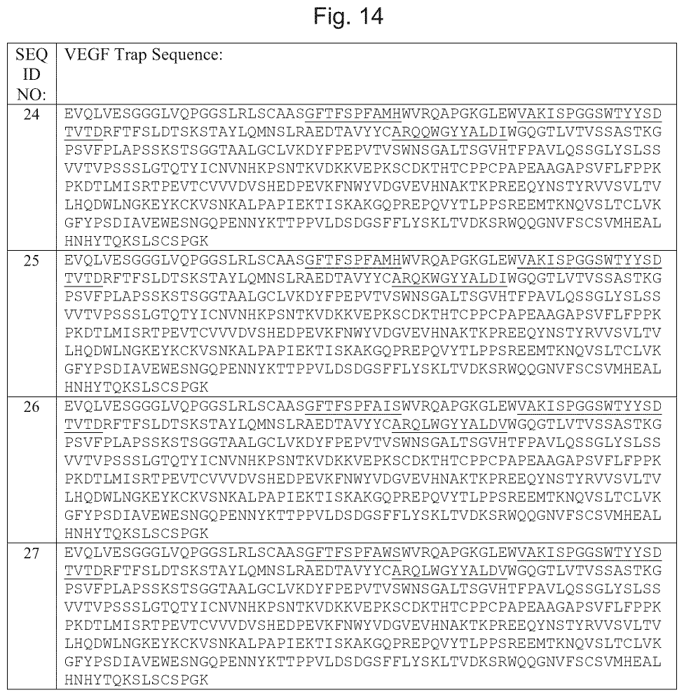

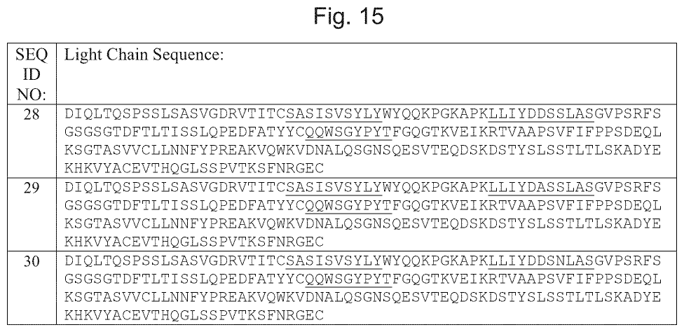

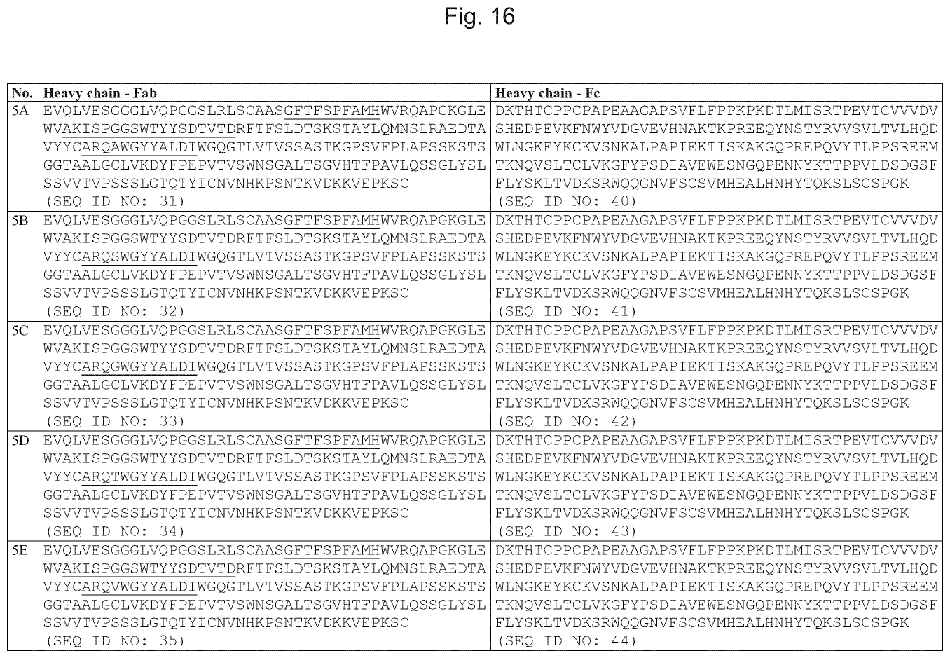

In some embodiments, any one or more of the sequences for the specified amino acid sequence in any one or more of FIGS. 8, 11-16 can be swapped into the corresponding structure of any of the other embodiments provided herein or exchanged with any of the other sequences provided herein. In some embodiments, the construct is a VEGFR-Anti-IL-6 configuration and it includes a combination of one of sequences 1A-1D (FIG. 12), linked to the linker (e.g., FIG. 12, sequence 2A), linked to a heavy chain IL-6 sequence (e.g., FIG. 13, sequence 3A or 3B), linked to a light chain sequence (e.g., FIG. 15, sequence 4A). In some embodiments, any one of sequences 1A-1D (FIG. 12), can be combined with a linker (FIG. 12 and with a heavy chain anti-IL-6 sequence (FIG. 13-14, sequences 3A-3I), and with a light chain anti-IL-6 sequence (FIG. 15, sequence 4A-4C). In some embodiments, any of the other corresponding sequences for any particular unit of construct provided herein can be swapped into or in place of any one of these units.

In some embodiments, any of the anti-IL-6 antibody constructs are contemplated for use as compositions, components, or therapies, including for example, those in tables 2C, 2D, 2E, 0.3 and/or 0.4., and FIGS. 8, 11-16 of the embodiments provided herein.

In some embodiments, the fusion protein comprises a mutation at position 94 in the VEGF Trap sequence, at position 95 in the VEGF Trap sequence, or at T941 and H951 in the VEGF Trap sequence.

In some embodiments, a VEGFR-Anti-IL-6 dual inhibitor is provided. The VEGFR-Anti-IL-6 dual inhibitor comprises a trap antibody fusion of Anti-IL 6 antibody and a VEGF (VEGFR1/2) trap, wherein the dual inhibitor includes at least one point mutation within a VEGFR sequence to reduce cleavage of the VEGFR sequence. In some embodiments, the VEGFR-Anti-IL-6 dual inhibitor has a molecular weight of 1.0 MDa.

In some embodiments, the VEGR-Anti-IL-6 dual inhibitor provides therapy for inflammatory retinal diseases.

In some embodiments, the VEGFR-Anti-IL-6 dual inhibitor comprises a constant heavy, constant light, a fragment antigen binding, a fragment crystallizable (Fc), vascular endothelial growth factor receptor (VEGFR), a variable heavy, a variable light and CDR regions.

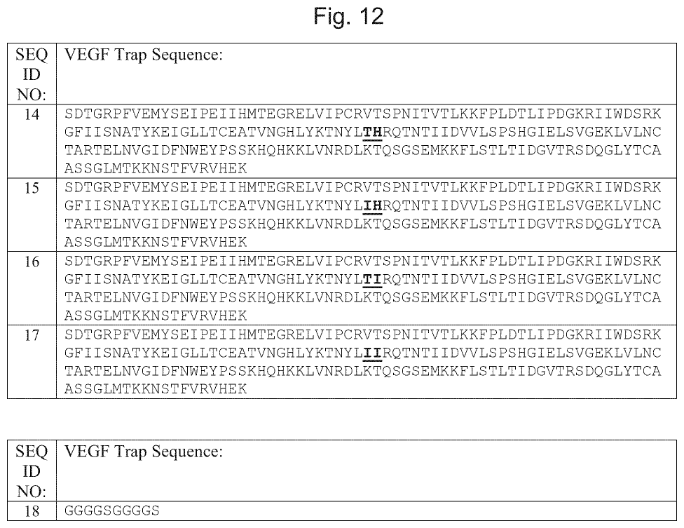

In some embodiments, the Anti-IL-6 heavy chain variable region sequences can be selected from SEQ ID NO: 7, 8, 9, 10, 11, 12, or 13 of FIG. 11.

In some embodiments, the VEGF trap sequence can be selected from at least one of SEQ ID NO: 14, 15, 16, or 17 of FIG. 12.

In some embodiments for the VEGR-anti-IL-6 dual inhibitor, the heavy chain sequence for anti-IL-6 molecules can be selected from at least one of SEQ ID NO: 18, 19, 20, 21, 22, 23, 24, 25, 26 or 27 of FIG. 13-14.

In some embodiments for the VEGFR-anti-IL-6 dual inhibitor the light chain sequence for Anti-IL-6 molecules comprises at least 1, 2, or 3 light chain CDRs from at least one of SEQ ID NO: 28, 29, or 30 of FIG. 15.

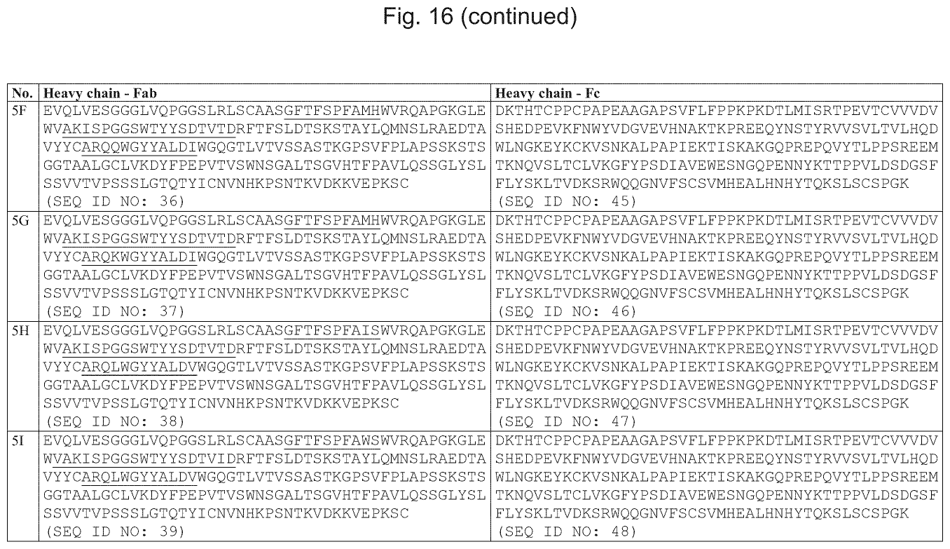

In some embodiments for the VEGFR-anti-IL-6 dual inhibitor the heavy chain sequence for the anti-IL-6 molecule comprises at least 1, 2, or 3 heavy chain CDRs from at least one of option 5A, 5B, 5C, 5D, 5E, 5F, 5G, 5H, or 5I of FIG. 16.

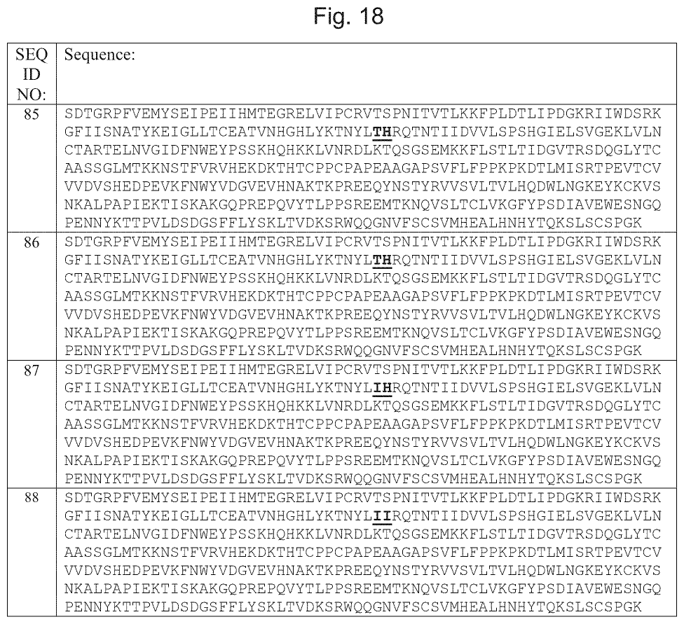

In some embodiments, the VEGFR-Anti-IL-6 dual inhibitor comprises a VEGFR-Fc sequence from at least one of SEQ ID NO: 85, 86, 87, or 88 of FIG. 18.

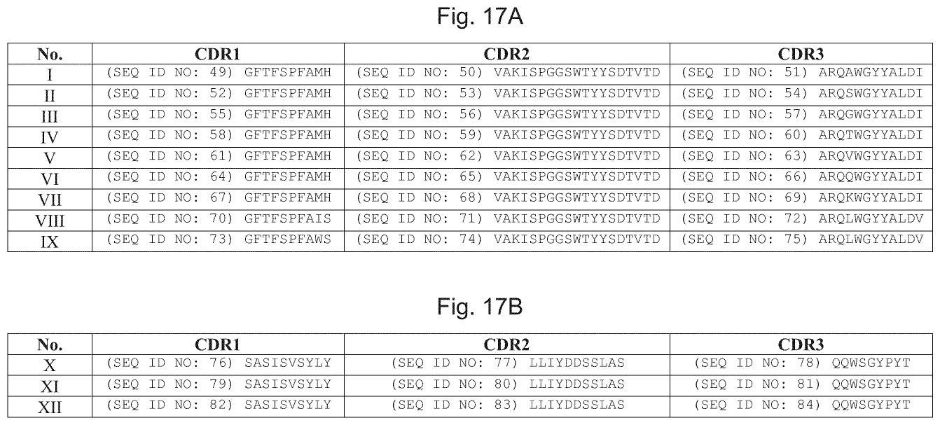

In some embodiments, the VEGFR-anti-IL-6 dual inhibitor comprises one or more of the sequences in FIGS. 11-18.

In some embodiments, the VEGFR-Anti-IL-6 dual inhibitor comprises an IL-6 VH, an IL-6 VL, an IL-6 Fc, a VEGF Trap, and a linker. In some embodiments, the IL-6 VH comprises a sequence from an IL6 VH sequence in FIG. 16 or 13-14. In some embodiments, the IL-6 VL comprises a sequence from an IL6 VL sequence in FIG. 15. In some embodiments, the Fc comprises a sequence from a Fc sequence in FIG. 16. In some embodiments, the VEGF Trap comprises a sequence from a VEGF trap sequence.

In some embodiments, a protein construct is provided that comprises: at least 3 heavy chain CDRs; at least 3 light chain CDRs; a VEGF trap sequence; and a linker sequence, wherein each of the sequences is selected from a sequence within FIGS. 11-18.

In some embodiments, a fusion protein is provided that comprises: an IL-6 VH, an IL-6 VL, an IL-6 Fc, a VEGF Trap, and wherein the fusion protein alters HUVEC proliferation. In some embodiments, each sequence is selected from a sequence within FIGS. 11-18.

In other embodiments, the method of the invention results in a percent reduction in HCP contaminants in the eluate that is at least about a 2-fold reduction, at least about a 3-fold reduction, at least about a 4-fold reduction, at least about a 5-fold reduction, at least about 6-fold reduction, at least about a 7-fold reduction, at least about an 8-fold reduction, at least about a 9-fold reduction, at least about a 10-fold reduction, at least about a 15-fold reduction, or at least about a 20-fold reduction.

Accordingly, in one aspect, the present disclosure provides a method of producing a purified protein (e.g., an antibody, antibody fragment, or protein comprising an Fc region (e.g., an Fc fusion protein)) using an affinity chromatography (AC) matrix to which the protein of interest is bound, the method comprising washing the AC matrix with a wash solution comprising magnesium chloride, or equivalent chaotropic agent. In some embodiments, the wash solution comprising magnesium chloride has a pH around 7.8, has a pH around 6, around 6.1, around 6.2, around 6.3, around 6.4, around 6.5, around 6.6, around 6.7, around 6.8, around 6.9, around 7, around 7.1, around 7.2, around 7.3, around 7.4, around 7.5, around 7.6, around 7.7, around 7.9, around 8, around 8.1, around 8.2, around 8.3, around 8.4, around 8.5, around 8.6, around 8.7, around 8.8, around 8.9, around 9 prior to elution of the protein of interest from the AC matrix. In some embodiments, the wash solution comprises magnesium chloride around 1 M, around 1.1 M, around 1.2 M, around 1.3 M, around 1.4 M, around 1.5 M, around 1.6 M, around 1.7 M, around 1.8 M, around 1.9 M, around 2 M, around 2.1 M, around 2.2 M, around 2.3 M, around 2.4 M, around 2.5 M, around 2.6 M, around 2.7 M, around 2.8 M, around 2.9 M, around 3 M, around 3.1 M, around 3.2 M, around 3.3 M, around 3.4 M, around 3.5 M, around 3.6 M, around 3.7 M, around 3.8 M, around 3.9 M, around 4 M, around 4.1 M, around 4.2 M, or above 4.2 M.

### Antibody Conjugates

Provided herein are anti-VEGF antibodies (including anti-VEGF proteins, e.g., aflibercept) and conjugates thereof. In some embodiments, the antibodies themselves are different from other anti-VEGF agents and provide superior results over other anti-VEGF agents. In some embodiments, the anti-VEGF antibody conjugate displays a surprising superiority over other antibodies and/or the expectation of the activity other antibody conjugates.

In some embodiments, the anti-VEGF antibody conjugate is KSI-301, which is an antibody conjugate comprising:

(1) an anti-VEGF-A antibody; and

(2) a phosphorylcholine containing polymer, wherein the polymer is covalently bonded to the anti-VEGF-A antibody at a cysteine outside a variable region of the anti-VEGF-A antibody, and wherein said cysteine replaces a non-cysteine amino acid that occurs in a same position in sequence, wherein the anti-VEGF-A antibody comprises a light chain and heavy chain, said heavy chain comprising an Fc region, wherein the cysteine is in the Fc region of the heavy chain, wherein the sequence of a heavy chain is at least one of SEQ ID NOs: 270, and wherein the sequence of the light chain comprises at least one of SEQ ID NOs: 275. (or any of the variants thereof in FIG. 8), wherein the antibody conjugate has the structure of Formula (I),

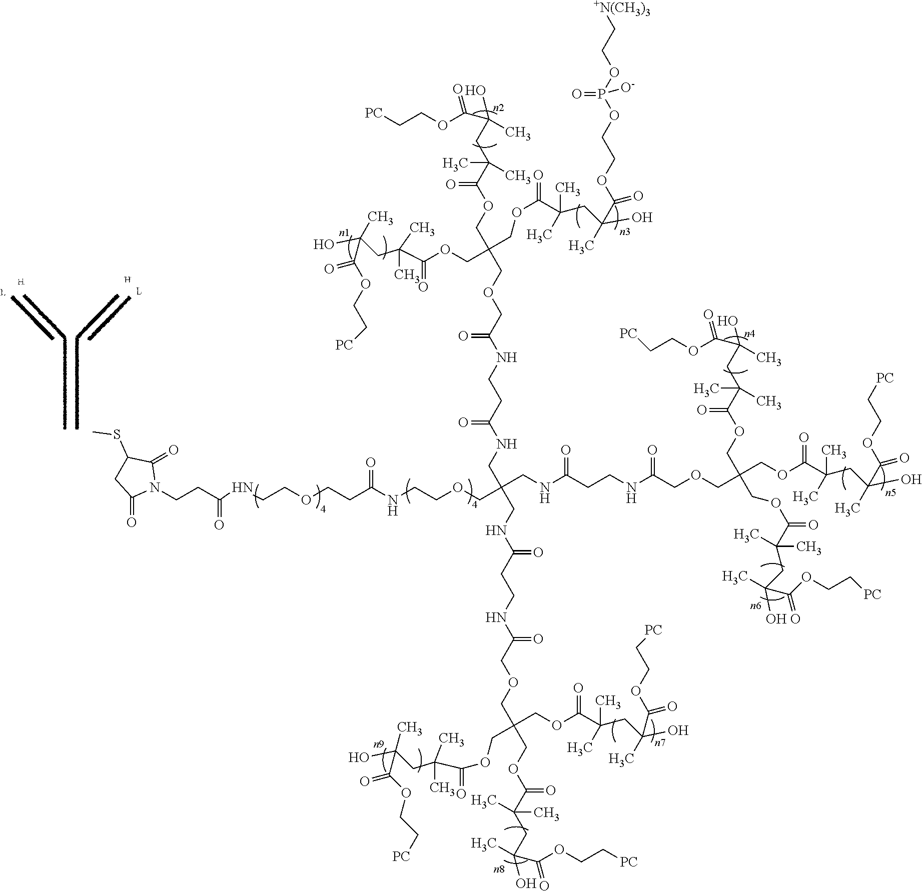

wherein: each heavy chain of the anti-VEGF-A antibody is denoted by the letter H, and each light chain of the anti-VEGF-A antibody is denoted by the letter L; wherein the polymer is bonded to the anti-VEGF-A antibody through a sulfhydryl at C443 according to EU numbering, which bond is depicted on one of the heavy chains above; wherein PC is

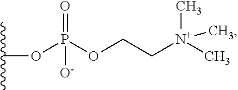 [SMILES: *OP(=O)([O-])OCC[N+](C)(C)C]

where the curvy line indicates the point of attachment to the rest of the polymer; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

Historically, conjugating a molecule to a protein often resulted in a decrease in the protein's binding interaction to its intended target. In some embodiments of the present disclosure, when conjugating to a location that is outside of the active site, the same level of decrease as might have been expected is not necessarily observed. The evidence provided herein shows the opposite effect as to what may have been expected. In some embodiments, and without intending to be limited by theory, the conjugate can be superior to the antibody alone. For example, the interaction of a ligand and its specific receptor is often driven through the stereospecific interaction of the ligand and the receptor, as directed by the interactions of the hydrophilic amino acids on the ligand with the hydrophilic amino acids on the receptor, and water molecules are front and center in those interactions. At the same time, this hydrophilic stereospecificity is further enhanced by de-emphasizing and/or suppressing non-specific hydrophobic interactions that might generally be mediated/created by hydrophobic-to-hydrophobic amino acids.

In some embodiments, an anti-VEGF antibody conjugate is provided that is capable of blocking at least 90% of an interaction between a VEGF ligand (“VEGFL”) and a VEGF-receptor (“VEGFR”). For example, it can block at least 90, 91, 92, 93, 94, 95, 96, 97, 98, 99, or effectively all of the interaction between VEGFR and VEGFL. In some embodiments, the noted blocking occurs at saturating concentrations. In some embodiments, an anti-VEGF antibody conjugate is provided that blocks at least 95% of an interaction between a VEGF ligand and a VEGF-receptor. An example of such superiority of blocking is the ability of the anti-VEGF antibody bioconjugate (an antibody conjugate provided herein, e.g., KSI-301) to block to a higher degree than Lucentis®(ranibizumab) or Avastin®(bevacizumab) or even the antibody OG1950 (unconjugated). Indeed, this result was unexpected in that while the addition of a polymer to an antibody (to form an antibody conjugate), could be expected to have some or no detrimental impact on binding/activity of the antibody, it was unexpected that it would actually improve the blocking ability of the antibody in this manner.

In some embodiments, the antibodies or conjugates thereof inhibit at least 70, 80, 85, 86, 87, 88, 89, 90, 91, 92, 93, 94, 95, 96, 97, 98, 99, or 100% of the activity and/or interaction between VEGFR and VEGFL. In some embodiments, the IC50 value can be 0.1, 1, 2, 3, 4, 5, 6, 7, 8, 9, 10, 20, 30, 40, 50, 100 nM or less than any one or more of the preceding values. In some embodiments, the KD can be 2*10\hat{ }-13, 1*10\hat{ }-13, 1*10\hat{ }-12, 1*10\hat{ }-11, 1*10\hat{ }-10M or less than any one of the preceding values. In some embodiments, the IC50 value can be 1, 5, 10, 20, 30, 40, 50, 60, 70 80, 90, 100, 200, 300, 400, 500, 600, 700, 800, 900, 1,000, 1,100, 1,200, 1,300, or less than any one of the preceding values.

In some embodiments, an anti-VEGF antibody is provided that blocks at least 90% of an interaction between a VEGF ligand and a VEGF-receptor. For example, it can block at least 91, 92, 93, 94, 95, 96, 97, 98, 99, or effectively all of the interaction between VEGFR and VEGFL. As example of such superiority of blocking, is the ability of OG1950 (and antibody provided herein) to block to a higher degree than Lucentis®(ranibizumab) or Avastin®(bevacizumab).

In some embodiments, other antibodies, such as Lucentis®(ranibizumab) or Avastin®(bevacizumab) can be conjugated to one or more of the polymers as described herein, by one or more of the processes described herein. In some embodiments, any antibody, or fragment thereof, can be conjugated to one or more of the polymers as described herein, by one or more of the processes described herein.

In some embodiments the antibody comprises a heavy chain amino acid variable region that comprises at least one of SEQ ID NOs: 7-13, 19-27, 89, 90, 256-262 and a light chain amino acid variable region that comprises at least one of SEQ ID NOs: 91-93, 28-30. In some embodiments, the antibody is conjugated to one or more of the polymers provided herein. In some embodiments, the conjugated antibody is at least 90% identical to at least one of SEQ ID NOs: 7-13, 19-27, 89, 90, 256-262 and/or at least one of SEQ ID NOs: 91-93, 28-30. In some embodiments, the antibody contains the 6 CDRs within at least one of SEQ ID NOs: 7-13, 19-27, 89, 90, 256-262 and at least one of SEQ ID NOs: 91-93, 28-30, as well as a point mutation of L443C (EU numbering, or 449C). In some embodiments, the conjugated antibody is at least 90% identical to at least one of SEQ ID NOs: 7-13, 19-27, 89, 90, 256-262 and/or at least one of SEQ ID NOs: 91-93, 28-30 and includes the following mutations: L234A, L235A, and G237A (EU numbering), and at least one of the following mutations: Q347C (EU numbering) or L443C (EU numbering).

In some embodiments an antibody that binds to VEGF-A is provided. The antibody comprises: a CDRH1 that is a CDRH1 in SEQ ID NO: 172; a CDRH2 that is a CDRH2 in SEQ ID NO: 173; a CDRH3 that is a CDRH3 in SEQ ID NO: 174: a CDRL1 that is a CDRL1 in SEQ ID NO: 199; a CDRL2 that is a CDRL2 in SEQ ID NO: 200; a CDRL3 that is a CDRL3 in SEQ ID NO: 201; at least one of the following mutations (EU numbering): L234A, L235A, and G237A; and at least one of the following mutations (EU numbering): Q347C or L443C.

As will be appreciated by one of skill in the art, in light of the present specification, any of the antibodies provided herein can be conjugated to any of the polymers provided herein and/or any antibody provided herein can have a cysteine added such that it allows for site specific conjugation to a polymer.

“VEGF” or “vascular endothelial growth factor” is a human vascular endothelial growth factor that affects angiogenesis or an angiogenic process. In particular, the term VEGF means any member of the class of growth factors that (i) bind to a VEGF receptor such as VEGFR-1 (Flt-1), VEGFR-2 (KDR/Flk-1), or VEGFR-3 (FLT-4); (ii) activates a tyrosine kinase activity associated with the VEGF receptor; and (iii) thereby affects angiogenesis or an angiogenic process.

The VEGF family of factors is made up of five related glycoproteins: VEGF-A (also known as VPE), -B, -C, -D and PGF (placental growth factor). Of these, VEGF-A is the most well studied and is the target of anti-angiogenic therapy. Ferrara et al, (2003) Nat. Med. 9:669-676. VEGF-A exists as a number of different isotypes which are generated both by alternative splicing and proteolysis: VEGF-A₂₀₆, VEGF-A₁₈₉, VEGF-A₁₆₅, and VEGF-A₁₂₁. The isoforms differ in their ability to bind heparin and non-signaling binding proteins called neuropilins. The isoforms are all biologically active as dimers.

The various effects of VEGF are mediated by the binding of a VEGF, e.g., VEGF-A (P15692), -B (P49766), -C(P49767) and -D (Q43915), to receptor tyrosine kinases (RTKs). The VEGF family receptors belong to class V RTKs and each carry seven Ig-like domains in the extracellular domain (ECD). In humans, VEGF binds to three types of RTKs: VEGFR-1 (Flt-1) (P17948), VEGFR-2 (KDR, Flk-1) (P935968) and VEGFR-3 (Flt-4) (P35916). Unless otherwise apparent from the context reference to a VEGF means any of VEGF-A, -B, -C, -D, and PGF, in any of the natural isoforms or natural variants or induced variants having at least 90, 95, 98 or 99% or 100% sequence identity to a natural form. In some embodiments, such VEGFs are human VEGFs. Likewise reference to a VEGFR means any of VEGR-1, R-2, or R-3, including any natural isoform or natural variant, or an induced variant having at least 90, 95, 98 or 99% or 100% sequence identity to a natural sequence.

VEGF antagonist therapies have been approved for the treatment of certain cancers and wet AMD. Bevacizumab (AVASTIN, Genentech/Roche) is a humanized mouse monoclonal antibody that binds to and neutralizes human VEGF, in particular to all isoforms of VEGF-A and to bioactive proteolytic fragments of VEGF-A. See, e.g., Ferrara N, Hillan K J, Gerber H P, Novotny W. 2004. Discovery and development of bevacizumab, an anti-VEGF antibody for treating cancer. Nat Rev Drug Discov. 3(5):391-400. Bevacizumab has been approved for the treatment of certain cancers.

Bevacizumab variable light chain CDRs are CDR_{L}1: SASQDISNYLN (SEQ ID NO: 749), CDR_{L}2: FTSSLHS (SEQ ID NO: 750), and CDR_{L}3: QQYSTVPWT (SEQ ID NO: 751). Bevacizumab variable heavy chain CDRs are CDR_{H}1: GYTFTNYGMN (SEQ ID NO: 752), CDR_{H}2: WINTYTGEPTYAADFKR (SEQ ID NO: 753), and CDR_{H}3: YPHYYGSSHWYFDV (SEQ ID NO: 755). CDRs are defined by Kabat except CDRH1 uses the composite Kabat/Chothia definition. In some embodiments, a cysteine can be added to the Bevacizumab sequence and the antibody (and/or a variant that includes the 6 CDRs of Bevacizumab) can be conjugated to any one or more of the polymers provided herein. In some embodiments, Bevacizumab or Ranibizumab CDRs may be used in the compositions and methods provided herein.

Another anti-VEGF molecule, derived from the same mouse monoclonal antibody as bevacizumab has been approved as a treatment for wet AMD: ranibizumab (LUCENTIS®(ranibizumab), Genentech/Roche). Ranibizumab is an antibody fragment or Fab. Ranibizumab was produced by affinity maturation of the variable heavy and light chains of bevacizumab. In some embodiments, a cysteine can be added to the ranibizumab sequence and the antibody (and/or a variant that includes the 6 CDRs of ranibizumab) can be conjugated to any one or more of the polymers provided herein.

The Ranibizumab CDRS are the same as Bevacizumab except where an improvement was added after affinity maturation: Ranibizumab variable light chain CDRs are CDRL1: SASQDISNYLN (SEQ ID NO: 749), CDR_{L}2: FTSSLHS (SEQ ID NO: 750), and CDRL3: QQYSTVPWT (SEQ ID NO: 751). Ranibizumab variable heavy chain CDRs are CDR_{H}1: GYDFTHYGMN (SEQ ID NO: 754), CDR_{H}2: WINTYTGEPTYAADFKR (SEQ ID NO: 753), and CDR_{H}3: YPYYYGTSHWYFDV (SEQ ID NO: 756).

In some embodiments, an antibody conjugate is presented having an anti-VEGF-A antibody bonded at a cysteine outside a variable region of the antibody to a phosphorylcholine containing polymer, wherein the cysteine has been added via recombinant DNA technology. In some embodiments, the polymer is bonded to a single cysteine. In some embodiments, “added by recombinant DNA technology” means that the cysteine residue replaces a non-cysteine amino acid that occurs in the same position in a known or existing antibody or in a consensus antibody sequence. Thus, for example where the antibody is an IgG1 and the heavy chain possess a leucine at EU position 443, the leucine is replaced via recombinant DNA technology with a cysteine (L443C, EU numbering, or 449C. Correspondingly, the native IgG1 sequence at EU position 347 is Q (glutamine) and the Q is replaced with cysteine via recombinant DNA technology to yield Q347C.

In some embodiments, the anti-VEGF-A antibody comprises a light chain and a heavy chain where the heavy chain has an Fc region. In some embodiments, the cysteine is in the Fc region and the anti-VEGF-A antibody is an immunoglobulin G (IgG). In some embodiments, the anti-VEGF-A heavy chain has CDRH1: GYDFTHYGMN (SEQ ID NO: 754), CDRH2: WINTYTGEPTYAADFKR (SEQ ID NO: 753), and CDRH3: YPYYYGTSHWYFDV (SEQ ID NO: 756), and position 221 is T, and the anti-VEGF-A light chain has CDR_{L}1: SASQDISNYLN (SEQ ID NO: 749), CDR_{L}2: FTSSLHS (SEQ ID NO: 750), and CDR_{L}3: QQYSTVPWT (SEQ ID NO: 751), and Kabat position 4 is L.

In some embodiments, the anti-VEGF-A heavy chain isotype is IgG1. In some embodiments, the IgG1 constant domain has one or more mutations relative to an IgG1 constant domain to modulate effector function. In some embodiments, the effector function mutations are one or more of the following: (EU numbering) E233X, L234X, L235X, G236X, G237X, A327X, A330X, and P331X wherein X is any natural or unnatural amino acid. In some embodiments, the mutations are selected from the group consisting of (EU numbering): E233P, L234V, L234A, L235A, G237A, A327G, A330S, and P331S. In some embodiments, the antibody conjugate has the following mutations (EU numbering): L234A, L235A, and G237A.

In some embodiments, the cysteine residue is in the anti-VEGF-A heavy chain and is Q347C (EU numbering) or L443C (EU numbering). In some embodiments, the cysteine residue is L443C (EU numbering, or 449C). In some embodiments, the sequence of the anti-VEGF-A heavy chain is SEQ ID NOs: 7-13, 19-27, 89, 90, 256-262 and the light chain sequence comprises at least one of SEQ ID NOs: 91-93, 28-30.

In some embodiments, the phosphorylcholine containing polymer comprises 2-(methacryloyloxyethyl)-2′-(trimethylammonium)ethyl phosphate (MPC) monomers as set forth below:

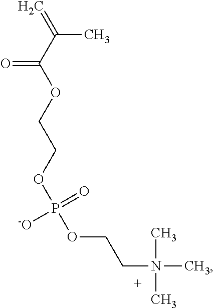

such that the polymer comprises the following repeating units:

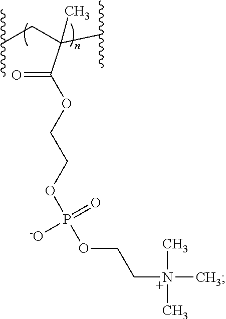

where n is an integer from 1 to 3000 and the wavy lines indicate the points of attachment between monomer units in the polymer.

In some embodiments, the polymer has three or more arms, or is synthesized with an initiator comprising 3 or more polymer initiation sites. In some embodiments, the polymer has 2, 3, 4, 5, 6, 7, 8, 9, 10, 11 or 12 arms, or is synthesized with an initiator comprising 2, 3, 4, 5, 6, 7, 8, 9, 10, 11, or 12 polymer initiation sites. More preferably, the polymer has 3, 6, or 9 arms, or is synthesized with an initiator comprising 3, 6, or 9 polymer initiation sites. In some embodiments, the polymer has 9 arms, or is synthesized with an initiator comprising 9 polymer initiation sites.

In some embodiments, the polymer that is added has a molecular weight between about 300,000 and about 1,750,000 Da (SEC-MALs). In some embodiments, the polymer has a molecular weight between about 500,000 and about 1,000,000 Da. In some embodiments, the polymer has a molecular weight of between about 600,000 to about 900,000 Da. In some embodiments, the polymer has a molecular weight of between about 750,000 to about 850,000 Da. In some embodiments, the polymer has a molecular weight of between about 800,000 to about 850,000 Da. In some embodiments, the polymer has a molecular weight of between about 750,000 to about 800,000 Da.

In some embodiments, any of the antibodies described herein can be further conjugated to a polymer to form a bioconjugate. The molecular weight of the bioconjugate (in total, SEC-MALs) can be between about 350,000 and 2,000,000 Daltons, for example, between about 450,000 and 1,900,000 Daltons, between about 550,000 and 1,800,000 Daltons, between about 650,000 and 1,700,000 Daltons, between about 750,000 and 1,600,000 Daltons, between about 850,000 and 1,500,000 Daltons, between about 900,000 and 1,400,000 Daltons, between about 950,000 and 1,300,000 Daltons, between about 900,000 and 1,000,000 Daltons, between about 1,000,000 and 1,300,000 Daltons, between about 850,000 and 1,300,000 Daltons, between about 850,000 and 1,000,000 Daltons, and between about 1,000,000 and 1,200,000 Daltons.

In some embodiments, the antibody conjugate is purified. In some embodiments, the polymer is aspect of the antibody conjugate is polydisperse, i.e. the polymer PDI is not 1.0. In some embodiments, the PDI is less than 1.5. In some embodiments, the PDI is less than 1.4. In some embodiments, the PDI is less than 1.3. In some embodiments the PDI is less than 1.2. In some embodiments the PDI is less than 1.1.

In some embodiments, the antibody conjugate has an anti-VEGF-A immunoglobulin G (IgG) bonded to a polymer, which polymer comprises MPC monomers, wherein the sequence of the anti-VEGF-A heavy chain is SEQ ID NOs: 7-13, 19-27, 89, 90, 256-262 and the light chain sequence comprises at least one of SEQ ID NOs: 91-93, 28-30, and wherein the antibody is bonded only at C449 to the polymer. In some embodiments, the polymer has 9 arms and has a molecular weight of between about 600,000 to about 1,000,000 Da.

In some embodiments, the antibody conjugate has an anti-VEGF-A immunoglobulin G (IgG) bonded to a polymer, which polymer comprises MPC monomers, wherein the sequence of the anti-VEGF-A heavy chain is SEQ ID NO. 7-13, 19-27, 89, 90, 256-262, and the sequence of the anti-VEGF-A light chain is SEQ ID NO. 91-93, 28-30, and wherein the antibody is bonded only at C443 (EU numbering, or 449C) to the polymer. In some embodiments, the polymer has 9 arms and has a molecular weight of between about 600,000 to about 1,000,000 Da.

In some embodiments, the antibody conjugate has the structure of Formula (I),

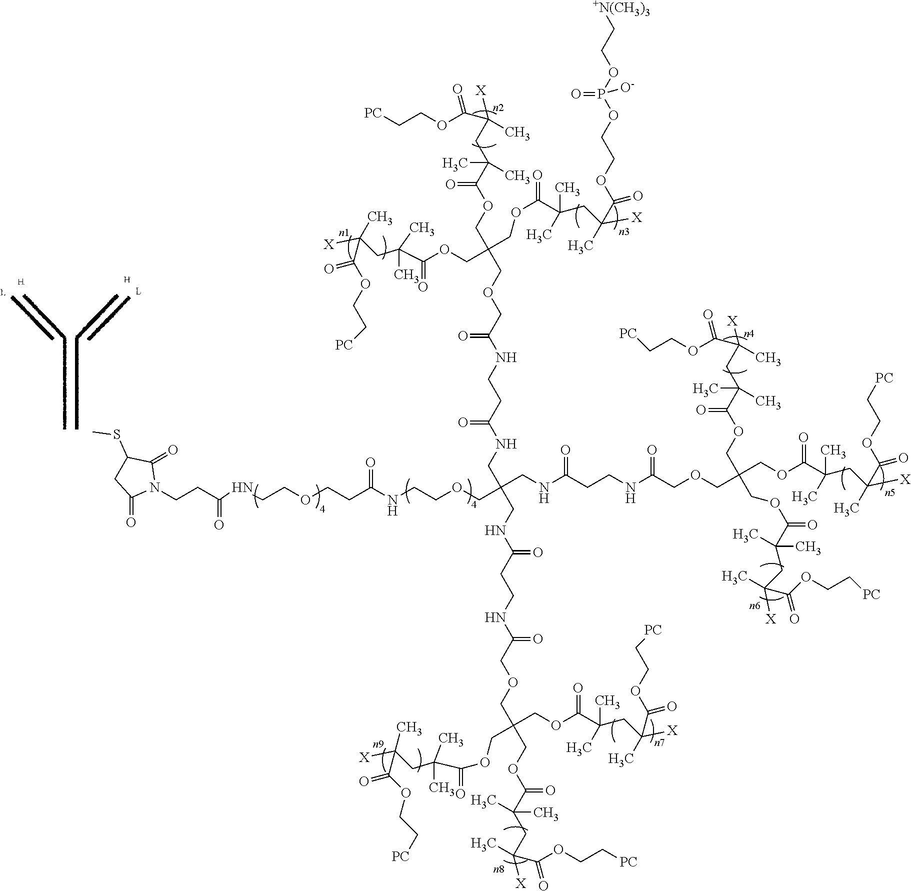

wherein: each heavy chain of the anti-VEGF-A antibody is denoted by the letter H, and each light chain of the anti-VEGF-A antibody is denoted by the letter L; wherein the polymer is bonded to the anti-VEGF-A antibody through the sulfhydryl of C449, which bond is depicted on one of the heavy chains; wherein PC is,

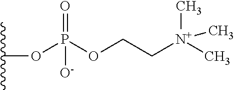 [SMILES: *OP(=O)([O-])OCC[N+](C)(C)C]

wherein the curvy line indicates the point of attachment to the rest of the polymer; wherein X is a) —OR where R is H, methyl, ethyl, propyl, or isopropyl, b) —H, c) any halogen, including —Br, —Cl, or —I, d) —SCN, or e) —NCS; and wherein n1, n2, n3, n4, n5, n6, n7, n8 and n9 are the same or different such that the sum of n1, n2, n3, n4, n5, n6, n7, n8 and n9 is 2500 plus or minus 15%. In some embodiments, n1, n2, n3, n4, n5, n6, n7, n8 and n9 are the same or different and are integers from 0 to 3000. In some embodiments, n1, n2, n3, n4, n5, n6, n7, n8 and n9 are the same or different and are integers from 0 to 500. In some embodiments, X is —OR, where R is a sugar, an aminoalkyl, mono-substituted, poly-substituted or unsubstituted variants of the following residues: saturated C₁-C₂₄ alkyl, unsaturated C₂-C₂₄ alkenyl or C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, amino, aminocarbonyl, aminocarboyloxy, nitro, azido, phenyl, hydroxy, alkylthio, arylthio, oxysulfonyl, carboxy, cyano, and halogenated alkyl including polyhalogenated alkyl, —CO—O—R₇, carbonyl —CCO—R₇, —CO—NR₈R₉, —(CH₂)ₙ—COOR₇, —CO—(CH)ₙ—COOR₇, —(CH₂)ₙ—NR₈R₉, ester, alkoxycarbonyl, aryloxycarbonyl, wherein n is an integer from 1 to 6, wherein each R₇, R8 and R₉ is separately selected from the group consisting of a hydrogen atom, halogen atom, mono-substituted, poly-substituted or unsubstituted variants of the following residues: saturated C₁-C₂₄ alkyl, unsaturated C₂-C₂₄ alkenyl or C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, amino, aminocarbonyl, aminocarboyloxy, nitro, azido, phenyl, hydroxy, alkylthio, arylthio, oxysulfonyl, carboxy, cyano, and halogenated alkyl including polyhalogenated alkyl, a 5-membered ring, and a 6-membered ring.

In some embodiments, the antibody conjugate has the structure of Formula (I),

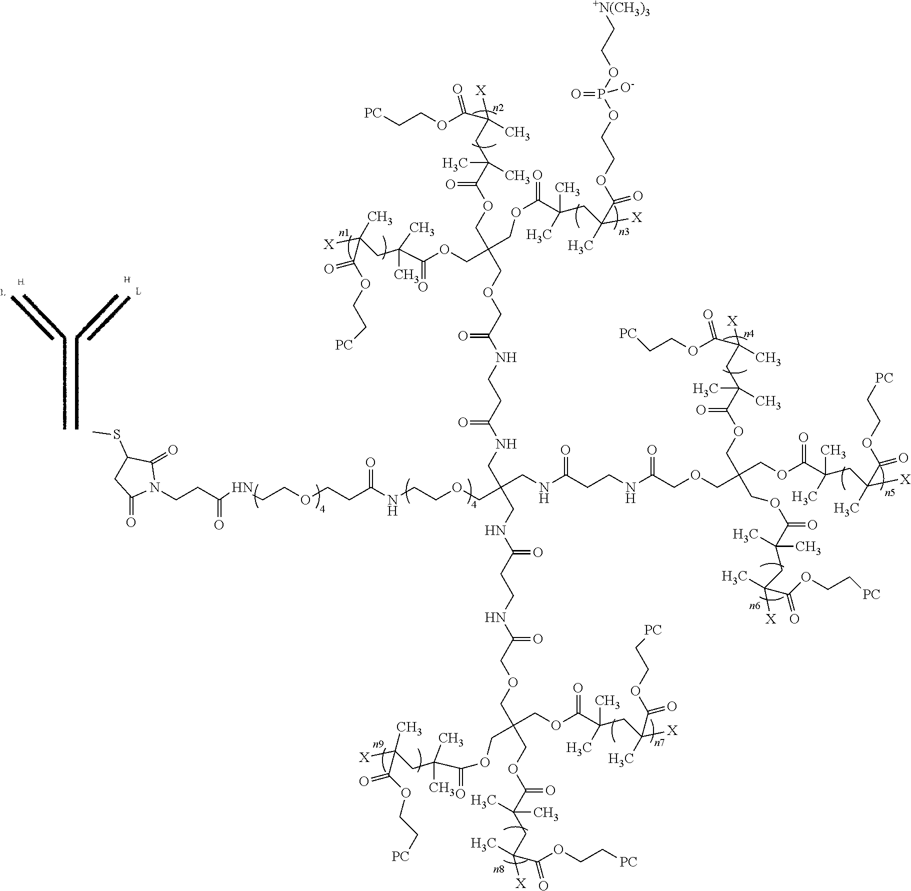

wherein: each heavy chain of the anti-VEGF-A antibody is denoted by the letter H, and each light chain of the anti-VEGF-A antibody is denoted by the letter L; wherein the polymer is bonded to the anti-VEGF-A antibody through the sulfhydryl of C443 (EU numbering, or 449C), which bond is depicted on one of the heavy chains; wherein PC is,

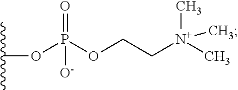 [SMILES: *OP(=O)([O-])OCC[N+](C)(C)C]

wherein the curvy line indicates the point of attachment to the rest of the polymer; wherein X is a) —OR where R is H, methyl, ethyl, propyl, or isopropyl, b) —H, c) any halogen, including Br, —Cl, or —I, d) —SCN, or e) —NCS; and wherein n1, n2, n3, n4, n5, n6, n7, n8 and n9 are the same or different such that the sum of n1, n2, n3, n4, n5, n6, n7, n8 and n9 is 2500 plus or minus 15%. In some embodiments, n1, n2, n3, n4, n5, n6, n7, n8 and n9 are the same or different and are integers from 0 to 3000. In some embodiments, n1, n2, n3, n4, n5, n6, n7, n8 and n9 are the same or different and are integers from 0 to 500. In some embodiments, X is —OR, where R is a sugar, an aminoalkyl, mono-substituted, poly-substituted or unsubstituted variants of the following residues: saturated C₁-C₂₄ alkyl, unsaturated C₂-C₂₄ alkenyl or C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, amino, aminocarbonyl, aminocarboyloxy, nitro, azido, phenyl, hydroxy, alkylthio, arylthio, oxysulfonyl, carboxy, cyano, and halogenated alkyl including polyhalogenated alkyl, —CO—O—R₇, carbonyl —CCO—R₇, —CO—NR₈R₉, —(CH₂)ₙ—COOR₇, —CO—(CH)ₙ—COOR₇, —(CH₂)ₙ—NR₈R₉, ester, alkoxycarbonyl, aryloxycarbonyl, wherein n is an integer from 1 to 6, wherein each R₇, R₈ and R₉ is separately selected from the group consisting of a hydrogen atom, halogen atom, mono-substituted, poly-substituted or unsubstituted variants of the following residues: saturated C₁-C₂₄ alkyl, unsaturated C₂-C₂₄ alkenyl or C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, amino, aminocarbonyl, aminocarboyloxy, nitro, azido, phenyl, hydroxy, alkylthio, arylthio, oxysulfonyl, carboxy, cyano, and halogenated alkyl including polyhalogenated alkyl, a 5-membered ring, and a 6-membered ring. In some embodiments, this construct is designated as KSI-301.

In some embodiments, the antibody conjugate is present in a liquid formulation. In some embodiments, the antibody conjugate is combined with a pharmaceutically acceptable carrier. In some embodiments, any of the methods provided herein can use the following drug formulations: a) 12.5 mM sodium phosphate buffer with 0.025% (w/w) polysorbate 20, pH 6.5 at a concentration of 50 mg/mL (based on antibody mass), equivalent to 324 mg/mL of total mass of the antibody-biopolymer conjugate, including 50 mg/mL OG1950 Antibody Intermediate and 274 mg/mL OG1802 Biopolymer Intermediate, b) 10-15 mM sodium phosphate buffer with 0.01-0.5% (w/w) polysorbate 20, pH 6.5 at a concentration of about 50 mg/mL (based on antibody mass), equivalent to about 324 mg/mL of total mass of the antibody-biopolymer conjugate, c) 12.5 mM sodium phosphate buffer with optional 0.025% (w/w) polysorbate 20, pH 6.5 at a concentration of 50 mg/mL (based on antibody mass), equivalent to 324 mg/mL of total mass of the antibody-biopolymer conjugate, including 50 mg/mL OG1950 Antibody Intermediate and 274 mg/mL OG1802 Biopolymer Intermediate, d) sodium phosphate buffer with polysorbate 20, pH 6.5 at a concentration of 50 mg/mL (based on antibody mass), equivalent to about 324 mg/mL of total mass of the antibody-biopolymer conjugate, including about 50 mg/mL OG1950 Antibody Intermediate and about 274 mg/mL OG1802 Biopolymer Intermediate, e) 10-15 mM sodium phosphate buffer with 0.01-0.5% (w/w) polysorbate 20, pH 6.5 at a concentration of about 40-55 mg/mL (based on antibody mass), equivalent to about 259-356 mg/mL of total mass of the antibody-biopolymer conjugate f) 10-15 mM sodium phosphate buffer with 0.01-0.5% (w/w) polysorbate 20, pH 6.5 at a concentration of about 45-52.5 mg/mL (based on antibody mass), equivalent to about 292-340 mg/mL of total mass of the antibody-biopolymer conjugate, or g) 10-15 mM sodium phosphate buffer with 0.01-0.5% (w/w) polysorbate 20, pH 6.5 at a concentration of about 40-55 mg/mL (based on antibody mass), equivalent to about 259-356 mg/mL of total mass of the antibody-biopolymer conjugate. In any of these formulations, the antibody can employ the VH and VL (or the 1, 2, 3, 4, 5, or all 6 CDRs within these VH and/or VL sequences) in FIG. 8, e.g., OG1950 (e.g., SEQ ID Nos: 1, 15-18 and 2.)

In some embodiments, an anti-VEGF-A antibody is presented. The anti-VEGF-A antibody heavy chain has at least the following CDR sequences: CDR_{H}1: that is a CDRH1 in SEQ ID NO: 172, CDR_{H}2: that is a CDRH2 in SEQ ID NO: 173, and CDR_{H}3: that is a CDRH3 in SEQ ID NO: 174. In some embodiments, the anti-VEGF-A heavy chain has those CDRs and in addition has threonine (T) at position 221. In some embodiments, the anti-VEGF-A light chain has at least the following CDRs: CDR_{L}1: that is a CDRL1 in SEQ ID NO: 199, CDR_{L}2: that is a CDRL2 in SEQ ID NO: 200 and CDR_{L}3: that is a CDRL3 in SEQ ID NO: 201. In some embodiments, the anti-VEGF-A antibody has those CDRs and in addition has leucine (L) at Kabat position 4. In some embodiments, the isotype of the anti-VEGF-A antibody heavy chain, is IgG1 and has a CH₁, hinge, CH₂ and CH₃ domains. In some embodiments the light chain isotype is kappa. In some embodiments, the anti-VEGF antibody conjugate (e.g., KSI-301) construct will have one or more of these CDRs.

In some embodiments, the IgG1 domain of the anti-VEGF-A antibody has one or more mutations to modulate effector function, such as ADCC, ADCP, and CDC. In some embodiments, the IgG1 mutations reduce effector function. In some embodiments the amino acids to use for effector function mutations include (EU numbering) E233X, L234X, L235X, G236X, G237X, G236X, D270X, K322X, A327X, P329X, A330X, A330X, P331X, and P331X, in which X is any natural or non-natural amino acid. In some embodiments, the mutations include one or more of the following: E233P, L234V, L234A, L235A, G237A, A327G, A330S and P331S (EU numbering). In some embodiments, the anti-VEGF-A heavy chain has the following mutations (EU numbering): L234A, L235A and G237A. In some embodiments, the number of effector function mutations relative to a natural human IgG1 sequence is no more than 10. In some embodiments the number of effector function mutations relative to a natural human IgG1 sequence is no more than 5, 4, 3, 2 or 1. In some embodiments, the antibody has decreased Fc gamma binding and/or complement C1q binding, such that the antibody's ability to result in an effector function is decreased. This can be especially advantageous for ophthalmic indications/disorders.

In some embodiments, the anti-VEGF-A antibody comprises one or more of the following amino acid mutations: L234A, L235A, G237A (EU numbering), and L443C (EU numbering, or 449C).

In some embodiments, the anti-VEGF-A antibody is or is part of a human immunoglobulin G (IgG1).

In some embodiments, the VEGF-A antibody comprises a heavy chain constant domain that comprises one or more mutations that reduce an immune-mediated effector function.

In some embodiments an anti-VEGF-A antibody is provided. The anti-VEGF-antibody comprises a heavy chain that comprises a CDRH1 Comprising the sequence that is a CDRH1 in SEQ ID NO: 172, a CDRH2 comprising the sequence that is a CDRH2 in SEQ ID NO: 173, a CDRH3 comprising the sequence that is a CDRH3 in SEQ ID NO: 174, a CDR_{L}1 comprising the sequence that is a CDRL1 in SEQ ID NO: 199, a CDR_{L}2 comprising the sequence that is a CDRL2 in SEQ ID NO: 200, and a CDR_{L}3 comprising the sequence that is a CDRL3 in SEQ ID NO: 201.

Alternatively, the IgG domain can be IgG2, IgG3 or IgG4 or a composite in which a constant region is formed from more than one of these isotypes (e.g., CH1 region from IgG2 or IgG4, hinge, CH2 and CH3 regions from IgG1). Such domains can contain mutations to reduce and/or modulate effector function at one or more of the EU positions mentioned for IgG1. Human IgG2 and IgG4 have reduced effector functions relative to human IgG1 and IgG3.

The anti-VEGF-A heavy chain has a cysteine residue added as a mutation by recombinant DNA technology which can be used to conjugate a half-life extending moiety. In some embodiments, the mutation is (EU numbering) Q347C (EU numbering) and/or L443C (EU numbering, or 449C). In some embodiments, the mutation is L443C (EU numbering, or 449C). In some embodiments, the stoichiometry of antibody to polymer is 1:1; in other words, a conjugate has one molecule of antibody conjugated to one molecule of polymer.

The half-life of the anti-VEGF-A antibodies can be extended by attachment of a “half-life (“half life”) extending moieties” or “half-life (“half life”) extending groups”. Half-life extending moieties include peptides and proteins which can be expressed in frame with the biological drug of issue (or conjugated chemically depending on the situation) and various polymers which can be attached or conjugated to one or more amino acid side chain or end functionalities such as —SH, —OH, —COOH, —CONH2, —NH2, or one or more N- and/or O-glycan structures. Half-life extending moieties generally act to increase the in vivo circulatory half-life of biologic drugs.

Examples of peptide/protein half-life extending moieties include Fc fusion (Capon D J, Chamow S M, Mordenti J, et al. Designing CD4 immunoadhesions for AIDS therapy. Nature. 1989. 337:525-31), human serum albumin (HAS) fusion (Yeh P, Landais D, Lemaitre M, et al. Design of yeast-secreted albumin derivatives for human therapy: biological and antiviral properties of a serum albumin-CD4 genetic conjugate. Proc Natl Acad Sci USA. 1992. 89:1904-08), carboxy terminal peptide (CTP) fusion (Fares F A, Suganuma N. Nishimori K, et al. Design of a long-acting follitropin agonist by fusing the C-terminal sequence of the chorionic gonadotropin beta subunit to the follitropin beta subunit. Proc Natl Acad Sci USA. 1992. 89:4304-08), genetic fusion of non-exact repeat peptide sequence (XTEN) fusion (Schellenberger V, Wang C W, Geething N C, et al. A recombinant polypeptide extends the in vivo half-life of peptides and proteins in a tunable manner. Nat Biotechnol. 2009. 27:1186-90), elastin like peptide (ELPylation) (MCpherson D T, Morrow C, Minehan D S, et al. Production and purification of a recombinant elastomeric polypeptide, G(VPGVG19-VPGV, from Escherichia coli. Biotechnol Prog. 1992. 8:347-52), human transferrin fusion (Prior C P, Lai C-H, Sadehghi H et al. Modified transferrin fusion proteins. Patent WO2004/020405. 2004), proline-alanine-serine (PASylation) (Skerra A, Theobald I, Schlapsky M. Biological active proteins having increased in vivo and/or vitro stability. Patent WO2008/155134 A1. 2008), homo-amino acid polymer (HAPylation) (Schlapschy M, Theobald I, Mack H, et al. Fusion of a recombinant antibody fragment with a homo-amino acid polymer: effects on biophysical properties and prolonged plasma half-life. Protein Eng Des Sel. 2007. 20:273-84) and gelatin like protein (GLK) fusion (Huang Y-S, Wen X-F, Zaro J L, et al. Engineering a pharmacologically superior form of granulocyte-colony-stimulating-factor by fusion with gelatin-like protein polymer. Eur J. Pharm Biopharm. 2010. 72:435-41).

Examples of polymer half-life extending moieties include polyethylene glycol (PEG), branched PEG, PolyPEG® (Warwick Effect Polymers; Coventry, UK), polysialic acid (PSA), starch, hydroxylethyl starch (HES), hydroxyalkyl starch (HAS), carbohydrate, polysaccharides, pullulane, chitosan, hyaluronic acid, chondroitin sulfate, dermatan sulfate, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polycarboxylate, polyvinylpyrrolidone, polyphosphazene, polyoxazoline, polyethylene-co-maleic acid anyhydride, polystyrene-co-maleic acid anhydride, poly(1-hydroxymethyethylene hydroxymethylformal) (PHF), a zwitterionic polymer, a phosphorylcholine containing polymer and a polymer comprising MPC, Poly (Glyₓ-Ser_{y}), Hyaluronic acid (HA), Heparosan polymers (HEP), Fleximers, Dextran, and Poly-sialic acids (PSA).

In one embodiment a half-life extending moiety can be conjugated to an antibody via free amino groups of the protein using N-hydroxysuccinimide (NHS) esters. Reagents targeting conjugation to amine groups can randomly react to ϵ-amine group of lysines, α-amine group of N-terminal amino acids, and δ-amine group of histidines.

However, the anti-VEGF-A antibodies disclosed herein have many amine groups available for polymer conjugation. Conjugation of polymers to free amino groups, thus, might negatively impact the ability of the antibody proteins to bind to VEGF.

In some embodiments, a half-life extending moiety is coupled to one or more free SH groups using any appropriate thiol-reactive chemistry including, without limitation, maleimide chemistry, or the coupling of polymer hydrazides or polymer amines to carbohydrate moieties of the antibody after prior oxidation. In some embodiments maleimide coupling is used. In some embodiments, coupling occurs at cysteines naturally present or introduced via genetic engineering.

In some embodiments, polymers are covalently attached to cysteine residues introduced into anti-VEGF-A antibodies by site directed mutagenesis. In some embodiments, the cysteine residues are employed in the Fc portion of the antibody. In some embodiments, the sites to introduce cysteine residues into an Fc region are provided in WO 2013/093809, U.S. Pat. No. 7,521,541, WO 2008/020827, U.S. Pat. Nos. 8,008,453, 8,455,622 and US2012/0213705, incorporated herein by reference for all purposes. In some embodiments, the cysteine mutations are Q347C (EU numbering) and L443C referring to the human IgG heavy chain by EU numbering.

In some embodiments, conjugates of antibody and high MW polymers serving as half-life extenders are provided. In some embodiments, a conjugate comprises an antibody that is coupled to a zwitterionic polymer wherein the polymer is formed from one or more monomer units and wherein at least one monomer unit has a zwitterionic group is provided. In some embodiments, the zwitterionic group is phosphorylcholine.

In some embodiments, one of the monomer units is HEMA-PC. In some embodiments, a polymer is synthesized from a single monomer which is HEMA-PC.

In some embodiments, some antibody conjugates have 2, 3, or more polymer arms wherein the monomer is HEMA-PC. In some embodiments, the conjugates have 2, 3, 4, 5, 6, 7, 8, 9, 10, 11, or 12 polymer arms wherein the monomer is HEMA-PC. In some embodiments, the conjugates have 3, 6 or 9 arms. In some embodiments, the conjugate has 9 arms.

In some embodiments, polymer-antibody conjugates have a polymer portion with a molecular weight of between 100,000 and 1,500,000 Da. In some embodiments, the conjugate has a polymer portion with a molecular weight between 500,000 and 1,000,000 Da. In some embodiments, the conjugate has a polymer portion with a molecular weight between 600,000 to 800,000 Da. In some embodiments, the conjugate has a polymer portion with a molecular weight between 600,000 and 850,000 Da and has 9 arms. When a molecular weight is given for an antibody conjugated to a polymer, the molecular weight will be the addition of the molecular weight of the protein, including any carbohydrate moieties associated therewith, and the molecular weight of the polymer.

In some embodiments, an anti-VEGF-A antibody has a HEMA-PC polymer which has a molecular weight measured by Mw of between about 100 kDa and 1650 kDa is provided. In some embodiments, the molecular weight of the polymer as measured by Mw is between about 500 kDa and 1000 kDa. In some embodiments, the molecular weight of the polymer as measured by Mw is between about 600 kDa to about 900 kDa. In some embodiments, the polymer molecular weight as measured by Mw is 750 kDa plus or minus 15%.

In some embodiments, the polymer is made from an initiator suitable for ATRP having one or more polymer initiation sites. In some embodiments, the polymer initiation site has a 2-bromoisobutyrate site. In some embodiments, the initiator has 3 or more polymer initiation sites. In some embodiments, the initiator has 3, 4, 5, 6, 7, 8, 9, 10, 11 or 12 polymer initiation sites. In some embodiments, the initiator has 3, 6 or 9 polymer initiation sites. In some embodiments, the initiator has 9 polymer initiation sites. In some embodiments, the initiator is OG1786.

The anti-VEGF-A antibodies can be produced by recombinant expression including (i) the production of recombinant DNA by genetic engineering, (ii) introducing recombinant DNA into prokaryotic or eukaryotic cells by, for example and without limitation, transfection, electroporation or microinjection, (iii) cultivating the transformed cells, (iv) expressing antibody, e.g. constitutively or on induction, and (v) isolating the antibody, e.g. from the culture medium or by harvesting the transformed cells, in order to (vi) obtain purified antibody.

The anti-VEGF-A antibodies can be produced by expression in a suitable prokaryotic or eukaryotic host system characterized by producing a pharmacologically acceptable antibody molecule. Examples of eukaryotic cells are mammalian cells, such as CHO, COS, HEK 293, BHK, SK-Hip, and HepG2. Other suitable expression systems are prokaryotic (e.g., E. coli with pET/BL21 expression system), yeast (Saccharomyces cerevisiae and/or Pichia pastoris systems), and insect cells.

A wide variety of vectors can be used for the preparation of the antibodies disclosed herein and are selected from eukaryotic and prokaryotic expression vectors. Examples of vectors for prokaryotic expression include plasmids such as, and without limitation, preset, pet, and pad, wherein the promoters used in prokaryotic expression vectors include one or more of, and without limitation, lac, trc, trp, recA, or araBAD. Examples of vectors for eukaryotic expression include: (i) for expression in yeast, vectors such as, and without limitation, pAO, pPIC, pYES, or pMET, using promoters such as, and without limitation, AOX1, GAP, GAL1, or AUG1; (ii) for expression in insect cells, vectors such as and without limitation, pMT, pAc5, pIB, pMIB, or pBAC, using promoters such as and without limitation PH, p10, MT, Ac5, OpIE2, gp64, or polh, and (iii) for expression in mammalian cells, vectors such as, and without limitation, pSVL, pCMV, pRc/RSV, pcDNA3, or pBPV, and vectors derived from, in one aspect, viral systems such as and without limitation vaccinia virus, adeno-associated viruses, herpes viruses, or retroviruses, using promoters such as and without limitation CMV, SV40, EF-1, UbC, RSV, ADV, BPV, and beta-actin.

### Method of Conjugating Proteins to Polymers

In some embodiments, a method is presented of preparing a therapeutic protein-half life extending moiety conjugate having the step of conjugating a therapeutic protein which has a cysteine residue added via recombinant DNA technology to a half-life extending moiety having a sulfhydryl specific reacting group selected from the group consisting of maleimide, vinylsulfones, orthopyridyl-disulfides, and iodoacetamides to provide the therapeutic protein-half life extending moiety conjugate.

In some embodiments a method of preparing the anti-VEGF antibody conjugate, e.g., KSI-301, from OG1950 is provided. The method comprises reducing the OG1950 protein with a 30× molar excess of the TCEP reducing agent. After reduction, the antibody is oxidized to produce a decapped OG1950 antibody where the inter- and intra-light and heavy chain disulfide bonds naturally occurring in the antibody are formed. The OG1950 is then conjugated by adding an excipient and adding 3-10× molar excess of a maleimide biopolymer. The biopolymer links to the OG1950 antibody through a covalent thiolether linkage. After conjugation, the anti-VEGF antibody conjugate, e.g., KSI-301, is purified with both unconjugated antibody and polymer removed.

The protein and process described above can be varied as well. Thus, in some embodiments, a process for preparing a conjugated protein (which need not be an antibody or an anti-VEGF antibody) is provided. The process includes reducing one or more cysteines in a protein to form a decapped protein in a solution. After reducing the one or more cysteines the decapped protein is reoxidized to restore at least one disulfide linkage in the reduced protein while ensuring that an engineered cysteine residue in the protein remains in a free thiol form to form a reoxidized decapped protein in the solution. At least one excipient is then added to the solution. The excipient reduces a polymer induced protein precipitation. After the excipient is added, a polymer is added to the solution, which is conjugated to the reoxidized decapped protein at the engineered cysteine residue to form a conjugated protein.

In some embodiments, the molar excess of the reducing agent can be altered to any amount that functions. In some embodiments 10, 20, 30, 40, 50, 60, 70, 80, 90× molar excess of the reducing agent (which need not be TCEP in all embodiments) can be employed. In some embodiments, any antibody (therapeutic or otherwise) can be employed. In some embodiments, 1, 2, 3, 4, 5, 6, 7, 8, 9, 10, 11, 12, 13, 14, 15× molar excess of a maleimide biopolymer can be employed. In some embodiments, there is an excess of decapped protein to polymer. In some embodiments, the amount of the reoxidized decapped, or decapped protein is less than the amount of the polymer. In some embodiments, the amount of the reoxidized decapped, or decapped protein is 90%, 80%, 70%, 60%, 50%, 40%, 30%, 20%, 10%, 5%, 4%, 3%, 2%, 1% of the amount of the polymer. In some embodiments, 2.5-4.5 times as much polymer is used as protein, as measured by molar excess. In some embodiments, 10-20 times as much polymer is used as protein, as measured by mass. In some embodiments the amount of the reduced antibody is greater than the amount of the polymer. In some embodiments the amount of the polymer is greater than the amount of the reduced antibody.

In some embodiments, the purification step is optional.

In some embodiments, the method of making an antibody conjugate comprises conjugating an anti-VEGF-A antibody to a phosphorylcholine containing polymer. In some embodiments the method comprises the steps of conjugating an anti-VEGF-A antibody to a phosphorylcholine containing polymer. The anti-VEGF-A antibody comprises an amino acid residue added via recombinant DNA technology. In some embodiments, the added amino acid residue is a cysteine residue. In some embodiments, the cysteine residue is added outside a variable region of the antibody. The cysteine residue can be added to either the heavy chain or light chain of the antibody.

In some embodiments, the polymer comprises or consists of a phosphorylcholine containing polymer. In some embodiments, the phosphorylcholine containing polymer comprises a sulfhydryl specific reacting group selected from the group consisting of a maleimide, a vinylsulfone, an orthopyridyl-disulfide, and an iodoacetamide. In some embodiments, the sulfhydryl specific reacting group on the phosphorylcholine containing polymer reacts with the cysteine residue on the anti-VEGF-A antibody to make the antibody conjugate.

In some embodiments, the protein to be conjugated can be an antibody, an antibody protein fusion, or a binding fragment thereof. In some embodiments, the protein is not an antibody but is an enzyme, a ligand, a receptor, or other protein or mutants or variants thereof. In some embodiments, the native protein contains at least one disulfide bond and at least one non-native cysteine.

In some embodiments, the excipient can be an acid or a base. In some embodiments, the excipient is a detergent, a sugar, or a charged amino acid. In some embodiments, the excipient assists in keeping the protein in solution during the conjugation to the polymer. In some embodiments, the excipient is added to the solution containing the protein, prior to the addition of the polymer to the solution that contains the protein.

In some embodiments, the reaction occurs under aqueous conditions between about pH 5 to about pH 9. In some embodiments, the reaction occurs between pH 6.0 and pH 8.5, between pH 6.5 and pH 8.0 or between pH 7.0 and pH 7.5. In some embodiments, the reaction occurs between pH 8 and pH 9.

In some embodiments, the polymer is conjugated to the protein at 2-37 degrees Celsius. In some embodiments, the conjugation occurs at 0-40 degrees Celsius, 5-35 degrees Celsius, 10-30 degrees Celsius, and 15-25 degrees Celsius. In some embodiments, the polymer is conjugated at 5-10 degrees Celsius.

In some embodiments, the conjugated proteins described herein can be contacted to an ion exchange medium or hydrophobic interaction chromatography or affinity chromatography medium for purification (to remove the conjugated from the unconjugated). In some embodiments, the ion exchange medium, hydrophobic interaction chromatography, and/or affinity chromatography medium separates the conjugated protein from the free polymer and from the reoxidized decapped protein.

In some embodiments, the polymers disclosed herein can comprise one or more of the following: a zwitterion, a phosphorylcholine, or a PEG linker bridging a center of a polymer branching point to the maleimide functional group. In some embodiments, any of the polymers provided herein can be added to a protein via the methods provided herein.

In some embodiments, any of the proteins provided herein can be conjugated to any of the polymers provided herein via one or more of the methods provided herein.

In some embodiments, the process(es) provided herein allow(s) for larger scale processing to make and purify protein and/or antibody conjugates. In some embodiments, the volume employed is at least 1 liter, for example 1, 10, 100, 1,000, 5,000, 10,000, liters or more. In some embodiments, the amount of the antibody conjugate produced and/or purified can be 0.1, 1, 10, 100, 1000, 2000, 2500, 3000, or more grams.

In some embodiments, the therapeutic protein may be any of the anti-VEGF-A antibodies described herein having a cysteine residue added via recombinant DNA technology. In some embodiments, the anti-VEGF antibody heavy chain has the following CDRs: CDR_{H}1: that is a CDRH1 in SEQ ID NO: 172, CDR_{H}2: that is a CDRH2 in SEQ ID NO: 173, and CDRH3: that is a CDRH3 in SEQ ID NO: 174. The heavy chain can also have threonine (T) at position 221. In some embodiments, the anti-VEGF light chain has the following CDRs: CDR_{L}1: that is a CDRL1 in SEQ ID NO: 199, CDR_{L}2: that is a CDRL2 in SEQ ID NO: 200, and CDR_{L}3: that is a CDRL3 in SEQ ID NO: 201. The anti-VEGF-A light chain can also have leucine (L) at Kabat position 4.

In some embodiments, the anti-VEGF-A antibody is IgG1. In some embodiments, the heavy chain has one or more mutations to modulate effector function. In some embodiments, the mutations are to one or more of the following amino acid positions (EU numbering): E233, L234, L235, G236, G237, A327, A330, and P331. In some embodiments, the mutations are selected from the group consisting of: E233P, L234V, L234A, L235A, G237A, A327G, A330S and P331S (EU numbering). In some embodiments, the mutations are (EU numbering) L234A, L235A and G237A.

In some embodiments, the cysteine residue added to the therapeutic protein via recombinant DNA technology should not be involved in Cys-Cys disulfide bond pairing. In this regard, therapeutic proteins may be dimeric. So for example, an intact anti-VEGF-A antibody has two light chains and two heavy chains. If a Cys residue is introduced into the heavy chain for instance, the intact antibody will have two such introduced cysteines at identical positions and the possibility exists that these cysteine residues will form intra-chain disulfide bonds. If the introduced cysteine residues form Cys-Cys disulfide bonds or have a propensity to do so, that introduced Cys residue will not be useful for conjugation. It is known in the art how to avoid positions in the heavy and light chains that will give rise to intra-chain disulfide pairing. See, e.g., U.S. Patent Application No. 2015/0158952.

In some embodiments, the cysteine residue introduced via recombinant DNA technology is selected from the group consisting of (EU numbering) Q347C and L443C. In some embodiments, the cysteine residue is L443C (EU numbering, or 449C). In some embodiments, the heavy chain the antibody has the amino acid sequence set forth in at least one of SEQ ID NOs: 7-13, 19-27, 89, 90, 256-262; and the light chain has the amino acid sequence of at least one of SEQ ID NOs: 91-93, 28-30.

In some embodiments, the sulfhydryl specific reacting group is maleimide.

In some embodiments, the half-life extending moiety is selected from the group consisting of polyethylene glycol (PEG), branched PEG, PolyPEG® (Warwick Effect Polymers; Coventry, UK), polysialic acid (PSA), starch, hydroxylethyl starch (HES), hydroxyalkyl starch (HAS), carbohydrate, polysaccharides, pullulane, chitosan, hyaluronic acid, chondroitin sulfate, dermatan sulfate, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polycarboxylate, polyvinylpyrrolidone, polyphosphazene, polyoxazoline, polyethylene-co-maleic acid anyhydride, polystyrene-co-maleic acid anhydride, poly(1-hydroxymethyethylene hydroxymethylformal) (PHF), a zwitterionic polymer, a phosphorylcholine containing polymer and a polymer comprising 2-methacryloyloxy-2′-ethyltrimethylammoniumphosphate (MPC).

In some embodiments, the half-life extending moiety is a zwitterionic polymer. In some embodiments, the zwitterion is phosphorylcholine, i.e. a phosphorylcholine containing polymer. In some embodiments, the polymer is composed of MPC units.

In some embodiments, the MPC polymer has three or more arms. In some embodiments, the MPC polymer has 2, 3, 4, 5, 6, 7, 8, 9, 10, 11 or 12 arms. In some embodiments, the MPC polymer has 3, 6, or 9 arms. In some embodiments, the MPC polymer has 9 arms. In some embodiments, the polymer is synthesized with an initiator comprising 2, 3, 4, 5, 6, 7, 8, 9, 10, 11, 12, or more polymer initiation sites

In some embodiments, the MPC polymer has a molecular weight between about 300,000 and 1,750,000 Da. In some embodiments, the MPC polymer has a molecular weight between about 500,000 and 1,000,000 Da or between about 600,000 to 900,000 Da.

In some embodiments, the method of preparing a therapeutic protein-half life extending moiety conjugate has an additional step of contacting the therapeutic protein with a thiol reductant under conditions that produce a reduced cysteine sulfhydryl group. As discussed above, it is preferable that the cysteine residue added via recombinant DNA technology are unpaired, i.e. are not involved in Cys-Cys intra chain disulfide bonds or are not substantially involved in such bonding. However, Cys residues which are not involved in such Cys-Cys disulfide bonding and are free for conjugation are known to react with free cysteine in the culture media to form disulfide adducts. See, e.g., WO 2009/052249. A cysteine so derivatized will not be available for conjugation. To free the newly added cysteine from the disulfide adduct, the protein after purification is treated with a reducing agent, e.g., dithiothreitol. However, such treatment with a reducing agent will reduce all of the cysteine residues in the therapeutic protein, including native cysteines many of which are involved in inter and intra chain Cys-Cys disulfides bonds. The native Cys-Cys disulfides are generally crucial to protein stability and activity and they should be reformed. In some embodiments, all native (e.g., inter and intra) Cys-Cys disulfides are reformed.

To reform native inter and intra-chain disulfide residues, after reduction to remove the cysteine disulfide adducts, the therapeutic protein is exposed to oxidizing conditions and/or oxidizing agents for a prescribed period of time, e.g., overnight. In some embodiments, ambient air exposure overnight can be used to achieve reformation of the native disulfide bonds. In some embodiments, an oxidizing agent is employed to restore the native disulfides. In some embodiments, the oxidizing agent is selected from the group consisting of aqueous CuSO4 and dehydroascorbic acid (DHAA). In some embodiments, the oxidizing agent is DHAA. In some embodiments, the range of DHAA used is in the range of 5-30 equivalents. In some embodiments, the range is 10-20 equivalents. In some embodiments, the range is 15 equivalents.

In some embodiments, the thiol reductant is selected from the group consisting of: Tris[2-carboxyehtyl]phosphine hydrochloride (TCEP), dithiothreitol (DTT), dithioerythritol (DTE), sodium borohydride (NaBH₄), sodium cyanoborohydride (NaCNBH3), β-mercaptoethanol (BME), cysteine hydrochloride and cysteine. In some embodiments, the thiol reductant is TCEP.

In some embodiments, the thiol reductant concentration is between 1- and 100-fold molar excess relative to the therapeutic protein concentration. In some embodiments, the thiol reductant concentration is between 20-to-50-fold molar excess relative to the therapeutic protein concentration. In some embodiments, the thiol reductant is removed following incubation with the therapeutic protein prior to oxidation of the therapeutic protein.

In some embodiments, the method for conjugating a therapeutic protein to a half-life extending moiety has a further step of purifying the therapeutic protein conjugate after conjugation. In some embodiments, the therapeutic protein conjugate is purified using a technique selected from the group consisting of ion exchange chromatography, hydrophobic interaction chromatography, size exclusion chromatography, and affinity chromatography or combinations thereof.

In some embodiments, the therapeutic protein conjugate retains at least 20% biological activity relative to unconjugated therapeutic protein. In some embodiments, the therapeutic protein conjugate retains at least 50% biological activity relative to unconjugated therapeutic protein. In some embodiments, the therapeutic protein conjugate retains at least 90% biological activity relative to native therapeutic protein.

In some embodiments, the therapeutic protein conjugate has an increased half-life relative to unconjugated therapeutic protein. In some embodiments, the therapeutic protein conjugate has at least a 1.5-fold increase in half-life relative to unconjugated therapeutic protein. In some embodiments, the therapeutic protein conjugate has at least a 5-fold increase in half-life relative to unconjugated therapeutic protein.

In some embodiments, the zwitterionic polymer of the method of conjugating a therapeutic protein to a half-life extending moiety is a radically polymerizable monomer having a zwitterionic group and the method has a further step of polymerizing the free radically polymerizable zwitterionic monomer in a polymerization medium to provide a polymer, the medium comprising: the radically polymerizable zwitterionic monomer; a transition metal catalyst Mₜ^{(q-1)+} wherein Mₜ is a transition metal, q is a higher oxidation state of the metal and q−1 is a lower oxidation state of the metal, wherein the metal catalyst is supplied as a salt of the form Mt^{(q-1)+}X′_{(q-1)} wherein X′ is a counterion or group or the transition metal catalyst is supplied in situ by providing the inactive metal salt at its higher oxidation state Mₜ^{q+}X′_{q} together with a reducing agent that is capable of reducing the transition metal from the oxidized inactive state to the reduced active state; a ligand; and an initiator.

To function as an ATRP transition metal catalyst, the transition metal should have at least two readily accessible oxidation states separated by one electron, a higher oxidation state and a lower oxidation state. In ATRP, a reversible redox reaction results in the transition metal catalyst cycling between the higher oxidation state and the lower oxidation state while the polymer chains cycle between having propagating chain ends and dormant chain ends. See, e.g., U.S. Pat. No. 7,893,173.

In some embodiments, the radically polymerizable zwitterionic monomer is selected from the group consisting of:

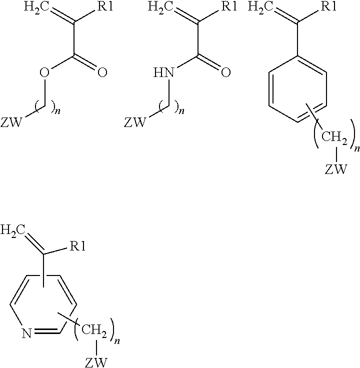

wherein R1 is H or C₁₋₆ alkyl, ZW is a zwitterion and n is an integer from 1-6.

In some embodiments, the radically polymerizable monomer is

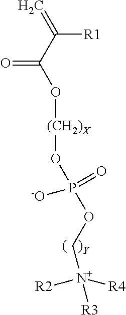

wherein R1 is H or C₁₋₆ alkyl, R2, R3, R4 are the same or different and are H or C₁₋₄ alkyl and X and Y are the same or different and are integers from 1-6. In some embodiments, R1, R2, R3 and R4 are each methyl and X and Y are each 2.

In some embodiments, the radically polymerizable monomer is

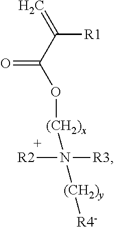

wherein R1 is H or C₁₋₆alkyl, R2 and R3 are the same or different and are H or C₁₋₄alkyl, R4 is PO₄—, SO₃— or CO₂— and X and Y are the same or different and are integers from 1-6. In some embodiments, R1, R2 and R3 are methyl, R4 is PO₄— and X and Y are each 2.

In some embodiments, the monomer is

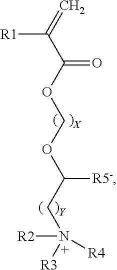

wherein R1 is H or C₁₋₆alkyl, R2, R3 and R4 are the same or different and are H or C₁₋₄alkyl, R5 is PO₄—, SO₃— or CO₂— and X and Y are the same or different and are integers from 1-6. In some embodiments, R1, R2, R3 and R4 are methyl, R5 is PO₄— and X and Y are 2.

In some embodiments, the transition metal Mt is selected from the group consisting of Cu, Fe, Ru, Cr, Mo, W, Mn, Rh, Re, Co, V, Zn, Au, and Ag. In some embodiments, the metal catalyst is supplied as a salt of the form Mt^{(q-1)+}X′_{(q-1)}. Mₜ^{(q-1)+} is selected from the group consisting of Cu¹⁺, Fe²⁺, Ru²⁺, Cr²⁺, Mo²⁺, W²⁺, Mn³⁺, Rh³⁺, Re²⁺, Co⁺, V²⁺, Zn⁺, Au⁺, and Ag⁺ and X′ is selected from the group consisting of halogen, C₁₋₆ alkoxy, (SO₄)_{1/2}, (PO₄)_{1/3}, (R7PO₄)_{1/2}, (R7₂PO₄), triflate, hexaluorophosphate, methanesulfonate, arylsulfonate, CN and R7CO₂, where R7 is H or a straight or branched C₁₋₆ alkyl group which may be substituted from 1 to 5 times with a halogen. In some embodiments, Mₜ^{(q-1)+} is Cu¹⁺ and X′ is Br.

In some embodiments, Mₜ^{(q-1)+} is supplied in situ. In some embodiments, Mₜ^{q+}X_{q} is CuBr₂. In some embodiments, the reducing agent is an inorganic compound. In some embodiments, the reducing agent is selected from the group consisting of a sulfur compound of a low oxidation level, sodium hydrogen sulfite, an inorganic salt comprising a metal ion, a metal, hydrazine hydrate and derivatives of such compounds. In some embodiments, the reducing agent is a metal. In some embodiments, the reducing agent is Cu⁰.

In some embodiments, the reducing agent is an organic compound. In some embodiments, the organic compound is selected from the group consisting of alkylthiols, mercaptoethanol, or carbonyl compounds that can be easily enolized, ascorbic acid, acetyl acetonate, camphosulfonic acid, hydroxy acetone, reducing sugars, monosaccharides, glucose, aldehydes, and derivatives of such organic compounds.

In some embodiments, the ligand is selected from the group consisting of 2,2′-bipyridine, 4,4′-Di-5-nonyl-2,2′-bipyridine, 4,4-dinonyl-2,2′-dipyridyl, 4,4′,4″-tris(5-nonyl)-2,2′:6′,2″-terpyridine, N,N,N′,N′,N″-Pentamethyldiethylenetriamine, 1,1,4,7,10,10-Hexamethyltriethylenetetramine, Tris(2-dimethylaminoethyl)amine, N,N-bis(2-pyridylmethyl)octadecylamine, N,N,N′,N′-tetra[(2-pyridal)methyl]ethylenediamine, tris[(2-pyridyl)methyl]amine, tris(2-aminoethyl)amine, tris(2-bis(3-butoxy-3-oxopropyl)aminoethyl)amine, tris(2-bis(3-(2-ethylhexoxy)-3-oxopropyl)aminoethyl)amine and Tris(2-bis(3-dodecoxy-3-oxopropyl)aminoethyl)amine. In some embodiments, the ligand is 2,2′-bipyridine.

In some embodiments the initiator has the structure:

R1-R2 [symbol] R3)ₛ,

wherein R1 is a nucleophilic reactive group, R2 comprises a linker, and R3 comprises a polymer synthesis initiator moiety having the structure

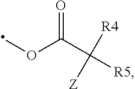

wherein R4 and R5 and are the same or different and are selected from the group consisting of alkyl, substituted alkyl, alkylene, alkoxy, carboxyalkyl, haloalkyl, cycloalkyl, cyclic alkyl ether, alkenyl, alkenylene, alkynyl, alkynylene, cycloalkylene, heterocycloalkyl, heterocycloalkylene, aryl, arylene, arylene-oxy, heteroaryl, amino, amido or any combination thereof; Z is a halogen, —OR (where R is —H, methyl, ethyl, propyl, or isopropyl), —SCN or —NCS; and s is an integer between 1 and 20.

In some embodiments, Z is Br and R4 and R5 are each methyl. In some embodiments, R1 is selected from the group consisting of —NH₂, —OH, and —SH.

In some embodiments R2 is alkyl, substituted alkyl, alkylene, alkoxy, carboxyalkyl, haloalkyl, cycloalkyl, cyclic alkyl ether, alkenyl, alkenylene, alkynyl, alkynylene, cycloalkylene, heterocycloalkyl, heterocycloalkylene, aryl, arylene, arylene-oxy, heteroaryl, amino, amido or any combination thereof. In some embodiments, R2 is

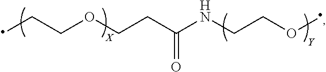

wherein X and Y are the same or different and are integers from 1-20. In some embodiments, X and Y are each 4.

In some embodiments, R3 is

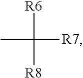

wherein R6, R7 and R8 are the same or different and are selected from the group consisting of

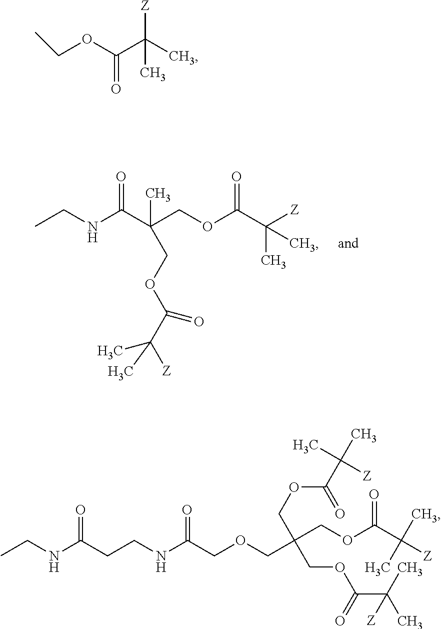

wherein Z is —OR (where R is —H, methyl, ethyl, propyl, or isopropyl), —SCN, —NCS, —F, —Cl, —Br or —I. In some embodiments, Z is —Br and R6, R7 and R8 are each

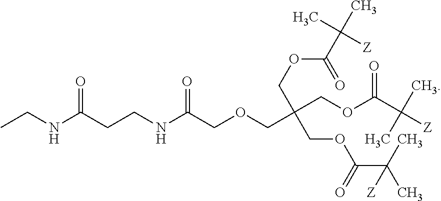

In some embodiments, the initiator has the structure:

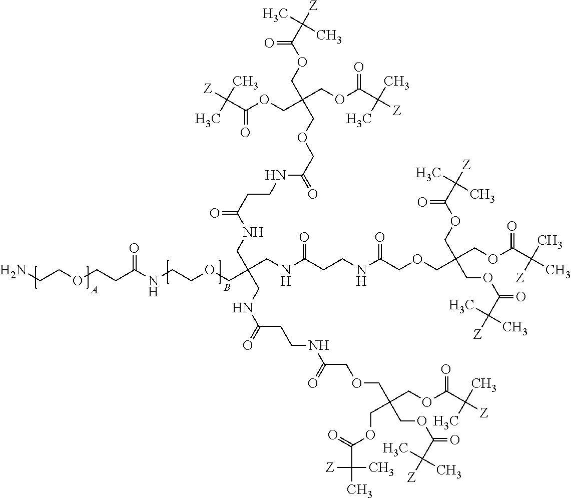

wherein A and B are the same or different and are integers from 2 to 12 and Z is any halide, for example Br. In some embodiments, A and B are each 4.

In some embodiments, the method further has the step of reacting the polymer with a maleimide reagent to provide a polymer having a terminal maleimide. In some embodiments, the maleimide compound is

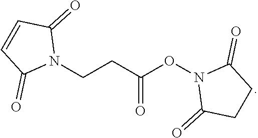 [SMILES: O=C(CCN1C(=O)C=CC1=O)ON1C(=O)CCC1=O]

### Pharmaceutical Compositions

Therapeutic proteins can be incorporated into a pharmaceutical composition with a pharmaceutically acceptable excipient. Pharmaceutical compositions adapted for oral administration may be presented as discrete units such as capsules, as solutions, syrups, or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions.

Pharmaceutical compositions can be adapted for nasal administration wherein the excipient is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the excipient is a liquid, for administration as a nasal spray or as nasal drops, include aqueous or oil solutions of the active ingredient. Pharmaceutical compositions adapted for administration by inhalation include fine particle dusts or mists which may be generated by means of various types of metered dose pressurized aerosols, nebulizers or insufflators.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets. Pharmaceutical compositions can be substantially isotonic, implying an osmolality of about 250-400 mOsm/kg water.

The pharmaceutical compositions may contain preserving agents, solubilizing agents, stabilizing agents, wetting agents, emulsifiers, sweeteners, colorants, odorants, salts (substances may themselves be provided in the form of a pharmaceutically acceptable salt), buffers, coating agents or antioxidants. They may also contain therapeutically active agents in addition to the substance. The pharmaceutical compositions may be employed in combination with one or more pharmaceutically acceptable excipients. Such excipients may include, but are not limited to, saline, buffered saline (such as phosphate buffered saline), dextrose, liposomes, water, glycerol, ethanol and combinations thereof.

The antibodies and pharmaceutical compositions containing them may be administered in an effective regime for treating or prophylaxis of a patient's disease including, for instance, administration by oral, intravitreal, intravenous, subcutaneous, intramuscular, intraosseous, intranasal, topical, intraperitoneal, and intralesional administration. Parenteral infusions include intramuscular, intravenous, intraarterial, intraperitoneal, or subcutaneous administration or routes among others. In therapy or as a prophylactic, the active agent may be administered to an individual as an injectable composition, for example as a sterile aqueous dispersion. In some embodiments the agent is isotonic or substantially isotonic.

For administration to mammals, and particularly humans, it is expected that the dosage of the active agent is from 0.01 mg/kg body weight, typically around 1 mg/kg. The physician can determine the actual dosage most suitable for an individual which depends on factors including the age, weight, sex and response of the individual, the disease or disorder being treated, and the age and condition of the individual being treated. The above dosages are exemplary of the average case. There can, of course, be instances where higher or lower dosages are merited. In some embodiments, the dosage can be 0.5 to 20 mg/eye, e.g., 1, 2, 3, 4, 5, 6, 7, 8, 9, 10, 11, 12, 13, 14, 15, 16, 17, 18, or 19 mg.

This dosage may be repeated as often as appropriate (e.g., weekly, fortnightly, monthly, once every two months, quarterly, twice a year, yearly). If side effects develop the amount and/or frequency of the dosage can be reduced, in accordance with normal clinical practice. In one embodiment, the pharmaceutical composition may be administered once every one to thirty days. In one embodiment, the pharmaceutical composition may be administered twice every thirty days. In one embodiment, the pharmaceutical composition may be administered once a week.

The antibodies and pharmaceutical compositions can be employed alone or in conjunction with other compounds, such as therapeutic compounds or molecules, e.g. anti-inflammatory drugs, analgesics or antibiotics. Such administration with other compounds may be simultaneous, separate or sequential. The components may be prepared in the form of a kit which may comprise instructions as appropriate.

The antibodies and pharmaceutical compositions disclosed herein can be used for treatment or prophylaxis of disease, particularly the ocular diseases or conditions described herein.

The anti-VEGF antibody conjugates, or anti-VEGF protein conjugates, and pharmaceutical compositions containing them may be formulated for and administered by ocular, intraocular, and/or intravitreal injection, and/or juxtascleral injection, and/or subretinal injection and/or subtenon injection, and/or superchoroidal injection and/or subconjunctival and/or topical administration in the form of eye drops and/or ointment. Such antibodies and compositions can be delivered by a variety of methods, e.g. intravitreally as a device and/or a depot that allows for slow release of the compound into the vitreous, including those described in references such as Intraocular Drug Delivery, Jaffe, Ashton, and Pearson, editors, Taylor & Francis (March 2006). In one example, a device may be in the form of a minipump and/or a matrix and/or a passive diffusion system and/or encapsulated cells that release the compound for a prolonged period of time (Intraocular Drug Delivery, Jaffe, Ashton, and Pearson, editors, Taylor & Francis (March 2006)).

Formulations for ocular, intraocular or intravitreal administration can be prepared by methods and using ingredients known in the art. A main requirement for efficient treatment is proper penetration through the eye. Unlike diseases of the front of the eye, where drugs can be delivered topically, retinal diseases require a more site-specific approach. Eye drops and ointments rarely penetrate the back of the eye, and the blood-ocular barrier hinders penetration of systemically administered drugs into ocular tissue. Accordingly, usually the method of choice for drug delivery to treat retinal disease, such as AMD and CNV, is direct intravitreal injection. Intravitreal injections are usually repeated at intervals which depend on the patient's condition, and the properties and half-life of the drug delivered.

Therapeutic antibodies and related conjugates generally are placed into a container having a sterile access port, for example, an intravenous solution bag or vial having a stopper pierceable by a hypodermic injection needle. Such compositions may also be supplied in the form of pre-filled syringes.

A “stable” formulation is one in which the protein or protein conjugated to a polymer of other half-life extending moiety therein essentially retains its physical stability and/or chemical stability and/or biological activity upon storage. By “stable” is also meant a formulation which exhibits little or no signs of instability, including aggregation and/or deamidation. For example, the formulations provided may remain stable for at least two year, when stored as indicated at a temperature of 5-8° C. Suitable formulations for an anti-VEGF antibody conjugate of the present disclosure are described in e.g., PCT publication number WO2017117464, which is incorporated by reference herein in its entirety.

Various analytical techniques for measuring protein stability are available in the art and are reviewed in Peptide and Protein Drug Delivery, 247-301 (Vincent Lee ed., New York, N.Y., 1991) and Jones, 1993 Adv. Drug Delivery Rev. 10: 29-90, for examples. Stability can be measured at a selected temperature for a selected time period. In some embodiments the storage of the formulations is stable for at least 6 months, 12 months, 12-18 months, or for 2 or more years.

A protein, such as an antibody or fragment thereof, “retains its physical stability” in a pharmaceutical formulation if it shows no signs of aggregation, precipitation, deamidation and/or denaturation upon visual examination of color and/or clarity, or as measured by UV light scattering or by size exclusion chromatography.

A protein “retains its chemical stability” in a pharmaceutical formulation, if the chemical stability at a given time is such that the protein is considered to still retain its biological activity. Chemical stability can be assessed by detecting and quantifying chemically altered forms of the protein. Chemical alteration may involve size modification (e.g., clipping), which can be evaluated using size exclusion chromatography, SDS-PAGE and/or matrix-assisted laser desorption ionization/time-of-flight mass spectrometry (MALDI/TOF MS), for examples. Other types of chemical alteration include charge alteration (e.g., occurring as a result of deamidation), which can be evaluated by ion-exchange chromatography, for example. An antibody “retains its biological activity” in a pharmaceutical formulation, if the biological activity of the antibody at a given time is within about 10% (within the errors of the assay) of the biological activity exhibited at the time the pharmaceutical formulation was prepared as determined in an antigen binding assay, for example.

A protein-polymer conjugate “retains its chemical stability” the chemical bond between the protein and the polymer is maintained intact, e.g., it is not hydrolyzed or otherwise disrupted. The protein part of the conjugate retains its chemical stability as described above.

By “isotonic” is meant that the formulation of interest has essentially the same osmotic pressure as human blood or the vitreous for intravitreal injections. Isotonic formulations will generally have an osmotic pressure from about 250 to 400 mOsm. Isotonicity can be measured using a vapor pressure or ice-freezing type osmometer, for example.

As used herein, “buffer” refers to a buffered solution that resists changes in pH by the action of its acid-base conjugate components. In some embodiments, the buffer has a pH from about 3.0 to about 8.0; for example from about 4.5 to 8; or about pH 6 to about 7.5; or about 6.0 to about 7.0, or about 6.5-7.0, or about pH 7.0 to about 7.5; or about 7.1 to about 7.4. A pH of any point in between the above ranges is also contemplated.

In some embodiments, “PBS” phosphate buffered saline, Tris based buffers and histidine-based buffers are used. In some embodiments, acetate buffers are used.

In some embodiments, the PBS buffer is made up of at least Na₂HPO₄, KH₂PO₄ and NaCl adjusted so as to provide the appropriate pH. In some embodiments, the buffer may contain other pharmaceutical excipients such as KCl and other salts, detergents and/or preservatives so as to provide a stable storage solution.

A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

In some embodiments, formulations, to be safe for human use or for animal testing, should have sufficiently low levels of endotoxin. “Endotoxin” is lipopolysaccharide (LPS) derived from the cell membrane of Gram-negative bacteria. Endotoxin is composed of a hydrophilic polysaccharide moiety covalently linked to a hydrophobic lipid moiety (lipid A). Raetz C R, Ulevitch R J, Wright S D, Sibley C H, Ding A, Nathan C F. 1991. Gram-negative endotoxin: an extraordinary lipid with profound effects on eukaryotic signal transduction. FASEB J. 5(12):2652-2660. Lipid A is responsible for most of the biological activities of endotoxin, i.e., its toxicity. Endotoxins are shed in large amount upon bacterial cell death as well as during growth and division. They are highly heat-stable and are not destroyed under regular sterilizing conditions. Extreme treatments with heat or pH, e.g., 180-250° C. and over 0.1 M of acid or base must be used (Petsch D, Anspach F. 2000. Endotoxin removal from protein solutions. J Biotechnol. 76: 97-119). Such conditions of course would be highly detrimental to biological drugs.

In the biotech and pharmaceutical industries, it is possible to find endotoxin during both production processes and in final products. As bacteria can grow in nutrient poor media, including water, saline and buffers, endotoxins are prevalent unless precautions are taken. Endotoxin injection into an animal or human causes a wide variety of pathophysiological effects, including endotoxin shock, tissue injury and even death. Ogikubo Y, Ogikubo Y, Norimatsu M, Noda K, Takahashi J, Inotsume M, Tsuchiya M, Tamura Y. 2004. Evaluation of the bacterial endotoxin test for quantifications of endotoxin contamination of porcine vaccines. Biologics 32:88-93.

Pyrogenic reactions and shock are induced in mammals upon intravenous injection of endotoxin at low concentrations (1 ng/mL) (Fiske J M, Ross A, VanDerMeid R K, McMichael J C, Arumugham. 2001. Method for reducing endotoxin in Moraxella catarrhalis UspA2 protein preparations. J Chrom B. 753:269-278). The maximum level of endotoxin for intravenous applications of pharmaceutical and biologic product is set to 5 endotoxin units (EU) per kg of body weight per hour by all pharmacopoeias (Daneshiam M, Guenther A, Wendel A, Hartung T, Von Aulock S. 2006. In vitro pyrogen test for toxic or immunomodulatory drugs. J Immunol Method 313:169-175). EU is a measurement of the biological activity of an endotoxin. For example, 100 pg of the standard endotoxin EC-5 and 120 pg of endotoxin from Escherichia coli O111:B4 have activity of 1 EU (Hirayama C, Sakata M. 2002. Chromatographic removal of endotoxin from protein solutions by polymer particles. J Chrom B 781:419-432). Meeting this threshold level has always been a challenge in biological research and pharmaceutical industry (Berthold W, Walter J. 1994. Protein Purification: Aspects of Processes for Pharmaceutical Products. Biologicals 22:135-150; Petsch D, Anspach F B. 2000. Endotoxin removal from protein solutions. J Biotech 76:97-119).

The presence of endotoxin in drugs to be delivered via intravitreal injection is of particular concern. Intravitreal injection of drug (penicillin) was first performed in 1945 by Rycroft. Rycroft B W. 1945. Penicillin and the control of deep intra-ocular infection. British J Ophthalmol 29 (2): 57-87. The vitreous is a chamber where high level of drug can be introduced and maintained for relatively long periods of time. The concentration of drug that can be achieved via intravitreal injection far exceeds what can be generated by topical administration or by systemic administration (e.g. intravenous).

One of the most dangerous complications potentially arising from intravitreal injections is endophthalmitis. Endophthalmitis falls into two classes: infectious and sterile. Infectious endophthalmitis is generally cause by bacteria, fungi or parasites. The symptoms of infectious endophthalmitis include severe pain, loss of vision, and redness of the conjunctiva and the underlying episclera. Infectious endophthalmitis requires urgent diagnosis and treatment. Possible treatments include intravitreal injection of antibiotics and pars plana vitrectomy in some cases. Enucleation may be called for to remove a blind and painful eye. See, e.g., Christy N E, Sommer A. 1979. Antibiotic prophylaxis of postoperative endophthalmitis. Ann Ophthalmol 11 (8): 1261-1265.

Sterile endophthalmitis in contrast does not involve an infectious agent and can be defined as the acute intraocular inflammation of the vitreous cavity that resolves without the need of intravitreal antibiotics and/or vitreoretinal surgery. If a vitreous microbiological study has been done, it needs to be negative culture proven to sustain a diagnosis of sterile endophthalmitis. Marticorena J, Romano V, Gomez-Ulla F. 2012 “Sterile Endophthalmitis after Intravitreal Injections” Med Inflam. 928123.

It has been observed that intravitreal injection of biological drugs contaminated with endotoxin can result in sterile endophthalmitis. Marticorena, et al. Bevacizumab (Avastin) is approved by the Food and Drug Administration for the treatment of glioblastoma and of metastatic colorectal cancer, advanced nonsquamous non-small-cell lung cancer and metastatic kidney cancer. Bevacizumab is also widely used off label as a treatment for wet AMD. Bevacizumab comes from the manufacturer as a 100 mg/4 ml. This solution cannot be directly used for intravitreal injection and should be compounded by a pharmacist. Clusters of sterile endophthalmitis have been observed and are theorized to be cause by inadvertent contamination of bevacizumab by endotoxin by the compounding pharmacist.

Given the dire clinical results of intravitreal injection of endotoxin, the total amount of endotoxin that can be given to a patient via intravitreal dosing is highly limited. In some embodiments, a solution having an antibody or antibody-conjugate is provided having an endotoxin level that does not exceed 5.0 EU/ml. In some embodiments, the endotoxin level does not exceed 1.0 EU/ml. In some embodiments, the endotoxin level does not exceed 0.5 EU/ml. In some embodiments, the endotoxin level does not exceed 0.2 EU/ml. In some embodiments, the endotoxin level does not exceed 2, 1, 0.5, 0.2, 0.1, 0.09, 0.08, 0.07, 0.06, 0.05, 0.04, 0.03, 0.02 or 0.01 EU/ml.

Two commonly used FDA-approved tests for the presence of endotoxin are the rabbit pyrogen test and Limulus Amoebodyte Lysate (LAL) assay (Hoffman S, et al. 2005. International validation of novel pyrogen tests based on human monocytoid cells J. Immunol. Methods 298:161-173; Ding J L, Ho B A. 2001. New era in pyrogen testing. Biotech. 19:277-281). The rabbit pyrogen test was developed in the 1920s and involves monitoring the temperature rise in a rabbit injected with a test solution. However, use of the rabbit pyrogen test has greatly diminished over the years due to expense and long turnaround time. Much more common is the LAL test. LAL is derived from the blood of a horseshoe crab and clots upon exposure to endotoxin.

One of the simplest LAL assays is the LAL gel-clot assay. Essentially, the LAL clotting assay is combined with a serial dilution of the sample in question. Formation of the gel is proportional to the amount of endotoxin in the sample. Serial dilutions are prepared from the sample and each dilution assayed for its ability to form LAL gel. At some point a negative reaction is contained. The amount of endotoxin in the original sample can be estimated from the dilution assay.

Other LAL tests have also been developed, including the turbidimetric LAL assay (Ong K G, Lelan J M, Zeng K F, Barrett G, Aourob M, Grimes C A. 2006. A rapid highly-sensitive endotoxin detection system. Biosensors and Bioelectronics 21:2270-2274) and the chromogenic LAL assay (Haishima Y, Hasegawa C, Yagami T, Tsuchiya T, Matsuda R, Hayashi Y. 2003. Estimation of uncertainty in kinetic-colorimetric assay of bacterial endotoxins. J Pharm Biomed Analysis. 32:495-503). The turbidimetric and chromogenic assays are much more sensitive and quantitative than the simple gel-clot dilution assay.

In some embodiments a method of reducing the amount of endotoxin in a composition having an antibody disclosed herein is provided. The method having the steps of contacting the composition with an affinity chromatography resin that binds to the antibody; eluting the antibody from the affinity chromatography resin to form an affinity chromatography eluent having the antagonist; contacting the affinity chromatography eluent with an ion-exchange resin that binds the antibody; and eluting the antibody from the ion-exchange resin, wherein the antibody eluted from the ion-exchange resin is substantially free from endotoxin.

The above method for reducing the amount of endotoxin, or other method or process recited herein, can be performed in the order described in the steps above or it can optionally be performed by varying the order of the steps or even repeating one or more of the steps. In one embodiment, the method of reducing the amount of endotoxin in a composition is performed in the order of the described steps. In some embodiments, the affinity chromatography resin contacting, washing and eluting steps are repeated in the same order more than one time before contacting the affinity chromatography eluent with the ion exchange resin. The method can also include a filtering step using, for example, a 0.1-micron, 0.20 micron, or 0.45-micron filter, that can be performed on either one or more of the eluents removed after each resin binding step.

In certain instances, the steps of contacting the composition with affinity chromatography resin, washing and eluting the antibody from the affinity chromatography resin can be repeated more than one time before contacting the first eluent with an ion-exchange resin. In one embodiment, the affinity chromatography resin comprises a recombinant Protein A (“rProteinA”) resin. One example of a suitable recombinant Protein A resin is MabSelect Sure and Mabselect Sure LX (Cytiva). In another embodiment, a suitable affinity chromatography resin would comprise a protein G chromatography resin. In other embodiments, a suitable affinity chromatography resin comprises a mixed Protein A/Protein G resin. In some embodiments, a suitable affinity chromatography resin comprises a protein L resin. In other embodiments, a suitable affinity chromatography resin comprises a hydrophobic charge induction resin that comprises a 4-mercaptoethylpyridine ligand such as a MEP HyperCel® resin (BioSepra, Cergy, Saint Christophe, France).

In some embodiments, the ion exchange resin comprises an anion-exchange resin. As will be known by the person skilled in the art, ion exchangers may be based on various materials with respect to the matrix as well as to the attached charged groups. For example, the following matrices may be used, in which the materials mentioned may be more or less cross-linked: POROS XS (ThermoFisher), POROS XQ (ThermoFisher), MacroCap Q (Cytiva, Piscataway, N.J.), agarose based (such as Sepharose CL-6B®, Sepharose Fast Flow® and Sepharose High Performance @), cellulose based (such as DEAE Sephacel®), dextran based (such as Sephadex®), silica based and synthetic polymer based. For the anion exchange resin, the charged groups, which are covalently attached to the matrix, may, for example, be diethylaminoethyl, quaternary aminoethyl, and/or quaternary ammonium. In some embodiments the anion-exchange resin comprises a quaternary amine group. An exemplarily anion-exchange resin that has a quaternary amine group for binding the anti-M-CSF antibody is a Q Sepharose® resin (Amersham, Piscataway, N.J.).

In other aspects, if the endotoxin levels are higher than desired after subjecting the composition to the aforementioned anion-exchange chromatography step, the composition may in the alternative be subjected to a cation exchange resin. In some embodiments, any endotoxin in the composition should have a differential binding to the ion-exchange resin than the protein in question to allow purification of the protein from the endotoxin. In this regard, endotoxin is negatively charged and will generally bind to an anion exchange resin. If both the protein and the endotoxin bind to the anion exchange resin, purification of one from the other may be effectuated by using a salt gradient to elute the two into different fractions. The relative binding of the protein to a particular resin may also be altered by changing the pH of the buffer relative to the pI of the protein. In some embodiments, cation-exchange chromatography is the sole ion-exchange chromatography employed.

In some embodiments, if the endotoxin levels are too high after the anion exchange resin, the composition may be further subjected to a second ion-exchange step, for example, by contacting the compositions with a cation exchange resin and followed by a wash step, then elution from the ion-exchange resin. In some embodiments, the cation exchange resin comprises a sulfonic group for binding. Exemplary cation exchange resins are SP Sepharose® resin FF (Amersham, Piscataway, N.J.) POROS XS (CEX) (ThermoFisher). In some embodiments, endotoxin removal is accomplished using hydrophobic interaction chromatography. In some embodiments, hydrophobic interaction chromatography is used when both endotoxin and mAb have the same or similar charges. In some embodiments, hydrophobic interaction chromatography can be carried out by exemplary systems, like using Sartobind Phenyl (Sartorius, Gottingen, Germany).

In some embodiments, after the solution of antibody protein is produced having the specified level of endotoxin, there are a number of steps prior to final formulation of the protein. In some embodiments, a half-life extending moiety is conjugated to the protein. The conjugate is then formulated into a final drug formulation which is injected into the patients. In some embodiments, the conjugate is again purified on an ion-exchange resin which can be a cation-exchange resin. In other embodiments, the protein is formulated. In all cases, normal laboratory procedures should be employed to prevent the introduction of endotoxin contaminants into the protein sample or into the protein-polymer conjugate.

In some embodiments, any of the following arrangements are contemplated herein:

A method of purifying a product using affinity chromatography, the method comprising loading an eluent into an affinity chromatography matrix, wherein the affinity chromatography matrix binds to a protein of interest; and washing the affinity chromatography matrix with a buffer solution comprising a chaotropic agent.

A method of purifying a product and reducing impurities from a load fluid comprising the protein and one or more impurities by passing the load fluid through an affinity chromatography matrix, followed by at least one wash solution comprising a chaotropic salt, and collecting the protein using an elution solution.

A method for separating impurities in an eluate comprising a protein of interest, the method comprising loading an eluent comprising a protein of interest onto an affinity chromatography matrix; and washing the affinity chromatography matrix with one or more buffer solutions comprising one or more of lithium and lithium salts, magnesium and magnesium salts, calcium and calcium salts and guanidinium and guanidinium salts.

A method of producing a product using affinity chromatography, the method comprising loading an eluent containing a protein of interest onto an affinity chromatography matrix, a first wash of the affinity chromatography matrix with a first buffer comprising sodium phosphate and a salt, and a second wash of the affinity chromatography matrix with a second buffer comprising a chaotropic agent.

A method of producing a product using affinity chromatography, the method comprising loading an eluent containing a protein of interest onto an affinity chromatography matrix, a first wash with a first buffer containing Tris and a salt, a second wash with a second buffer containing Tris and a chaotropic agent, wherein the second buffer chaotropic agent is not the same salt as contained in the first buffer.

A method of producing a product, the method comprising collecting a load fluid, wherein the load fluid comprises a protein of interest, loading the load fluid onto an affinity chromatography matrix, wherein the affinity chromatography matrix binds to the protein of interest, washing the affinity chromatography matrix with a buffer solution comprising a chaotropic salt, eluting the bound protein of interest; and collecting an eluate, wherein the eluate contains the protein of interest.

A method of producing a product, the method comprising collecting a load fluid, wherein the load fluid comprises a protein of interest, loading the load fluid onto an affinity chromatography matrix, wherein the affinity chromatography matrix binds to the protein of interest, feeding to the affinity chromatography matrix a buffer solution comprising a chaotropic salt, eluting the bound protein of interest; and collecting an eluate, wherein the eluate contains the protein of interest.

A method of producing a product, the method comprising: collecting a conjugate protein, wherein the conjugate protein comprises an antibody bound to a conjugate polymer loading the conjugate protein onto an affinity chromatography matrix, wherein the affinity chromatography matrix binds to the conjugate protein, washing the affinity chromatography matrix with a buffer solution comprising a chaotropic salt, eluting the conjugate protein, and collecting an eluate, wherein the eluate contains the conjugate protein.

A method of producing a product, the method comprising washing an affinity chromatography matrix bound to a target protein of interest with a buffer comprising a chaotropic salt, eluting and collecting an eluate, wherein the eluate contains the target protein of interest, and removing viral contaminants from the eluate. The method may further include wherein removing viral contaminants from the eluate comprises one or more of low pH inactivation, detergent inactivation, polishing chromatography steps, viral filtration (VF), ultrafiltration (UF) and/or diafiltration (DF). The method may further include wherein the eluate is further combined with an acceptable pharmaceutical excipient to form a pharmaceutical composition. The method may further include wherein a buffer solution is added to the pharmaceutical composition. The method may further include wherein a preservative solution is added to the pharmaceutical composition. The method may further include wherein the pharmaceutical composition is further refined for intravitreal injection.

A method of producing a product, the method comprising washing an affinity chromatography matrix bound to a target protein of interest with a buffer comprising a chaotropic salt, removing the chaotropic salt, and eluting and collecting an eluate, wherein the eluate contains the target protein of interest.

A method of producing a product, the method comprising collecting a load fluid, wherein the load fluid is comprised of a protein of interest, loading the load fluid into an affinity chromatography matrix, wherein the affinity chromatography matrix binds to the protein of interest, washing the affinity chromatography matrix with a buffer solution comprising a chaotropic salt, eluting and collecting an eluate, wherein eluate contain the target protein of interest, and removing viral contaminants from the eluate.

A method of producing a product, the method comprising loading an eluent into an affinity chromatography matrix, washing with a first wash buffer washing with a second wash buffer comprising a chaotropic salt, washing with a third wash buffer, wherein the third wash buffer removes the chaotropic salt, eluting with an elution buffer, wherein an eluate is collected, wherein the eluate comprises a protein product. The method may further include wherein the first wash buffer comprises 50 mM Na-Phosphate. The method may further include wherein the first wash buffer further comprises 250 mM NaCl. The method may further include wherein the first wash buffer comprises Tris and a salt. The method may further include removing viral contaminants from the eluate. The method may further include wherein removing viral contaminants comprises: one or more of low pH inactivation, detergent inactivation, polishing chromatography steps, viral filtration (VF), ultrafiltration (UF), or diafiltration (DF). The method may further include wherein the eluent comprises a protein of interest. The method may further include wherein the protein of interest is an antibody. The method may further include wherein the antibody is further conjugated to a polymer to form an antibody conjugate. The method may further include wherein the antibody conjugate comprises a bispecific antibody. The method may further include wherein the bispecific antibody comprises anti-VEGF and anti IL-6 binding moieties. The method may further include wherein the antibody conjugate has the following structure:

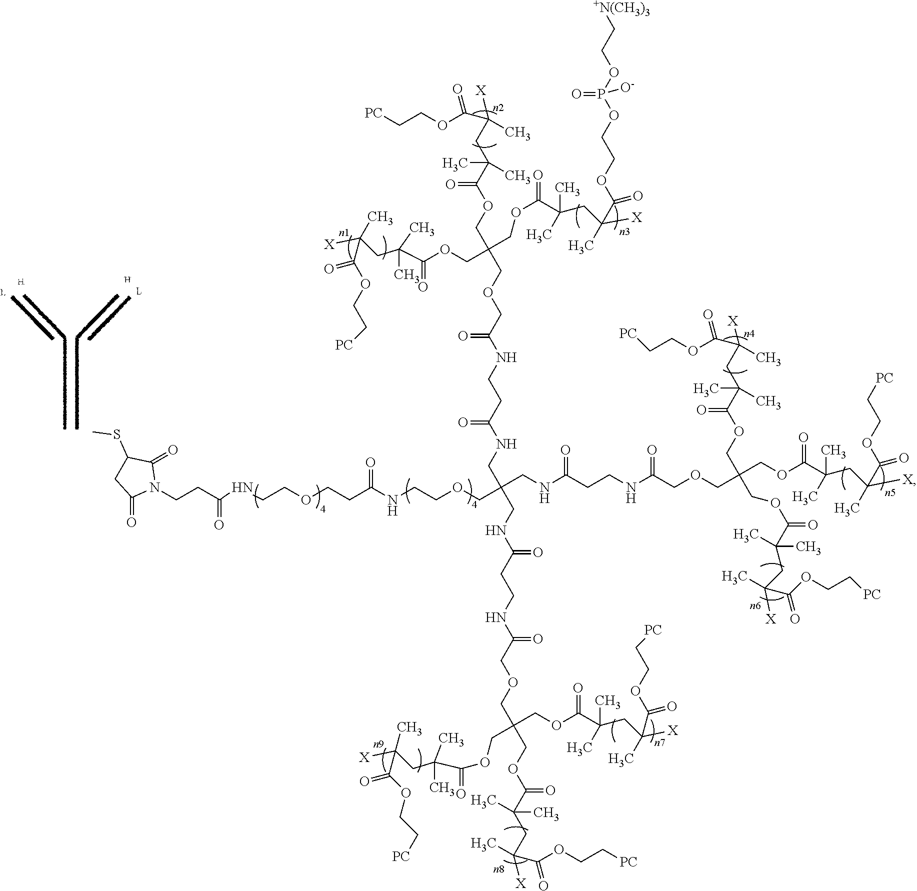

wherein each heavy chain of the anti-VEGF-A antibody is denoted by the letter H, and each light chain of the anti-VEGF-A antibody is denoted by the letter L; the polymer is bonded to the anti-VEGF-A antibody through the sulfhydryl of C443 (EU numbering), which bond is depicted on one of the heavy chains; PC is:

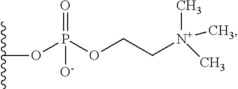 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

A method of producing a product, the method comprising recovering a cell culture supernatant, wherein the cell culture supernatant comprises a protein of interest, processing the cell culture supernatant into an eluent, wherein the eluent comprises the protein of interest, loading the eluent into an affinity chromatography matrix, washing with a first wash buffer comprising Tris or Sodium Phosphate, washing with a second wash buffer comprising a chaotropic salt, eluting with an elution buffer, wherein an eluate is collected, wherein the eluate comprises a protein product, inactivating viral contaminants present in the eluate with a low pH viral buffer to yield a viral inactivated eluate, filtering the viral inactivated eluate, performing at least one round of ion exchange chromatography on the viral inactivated eluate, and filtering the viral inactivated eluate to yield a retentate, wherein the retentate comprises the protein of interest. The method may further include wherein the cell culture supernatant was produced in a bioreactor using animal component free cell culture. The method may further include wherein processing the cell culture supernatant comprises harvesting cell products from a cell culture. The method may further include wherein the cell culture is clarified to remove cells and cellular debris. The method may further include wherein the eluent comprises the clarified cell culture supernatant. A method of purifying a protein using affinity chromatography, the method comprising contacting a load fluid with a medium, wherein the medium is an affinity chromatography matrix that binds a protein of interest, washing the medium with a buffer solution comprising a chaotropic agent, wherein the chaotropic agent is a salt, and contacting the washed medium with an elution solution under conditions suitable for eluting the protein of interest.

A method of producing a product, the method comprising applying the solution containing a protein of interest onto an affinity chromatography matrix, washing the affinity chromatography matrix with a first buffer, washing the affinity chromatography matrix with a second buffer containing a chaotropic agent, washing the affinity chromatography matrix with a third buffer to remove the chaotropic agent, eluting with an elution buffer, wherein an eluate is collected, wherein the eluate comprises a protein product.

A system for protein purification, comprising a column having a first antigen binding protein bound to the column; a phosphate wash buffer comprising sodium phosphate and a salt, an intermediate wash buffer comprising tris, a second wash buffer comprising magnesium chloride, and an elution buffer comprising sodium formate.

A system for protein purification, comprising a column having a first antigen binding protein bound to the column; a first tris wash buffer comprising tris and a salt, an intermediate tris wash buffer, a second wash buffer comprising magnesium chloride, and an elution buffer comprising sodium formate, The system may further include wherein the column comprises a ligand for affinity chromatography. The system may further include wherein the ligand comprises Protein A or Protein G. The system may further include wherein the first wash buffer comprising sodium phosphate and a salt has a pH between 5.5 and 9.5. The system may further include wherein the phosphate wash buffer comprising sodium phosphate and a salt comprises about 50 mM sodium phosphate. The system may further include wherein the phosphate wash buffer comprising sodium phosphate and a salt comprises about 250 mM NaCl. The system may further include wherein the first tris wash buffer comprises about 50 mM Tris. The system may further include wherein the first tris wash buffer further comprises about 250 mM NaCl. The system may further include wherein the intermediate tris wash buffer comprises about 50 mM Tris. The system may further include wherein the pH of the first tris wash buffer is about 7.2. The system may further include wherein the pH of the second wash buffer is about 7.8. The system may further include wherein the concentration of magnesium chloride in the second wash buffer is about 2.8 M. The system may further include wherein the concentration of sodium formate in the elution buffer comprises 10 mM.

A system for antibody purification, comprising a column having a protein A resin bound to an antibody, wherein the antibody comprises a light and heavy chain of at least one of SEQ ID NOs: 91-93, 28-30, and at least one of SEQ ID NOs: 7-13, 19-27, 89, 90, 256-262, respectively, a chaotropic wash buffer comprising a chaotropic salt, and an elution buffer comprising sodium formate.

The methods described herein may further include wherein the protein of interest is a bispecific antibody. The methods described herein may further include wherein the bispecific antibody is specific for VEGF and IL-6. The methods described herein may further include wherein the bispecific antibody is specific for VEGF and IL-6. The methods described herein may further include wherein the protein of interest is an antibody conjugate. The methods described herein may further include wherein the affinity chromatography matrix is a protein A chromatography matrix. The methods described herein may further include wherein the chaotropic agent in the buffer solution is comprised of a magnesium salt. The methods described herein may further include wherein the concentration of magnesium salt is between 1.5-3.5 M. The methods described herein may further include wherein the chaotropic agent in the buffer solution is comprised of a calcium salt. The methods described herein may further include wherein the concentration of the calcium salt is between 1-3 M. The methods described herein may further include wherein the chaotropic agent in the buffer solution is comprised of a guanidinium salt. The methods described herein may further include wherein the concentration of the guanidinium salt is between 0.05-3 M. The methods described herein may further include wherein the buffer solution further comprises tris. The methods described herein may further include wherein the concentration of tris in the buffer solution is at least 5 mM. The methods described herein may further include wherein the pH of the buffer solution is greater than 5.5. The methods described herein may further include wherein the eluate further contains viral impurities. The methods described herein may further include removing the viral impurities. The methods described herein may further include inactivating the viral impurities. The methods described herein may further include washing the affinity chromatography matrix loaded with the load fluid with a prewash buffer solution prior to washing with the buffer solution. The methods described herein may further comprise the step of washing the affinity chromatography matrix loaded with the eluent with a postwash buffer solution after washing with buffer solution. The methods described herein may further include wherein the prewash buffer solution comprises sodium phosphate. The methods described herein may further include wherein the prewash buffer solution comprises Tris and a salt.

The methods described herein may further include wherein the antibody conjugate has the following structure:

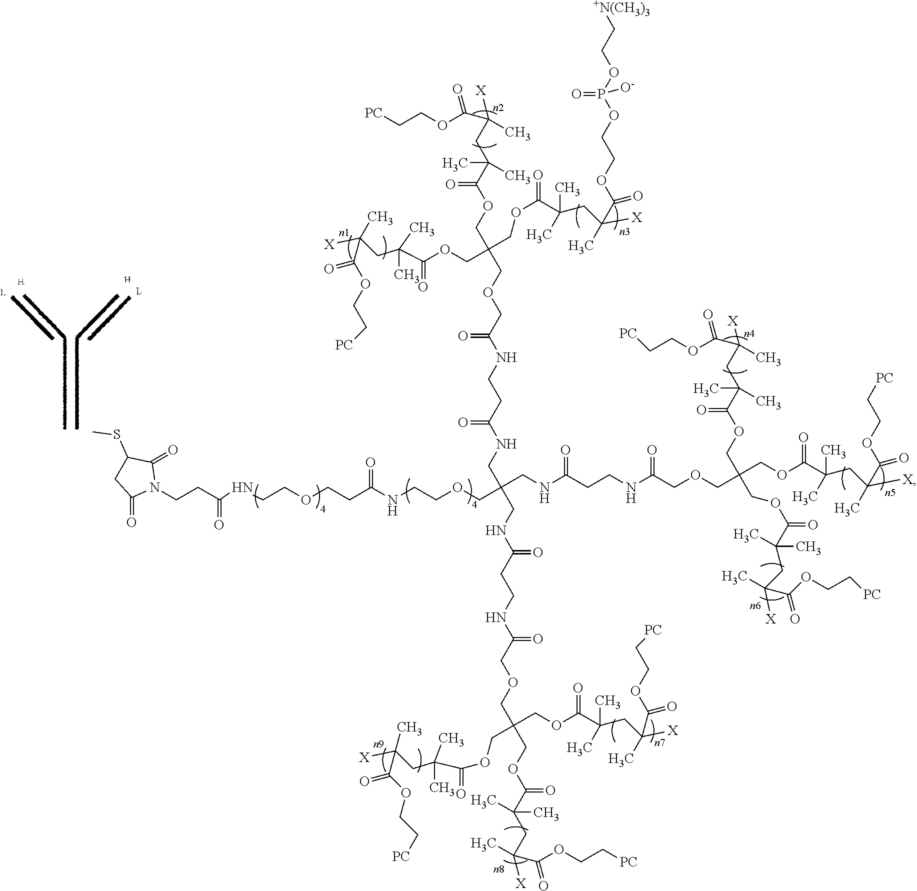

wherein each heavy chain of the anti-VEGF-A antibody is denoted by the letter H, and each light chain of the anti-VEGF-A antibody is denoted by the letter L; the polymer is bonded to the anti-VEGF-A antibody through the sulfhydryl of C443 (EU numbering), which bond is depicted on one of the heavy chains; PC is:

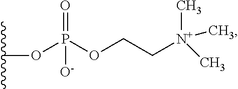 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

The methods described herein may further include wherein the antibody conjugate comprises an anti-VEGF antibody conjugate comprising an anti-VEGF-A light chain and an anti-VEGF-A heavy chain, wherein the anti-VEGF-A antibody heavy chain comprises CDRH1: that is a CDRH1 in SEQ ID NO: 172, CDRH2: that is a CDRH2 in SEQ ID NO: 173, and CDRH3: that is a CDRH3 in SEQ ID NO: 174, and the anti-VEGF-A antibody light chain comprises CDRL1: that is a CDRL1 in SEQ ID NO: 199, CDRL2: that is a CDRL2 in SEQ ID NO: 200, and CDRL3: that is a CDRL3 in SEQ ID NO: 201. The methods described herein may further include wherein the anti-VEGF antibody conjugate comprises: an antibody conjugate comprising an anti-VEGF-A immunoglobulin G (IgG) bonded to a polymer, which polymer comprises MPC monomers, wherein the sequence of the anti-VEGF-A antibody heavy chain is at least one of SEQ ID NOs: 7-13, 19-27, 89, 90, 256-262, and the sequence of the anti-VEGF-A antibody light chain is at least one of SEQ ID NOs: 91-93, 28-30, and wherein the antibody is bonded at C449 to the polymer. The methods described herein may further include wherein the target protein of interest is produced by a cell culture. The methods described herein may further include wherein the cell culture comprises CHO cells. The methods described herein may further include the step of washing the affinity chromatography matrix loaded with the eluent with a postwash buffer solution after washing with buffer solution. The methods described herein may further include washing the affinity chromatography matrix with the buffer solution removes nucleic acids, endotoxins, antifoam agents, or other small molecules other than the target protein of interest. The methods described herein may further include washing the affinity chromatography matrix with the buffer solution removes impurities while keeping the target protein of interest bound to the affinity chromatography matrix. The methods described herein may further include wherein washing the affinity chromatography matrix with the buffer solution removes host cell proteins besides the target protein of interest. The methods described herein may further include wherein the addition of chaotropic agent in the buffer solution does not elute the target protein of interest. The methods described herein may further include one or more of virus inactivation, tangential flow filtration, diafiltration, ultrafiltration, ion exchange chromatography, or virus reduction filtration. The methods described herein may further include wherein the eluent was produced in a bioreactor using animal component free cell culture. The methods described herein may further include wherein the product is a protein of interest. The methods described herein may further include wherein impurities comprise host cell protein impurities.

The methods described herein may further include wherein the first wash buffer comprises a salt. The methods described herein may further include wherein the first wash buffer comprises a phosphate-based species. The methods described herein may further include wherein the first wash buffer comprises Na-Phosphate. The methods described herein may further include wherein the first wash buffer comprises between about 0.1 and about 250 mM phosphate salt, including about 0.1 mM, about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 150 mM, about 200 mM, about 250 mM, or any integer that is between 0.1 and 250 mM. The methods described herein may further include wherein the first wash buffer comprises 10 mM Na-Phosphate.

The methods described herein may further include wherein the second wash buffer comprises a salt. The methods described herein may further include wherein the second wash buffer comprises a phosphate-based species. The methods described herein may further include wherein the second wash buffer comprises Na-Phosphate. The methods described herein may further include wherein the second wash buffer comprises between about 0.1 and about 2500 mM phosphate salt, including about 0.1 mM, about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 150 mM, about 200 mM, about 250 mM, or any integer that is between 0.1 and 250 mM. The methods described herein may further include wherein the second wash buffer comprises 10 mM Na-Phosphate.

The methods described herein may further include wherein the wash buffer comprises a salt. The methods described herein may further include wherein the wash buffer comprises a phosphate-based species. The methods described herein may further include wherein the wash buffer comprises Na-Phosphate. The methods described herein may further include wherein the wash buffer comprises between about 0.1 and about 250 mM phosphate salt, including about 0.1 mM, about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 150 mM, about 200 mM, about 250 mM, or any integer that is between 0.1 and 250 mM. The methods described herein may further include wherein the wash buffer comprises 10 mM Na-Phosphate.

In some embodiments, the buffering system can employ a system other than Tris. In some embodiments, the buffering system can employ a system other than Na-Phosphate. In some embodiments, the buffer can be one or more of the following: Acetate, Citrate, ACES, BES, Bicine, HEPES, MES, MOPS, MOPSO, TAPS, Tricine, Bis-Tris, Bis-Tris propane, Cacodylate, CAPS, CAPSO, CHES, Glycine, Glycylglycine, Imidazole, PIPES, TEA, or TES. In some embodiments, the buffering system can employ pH values other than those provided. In some embodiments, the buffering system can employ pH values between 2-13. In some embodiments, the elution buffer used is not Na-Formate. In some embodiments, the elution buffer is basic. In some embodiments, the elution buffer is acidic.

### Arrangements

In some embodiments, any of the following arrangements are contemplated herein:

1. A method of purifying a product using affinity chromatography, the method comprising: loading an eluent into an affinity chromatography matrix, wherein the affinity chromatography matrix binds to a protein of interest; and washing the affinity chromatography matrix with a buffer solution comprising a chaotropic agent.

2. A method of purifying a product and reducing impurities from a load fluid comprising the protein and one or more impurities by passing the load fluid through an affinity chromatography matrix, followed by at least one wash solution comprising a chaotropic salt, and collecting the protein using an elution solution.

3. A method for separating impurities in an eluate comprising a protein of interest, the method comprising:
loading an eluent comprising a protein of interest onto an affinity chromatography matrix; and washing the affinity chromatography matrix with one or more buffer solutions comprising one or more of lithium and lithium salts, magnesium and magnesium salts, calcium and calcium salts and guanidinium and guanidinium salts.

4. A method of producing a product using affinity chromatography, the method comprising:
loading an eluent containing a protein of interest onto an affinity chromatography matrix,
a first wash of the affinity chromatography matrix with a first buffer comprising sodium phosphate and a salt, and
a second wash of the affinity chromatography matrix with a second buffer comprising a chaotropic agent.

5. A method of producing a product using affinity chromatography, the method comprising:
loading an eluent containing a protein of interest onto an affinity chromatography matrix,
a first wash with a first buffer containing Tris and a salt,
a second wash with a second buffer containing Tris and a chaotropic agent,
wherein the second buffer chaotropic agent is not the same salt as contained in the first buffer.

6. A method of producing a product, the method comprising:
collecting a load fluid, wherein the load fluid comprises a protein of interest, loading the load fluid onto an affinity chromatography matrix,
wherein the affinity chromatography matrix binds to the protein of interest,
washing the affinity chromatography matrix with a buffer solution comprising a chaotropic salt, eluting the bound protein of interest; and collecting an eluate,
wherein the eluate contains the protein of interest.

7. A method of producing a product, the method comprising:
collecting a load fluid, wherein the load fluid comprises a protein of interest,
loading the load fluid onto an affinity chromatography matrix,
wherein the affinity chromatography matrix binds to the protein of interest,
feeding to the affinity chromatography matrix a buffer solution comprising a chaotropic salt,
eluting the bound protein of interest; and
collecting an eluate, wherein the eluate contains the protein of interest.

8. A method of producing a product, the method comprising:
collecting a conjugate protein, wherein the conjugate protein comprises an antibody bound to a conjugate polymer loading the conjugate protein onto an affinity chromatography matrix, wherein the affinity chromatography matrix binds to the conjugate protein,
washing the affinity chromatography matrix with a buffer solution comprising a chaotropic salt,
eluting the conjugate protein, and
collecting an eluate, wherein the eluate contains the conjugate protein.

9. A method of producing a product, the method comprising:
washing an affinity chromatography matrix bound to a target protein of interest with a buffer comprising a chaotropic salt, eluting and collecting an eluate,
wherein the eluate contains the target protein of interest,
and removing viral contaminants from the eluate.

10. The method of arrangement 9, wherein removing viral contaminants from the eluate comprises:
one or more of low pH inactivation, detergent inactivation, polishing chromatography steps, viral filtration (VF), ultrafiltration (UF) and/or diafiltration (DF).

11. A method of producing a product, the method comprising:
washing an affinity chromatography matrix bound to a target protein of interest with a buffer comprising a chaotropic salt, and
removing the chaotropic salt, and eluting and collecting an eluate,
wherein the eluate contains the target protein of interest.

12. The method of 11, wherein the eluate is further combined with an acceptable pharmaceutical excipient to form a pharmaceutical composition.

13. The method of arrangement 12, wherein a buffer solution is added to the pharmaceutical composition.

14. The method of arrangement 12, wherein a preservative solution is added to the pharmaceutical composition.

15. The method of arrangement 12, wherein the pharmaceutical composition is further refined for intravitreal injection.

16. A method of producing a product, the method comprising:
collecting a load fluid, wherein the load fluid is comprised of a protein of interest, loading the load fluid into an affinity chromatography matrix, wherein the affinity chromatography matrix binds to the protein of interest,
washing the affinity chromatography matrix with a buffer solution comprising a chaotropic salt,
eluting and collecting an eluate, wherein eluate contain the target protein of interest, and
removing viral contaminants from the eluate.

17. A method of producing a product, the method comprising:
loading an eluent into an affinity chromatography matrix,
washing with a first wash buffer,
washing with a second wash buffer comprising a chaotropic salt,
washing with a third wash buffer, wherein the third wash buffer removes the chaotropic salt, and
eluting with an elution buffer, wherein an eluate is collected, wherein the eluate comprises a protein product.

18. The method of arrangement 17, wherein the first wash buffer comprises 50 mM Na-Phosphate.

19. The method of arrangement 17, wherein the first wash buffer further comprises 250 mM NaCl.

20. The method of arrangement 17, wherein the first wash buffer comprises Tris and a salt.

21. The method of arrangement 17, further comprising removing viral contaminants from the eluate.

22. The method of arrangement 21, wherein removing viral contaminants comprises: one or more of low pH inactivation, detergent inactivation, polishing chromatography steps, viral filtration (VF), ultrafiltration (UF), or diafiltration (DF).

23. The method of arrangement 17, wherein the eluent comprises a protein of interest.

24. The method of arrangement 23, wherein the protein of interest is an antibody.

25. The method of arrangement 24, wherein the antibody is further conjugated to a polymer to form an antibody conjugate.

26. The method of arrangement 24, wherein the antibody conjugate has the following structure:

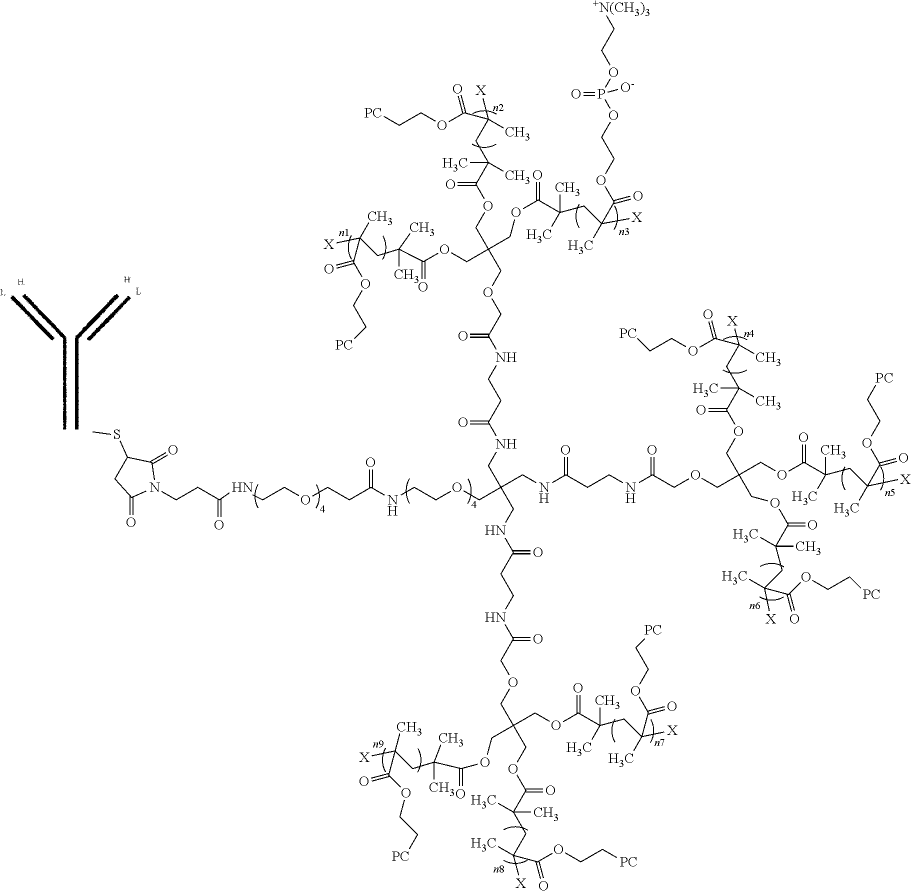

wherein:
each heavy chain of the anti-VEGF-A antibody is denoted by the letter H, and
each light chain of the anti-VEGF-A antibody is denoted by the letter L;
the polymer is bonded to the anti-VEGF-A antibody through the sulfhydryl of C443 (EU numbering), which bond is depicted on one of the heavy chains;

PC is:

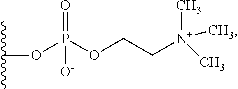 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

27. The method of arrangement 25, wherein the antibody conjugate comprises a bispecific antibody.

28. The method of arrangement 24, wherein the bispecific antibody comprises anti-VEGF and anti-IL-6 binding moieties.

29. A method of producing a product, the method comprising:
recovering a cell culture supernatant, wherein the cell culture supernatant comprises a protein of interest,
processing the cell culture supernatant into an eluent, wherein the eluent comprises the protein of interest,
loading the eluent into an affinity chromatography matrix,
washing with a first wash buffer comprising Tris or Sodium Phosphate
washing with a second wash buffer comprising a chaotropic salt,
eluting with an elution buffer, wherein an eluate is collected, wherein the eluate comprises a protein product,
inactivating viral contaminants present in the eluate with a low pH viral buffer to yield a viral inactivated eluate,
filtering the viral inactivated eluate,
performing at least one round of ion exchange chromatography on the viral inactivated eluate, and
filtering the viral inactivated eluate to yield a retentate, wherein the retentate comprises the protein of interest.

30. The method of arrangement 29, wherein the cell culture supernatant was produced in a bioreactor using animal component free cell culture.

31. The method of arrangement 29, wherein processing the cell culture supernatant comprises harvesting cell products from a cell culture.

32. The method of arrangement 31, wherein the cell culture is clarified to remove cells and cellular debris.

33. The method of arrangement 29, wherein the eluent comprises the clarified cell culture supernatant

34. A method of purifying a protein using affinity chromatography, the method comprising:
contacting a load fluid with a medium, wherein the medium is an affinity chromatography matrix that binds a protein of interest, washing the medium with a buffer solution comprising a chaotropic agent, wherein the chaotropic agent is a salt, and contacting the washed medium with an elution solution under conditions suitable for eluting the protein of interest.

35. A method of producing a product, the method comprising:
applying the solution containing a protein of interest onto an affinity chromatography matrix,
washing the affinity chromatography matrix with a first buffer
washing the affinity chromatography matrix with a second buffer containing a chaotropic agent,
washing the affinity chromatography matrix with a third buffer to remove the chaotropic agent, and
eluting with an elution buffer, wherein an eluate is collected, wherein the eluate comprises a protein product.

36. A system for protein purification, comprising:
a column having a first antigen binding protein bound to the column;
a phosphate wash buffer comprising sodium phosphate and a salt,
an intermediate wash buffer comprising tris,
a second wash buffer comprising magnesium chloride, and
an elution buffer comprising sodium formate.

37. A system for protein purification, comprising:
a column having a first antigen binding protein bound to the column;
a first tris wash buffer comprising tris and a salt,
an intermediate tris wash buffer,
a second wash buffer comprising magnesium chloride, and
an elution buffer comprising sodium formate.

38. The system of arrangement 36, wherein the column comprises a ligand for affinity chromatography.

39. The system of arrangement 36, wherein the ligand comprises protein A or Protein G

40. The system of arrangement 36, wherein the first wash buffer comprising sodium phosphate and a salt has a pH between 5.5 and 9.5.

41. The system of arrangement 36, wherein the phosphate wash buffer comprising sodium phosphate and a salt comprises about 50 mM sodium phosphate.

42. The system of arrangement 36, wherein the phosphate wash buffer comprising sodium phosphate and a salt comprises about 250 mM NaCl.

43. The system of arrangement 36, wherein the first tris wash buffer comprises about 50 mM Tris.

44. The system of arrangement 43, wherein the first tris wash buffer further comprises about 250 mM NaCl.

45. The system of arrangement 36, wherein the intermediate tris wash buffer comprises about 50 mM Tris.

46. The system of arrangement 36, wherein the pH of the first tris wash buffer is about 7.2.

47. The system of arrangement 36, wherein the pH of the second wash buffer is about 7.8.

48. The system of arrangement 36, wherein the concentration of magnesium chloride in the second wash buffer is about 2.8 M.

49. The system of arrangement 36, wherein the concentration of sodium formate in the elution buffer comprises 10 mM.

50. A system for antibody purification, comprising:
a column having a protein A resin bound to an antibody,
wherein the antibody comprises a light and heavy chain of at least one of SEQ ID NOs: 91-93, 28-30., and at least one of SEQ ID NOs: 7-13, 19-27, 89, 90, 256-262, respectively, and
a chaotropic wash buffer comprising a chaotropic salt, and
an elution buffer comprising sodium formate.

51. The method of arrangement 1, wherein the protein of interest is a bispecific antibody.

52. The method of arrangement 51, wherein the bispecific antibody is specific for VEGF and IL-6.

53. The method of arrangement 51, wherein the bispecific antibody is OG2072.

54. The method of arrangement 1, wherein the protein of interest is an antibody conjugate.

55. The method of arrangement 1, wherein the affinity chromatography matrix is a protein A chromatography matrix.

56. The method of arrangement 1, wherein the chaotropic agent in the buffer solution is comprised of a salt selected from the group consisting of: a magnesium salt, a calcium salt, and a guanidinium, salt.

57. The method of arrangement 56, wherein the concentration of the salt is between 0.05-3.5 M.

58. The method of Arrangement 1, wherein the buffer solution further comprises tris.

59. The method of arrangement 58, wherein the concentration of tris in the buffer solution is at least 5 mM.

60. The method of arrangement 1, wherein the pH of the buffer solution is greater than 5.5.

61. The method of arrangement 3, wherein the eluate further contains viral impurities.

62. The method of arrangement 61, further comprising removing the viral impurities.

63. The method of arrangement 62, further comprising inactivating the viral impurities.

64. The method of arrangement 3, further comprising the step of washing the affinity chromatography matrix loaded with the load fluid with a prewash buffer solution prior to washing with the buffer solution.

65. The method of 64, further comprising the step of washing the affinity chromatography matrix loaded with the eluent with a postwash buffer solution after washing with buffer solution.

66. The method of 64, where the prewash buffer solution comprises sodium phosphate.

67. The method of arrangement 64, where the prewash buffer solution comprises Tris and a salt.

68. The method of arrangement 25, wherein the antibody conjugate has the following structure:

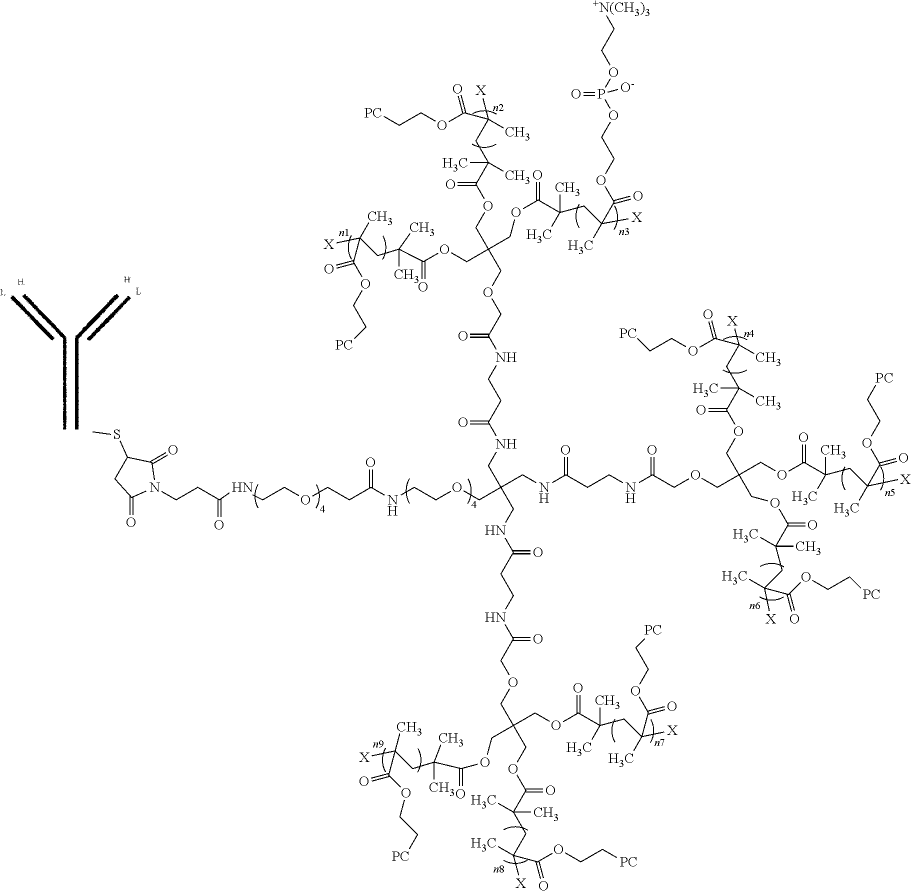

wherein:
each heavy chain of the anti-VEGF-A antibody is denoted by the letter H, and
each light chain of the anti-VEGF-A antibody is denoted by the letter L;
the polymer is bonded to the anti-VEGF-A antibody through the sulfhydryl of C443 (EU numbering), which bond is depicted on one of the heavy chains; PC is:

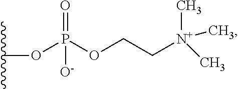 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

69. The method of arrangement 68, wherein the antibody conjugate comprises an anti-VEGF antibody conjugate comprising an anti-VEGF-A light chain and an anti-VEGF-A heavy chain, wherein the anti-VEGF-A antibody heavy chain comprises CDRH1: that is a CDRH1 in SEQ ID NO: 172, CDRH2: that is a CDRH2 in SEQ ID NO: 173, and CDRH3: that is a CDRH3 in SEQ ID NO: 174, and the anti-VEGF-A antibody light chain comprises CDRL1: that is a CDRL1 in SEQ ID NO: 199, CDRL2: that is a CDRL2 in SEQ ID NO: 200, and CDRL3: that is a CDRL3 in SEQ ID NO: 201.

70. The method of arrangement 69, wherein the anti-VEGF antibody conjugate comprises: an antibody conjugate comprising an anti-VEGF-A immunoglobulin G (IgG) bonded to a polymer, which polymer comprises MPC monomers, wherein the sequence of the anti-VEGF-A antibody heavy chain is at least one of SEQ ID NOs: 7-13, 19-27, 89, 90, 256-262, and the sequence of the anti-VEGF-A antibody light chain is at least one of SEQ ID NOs: 91-93, 28-30, and wherein the antibody is bonded at C449 to the polymer.

71. The method according to arrangement 1, wherein the target protein of interest is produced by a cell culture.

72. The method according to arrangement 71, wherein the cell culture comprises CHO cells.

73. The method of arrangement 3, further comprising the step of washing the affinity chromatography matrix loaded with the eluent with a postwash buffer solution after washing with buffer solution.

74. The method of arrangement 3, wherein washing the affinity chromatography matrix with the buffer solution removes nucleic acids, endotoxins, antifoam agents, or other small molecules other than the target protein of interest.

75. The method of arrangement 3, wherein washing the affinity chromatography matrix with the buffer solution removes impurities while keeping the target protein of interest bound to the affinity chromatography matrix.

76. The method of arrangement 3, wherein washing the affinity chromatography matrix with the buffer solution removes host cell proteins besides the target protein of interest.

77. The method of arrangement 4, wherein the addition of chaotropic agent in the buffer solution does not elute the target protein of interest.

78. The method of arrangement 3, further comprising one or more of virus inactivation, tangential flow filtration, diafiltration, ultrafiltration, ion exchange chromatography, or virus reduction filtration.

79. The method of arrangement 3, wherein the eluent was produced in a bioreactor using animal component free cell culture.

80. The method of arrangement 3, wherein the product is a protein of interest.

81. The method of arrangement 3, wherein impurities comprise host cell protein impurities.

82. The method of arrangement 17, wherein the first wash buffer comprises 10 mM Na-Phosphate.

83. The method of arrangement 17, wherein the first wash buffer comprises a phosphate-based species.

84. The method of arrangement 17, wherein the first wash buffer further comprises 50 mM NaCl.

85. A method for processing a product, comprising:
loading an eluent into an affinity chromatography matrix; and
washing with a wash buffer comprising a chaotropic salt to collect an eluate,
wherein the concentration of the chaotropic salt is increased from a first concentration to a second concentration, wherein the eluate is collected in at least one fraction, wherein the at least one fraction comprises a product of interest.

86. The method of arrangement 85, wherein the concentration of the chaotropic salt at the first concentration is 0 M, wherein the concentration of the chaotropic salt at the second concentration is 4.0 M.

87. The method of arrangement 85, wherein the chaotropic salt is Magnesium based.

88. The method of arrangement 86, wherein the chaotropic salt is MgCl2.

89. The method of Arrangement 1, wherein the buffer solution further comprises one or more of the following: Acetate, Citrate, ACES, BES, Bicine, HEPES, MES, MOPS, MOPSO, TAPS, Tricine, Bis-Tris, Bis-Tris propane, Cacodylate, CAPS, CAPSO, CHES, Glycine, Glycylglycine, Imidazole, PIPES, TEA, or TES.

90. The method of arrangement 1, wherein the molecule of interest is a protein.

91. The method of arrangement 90, wherein the protein is an antibody or an antibody-like construct e.g. Fc-fusion protein.

### EXAMPLES

### Example 1: Protocol for Overall Purification of OG2072

A process 100 for the purification of OG2072 antibody from an animal component free cell culture process is disclosed (FIG. 1). The process includes three chromatography steps, and two TFF (tangential flow filtration) steps, as well as a low pH viral inactivation. First, a clarified cell supernatant was collected from a CHO cell line 110. Next, Affinity chromatography 120 was performed on the clarified cell supernatant. Low pH Viral inactivation 130, followed by intermediate filtration 140 were then run on an output eluate collected during Affinity Chromatography. Further rounds of chromatography, including Anionic Exchange Chromatography 150, then Cationic Exchange Chromatography 160 were then performed. Viral reduction filtration 170, and a final ultrafiltration/diafiltration 180 followed.

The low pH viral inactivation included holding a solution at pH 3.5 for 240 minutes followed by neutralization to pH 7. MabSelect SuRe LX affinity chromatography (MSS LX) was followed by a virus inactivation/neutralization step and then a first TFF (TFF1) to condition the antibody for Sartobind Q anionic exchange chromatography (AEX chromatography). POROS XS cationic exchange chromatography (CEX chromatography), and a viral reduction filtration (Planova 20N) were then run. POROSXS comprises binding at 10 mM sodium phosphate, pH 5, 40 mM NaCl, plus acetate as supplement (<15 mS/cm), followed by gradient elution at 10 CVs from 50 mM Na-Acetate pH 6, at 10 mM NaCl, up to 50 mM Na-Acetate, pH 6, 300 mM NaCl. An additional wash was performed during POROS XS chromatography comprising 2 CV of 50 mM Na-Acetate, pH 5.0, 10 mM NaCl, then 5 CV of 18.8 mM Sodium Phosphate, pH 7.0, 22.5 mM NaCl, then 3 CV of 50 mM Na-Acetate, pH 5.0, 10 mM NaCl, followed by 2 CV of 50 mM Sodium Acetate, pH 6.0, 10 mM NaCl. Last, a second TFF (TFF2) was run to formulate the antibody and obtain an antibody intermediate. However, HCP levels are noted as too high at 27 ng/mg for ophthalmologic applications.

FIG. 2 likewise illustrates a process 200 for purification of a biomolecule. First a cell culture was grown and cultured using a fermentation process 210. The cell culture was then collected at harvest 220 to yield a clarified cell concentrate. The clarified cell concentrate was then purified using affinity chromatography and Viral inactivation/neutralization 230, the output of the process is a product retentate. A tangential flow filtration 240 was then run on the product retentate collected, whereupon an anion exchange chromatography 250 step and a cation exchange chromatography 260 step were run sequentially. The processed product retentate was then subject to a viral reduction filtration 270, wherein a second tangential flow filtration 280 was then run to yield a purified product.

### Example 2: Protocol for Purification of OG2072 During Column Chromatography: General Na-Phosphate Protocol

A protocol to assess fold-reduction of measurable HCP species was developed to assess yield and purity of a desirable molecular species. A process 300 for the column purification of OG2072 antibody from an animal component free cell culture process is disclosed (FIG. 3) (Table 6A). The process comprises an equilibration 310, followed by loading 320 the column with clarified cell culture fluid (CCCF). The column is then treated with a series of washes comprising Wash 1 330, followed by Wash 2 340, Wash 3 350, and Wash 4 360. Wash 2 340 may comprise subwashes 340A, 340B, and 340C. Following Wash 4 360, elution 370 is performed, whereby post-elution 380 is run on the column. The process includes equilibration 310 of a protein A column with 50 mM Na-phosphate and 250 mM NaCl at pH 7, then a load 320 step, wherein the column is loaded with Clarified cell culture fluid (CCCF), followed by a series of washes 330-360, including a first wash 330 with 50 mM Na/phosphate and 250 mM NaCl at pH 7. Wash 2 340 may comprise various wash buffers and conditions as described in Table 6C. Wash 3 350 comprises a wash with 50 mM Na-phosphate and 2 M NaCl, at pH 7, while Wash 4 360 comprises 50 mM Na-phosphate, 250 mM NaCl, at pH 7. Elution 370 was accomplished with 10 mM Na-Formate at pH 3.5, with post elution run through the column at 100 mM Citric Acid at pH 2.1. Flow rates and column volume (CVs) of various solutions are disclosed on the right-hand side of Table 6A. Buffer compositions are disclosed in Table 6A.

TABLE 6A
General Chromatography Purification.

| | | | Flow |
|---|---|---|---|
| | | | rate |
| Step | Buffer | CV | (cm/h) |
| Equilibration | 50 mM Na-phosphate, 250 mM NaCl, pH 7 | 5 | 400 |
| Load | | | 200 |
| Wash 1 | 50 mM Na-phosphate, 250 mM NaCl, pH 7 | 2 | 200 |
| Wash 2— | Varies. Described in Table 6C. | 3 | 400 |
| Additive | | | |
| Concen- | | | |
| tration 1 | | | |
| Wash 2— | | 3 | 400 |
| Additive | | | |
| Concen- | | | |
| tration 2 | | | |
| Wash 2— | | 3 | 400 |
| Additive | | | |
| Concen- | | | |
| tration 3 | | | |
| Wash 3 | 50 mM Na-phosphate, 2 M NaCl, pH 7 | 3 | 400 |
| Wash 4 | 50 mM Na-phosphate, 250 mM NaCl, pH 7 | 3 | 400 |
| Elution | 10 mM Na-Formate pH 3.5 | 4 | 400 |
| Post Elution | 100 mM Citric Acid, pH 2.1 | 2 | 400 |
| CIP | 0.1 NaOH (15 min hold after 2 CV) | 3 | 400 |

### Example 2.5: Purification of OG2072 During Column Chromatography

The steps of column chromatography are performed as described in Table 6A. A column was equilibrated with a buffer comprising 50 mM Na-phosphate and 250 mM NaCl at pH 7. The column volume (CV) during equilibration was 5, and is fed at a flow rate of 400 cm/h. Loading the column followed, at a rate of 200 cm/h. A series of washes were performed as described in Table 6A. The column was then eluted using 10 mM Na-Formate at pH 3.5. Elution involves a CV of 4, and a flow rate of 400 cm/h.

### Example 3: Protocol for Reducing Host-Cell Protein Levels from Cell Extracts

A process for the purification of an antibody using affinity chromatography is disclosed (Table 6B). Prior to column purification, G2072 was produced in a 10 L bioreactor using animal component free cell culture process and fermentation. Clarified cell culture fluid (CCCF; Supernatant SG01174/A1) collected from the bioreactor was determined to be 4.118 mg/mL by Protein A-HPLC. The materials were aliquoted to 280 mL portions and run 5 times at a resin charge of 18 g/L resin on a protein A column. The protein solution had a pH of 7.4 and a conductivity of 13.9 mS/cm.

TABLE 6B
Chaotropic Chromatography Purification (Magnesium Chloride)

| | | | Flow |
|---|---|---|---|
| | | | rate |
| Step | Buffer | CV | (cm/h) |
| Equilibration | 50 mM Na-phosphate, 250 mM NaCl, pH 7 | 5 | 300 |
| Load | | | 200 |
| Wash 1 | 50 mM Na-phosphate, 250 mM NaCl, pH 7 | 2 | 200 |
| Wash 2— | 50 mM Tris, pH 8.8 | 4 | 300 |
| Additive | | | |
| Concen- | | | |
| tration 1 | | | |
| Wash 2— | 100 mM Tris, 2.8 M MgCl₂ | 4 | 100 |
| Additive | | | |
| Concen- | | | |
| tration 2 | | | |
| Wash 2— | 50 mM Tris, pH 8.8 | 1.2 | 100 |
| Additive | | | |
| Concen- | | | |
| tration 3 | | | |
| Wash 3 | 50 mM Tris, pH 8.8 | 2.8 | 300 |
| Wash 4 | 50 mM Na-phosphate, 250 mM NaCl, pH 7 | 2 | 300 |
| Elution | 10 mM Na-Formate pH 3.5 | 5 | 300 |
| Post Elution | 100 mM Citric Acid, pH 2.1 | 2 | 300 |
| CIP | 0.1 NaOH (15 min hold after 2 CV) | 3 | 300 |

The steps of column chromatography are performed as described in Table 6B. A column was equilibrated with a buffer comprising 50 mM Na-phosphate and 250 mM NaCl at pH 7. The column volume (CV) during equilibration was 5, and was fed at a flow rate of 400 cm/h. Loading the column followed, at a rate of 200 cm/h. A series of washes were performed as described in Table 6B. A wash comprising 100 mM Tris and 2.8 M MgCl₂ is described. The wash comprising 100 mM Tris and 2.8 M MgCl₂ further included a CV of 4 and a flow rate of 100 cm/h. The column was then eluted using 10 mM Na-Formate at pH 3.5. Elution involved a CV of 5, and a flow rate of 300 cm/h.

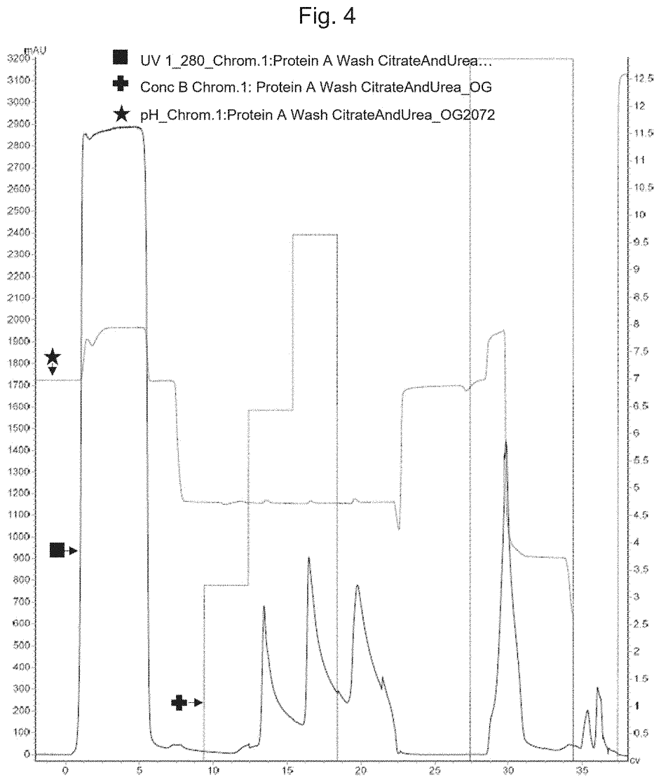

Equilibration was conducted using 50 mM Na-phosphate, 250 mM NaCl, at pH 7, whereafter CCCF was loaded onto the column. A first wash at 50 mM Na-phosphate, 250 mM NaCl, followed by a second wash with 50 mM Tris at pH 8.8 was conducted. A third wash with 100 mM Tris, as well as 2.8 M MgCl2 followed. Washes 4 and 5 were then performed, comprising 50 mM Tris at pH 8.8 at varying flow rates (See FIG. 4). A final wash 6 was then performed at 50 mM Na-phosphate, 250 mM NaCl, pH 7. Following the washes, the protein of interest was eluted using 10 mM Na-Formate at pH 3.5, whereinafter 100 mM Citric acid pH 2.1 was run as a post-elution solution. Flow rates of various fluids are disclosed on the right-hand side of Table 6B. Results indicate an 8-fold reduction in HCP levels compared to a reference run. It is conceivable that the wash step described not only helps to decrease the levels of HCPs, but also other impurities (e.g., endotoxin and nucleic acids).

### Example 4: Experimental Variations on Reducing Host-Cell Protein Levels

The following study design and methods follow the protocol laid out in Example 2, unless indicated otherwise. Host-Cell Protein (HCP) levels were reduced following changes to the wash procedure. Table 6C illustrates variations on Wash 2 according to some embodiments of the present disclosure. Table 6C further discloses results of a reference run, which followed the protocol disclosed in Example 2 without the Wash 2 steps. Compared to the reference, which disclosed a step yield of 89.3%, and an HCP concentration of 1842.71 ng/mg, the Tris+2.8M MgCl2 run yielded a step yield of 87.9% with an eight-fold decrease in concentration of HCP to 226.27 ng/mg.

TABLE 6C
Chromatography Purification Results.

| | | Step | | | | | anti- | anti- | |
|---|---|---|---|---|---|---|---|---|---|
| | Sample | Yield | GP-HPLC | GP-HPLC | GP-HPLC | HCP | VEGF | IL6 | |
| Run | ID | (%) | (% HMW2) | (% HMW1) | (% Purity) | (ng/mg) | RP (%) | RP(%) | pH |
| Wash 2: | KV00080 | 90.8 | 0.78 | 4.71 | 94.52 | 1086.7 | 98.7 | 97.9 | 6.76 |
| Tris + 1.2M | | | | | | | | | |
| Urea | | | | | | | | | |
| Wash 2: | KV00097 | 87.7 | 1.86 | 6.15 | 92 | 438.9 | 99.6 | 96.9 | 6.71 |
| NaPO4 + 3.0M | | | | | | | | | |
| Urea + IPA | | | | | | | | | |
| Wash 2: | KV00082 | 88.3 | 1.14 | 3.34 | 95.53 | 1198.3 | 93.5 | 95.2 | 4.81 |
| NaCitrate + | | | | | | | | | |
| 0.3M | | | | | | | | | |
| Urea | | | | | | | | | |
| Wash 2: | KV00083 | 81.1 | 0.67 | 4.48 | 94.86 | 531.4 | 96.1 | 101.8 | 7.07 |
| Tris + | | | | | | | | | |
| Arginine | | | | | | | | | |
| Reference | KV00084 | 89.3 | 1.07 | 4.52 | 94.42 | 1842.7 | 100 | 100 | 6.28 |
| Run | | | | | | | | | |
| Eshmuno A- | KV00087 | 82.9 | 0.24 | 1.51 | 98.26 | 2427.3 | 102.3 | 99 | 4.121 |
| Reference | | | | | | | | | |
| Run | | | | | | | | | |
| Eshmuno A- | KV00088 | 85.3 | 0.34 | 2.19 | 97.47 | 488.0 | 97.5 | 101.9 | 4.194 |
| Wash 2: | | | | | | | | | |
| Tris + 3M | | | | | | | | | |
| MgCl2 | | | | | | | | | |
| Eshmuno A - | KV00101D | 107.3 | 7.24 | 15.48 | 77.29 | 230.9 | 97.2 | 88 | 6.92 |
| Wash 2: | | | | | | | | | |
| Tris + 3M | | | | | | | | | |
| MgCl2, | | | | | | | | | |
| Elution | | | | | | | | | |
| 4M | | | | | | | | | |
| MgCl2 | | | | | | | | | |
| Wash 2: | KV00103 | 87.9 | 1.14 | 4.58 | 94.3 | 226.3 | 105.3 | 100.6 | 6.15 |
| Tris + 2.8M | | | | | | | | | |
| MgCl2 | | | | | | | | | |
| Wash 2: | KV00105D | 81 | 1 | 6.1 | 92.9 | 696.1 | 111.9 | 113 | 7.189 |
| Tris + MgCl2, | | | | | | | | | |
| MgCL2 | | | | | | | | | |
| Gradient | | | | | | | | | |
| Elution | | | | | | | | | |
| Wash 2: | KV00106D | | 0.92 | 5.88 | 93.2 | 670.0 | 110.2 | 111.9 | |
| Tris + MgCl2, | | | | | | | | | |
| MgCL2 | | | | | | | | | |
| Gradient | | | | | | | | | |
| Elution | | | | | | | | | |

### Example 5: Experimental Runs with a Variety of Wash Buffers

A variety of variable wash conditions were tested to assess purification conditions and buffer compositions. Table 6D illustrates a variety of wash buffers. Run 1 was an investigatory run to determine the lowest pH value possible for a wash step. A pH elution gradient was performed from pH 6 to pH 3 after the wash 1 step as described in FIG. 3. Table 6F shows HCP and yield levels following elution with each described wash buffer, each wash buffer corresponding to runs described in Table 6D. Results indicated various wash and column combinations that yield differing product purity and HCP concentrations.

TABLE 6D
Chromatography Purification Runs

| Run | Exp. | Buffer |
|---|---|---|
| 1 | 0001 | MSS LX: Elution gradient from pH 6.0 to pH 3.0 |
| | | (50 mM Na-citrate) |
| 2 | 0002 | MSS LX: 50 mM Tris, pH 8.8 followed by an |
| | | incremental increase of urea (1, 2, 3M) |
| 3 | 0003 | MSS LX: 50 mM Tris, pH 8.8, 1.2M urea |
| 4 | 0004 | MSS LX: Wash with 50 mM Na-phosphate, pH 7 |
| | | followed by an incremental increase of urea (1, |
| | | 2 & 3M) and 50 mM Na-phosphate pH 6.5, 3M |
| | | urea, 10% IPA |
| 5 | 0005 | no follow up run performed |
| 6 | 0006 | MSS LX: Wash with 50 mM Citrate pH 4.7 |
| | | followed by an incremental increase of urea |
| | | (1, 2, 3M) and 50 mM Na-citrate, pH 4.6, 3M |
| | | Urea, 10% IPA |
| 7 | 0007 | MSS LX: 50 mM Citrate pH 4.7, 0.3M urea |
| 8 | 0008 | MSS LX: Wash with 50 mM Tris pH 8.8 followed |
| | | by an incremental increase of arginine (0.25, |
| | | 0.5, 0.75M); No follow up run performed |
| 9 | 0009 | MSS LX: Reference run (as described in Table 6B |
| | | but without wash 2 step) |
| 10 | 0010 | MSS LX: Wash with 50 mM Tris pH 8.8 followed |
| | | by an incremental increase of MgCl2 (1, 2, 3, 4M) |
| | | applying 100 mM Tris base, 4.0M MgCl2, pH 6.6 |
| 11 | 0011 | MSS LX: 50 mM Tris Base, 3M MgCl2 was |
| | | attempted but column was clogged. The |
| | | chromatography load material was for 3 days at |
| | | ambient and formed precipitates. Container was |
| | | non-transparent, and precipitation was not noticed. |
| | | Also, previous run (0010) had a change from 50 mM |
| | | Trist Base, 4M MgCl2 to 50 mM Na-phosphate 2.0M |
| | | NaCl, pH 7, which can cause on-column precipitation |
| | | as magnesium phosphate readily crystallizes. New |
| | | Column ordered. Continued with screening of |
| | | Eshmuno A resin. |
| 12 | 0012 | Eshmuno A: Reference conditions (as described in |
| | | Table 6B, but without wash 2 steps) |
| 13 | 0013 | Eshmuno A: 50 mM Tris base followed by an |
| | | incremental increase MgCl2 (1, 2, 3, 3.6, 0M) |
| | | applying 100 mM Tris base, 4.0M MgCl2, pH 6.6 |
| 14 | 0014 | Eshmuno A: 50 mM Tris, pH 8.8 followed by 50 mM |
| | | Tris base, 3M MgCl2 and 50 mM Tris, pH 8.8 |
| 15 | 0014b | Eshmuno A: 50 mM Tris, pH 8.8 followed by 50 mM |
| | | Tris base, 3M MgCl2 and elution with 100 mM |
| | | Tris base, 4M MgCl2, pH 6.6 |
| 16 | 0014c | Eshmuno A: 50 mM Na-citrate, pH 4.7 followed by |
| | | an incremental increase of magnesium chloride |
| | | (1, 2, 3, 0M) |
| 17 | 0014d | Eshmuno A: Resin Capacity |
| 18 | 0015 | MSS LX: 50 mM Tris, pH 8.8 followed by 50 mM |
| | | Tris Base, 2.8M MgCl2 and 50 mM Tris, pH 8.8 |
| 19 | 0016 | MSS LX: 50 mM Tris, pH 8.8 followed by 50 mM |
| | | Tris Base, 2.8M MgCl2 and 50 mM Tris, pH 8.8 |
| | | (duplicate run) |
| 20 | 0017 | MSS LX: 50 mM Tris, pH 8.8 followed by 50 mM |
| | | Tris Base, 2.8M MgCl2 and gradient elution from |
| | | 2.8M to 4M MgCl2 |

TABLE 6F
Chromatography Purification Results.

| | | | | | | | anti- | anti- | |
|---|---|---|---|---|---|---|---|---|---|
| | Run | Yield | GP-HPLC | GP-HPLC | GP-HPLC | HCP | VEGF | IL6 | |
| # | Description | (%) | (% HMW2) | (% HMW1) | (% Purity) | (ng/mg) | RP (%) | RP(%) | pH |
| 3 | MSS LX - | 90.8 | 0.78 | 4.71 | 94.52 | 1086.7 | 98.7 | 97.9 | 6.76 |
| | Wash 2: | | | | | | | | |
| | Tris + 1.2M | | | | | | | | |
| | Urea | | | | | | | | |
| 4 | MSS LX - | 87.7 | 1.86 | 6.15 | 92 | 438.9 | 99.6 | 96.9 | 6.71 |
| | Wash 2: | | | | | | | | |
| | NaPo4 + 3.0M | | | | | | | | |
| | Urea + IPA | | | | | | | | |
| 7 | MSS LX - | 88.3 | 1.14 | 3.34 | 95.53 | 1198.3 | 93.5 | 95.2 | 4.81 |
| | Wash2: | | | | | | | | |
| | NaCitrate + | | | | | | | | |
| | 0.3M Urea | | | | | | | | |
| 8 | MSS LX - | 81.1 | 0.67 | 4.48 | 94.86 | 531.4 | 96.1 | 101.8 | 7.07 |
| | Wash 2: | | | | | | | | |
| | Tris + Arginine | | | | | | | | |
| 9 | MSS LX - | 89.3 | 1.07 | 4.52 | 94.42 | 1842.7 | 100 | 100 | 6.28 |
| | Reference Run | | | | | | | | |
| 12 | Eshmuno A - | 82.9 | 0.24 | 1.51 | 98.26 | 2427.3 | 102.3 | 99 | 4.12 |
| | Reference Run | | | | | | | | |
| 14 | Eshmuno A - | 85.3 | 0.34 | 2.19 | 97.47 | 488.0 | 97.5 | 101.9 | 4.19 |
| | Wash 2: | | | | | | | | |
| | Tris + 3M | | | | | | | | |
| | MgCl2 | | | | | | | | |
| 15 | Eshmuno A - | 107.3 | 7.24 | 15.48 | 77.29 | 230.9 | 97.2 | 88 | 6.92 |
| | Wash 2: | | | | | | | | |
| | Tris + 3M | | | | | | | | |
| | MgCl2, | | | | | | | | |
| | Elution 4M | | | | | | | | |
| | MgCl2 | | | | | | | | |
| 18 | MSS LX - | 87.9 | 1.14 | 4.58 | 94.3 | 226.3 | 105.3 | 100.6 | 6.15 |
| | Wash2: | | | | | | | | |
| | Tris + 2.8M | | | | | | | | |
| | MgCl2 | | | | | | | | |
| 20 | MSS LX - | 81 | 1 | 6.1 | 92.9 | 696.1 | 111.9 | 113 | 7.19 |
| | Wash 2: | | | | | | | | |
| | Tris + MgCl2. | | | | | | | | |
| | MgCl2 | | | | | | | | |
| | Gradient | | | | | | | | |
| | Elution | | | | | | | | |

### Example 6: Experimental Runs with a Variety of Wash Buffers

FIG. 4 illustrates a chromatography profile of run 6 as described in Table 6D. MabSelect Sure LX with urea wash conditions for step 2 in 50 mM Na-citrate, pH 4.7, followed by an incremental increase of urea (1, 2, & 3 M) and 50 mM Na-citrate pH 4.6, 3 M Urea, 10% IPA. Urea and IPA were removed with 50 mM Na-phosphate, pH 7, 2.0 M NaCl. Based on the graph, urea shows some A280 activity at the end of the wash. At pH 4.7, 2M urea and more causes significant elution of OG2072. At pH 7 and pH 8.8, 3 M urea does not lead to pre-mature elution of OG2072. As depicted, lines are identified and labeled with arrows as the following: Square, A280; plus, % Pump B activity; star, and pH. Therefore, the example demonstrates the results of chromatography run using varied urea wash conditions.

### Example 7: Experimental Runs with a Variety of Wash Buffers

FIG. 5 illustrates a chromatography profile of run 7 as described in Table 6D. MabSelect Sure LX with 50 mM Na-citrate, pH 4.7, 0.3 M urea wash. Urea and IPA were removed with 50 mM Na-phosphate, pH 7, 2.0 M NaCl. Based on the graph, not much A280 activity was found in the wash. As depicted, lines are identified and labeled with arrows as the following: Square, A280; plus, % Pump B activity; star, pH. Therefore, the example demonstrates the results of chromatography run using varied urea wash conditions.

### Example 8: Experimental Runs with a Variety of Wash Buffers

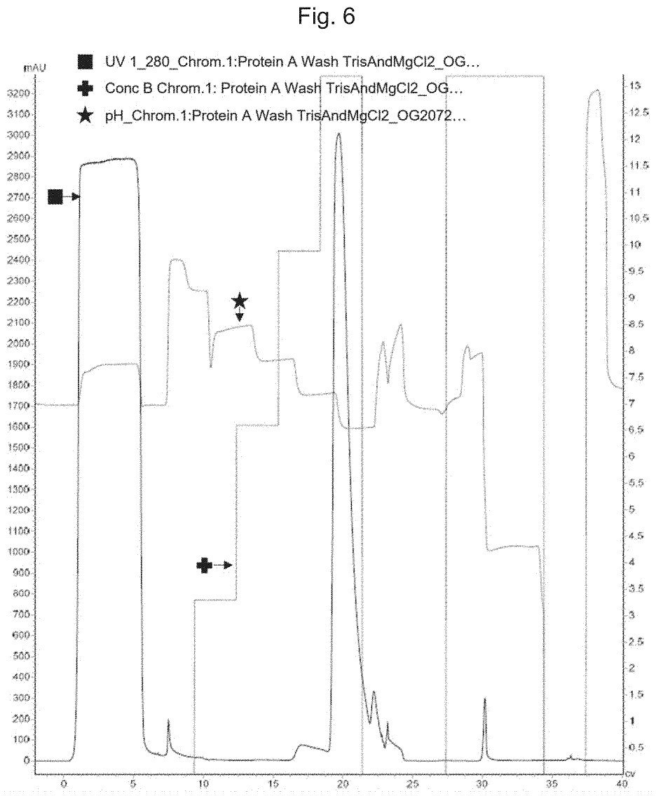

FIG. 6 illustrates a chromatography profile of run 10 as described in Table 6D. Specifically, FIG. 6 illustrates a chromatography profile wherein increasing concentrations of MgCl2 are applied, and wherein the chaotrop can be used to elute the protein of interest. MabSelect Sure LX with 50 mM Tris pH 8.8, followed by 50 mM Tris with incremental increases of MgCl2 (1, 2, 3, 4 M). MgCl2 was removed with 50 mM Na-phosphate, pH 7, 2.0 M NaCl. Before elution, the column was equilibrated with 50 mM Na-phosphate, pH 7, 250 mM NaCl. During the transition from 50 mM Tris Base, 4.0 M MgCl2 and 50 mM Na-phosphate crystal formation occurred. Phosphate ions and Magnesium ions cannot be present at the same time otherwise magnesium phosphate crystals form, which leads to a clogging of the column. The profile demonstrates that 3 M MgCl2 represents the upper limit of Chaotrop concentration without elution of the product of interest, whereas the presence of at least 4 M MgCl2 leads to quantitative elution of the product of interest. As depicted, lines are identified and labeled with arrows as the following: Square, A280; plus, % Pump B activity; star, pH. Therefore, the example demonstrates the results of chromatography run using varied MgCl2 concentrations.

### Example 9: Experimental Runs with a Variety of Wash Buffers

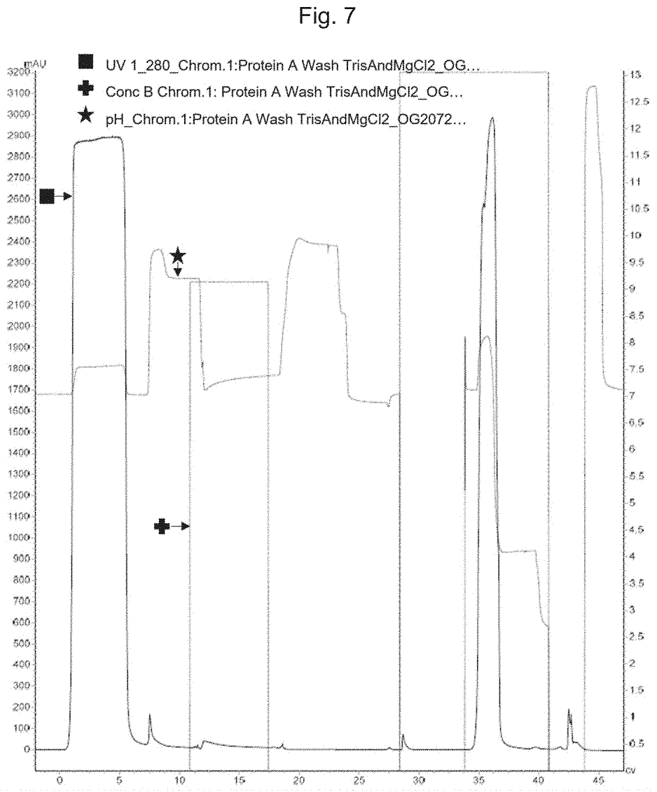

FIG. 7 illustrates a chromatography profile of run 18 as described in FIG. 6. MabSelect Sure LX with 50 mM Tris pH 8.8, followed by 50 mM Tris with 2.8 M MgCl2. MgCl2 was removed with 50 mM Tris, pH 8.8 before 50 mM Na-phosphate, pH 7, 2 M NaCl was applied. Before elution, the column was equilibrated with 50 mM Na-phosphate, pH 7, 250 mM NaCl. As depicted, lines are identified and labeled with arrows as the following: Square, A280; plus, % Pump B activity; star, pH. Therefore, the example demonstrates the results of chromatography run using a set MgCl2 concentration.

### Example 10: Column Purification Protocol Using Tris-Based Buffers

Alternative buffer compositions were contemplated and analyzed during column purification. Table 6G illustrates an embodiment of the process for the column purification of OG2072 antibody from an animal component free cell culture process. Buffers for equilibration and wash steps are comprised of Tris, alone or in combination with either NaCl or MgCl2. Equilibration is carried out with 50 mM Tris, pH 7.2, and 250 mM NaCl. Wash 1 involves 50 mM Tris, pH 7.2, 250 mM NaCl, whereas wash 2 comprises 50 mM Tris, pH 7.8, 2.8 M MgCl2, and wash 3 is carried out by 50 mM Tris, pH 8.8. Wash 4 then proceeds with 50 mM Tris, pH 7.2, 250 mM NaCl. Results demonstrate that an embodiment of the column protocol may be achieved using alternative buffers. In some embodiments, the alternative buffers are primarily comprised of Tris. Therefore, the example demonstrates the use of Tris based buffers.

TABLE 6G
Chaotropic Chromatography
Purification WITH ALTERNATIVE BUFFER.

| | | | Flow |
|---|---|---|---|
| Step | Buffer | CV | rate (cm/h) |
| Equilibration | 50 mM Tris, 250 mM NaCl, pH 7.2 | 5 | 300 |
| Load | | | 200 |
| Wash 1 | 50 mM Tris, 250 mM NaCl, pH 7.2 | 2 | 200 |
| Wash 2 | 50 mM Tris, pH 7.8, 2.8 M MgCl₂ | 3 | 100 |
| Wash 3 | 50 mM Tris, pH 8.8 | 3 | 100 |
| Wash 4 | 50 mM Tris, 250 mM NaCl, pH 7.2 | 3 | 300 |
| Elution | 10 mM Na-Formate pH 3.5 | 4 | 300 |
| Post Elution | 100 mM Citric Acid, pH 2.1 | 2 | 300 |
| CIP | 0.1 NaOH | 3 | 300 |

### Example 11

This example illustrates a method of purifying a product using affinity chromatography. One first loads an eluent into an affinity chromatography matrix, where the affinity chromatography matrix binds to a protein of interest. One then washes the affinity chromatography matrix with a buffer solution comprising a chaotropic agent. Fluid collected from the wash comprises HCP species. The protein is then collected using an elution solution. Thus, the example demonstrates a method of purifying a product.

### Example 12

This example illustrates a method of purifying a product and reducing impurities from a load fluid. The load fluid comprises a protein and one or more impurities, and the load fluid is passed through an affinity chromatography matrix. Next, at least one wash solution comprising a chaotropic salt is applied to the matrix. The protein is then collected using an elution solution. Thus, the example demonstrates a method of purifying a product.

### Example 13

This example illustrates a method of separating impurities in an eluate comprising a protein of interest. The method comprises loading an eluent comprising a protein of interest onto an affinity chromatography matrix. One then washes the affinity chromatography matrix with one or more buffer solutions comprising magnesium or a magnesium salt. Fluid collected from the wash comprises HCP species. The protein is then collected using an elution solution. Thus, the example demonstrates a method of separating impurities in an eluate comprising a protein of interest.

### Example 14

This example illustrates a method of producing a product using affinity chromatography. First, one loads an eluent containing a protein of interest onto an affinity chromatography matrix. One then performs a first wash of the affinity chromatography matrix with a first buffer comprising sodium phosphate and a salt. One then performs a second wash of the affinity chromatography matrix with a second buffer comprising a chaotropic agent. Fluid collected from the wash comprises HCP species. The protein is then collected using an elution solution. Thus, the example demonstrates a method of producing a product using affinity chromatography.

### Example 15

This example illustrates a method producing a product using affinity chromatography. One first loads an eluent containing a protein of interest onto an affinity chromatography matrix. One then performs a first wash with a first buffer containing Tris and a salt. Then, one performs a second wash with a second buffer containing Tris and a chaotropic agent, wherein the second buffer chaotropic agent is not the same salt as contained in the first buffer. Fluid collected from the wash comprises HCP species. The protein is then collected using an elution solution. Thus, the example demonstrates a method of producing a product using affinity chromatography.

### Example 16

This example illustrates a method of producing a product. First, one collects a load fluid, wherein the load fluid comprises a protein of interest. One then loads the load fluid onto an affinity chromatography matrix, wherein the affinity chromatography matrix binds to the protein of interest. One then washes the affinity chromatography matrix with a buffer solution comprising a chaotropic salt. One then elutes the bound protein of interest, and collects an eluate, wherein the eluate contains the protein of interest. Thus, the example demonstrates a method of producing a product.

### Example 17

This example illustrates a method of producing a product. First, one collects a load fluid, wherein the load fluid comprises a protein of interest. One then loads the load fluid onto an affinity chromatography matrix, wherein the affinity chromatography matrix binds to the protein of interest. One then feeds the affinity chromatography matrix with a buffer solution comprising a chaotropic salt. One then elutes the bound protein of interest, and collects an eluate, wherein the eluate contains the protein of interest. Thus, the example demonstrates a method of producing a product.

### Example 18

This example illustrates a method of producing a product. First, one collects conjugate protein, wherein the conjugate protein comprises an antibody bound to a conjugate polymer. One then loads the conjugate protein onto an affinity chromatography matrix, wherein the affinity chromatography matrix binds to the conjugate protein, then one washes the affinity chromatography matrix with a buffer solution comprising a chaotropic salt. Then, one elutes the conjugate protein, and collects an eluate, wherein the eluate contains the conjugate protein. Thus, the example demonstrates a method of producing a product.

### Example 19

This example illustrates a method of producing a product. First, one washes an affinity chromatography matrix bound to a target protein of interest, the wash is with a buffer comprising a chaotropic salt. One then elutes and collects an eluate, where the eluate contains the target protein of interest. One then removes viral contaminants from the eluate. Thus, the example demonstrates a method of producing a product.

### Example 20

This example illustrates a method of producing a product. One first washes an affinity chromatography matrix bound to a target protein of interest with a buffer comprising a chaotropic salt. One then removes the chaotropic salt, and elutes the column to collect an eluate, wherein the eluate contains the target protein of interest. Thus, the example demonstrates a method of producing a product.

### Example 21

This example illustrates a method of producing a product. First, one collects a load fluid, wherein the load fluid is comprised of a protein of interest. Then, one loads the load fluid into an affinity chromatography matrix, wherein the affinity chromatography matrix binds to the protein of interest. One then washes the affinity chromatography matrix with a buffer solution, the buffer solution comprising a chaotropic salt. One then elutes and collects an eluate, wherein the eluate contains the target protein of interest. One then removes viral contaminants from the eluate. Thus, the example demonstrates a method of producing a product.

### Example 22

This example illustrates a method of producing a product. First, one loads an eluent into an affinity chromatography matrix. One then washes with a first wash buffer. One then washes with a second wash buffer comprising a chaotropic salt. One then washes with a third wash buffer, wherein the third wash buffer removes the chaotropic salt. One then elutes with an elution buffer, wherein an eluate is collected, wherein the eluate comprises a protein product. Thus, the example demonstrates a method of producing a product.

### Example 23

This example illustrates a method of producing a product. First, one recovers a cell culture supernatant, wherein the cell culture supernatant comprises a protein of interest. One then processes the cell culture supernatant into an eluent, wherein the eluent comprises the protein of interest. One then loads the eluent into an affinity chromatography matrix. One then washes with a first wash buffer comprising Tris or Sodium Phosphate. One then washes with a second wash buffer comprising a chaotropic salt. One then elutes with an elution buffer, wherein an eluate is collected, wherein the eluate comprises a protein product. One then inactivates the viral contaminants present in the eluate with a low pH viral buffer to yield a viral inactivated eluate. One then filters the viral inactivated eluate, performing at least one round of ion exchange chromatography on the viral inactivated eluate, and filtering the viral inactivated eluate to yield a retentate, wherein the retentate comprises the protein of interest. Thus, the example demonstrates a method of producing a product.

### Example 24

This example illustrates a method of purifying a protein using affinity chromatography. One first contacts a load fluid with a medium, wherein the medium is an affinity chromatography matrix that binds a protein of interest. One then washes the medium with a buffer solution comprising a chaotropic agent, wherein the chaotropic agent is a salt. One then contacts the washed medium with an elution solution under conditions suitable for eluting the protein of interest. Thus, the example demonstrates a method of purifying a protein.

### Example 25

This example illustrates a method of producing a product. First, one applies a solution containing a protein of interest onto an affinity chromatography matrix. One then washes the affinity chromatography matrix with a first buffer. One then washes the affinity chromatography matrix with a second buffer containing a chaotropic agent. One then washes the affinity chromatography matrix with a third buffer to remove the chaotropic agent. One then elutes with an elution buffer, wherein an eluate is collected, wherein the eluate comprises a protein product. Thus, the example demonstrates a method of producing a product.

### Example 26

This example illustrates a system for protein purification. The system comprises a column having a first antigen binding protein bound to the column, a phosphate wash buffer comprising sodium phosphate and a salt, an intermediate wash buffer comprising tris, a second wash buffer comprising magnesium chloride, and an elution buffer comprising sodium formate, the system is configured to reduce HCP species and purify a protein. A protein is collected wherein HCP species are reduced following use of said system. Thus, the example demonstrates a system for protein purification.

### Example 27

This example illustrates a system for protein purification. The system comprises a column having a having a first antigen binding protein bound to the column, a first tris wash buffer comprising tris and a salt, an intermediate tris wash buffer, a second wash buffer comprising magnesium chloride, and an elution buffer comprising sodium formate, the system is configured to reduce HCP species and purify a protein. A protein is collected wherein HCP species are reduced following use of said system. Thus, the example demonstrates a system for protein purification.

### Example 28

This example illustrates a system for antibody purification. The system comprises a column having a protein A resin bound to an antibody. Wherein the antibody comprises a light and heavy chain of at least one of SEQ ID NOs: 91-93, 28-30, and at least one of SEQ ID NOs: 7-13, 19-27, 89, 90, 256-262, respectively, and a chaotropic wash buffer comprising a chaotropic salt, and an elution buffer comprising sodium formate. The system is configured to reduce HCP species and purify a protein. A protein is collected wherein HCP species are reduced following use of said system. Thus, the example demonstrates a system for antibody purification.

### Example 29

This example illustrates a method for processing a product. First, one loads an eluent into an affinity chromatography matrix. One then washes with a wash buffer comprising a chaotropic salt to collect an eluate, wherein the concentration of the chaotropic salt is increased from a first concentration to a second concentration, wherein the eluate is collected in at least one fraction, and wherein the at least one fraction comprises a product of interest. Thus, the example demonstrates a method for the elution and collection of a product of interest from a fraction of an eluate.

### Example 30

A reference run to purify both OG1950 and virus inactivated OG1950 was performed according to the steps laid out in Table 6H, while a set of experimental runs was performed to purify both OG1950 and virus inactivated OG1950 according to the steps presented in Table 61.

For the reference runs, a cleaning in place step was run using 100 mM NaOH, followed by an equilibration using 50 mM Na-Phosphate, 250 mM NaCl, at pH 7. Protein sample was then loaded onto the column. Washes 1-3 were then performed sequentially according to Table 6H. Elution followed using 10 mM Na-Formate at pH 3.5, wherein a strip and cleaning in place process were then performed. CV and flow rates conditions are noted in Table 6H. A cleaning in place step was run using 100 mM NaOH, followed by an equilibration using 50 mM Na-Phosphate, 250 mM NaCl, at pH 7. Protein sample was then loaded onto the column. Washes 1-6 were then performed sequentially according to Table 61. Elution followed using 10 mM Na-Formate at pH 3.5, wherein a strip and cleaning in place process were then performed.

Table 6J describes the fold HCP reduction and collected protein concentration (OG1950 conc. [mg/mL]). The experimental run using magnesium chloride-based wash buffer reduced HCP content approximately 5-fold compared to reference run conditions.

TABLE 6H
Reference Run

| | | | Flow |
|---|---|---|---|
| | | | rate |
| Step | Buffer | CV | [cm/h] |
| CIP 1 | 100 mM NaOH (15 min. hold after 2 CV) | 3 | 400 |
| Equilibration | 50 mM Na-Phosphate, 250 mM NaCl, pH 7.0 | 5 | 400 |
| Load | | n/a | 400 |
| Wash 1 | 50 mM Na-Phosphate, 250 mM NaCl, pH 7.0 | 2 | 400 |
| Wash 2 | 50 mM Na-Phosphate, 2 M NaCl, pH 7.0 | 4 | 400 |
| Wash 3 | 50 mM Na-Phosphate, 250 mM NaCl, pH 7.0 | 2 | 400 |
| Elution | 10 mM Na-Formate, pH 3.5 | 5 | 400 |
| Strip | 100 mM Citric acid, pH 2.1 | 2 | 400 |
| CIP 2 | 100 mM NaOH (15 min. hold after 2 CV) | 3 | 400 |

TABLE 6I
Experimental Run

| | | | Flow |
|---|---|---|---|
| | | | rate |
| Step | Buffer | CV | [cm/h] |
| CIP 1 | 100 mM NaOH (15 min. hold after 2 CV) | 3 | 400 |
| Equilibration | 50 mM Na-Phosphate, | 5 | 400 |
| Load | 250 mM NaCl, pH 7.0 | n/a | 400 |
| Wash 1 | 50 mM Na-Phosphate, 250 mM NaCl, pH 7.0 | 2 | 400 |
| Wash 2 | 50 mM Tris, pH 8.8 | 4 | 400 |
| Wash 3 | 100 mM Tris base, 2.8 M MgC12 | 4 | 100 |
| Wash 4 | 50 mM Tris, pH 8.8 | 1.2 | 100 |
| Wash 5 | 50 mM Tris, pH 8.8 | 2.8 | 400 |
| Wash 6 | 50 mM Na-Phosphate, 250 mM NaCl, pH 7.0 | 2 | 400 |
| Elution | 10 mM Na-Formate, pH 3.5 | 5 | 400 |
| Strip | 100 mM Citric acid, pH 2.1 | 2 | 400 |
| CIP 2 | 100 mM NaOH (15 min. hold after 2 CV) | 3 | 400 |

TABLE 6J
HCP and Protein Concentration Results

| | OG1950 | HCP |
|---|---|---|
| Sample description | conc. [mg/mL] | [ng/mg] |
| Mab Select Sure Eluate—Reference run | 13.42 | >1001 |
| Virus inactivated—Reference run | 11.76 | 1023 |
| Mab Select Sure Eluate—MgCl2 wash | 13.24 | 266 |
| Virus inactivated—MgCl2 wash | 11.57 | 215 |

### Example 31

The platform process previously used is as shown in Table 7A. Further embodiments of the present disclosure is as shown in Table 7B.

TABLE 7A
Platform process for Protein A affinity Chromotography

| | | | | | Flow | |
|---|---|---|---|---|---|---|
| | | | pump B | | rate | |
| Step | Inlet | Buffer | [%] | CV | [cm/h] | Outlet |
| Rinse 1 | B4 | Milli Q H20 | 100% | 2 | 200 | W |
| CIP 1 | A3 | 0.1M NaOH (15 min. hold after 2 CV) | 0% | 3 | 300 | W |
| Equilibration | A1 | 50 mM Na-Phosphate, 250 mM NaCl, pH 7.0 | 0% | 5 | 300 | W |
| Load | S3 | | 186.2 | mL (LV) | 200 | W |
| Wash 1 | A1 | 50 mM Na-Phosphate, 250 mM NaCl, pH 7.0 | 0% | 2 | 200 | W |
| Wash 2 | A4 | 50 mM Na-Phosphate, 2.0M NaCl, pH 7.0 | 0% | 4 | 300 | W |
| Wash 6 | A1 | 50 mM Na-Phosphate, 250 mM NaCl, pH 7.0 | 0% | 2 | 300 | W |
| Elution | B1 | 10 mM Na-Formate pH 3.5 | 100% | 5 | 300 | O1 |
| post Elution | B3 | 100 mM Citric acid pH 2.1 | 100% | 2 | 300 | W |
| CIP 2 | A3 | 0.1M NaOH (15 min. Hold after 2 CV) | 0% | 3 | 300 | W |

TABLE 7B
Platform process 2 for Protein A affinity Chromotography

| | | | | | Flow | |
|---|---|---|---|---|---|---|
| | | | pump B | | rate | |
| Step | Inlet | Buffer | [%] | CV | [cm/h] | Outlet |
| Rinse 1 | B4 | Milli Q H20 | 100% | 2 | 200 | W |
| CIP 1 | A3 | 0.1M NaOH (15 min. hold after 2 CV) | 0% | 3 | 300 | W |
| Equilibration | A1 | 50 mM Na-Phosphate, 250 mM NaCl, pH 7.0 | 0% | 5 | 300 | W |
| Load | S3 | | 186.2 | mL (LV) | 200 | W |
| Wash 1 | A1 | 50 mM Na-Phospate, 250 mM NaCl, pH 7.0 | 0% | 2 | 200 | W |
| Wash 2 | A4 | 50 mM Tris, pH 8.8 | 0% | 4 | 300 | W |
| Wash 3 | A2 | 100 mM Tris base, pH 7.7; 2.8M MgCl2 | 0% | 4 | 100 | W |
| Wash 4 | A4 | 50 mM Tris, pH 8.8 | 0% | 1.2 | 100 | W |
| Wash 5 | A4 | 50 mM Tris, pH 8.8 | 0% | 2.8 | 300 | W |
| Wash 6 | A1 | 50 mM Na-Phosphate, 250 mM NaCl, pH 7.0 | 0% | 2 | 300 | W |
| Elution | B1 | 10 mM Na-Formate pH 3.5 | 100% | 5 | 300 | O1 |
| post Elution | B3 | 100 mM Citric acid pH 2.1 | 100% | 2 | 300 | W |
| CIP 2 | A3 | 0.1M NaOH (15 min. Hold after 2 CV) | 0% | 3 | 300 | W |

The process as outlined in Table 7B was then further refined as described herein. For solutions whereby crystallization is an issue, the process as outline in Table 7B would be the preferred process. For example, when using magnesium chloride in solution the number of wash steps is higher to prevent the interaction between phosphate buffer and magnesium salts leading to rapid crystallization. The number of wash steps is also high when using calcium salts, in order to prevent interaction between phosphate buffer and calcium salts. However, for guanidinium salts, crystallization is not observed to be a significant issue, so a process such as that depicted in Table 7A can be utilized, wherein the second wash step is replaced with 1 M GuHCl.

### Establishing Ranges for Equilibration and Wash Buffer Parameters

A platform process was performed by applying the antibody construct to a Protein A affinity resin in physiological conditions, as shown in Table 8A.

TABLE 8A

| | | | | Chromatography | | Virus inactivated | | | |
|---|---|---|---|---|---|---|---|---|---|
| | | | | Eluate | | protein solution | | | |
| | | | | SEC- | | SEC- | | | |
| | | | | HPLC | | HPLC | | | Relative |
| | | | | purity | HCP | purity | HCP | Potency | potency |
| Run | Protein | Resin | Purification strategy | (%) | (ng/mg) | (%) | (ng/mg) | (pM) | (%) |
| 1b | OG2072 | MSS | Equil + Wash 1: 50 mM Na-Phosphate, | 98.4 | 241 | 98.3 | 193 | 304 | 121 |
| | | LX | pH 7.0, 250 mM NaCl | | | | | | |
| | | | Wash 3: 100 mM Tris base, 2.8M MgCl2 | | | | | | |
| 2 | OG2072 | MSS | Equil + Wash 1: 5 mM Na-Phosphate, | 98.1 | 386 | 98.1 | 355 | n/a | n/a |
| | | LX | pH 7.0, 50 mM NaCl | | | | | | |
| | | | Wash 3: 100 mM Tris base, 2.8M MgCl2 | | | | | | |
| 3 | OG2072 | MSS | Equil + Wash 1: 200 mM Na-Phosphate, | 98.2 | 410 | 98.1 | 279 | n/a | n/a |
| | | LX | pH 7.0, 2000 mM NaCl | | | | | | |
| | | | Wash 3: 100 mM Tris base, 2.8M MgCl2 | | | | | | |
| 4 | OG2072 | MSS | Equil + Wash 1: 50 mM Na-Phosphate, | 98.2 | 259 | 98.2 | 254 | n/a | n/a |
| | | LX | pH 6.0, 250 mM NaCl | | | | | | |
| | | | Wash 3: 100 mM Tris base, 2.8M MgCl2 | | | | | | |
| 5 | OG2072 | MSS | Equil + Wash 1: 50 mM Na-Phosphate, | 97.9 | 327 | 98.2 | 311 | n/a | n/a |
| | | LX | pH 9.0, 250 mM NaCl | | | | | | |
| | | | Wash 3: 100 mM Tris base, 2.8M MgCl2 | | | | | | |
| 6 | OG2072 | MSS | Equil + Washes (Tris only): 50 mM Tris, | 97.9 | 340 | 97.8 | 296 | 336 | 110 |
| | | LX | pH 7.2, 250 mM NaCl | | | | | | |
| | | | Wash 3: 100 mM Tris base, 2.8M MgCl2 | | | | | | |
| 22b | OG2072 | MSS | Platform process: Wash with 50 mM | 98.4 | 1285 | 98.5 | 562 | 330 | 114 |
| | | LX | Na-Phosphate, pH 7.0, 2.0M NaCl | | | | | | |
| | | | (no MgCl2 in any wash buffer) | | | | | | |

The equilibration and wash 1 buffer was composed of 50 mM Na-Phosphate, 250 mM NaCl, pH 7.0. The resin was then washed by applying a high salt wash buffer 2 (50 mM Na-Phosphate, 2.0 M NaCl, pH 7.0) followed by re-equilibrating the resin with the first buffer before the antibody is eluted (10 mM Na-Formate pH 3.5). It was attempted to alleviate the HCP levels in the eluate of this platform process. It was shown that magnesium chloride is effective in further removing HCPs. As magnesium forms crystals in the presence of phosphate, a wash was introduced (50 mM Tris, pH 8.8) to remove the phosphate before the magnesium-containing buffer was applied. After the wash with magnesium-containing buffer was completed, the same wash was applied again to remove the magnesium from the column before the sodium phosphate buffer was applied. This measure was needed to allow for the magnesium wash to be introduced on the existing platform process. A concentration of 2.8 M MgCl2 was found to work well for the purpose (run 1b). In an initial set of experiments buffer concentrations and salt concentrations were tested to define suitable operating ranges for the buffer system and salt concentration.

In run 2 and 3, 5 mM Na-Phosphate, 50 mM NaCl, pH 7.0 and 200 mM Na-Phosphate, 2.0 M NaCl, pH 7.0 were tested for equilibration and post-load wash, while keeping the 100 mM Tris base, 2.8 M MgCl2 for the wash 3 step. The higher and lower buffer concentrations as well as NaCl concentrations showed comparable levels of HCP for the eluate of the affinity chromatography step and the efficiency in HCP removal is not impacted. This shows that a range of 5 mM to 200 mM works for the buffer system and 50 mM to 2.0 M for the NaCl.

In run 4 and 5, 50 mM Na-Phosphate, 250 mM NaCl, pH 6.0 and 50 mM Na-Phosphate, 250 mM NaCl, pH 9.0 were tested for equilibration and post-load wash, while keeping the 100 mM Tris base, 2.8 M MgCl2 for the wash 3 step. The eluate of the runs with the higher and lower pH values applied for equilibration and wash showed comparable HCP levels. There is no impact on the efficiency of HCP and shows that a range of pH 6.0-9.0 for the buffer system works well.

The current platform uses 50 mM Na-Phosphate, 250 mM NaCl, pH 7.0 for equilibration and post-load wash steps. An experiment (run 6) was performed to test the feasibility of the chromatography step with Tris buffer only. In this experiment 50 mM Tris, 250 mM NaCl, pH 7.2 was used for equilibration and wash (instead of 50 mM Na-Phosphate, 250 mM NaCl, pH 7.0). The results looked comparable and demonstrates that the extra washes (50 mM Tris, pH 8.8)—to remove the phosphate ions before applying the magnesium ions—can be avoided can be avoided when the affinity chromatography step is performed with Tris buffer only. This simplifies the chromatography lay-out and reduces buffer volumes.

In summary, the higher and lower buffer concentrations and NaCl concentrations as well as higher and lower pH values for the equilibration and wash steps showed comparable HCP removal efficiency. This establishes an operational range for (1) the strength of the buffer system of 5 mM to 200 mM; (2) the NaCl concentration of 50 mM to 2.0 M for the NaCl; (3) pH of 6.0-9.0 and (4) Tris and phosphate buffers can be used. An additional experiment with wash 3: 0.1 M Bis-Tris, pH 6.0, 2.8 M MgCl2 was performed and worked well albeit a low step yield. For this buffer system a lower MgCl2 would need to be established.

### Establishing Range for the Magnesium Chloride Concentration

Then, the inventors tested the impact of MgCl2 concentrations in the wash 3 step on the HCP content of the eluate, as shown in Table 8B.

TABLE 8B

| | | | | Chromatography | | | | | |
|---|---|---|---|---|---|---|---|---|---|
| | | | | Eluate | | Virus inactivated protein solution | | | |
| | | | | SEC- | | SEC- | | | |
| | | | | HPLC | | HPLC | | | Relative |
| | | | | purity | HCP | purity | HCP | Potency | potency |
| Run | Protein | Resin | Purification strategy | (%) | (ng/mg) | (%) | (ng/mg) | (pM) | (%) |
| 7 | OG2072 | MSS | Wash 3: 0.1M Tris base, 1.5M MgCl2 | 97.8 | 415 | 97.8 | 368 | n/a | n/a |
| | | LX | | | | | | | |
| 9 | OG2072 | MSS | Wash 3: 0.1M Tris base, 2.0M MgCl2 | 98.2 | 255 | 98.1 | 230 | n/a | n/a |
| | | LX | | | | | | | |
| 10 | OG2072 | MSS | Wash 3: 0.1M Tris base, 2.5M MgCl2 | 98.3 | 276 | 98.2 | 221 | n/a | n/a |
| | | LX | | | | | | | |
| 1b | OG2072 | MSS | Wash 3: 100 mM Tris base, 2.8M MgCl2 | 98.4 | 241 | 98.3 | 193 | 304 | 121 |
| | | LX | | | | | | | |
| 11 | OG2072 | MSS | Wash 3: 0.1M Tris base, 3.0M MgCl2 | 98.5 | 158 | 98.3 | 136 | 314 | 117 |
| | | LX | | | | | | | |
| 22b | OG2072 | MSS | Platform process: Wash with 50 mM | 98.4 | 1285 | 98.5 | 562 | 330 | 114 |
| | | LX | Na-Phosphate, pH 7.0, 2.0M NaCl | | | | | | |

Run 7 to run 11 showed that there is an inverse correlation of the MgCl2 concentration in the wash step and the HCP content in the eluate. The yield is good for all runs except run 11 as 3.0 M MgCl2 is somewhat too high and has a yield of 66% for the affinity chromatography step. All other runs had yields >85%. The SEC-HPLC purity is comparable for all runs. Run 12 data not shown as 3.5 M MgCl2 was too high and OG2072 eluted in the wash fraction. From these experiments with MgCl2 and the effect of the concentration, the inventors observed that concentration has an effect, but presence of magnesium (in lieu of sodium) was more important than the concentration. the inventors conclude this as 1.5 M MgCl2 showed less HCP in the eluate than 2.0 M NaCl.

Run 13 was performed with 2.8 M MgCl2 supplemented to a Bis-Tris buffered wash buffer at pH 6.0. The HCP content is 94 and ng/mg for the eluate pool and after VI, respectively. This is 12 and 5-fold less as compared to the platform conditions. However, the yield was low at 48.4% as a significant amount of OG2072 was eluting in the wash step. This is thereby not an economically viable wash step. The MgCl2 content would need to be lowered and fine-tuned if the magnesium/containing wash buffer has a pH 6.0. Note, run 8 was performed with 2.8 M MgCl2 supplemented to a 100 mM Tris buffer at pH 7.0 and a good step yield was observed.

There was an inverse correlation of the MgCl2 concentration of the wash 3 buffer and the HCP content in the eluate. The additional efficiency in removing HCP by increasing the MgCl2 concentration (beyond 2.0 M MgCl2) was rather modest. The yield was good for all runs except for the run 3.0 M MgCl2 in the wash 3 buffer, where some of the antibody was eluting in the wash buffer. The SEC-HPLC purity was comparable for all runs. From these experiments with MgCl2 and the effect of the concentration, the inventors observed that concentration has an effect, but that the presence of magnesium (in lieu of sodium) was more important than the concentration. The inventors concluded that a buffer containing 1.5 M MgCl2 showed less HCP in the eluate than 2.0 M NaCl.

### Example 32: Screening of Salts of Different Chaotropic Strengths

Then, a test was run to determine whether the removal of HCP correlates with the strength of the chaotrop following the Hofmeister series.

### Screening of Chaotropic Cations

The experimental conditions were as shown in Table 9A.

TABLE 9A

| | | | | Chromatography | | | | | |
|---|---|---|---|---|---|---|---|---|---|
| | | | | Eluate | | Virus inactivated protein solution | | | |
| | | | | SEC- | | SEC- | | | |
| | | | | HPLC | | HPLC | | | Relative |
| | | | | purity | HCP | purity | HCP | Potency | potency |
| Run | Protein | Resin | Purification strategy | (%) | (ng/mg) | (%) | (ng/mg) | (pM) | (%) |
| 19 | OG2072 | MSS | Wash 3: 0.1M Tris base, 2.8M Li- | 98.1 | 444 | 98.1 | 390 | 321 | 117 |
| | | LX | Acetate | | | | | | |
| 14 | OG2072 | MSS | Wash 3: 0.1M Tris base, 2.8M LiCl | 98.1 | 408 | 97.8 | 353 | n/a | n/a |
| | | LX | | | | | | | |
| 1b | OG2072 | MSS | Wash 3: 100 mM Tris base, 2.8M MgCl2 | 98.4 | 241 | 98.3 | 193 | 304 | 121 |
| | | LX | | | | | | | |
| 23 | OG2072 | MSS | Wash 3: 0.1M Tris base, 2.0M CaCl2° | 97.6 | 157 | 98.1 | 147 | 299 | 126 |
| | | LX | | | | | | | |
| 29 | OG2072 | MSS | Wash 3: 0.1M Tris base, 1.0M | 98.9 | 86 | 98.7 | 86 | n/a | n/a |
| | | LX | Guanidinium chloride* | | | | | | |
| 22b | OG2072 | MSS | Platform process: Wash with 50 mM Na- | 98.4 | 1285 | 98.5 | 562 | 330 | 114 |
| | | LX | Phosphate, pH 7.0, 2.0M NaCl | | | | | | |

For this, a wash step with 5.0 M CaCl2) supplemented to Tris base was performed (data not shown), but the run had a low yield as OG2072 eluted in the wash fraction demonstrating that the chaotropic effect is too strong at 5.0 M. A follow-up run was performed where the wash buffer was supplemented with 2.0 M CaCl2). This run showed a good yield on the affinity chromatography step. There was no evidence of aggregation due to the presence of 2.0 M CaCl2). The HCP content was much lower as compared to the platform process that uses 2.0 M NaCl for the wash step. This shows that the benefit of the CaCl2) wash comes from the calcium component. Calcium is a more chaotropic cation than sodium. Importantly, the eluate of the run with 2.0 M CaCl2) had a lower HCP content than the eluate of the run with 2.8 M MgCl2 in the wash step. This supports the hypothesis that stronger chaotrops have a more beneficial effect on the HCP removal as calcium is a stronger chaotrop than magnesium per the Hofmeister series.

The strongest chaotropic cation used in the industry is guanidinium. Run 16 showed that 3.0 M Guanidinium chloride is too high for the wash step. It had a low yield and resulted in elevated aggregate levels. Run 24 showed that 1.65 M is still a slightly too high concentration of Guanidinium chloride as the SEC-HPLC purity is at 95.8% as compared to approx. 98% for conditions that did not have an impact on the SEC-HPLC and are comparable to the platform process. Run 29 showed that 1.0 M Guanidinium chloride is appropriate as this run showed good yields and purity as measured by SEC-HPLC. The HCP content of the eluate was 86 ng/mg and about 15-fold lower than the platform process.

In conclusion, using sodium as chaotropic cation as part of the salt in the wash buffer resulted in a HCP content of 1285 ng/mg, lithium as chaotropic cation as part of the salt in the wash buffer resulted in HCP contents of 408 ng/mg, magnesium as chaotropic cation as part of the salt in the wash buffer resulted in HCP contents of 241 ng/mg, calcium as chaotropic cation as part of the salt in the wash buffer resulted in HCP contents of 157 ng/mg and guanidinium as chaotropic cation as part of the salt in the wash buffer resulted in HCP contents of 86 ng/mg. This follows the chaotropic strength as described in the Hofmeister series and suggests that the HCP removal is a function of the strength of the chaotropic cation as part of the salt used in the wash buffer. In other words, the stronger chaotropic agents have a higher HCP removal efficiency (Na+<Li+<Mg2+<Ca2+<Guanidinium+). Of note, Li-Acetate and LiCl showed a comparable efficiency in HCP removal with chloride performing slightly better, but the impact of the anion appeared less pronounced.

### Screening of Chaotropic Anions

The experimental conditions were as shown in Table 9B.

TABLE 9B

| | | | | Chromatography | | | | | |
|---|---|---|---|---|---|---|---|---|---|
| | | | | Eluate | | Virus inactivated protein solution | | | |
| | | | | SEC- | | SEC- | | | |
| | | | | HPLC | | HPLC | | | Relative |
| | | | | purity | HCP | purity | HCP | Potency | potency |
| Run | Protein | Resin | Purification strategy | (%) | (ng/mg) | (%) | (ng/mg) | (pM) | (%) |
| 19 | OG2072 | MSS | Wash 3: 0.1M Tris base, 2.8M Li- | 98.1 | 444 | 98.1 | 390 | 321 | 117 |
| | | LX | Acetate | | | | | | |
| 14 | OG2072 | MSS | Wash 3: 0.1M Tris base, 2.8M LiCl | 98.1 | 408 | 97.8 | 353 | n/a | n/a |
| | | LX | | | | | | | |
| 22b | OG2072 | MSS | Platform process: Wash with 50 mM Na- | 98.4 | 1285 | 98.5 | 562 | 330 | 114 |
| | | LX | Phosphate, pH 7.0, 2.0M NaCl | | | | | | |
| 18 | OG2072 | MSS | Wash 3 gradient: 0.1M Tris base, | 98.0 | 471 | 98.0 | 306 | 283 | 130 |
| | | LX | 7.0 M NaNO3 | | | | | | |
| 17b | OG2072 | MSS | Wash 3: 0.1M Tris base, 2.8M MgSO4 | 98.1 | 412 | 97.9 | 319 | 325 | 113 |
| | | LX | | | | | | | |
| 1b | OG2072 | MSS | Wash 3: 100 mM Tris base, 2.8M MgCl2 | 98.4 | 241 | 98.3 | 193 | 304 | 121 |
| | | LX | | | | | | | |

Then, a test was run to determine the impact of the anions on the removal of HCPs. To elucidate whether the beneficial effect on the HCP content is contributed by the magnesium or from the chloride, MgSO4 was tested as salt. Sulfate is a strong kosmotropic anion and often used as ammonium sulfate or sodium sulfate to induce higher order oligomeric structures e.g. for the precipitation or crystallization of proteins. In contrast, chaotropes can be used to keep molecules in solution and prevent unfavorable or unspecific interactions. In Run 17b a wash step with 2.8 M MgSO4 was applied and the eluate contained OG2072 with a lower HCP content than the platform process and a comparable HCP content to the reference run with 2.8 M MgCl2. This run also showed good yields and purity as measured by SEC-HPLC. This suggests that the main benefit of the MgCl2 wash comes from the magnesium component and not from the chloride component. Magnesium is a stronger chaotropic cation then sodium and is the likely the reason why this wash strategy is better than the platform strategy. Importantly, it shows that the main benefit comes from the cation and not from the anion. The kosmotropic property of the sulfate anion cannot revert the chaotropic effect of the magnesium.

In run 18, the wash step included 7.0 M NaNO3 as salt, where the nitrate is more chaotropic than the chloride in the platform process. The eluate contained OG2072 with a lower HCP content than the platform process, but a higher HCP content compared to the reference run with 2.8 M MgCl2 despite the much higher concentration of 7.0 M NaNO3 applied. This further corroborates the hypothesis that the cation is the more potent parameter in the HCP removal as compared to the anion. To further test this hypothesis, in run 19 the wash buffer was prepared including 2.8 M Li-acetate. Lithium is more chaotropic cation as compared to sodium, but acetate is less chaotropic than chloride. The eluate contained OG2072 with a lower HCP content than the platform process with NaCl. This shows the beneficial effect of the cation that is not reversed by the more kosmotropic anion present.

In summary, using 2.8 M MgSO4 as wash buffer supplement resulted in an eluate with a lower HCP content than the platform process and somewhat higher HCP content when compared to the run with 2.8 M MgCl2 as wash buffer supplement. This shows that the main benefit in HCP removal comes from the cation and not from the anion. The kosmotropic property of the sulfate anion cannot revert the chaotropic effect of the magnesium. A similar observation was made when comparing Li-acetate to LiCl. Furthermore, using 7.0 M NaNO3 as a wash buffer supplement (where the nitrate is more chaotropic than the chloride) resulted in an eluate with a lower HCP content than the platform process, but a higher HCP content compared to the reference run with 2.8 M MgCl2 despite the much higher concentration of 7.0 M NaNO3 applied. This further corroborates the hypothesis that the cation is the more potent parameter in the HCP removal as compared to the anion.

It was concluded that including a chaotropic salt in an affinity chromatography step helps to reduce HCP levels and that the effect is mainly driven by the nature of the cation. the inventors wanted to assess whether this observation can also be applied to other molecules and resins.

In run 25 and 26 OG1950 and applied it to MabSelect Sure (note all other experiments described above were performed with MabSelect Sure LX). OG1950 is a monovalent more classical antibody whereas OG2072 is a bivalent fusion antibody. In run 26, the platform process was applied with a wash step including 2.0 M NaCl, whereas in run 25, the 2.8 M MgCl2-containing wash buffer was applied. The eluate of the run where 2.8 M MgCl2 was used (100 ng/mg) showed a 4-fold lower HCP as compared to the run where 2.0 M NaCl was used in the wash buffer (423 ng/mg). Both runs showed comparable SEC-HPLC purities and potency demonstrating that the MgCl2 did not have an adverse effect on the structure and function of the OG1950 antibody. For comparison, a run with a high pH wash (run with 50 mM Na-Phosphate, 2.0 M NaCl, pH 9.0) was performed and no comparable effect on HCP removal (341 ng/mg) was observed when compared to the 2.8 M MgCl2-containing wash buffer. This shows that the effect of the HCP removal is linked to the nature of the chaotropic salt and cannot be compensated by a high pH. For run 25, some OG1950 eluted in the wash step suggesting that 2.8 M MgCl2 is somewhat too high. In run 28, 2.0 M MgCl2 was applied, and it was shown that the 2.0 M MgCl2 is a suitable concentration for OG1950 and no A280 was seen in the 2.0 M MgCl2 wash step. This suggests that the MgCl2 needs to be optimized on a per molecule basis. The lower MgCl2 did not have a significant impact on the HCP level in the eluate (138 ng/mg). The SEC-HPLC purity is lower for this run as compared to the run with the 2.8 M MgCl2-containing wash buffer. This result was unexpected.

In run 31 and 32, Aflibercept was applied to MabSelect Sure LX resin. Aflibercept is a soluble fusion protein which combines the ligand-binding elements from VEGF receptor fused to the Fc portion of IgG. In run 31, the platform process was applied with a wash step including 2.0 M NaCl, whereas in run 32, the wash buffer was supplemented with 2.8 M MgCl2. The eluate of the run where MgCl2 was used showed an at least three-fold lower HCP as compared to the run where NaCl was used in the wash buffer. Both runs showed comparable SEC-HPLC purities demonstrating that the MgCl2 did not have an adverse effect on the structure of Aflibercept. There was no A280 activity observed in the 2.8 M MgCl2 wash step and 2.8 M MgCl2 was appropriate for this molecule.

In run 33 and 34, OG2127 was applied to MabSelect Sure LX resin. OG2127 is also a soluble fusion protein which combines ligand-binding elements from the VEGF receptor fused to an Fc portion. In run 33, the platform process was applied with a wash step including 2.0 M NaCl, whereas in run 34, the wash buffer was supplemented with 2.8 M MgCl2. The eluate of the run where MgCl2 was used showed an at least five-fold lower HCP as compared to the run where NaCl was used in the wash buffer. Both runs showed comparable SEC-HPLC purities demonstrating that the MgCl2 did not have an adverse effect on the structure of OG2127. There was again no A280 activity observed in the 2.8 M MgCl2 wash step and 2.8 M MgCl2 was appropriate for this molecule.

These experiments showed that the concept works for different antibody types and constructs like monovalent and bivalent antibody constructs and Fc fusion proteins. Also two different type of affinity resins were tested. This demonstrates that concept can be applied generically and not only to OG2072.

### Example 33: Screening of Different Proteins and Resins

Then, tests were run to determine the affinity chromatography process as disclosed herein with two different affinity resins and a variety of antibodies and proteins, as shown in Table 10.

TABLE 10

| | | | | Chromatography | | | | | |
|---|---|---|---|---|---|---|---|---|---|
| | | | | Eluate | | Virus inactivated protein solution | | | |
| | | | | SEC- | | SEC- | | | |
| | | | | HPLC | | HPLC | | | Relative |
| | | | | purity | HCP | purity | HCP | Potency | potency |
| Run | Protein | Resin | Purification strategy | (%) | (ng/mg) | (%) | (ng/mg) | (pM) | (%) |
| 22b | OG2072 | MSS | Platform process: Wash with 50 mM | 98.4 | 1285 | 98.5 | 562 | 330 | 114 |
| | | LX | Na-Phosphate, pH 7.0, 2.0M NaCl | | | | | | |
| 1b | OG2072 | MSS | Wash 3: 100 mM Tris base, 2.8M | 98.4 | 241 | 98.3 | 193 | 304 | 121 |
| | | LX | MgCl2 | | | | | | |
| 26 | OG1950 | MSS | Platform process: Wash with 50 mM | 96.2 | 423 | 96.5 | 281 | 1607 | 96 |
| | | | Na-Phosphate, pH 7.0, 2.0M NaCl | | | | | | |
| 25 | OG1950 | MSS | Wash 3: 100 mM Tris base, 2.8M | 96.3 | 100 | 96.3 | 63 | 1755 | 88 |
| | | | MgCl2 | | | | | | |
| 28 | OG1950 | MSS | Wash 3: 100 mM Tris base, 2.0M | 93.9 | 138 | 93.9 | 94 | 1610 | 96 |
| | | | MgCl2 | | | | | | |
| 31 | Aflibercept | MSS | Platform process: Wash with 50 mM | 98.8 | n/a | 98.7 | >21,538° | n/a | n/a |
| | | LX | Na-Phosphate, pH 7.0, 2.0M NaCl | | | | | | |
| 32 | Aflibercept | MSS | Wash 3: 100 mM Tris base, 2.8M | 98.2 | n/a | 98.1 | 7,611° | n/a | n/a |
| | | LX | MgCl2 | | | | | | |
| 33 | OG2127I | MSS | Platform process: Wash with 50 mM | 100.0 | n/a | 99 | >66,667° | n/a | n/a |
| | | LX | Na-Phosphate, pH 7.0, 2.0M NaCl | | | | | | |
| 34 | OG2127 | MSS | Wash 3: 100 mM Tris base, 2.8M | 98.8 | n/a | 98.8 | 12,482° | n/a | n/a |
| | | LX | MgCl2 | | | | | | |

As is shown in the above Table 10, the methodology disclosed herein was broadly effective across various antibodies and proteins. The improved wash strategy worked for both monovalent and bivalent antibody constructs, as well as for different Fc fusion proteins. Therefore, the wash strategy is envisioned by the inventors to be able to apply generically to any type of antibody and/or protein.

What is claimed is:

1. A method of purifying a product using affinity chromatography, the method comprising: loading an eluent into an affinity chromatography matrix, wherein the affinity chromatography matrix binds to a protein of interest; and washing the affinity chromatography matrix with a buffer solution comprising a chaotropic agent.

2. A method of purifying a product and reducing impurities from a load fluid comprising the protein and one or more impurities by passing the load fluid through an affinity chromatography matrix, followed by at least one wash solution comprising a chaotropic salt, and collecting the protein using an elution solution.

3. A method for separating impurities in an eluate comprising a protein of interest, the method comprising:
loading an eluent comprising a protein of interest onto an affinity chromatography matrix; and washing the affinity chromatography matrix with one or more buffer solutions comprising magnesium or a magnesium salt.

4. A method of producing a product using affinity chromatography, the method comprising:
loading an eluent containing a protein of interest onto an affinity chromatography matrix,
a first wash of the affinity chromatography matrix with a first buffer comprising sodium phosphate and a salt, and
a second wash of the affinity chromatography matrix with a second buffer comprising a chaotropic agent.

5. A method of producing a product using affinity chromatography, the method comprising:
loading an eluent containing a protein of interest onto an affinity chromatography matrix,
a first wash with a first buffer containing Tris and a salt,
a second wash with a second buffer containing Tris and a chaotropic agent,
wherein the second buffer chaotropic agent is not the same salt as contained in the first buffer.

6. A method of producing a product, the method comprising:
(i) collecting a load fluid, wherein the load fluid comprises a protein of interest,
(ii) loading the load fluid onto an affinity chromatography matrix, wherein the affinity chromatography matrix binds to the protein of interest,
(iii) washing the affinity chromatography matrix with a buffer solution comprising a chaotropic salt,
(iv) eluting the bound protein of interest; and
(v) collecting an eluate, wherein the eluate contains the protein of interest.

7. A method of producing a product, the method comprising:
collecting a load fluid, wherein the load fluid comprises a protein of interest, loading the load fluid onto an affinity chromatography matrix,
wherein the affinity chromatography matrix binds to the protein of interest, feeding to the affinity chromatography matrix a buffer solution comprising a chaotropic salt,
eluting the bound protein of interest; and
collecting an eluate, wherein the eluate contains the protein of interest.

8. A method of producing a product, the method comprising:
collecting a conjugate protein, wherein the conjugate protein comprises an antibody bound to a conjugate polymer loading the conjugate protein onto an affinity chromatography matrix, wherein the affinity chromatography matrix binds to the conjugate protein,
washing the affinity chromatography matrix with a buffer solution comprising a chaotropic salt,
eluting the conjugate protein, and
collecting an eluate, wherein the eluate contains the conjugate protein.

9. A method of producing a product, the method comprising:
washing an affinity chromatography matrix bound to a target protein of interest with a buffer comprising a chaotropic salt, eluting and collecting an eluate,
wherein the eluate contains the target protein of interest,
and removing viral contaminants from the eluate.

10. The method of claim 9, wherein removing viral contaminants from the eluate comprises:
one or more of low pH inactivation, detergent inactivation, polishing chromatography steps, viral filtration (VF), ultrafiltration (UF) and/or diafiltration (DF).

11. A method of producing a product, the method comprising:
washing an affinity chromatography matrix bound to a target protein of interest with a buffer comprising a chaotropic salt,
removing the chaotropic salt, and eluting and collecting an eluate, wherein the eluate contains the target protein of interest.

12. The method of 11, wherein the eluate is further combined with an acceptable pharmaceutical excipient to form a pharmaceutical composition.

13. The method of claim 12, wherein a buffer solution is added to the pharmaceutical composition.

14. The method of claim 12, wherein a preservative solution is added to the pharmaceutical composition.

15. The method of claim 12, wherein the pharmaceutical composition is further refined for intravitreal injection.

16. A method of producing a product, the method comprising:
collecting a load fluid, wherein the load fluid is comprised of a protein of interest, loading the load fluid into an affinity chromatography matrix, wherein the affinity chromatography matrix binds to the protein of interest,
washing the affinity chromatography matrix with a buffer solution comprising a chaotropic salt,
eluting and collecting an eluate, wherein eluate contain the target protein of interest, and
removing viral contaminants from the eluate.

17. A method of producing a product, the method comprising:
loading an eluent into an affinity chromatography matrix,
washing with a first wash buffer
washing with a second wash buffer comprising a chaotropic salt,
washing with a third wash buffer, wherein the third wash buffer removes the chaotropic salt
eluting with an elution buffer, wherein an eluate is collected, wherein the eluate comprises a protein product.

18. The method of claim 17, wherein the first wash buffer comprises 50 mM Na-Phosphate.

19. The method of claim 17, wherein the first wash buffer further comprises 250 mM NaCl.

20. The method of claim 17, wherein the first wash buffer comprises Tris and a salt.

21. The method of claim 17, further comprising removing viral contaminants from the eluate.

22. The method of claim 21, wherein removing viral contaminants comprises: one or more of low pH inactivation, detergent inactivation, polishing chromatography steps, viral filtration (VF), ultrafiltration (UF), or diafiltration (DF).

23. The method of claim 17, wherein the eluent comprises a protein of interest.

24. The method of claim 23, wherein the protein of interest is an antibody.

25. The method of claim 24, wherein the antibody is further conjugated to a polymer to form an antibody conjugate.

26. The method of claim 24, wherein the antibody conjugate has the structure of Formula (I): 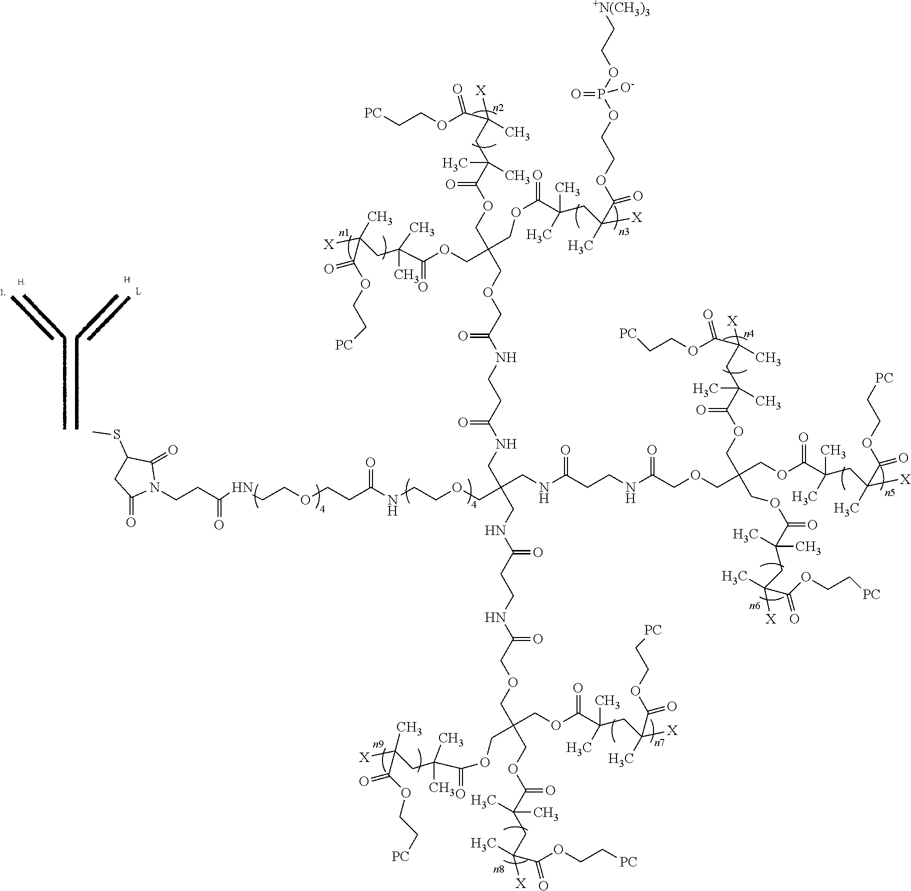
wherein:
each heavy chain of the anti-VEGF-A antibody is denoted by the letter H, and
each light chain of the anti-VEGF-A antibody is denoted by the letter L;
the polymer is bonded to the anti-VEGF-A antibody through the sulfhydryl of C443 (EU numbering), which bond is depicted on one of the heavy chains;
PC is: 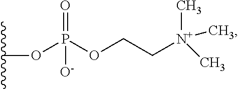 [SMILES: *OP(=O)([O-])OCC[N+](C)(C)C]
wherein the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and
either i) wherein n1, n2, n3, n4, n5, n6, n7, n8 and n9 are the same or different and are integers from 0 to 3000; or ii) wherein n1, n2, n3, n4, n5, n6, n7, n8 and n9 are the same or different such that the sum of n1, n2, n3, n4, n5, n6, n7, n8 and n9 is 2500 plus or minus 15%.

27. The method of claim 25, wherein the antibody conjugate comprises a bispecific antibody.

28. The method of claim 24, wherein the bispecific antibody comprises anti-VEGF and anti IL-6 binding moieties.

29. A method of producing a product, the method comprising:
recovering a cell culture supernatant, wherein the cell culture supernatant comprises a protein of interest,
processing the cell culture supernatant into an eluent, wherein the eluent comprises the protein of interest
loading the eluent into an affinity chromatography matrix,
washing with a first wash buffer comprising Tris or Sodium Phosphate washing with a second wash buffer comprising a chaotropic salt,
eluting with an elution buffer, wherein an eluate is collected, wherein the eluate comprises a protein product,
inactivating viral contaminants present in the eluate with a low pH viral buffer to yield a viral inactivated eluate,
filtering the viral inactivated eluate,
performing at least one round of ion exchange chromatography on the viral inactivated eluate, and
filtering the viral inactivated eluate to yield a retentate, wherein the retentate comprises the protein of interest.

30. The method of claim 29, wherein the cell culture supernatant was produced in a bioreactor using animal component free cell culture.

31. The method of claim 29, wherein processing the cell culture supernatant comprises harvesting cell products from a cell culture.

32. The method of claim 31, wherein the cell culture is clarified to remove cells and cellular debris.

33. The method of claim 29, wherein the eluent comprises the clarified cell culture supernatant

34. A method of purifying a protein using affinity chromatography, the method comprising:
contacting a load fluid with a medium, wherein the medium is an affinity chromatography matrix that binds a protein of interest, washing the medium with a buffer solution comprising a chaotropic agent, wherein the chaotropic agent is a salt, and contacting the washed medium with an elution solution under conditions suitable for eluting the protein of interest.

35. A method of producing a product, the method comprising:
applying the solution containing a protein of interest onto an affinity chromatography matrix,
washing the affinity chromatography matrix with a first buffer
washing the affinity chromatography matrix with a second buffer containing a chaotropic agent,
washing the affinity chromatography matrix with a third buffer to remove the chaotropic agent,
eluting with an elution buffer, wherein an eluate is collected, wherein the eluate comprises a protein product.

36. A system for protein purification, comprising:
a column having a first antigen binding protein bound to the column;
a phosphate wash buffer comprising sodium phosphate and a salt,
an intermediate wash buffer comprising tris,
a second wash buffer comprising magnesium chloride, and
an elution buffer comprising sodium formate,

37. A system for protein purification, comprising:
a column having a first antigen binding protein bound to the column;
a first tris wash buffer comprising tris and a salt,
an intermediate tris wash buffer,
a second wash buffer comprising magnesium chloride, and
an elution buffer comprising sodium formate,

38. The system of claim 36, wherein the column comprises a ligand for affinity chromatography.

39. The system of claim 36, wherein the ligand comprises protein A or Protein G

40. The system of claim 36, wherein the first wash buffer comprising sodium phosphate and a salt has a pH between 5.5 and 9.5.

41. The system of claim 36, wherein the phosphate wash buffer comprising sodium phosphate and a salt comprises about 50 mM sodium phosphate.

42. The system of claim 36, wherein the phosphate wash buffer comprising sodium phosphate and a salt comprises about 250 mM NaCl.

43. The system of claim 36, wherein the first tris wash buffer comprises about 50 mM Tris.

44. The system of claim 43, wherein the first tris wash buffer further comprises about 250 mM NaCl

45. The system of claim 36, wherein the intermediate tris wash buffer comprises about 50 mM Tris.

46. The system of claim 36, wherein the pH of the first tris wash buffer is about 7.2.

47. The system of claim 36, wherein the pH of the second wash buffer is about 7.8.

48. The system of claim 36, wherein the concentration of magnesium chloride in the second wash buffer is about 2.8 M.

49. The system of claim 36, wherein the concentration of sodium formate in the elution buffer comprises 10 mM.

50. A system for antibody purification, comprising:
a column having a protein A resin bound to an antibody,
wherein the antibody comprises a light and heavy chain of at least one of SEQ ID NOs: 91-93, 28-30, and at least one of SEQ ID NOs: 7-13, 19-27, 89, 90, 256-262, respectively, and;
a chaotropic wash buffer comprising a chaotropic salt, and
an elution buffer comprising sodium formate.

51. The method of claim 1, wherein the protein of interest is a bispecific antibody

52. The method of claim 51, wherein the bispecific antibody is specific for VEGF and IL-6.

53. The method of claim 51, wherein the bispecific antibody is OG2072

54. The method of claim 1, wherein the protein of interest is an antibody conjugate.

55. The method of claim 1, wherein the affinity chromatography matrix is a protein A chromatography matrix.

56. The method of claim 1, wherein the chaotropic agent in the buffer solution is comprised of one or more of a lithium, lithium salt, magnesium, magnesium salt, calcium, calcium salt, guanidinium, and/or guanidinium salt.

57. The method of claim 56, wherein the concentration of the one or more of a lithium, lithium salt, magnesium, magnesium salt, calcium, calcium salt, guanidinium, and/or guanidinium salt is between 0.05-3.5 M, respectively.

58. The method of claim 1, wherein the buffer solution further comprises tris.

59. The method of claim 58, wherein the concentration of tris in the buffer solution is at least 5 mM

60. The method of claim 1, wherein the pH of the buffer solution is greater than 5.5.

61. The method of claim 3, wherein the eluate further contains viral impurities.

62. The method of claim 61, further comprising removing the viral impurities.

63. The method of claim 62, further comprising inactivating the viral impurities.

64. The method of claim 3, further comprising the step of washing the affinity chromatography matrix loaded with the load fluid with a prewash buffer solution prior to washing with the buffer solution.

65. The method of 64, further comprising the step of washing the affinity chromatography matrix loaded with the eluent with a postwash buffer solution after washing with buffer solution.

66. The method of 64, where the prewash buffer solution comprises sodium phosphate

67. The method of claim 64, where the prewash buffer solution comprises Tris and a salt.

68. The method of claim 25, wherein the antibody conjugate has the structure of Formula (I), 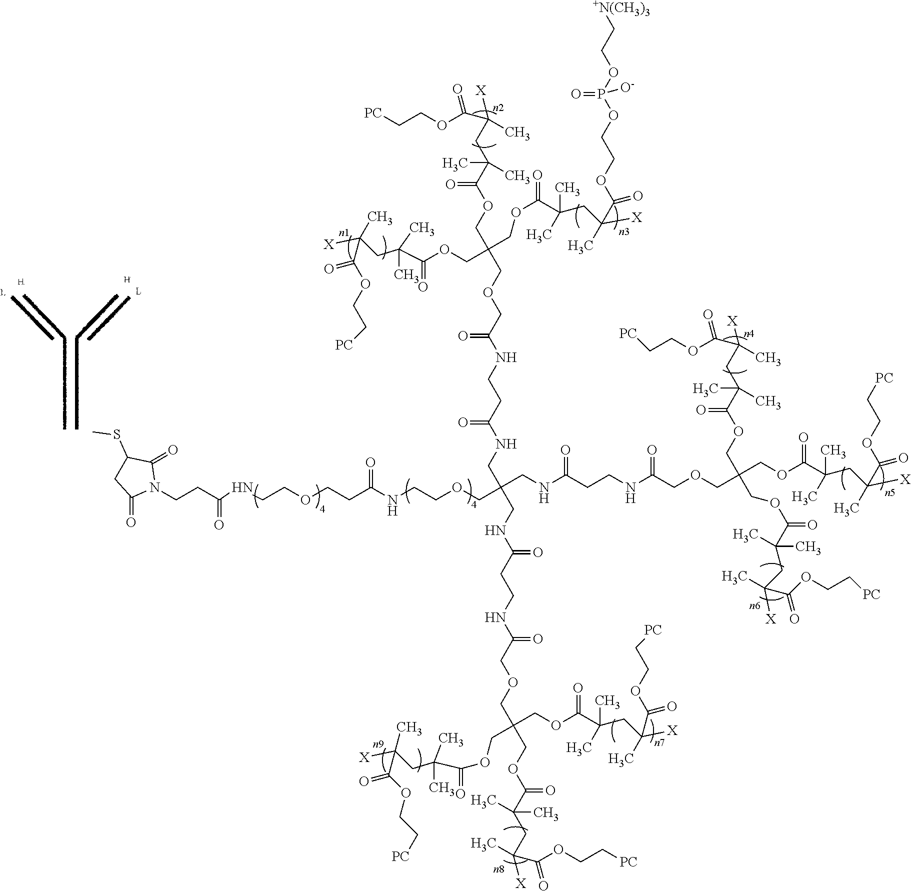
wherein:
each heavy chain of the anti-VEGF-A antibody is denoted by the letter H, and each light chain of the anti-VEGF-A antibody is denoted by the letter L;
the polymer is bonded to the anti-VEGF-A antibody through the sulfhydryl of C443 (EU numbering), which bond is depicted on one of the heavy chains;
PC is: 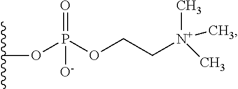 [SMILES: *OP(=O)([O-])OCC[N+](C)(C)C]
wherein the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and
wherein n1, n2, n3, n4, n5, n6, n7, n8 and n9 are the same or different and are integers from 0 to 3000.

69. The method of claim 68, wherein the antibody conjugate comprises an anti-VEGF antibody conjugate comprising an anti-VEGF-A light chain and an anti-VEGF-A heavy chain, wherein the anti-VEGF-A antibody heavy chain comprises CDRH1: that is a CDRH1 in SEQ ID NO: 172, CDRH2: that is a CDRH2 in SEQ ID NO: 173, and CDRH3: that is a CDRH3 in SEQ ID NO: 174, and the anti-VEGF-A antibody light chain comprises CDRL1: that is a CDRL1 in SEQ ID NO: 199, CDRL2: that is a CDRL2 in SEQ ID NO: 200, and CDRL3: that is a CDRL3 in SEQ ID NO: 201.

70. The method of claim 69, wherein the anti-VEGF antibody conjugate comprises: an antibody conjugate comprising an anti-VEGF-A immunoglobulin G (IgG) bonded to a polymer, which polymer comprises MPC monomers, wherein the sequence of the anti-VEGF-A antibody heavy chain is at least one of SEQ ID NOs: 7-13, 19-27, 89, 90, 256-262, and the sequence of the anti-VEGF-A antibody light chain is at least one of SEQ ID NOs: 91-93, 28-30, and wherein the antibody is bonded at C449 to the polymer.

71. The method according to claim 1, wherein the target protein of interest is produced by a cell culture.

72. The method according to claim 71, wherein the cell culture comprises CHO cells.

73. The method of claim 3, further comprising the step of washing the affinity chromatography matrix loaded with the eluent with a postwash buffer solution after washing with buffer solution.

74. The method of claim 3, wherein washing the affinity chromatography matrix with the buffer solution removes nucleic acids, endotoxins, antifoam agents, or other small molecules other than the target protein of interest.

75. The method of claim 3, wherein washing the affinity chromatography matrix with the buffer solution removes impurities while keeping the target protein of interest bound to the affinity chromatography matrix.

76. The method of claim 3, wherein washing the affinity chromatography matrix with the buffer solution removes host cell proteins besides the target protein of interest.

77. The method of claim 4, wherein the addition of chaotropic agent in the buffer solution does not elute the target protein of interest.

78. The method of claim 3, further comprising one or more of virus inactivation, tangential flow filtration, diafiltration, ultrafiltration, ion exchange chromatography, or virus reduction filtration.

79. The method of claim 3, wherein the eluent was produced in a bioreactor using animal component free cell culture.

80. The method of claim 3, wherein the product is a protein of interest.

81. The method of claim 3, wherein impurities comprise host cell protein impurities.

82. The method of claim 17, wherein the first wash buffer comprises 10 mM Na-Phosphate.

83. The method of claim 17, wherein the first wash buffer comprises a phosphate-based species.

84. The method of claim 17, wherein the first wash buffer further comprises 50 mM NaCl.

85. A method for processing a product, comprising:
loading an eluent into an affinity chromatography matrix; and
washing with a wash buffer comprising a chaotropic salt to collect an eluate, wherein the concentration of the chaotropic salt is increased from a first concentration to a second concentration, wherein the eluate is collected in at least one fraction, wherein the at least one fraction comprises a product of interest.

86. The method of claim 85, wherein the concentration of the chaotropic salt at the first concentration is 0 M, wherein the concentration of the chaotropic salt at the second concentration is 4.0 M.

87. The method of claim 85, wherein the chaotropic salt is one or more of a lithium, lithium salt, magnesium, magnesium salt, calcium, calcium salt, guanidinium, and/or guanidinium salt.

88. The method of claim 87, wherein the chaotropic salt is selected from magnesium chloride, calcium chloride, lithium chloride, and guanidinium hydrochloride.

89. The method of claim 1, wherein the buffer solution further comprises one or more of the following: Acetate, Citrate, ACES, BES, Bicine, HEPES, MES, MOPS, MOPSO, TAPS, Tricine, Bis-Tris, Bis-Tris propane, Cacodylate, CAPS, CAPSO, CHES, Glycine, Glycylglycine, Imidazole, PIPES, TEA, or TES.
